(19) United States
(12) Patent Application Publication
(10) Pub. No.: US 2026/0115199 A1
(43) Pub. Date: Apr. 30, 2026

(54) TRICYCLIC COMPOUNDS FOR THE TREATMENT OF CANCER

(71) Applicant: Hoffmann-La Roche Inc., Little Falls, NJ (US)

(72) Inventors: Jianguo CHEN, Shanghai (CN); Lei GUO, Shanghai (CN); Hong SHEN, Shanghai (CN); Xiangyu YAO, Shanghai (CN); Weixing ZHANG, Shanghai (CN); Dan ZHAO, Shanghai (CN)

(73) Assignee: Hoffmann-La Roche Inc., Little Falls, NJ (US)

(21) Appl. No.: 19/373,372

(22) Filed: Oct. 29, 2025

(30) Foreign Application Priority Data
Oct. 31, 2024 (WO) PCT/CN2024/128983
Nov. 8, 2024 (WO) PCT/CN2024/130934
Apr. 22, 2025 (WO) PCT/CN2025/090361
Aug. 4, 2025 (WO) PCT/CN2025/112475

(51) Int. Cl. A61K 31/5383; A61K 31/504; A61K 31/5377; A61P 35/00; C07D 513/22; C07D 519/00

(52) U.S. Cl. CPC A61K 31/5383; A61K 31/504; A61K 31/5377; A61P 35/00; C07D 513/22; C07D 519/00

(57) ABSTRACT

The present invention relates to compounds of formula (I),

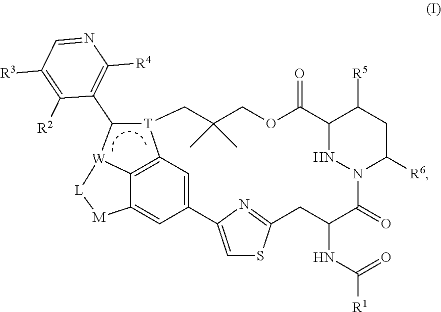

wherein R¹ to R⁶, M, L, T and W are as described herein, and their pharmaceutically acceptable salt, enantiomers and diastereomers thereof, and compositions including the compounds and methods of using the compounds.

### CROSS-REFERENCE TO RELATED APPLICATIONS

This application claims the benefit of foreign priority to Chinese Application No. PCT/CN2024/128983, filed Oct. 31, 2024, Chinese Application No. PCT/CN2024/130934, filed Nov. 8, 2024, Chinese Application No. PCT/CN2025/090361, filed Apr. 22, 2025, and Chinese Application No. PCT/CN2025/112475, filed Aug. 4, 2025, the disclosure of each of which is incorporated by reference herein in its entirety.

The present invention relates to organic compounds useful for therapy and/or prophylaxis in a mammal, and in particular to inhibition of KRAS G12V mutant useful for treating cancers.

### FIELD OF THE INVENTION

RAS is one of the most well-known proto-oncogenes. Approximately 30% of human cancers contain mutations in three most notable members, KRAS, HRAS, and NRAS, making them the most prevalent oncogenic drivers. KRAS mutations are generally associated with poor prognosis, especially in colorectal cancer, pancreatic cancer, lung cancers. As the most frequently mutated RAS isoform, KRAS has been intensively studied in the past years leading to the discovery of commonly occurring KRAS alleles, including G12D, G12V, G12C, G13D, G12R, G12A, G12S, Q61H, etc.

KRAS G12V, in particular, represents a significant unmet medical need with a high prevalence across multiple cancer types. Globally, there are approximately 160,000 colorectal cancer (CRC) patients (10.3%), 130,000 pancreatic ductal adenocarcinoma (PDAC) patients (28.3%), and 120,000 non-small cell lung cancer (NSCLC) patients (5.4%) harboring the G12V mutation (AACR GENIE v15.0 and WHO Cancer Today). Despite the high prevalence, effective treatments for RAS-driven metastatic CRC (mCRC) and PDAC are lacking, underscoring the urgent need for novel therapeutic strategies. RAS-targeting therapies have the potential to revolutionize first-line treatment for RAS-mutated tumors, offering new hope for patients with these challenging cancers. The KRAS G12V mutation is a clinically validated foundational target, with strong human genetic evidence linking it to cancer. Significant correlations between genetic and pharmacological perturbations and disease risk further emphasize its importance. Clinical support for targeting KRAS G12V is bolstered by positive Phase 1 data in PDAC from panRAS inhibitor trials and the approval of G12C-targeting drugs, which indicate a clinically meaningful impact.

The necessity for selectivity for improved combinability is highlighted by the inadequate response in CRC, which necessitates combination therapy due to mutant KRAS requiring APC loss and strong EGFR-mediated feedback. PanRAS inhibitors share common overlapping toxicities with Cetuximab (GI toxicity, rash) and Bevacizumab (GI toxicity), suggesting that enhanced selectivity may improve combinability and therapeutic outcomes.

The intrinsic G12V target biology underscores the promising therapeutic benefits of selective inhibitors. The high GTP binding affinity and slower hydrolysis rates of G12V (compared to G12C and G12D) prolong RAS activation, necessitating the targeting of the active G12V state. Additionally, the high transforming and migration potentials, along with a worse prognosis in PDAC, underscore G12V addiction and the significant clinical impact of its inhibition.

The promising efficacy of combined immune checkpoint inhibitors (ICIs) in non-small cell lung cancer (NSCLC) and the improved progression-free survival (PFS) and overall survival (OS) observed with the use of metastatic surgery and Bevacizumab in metastatic colorectal cancer (mCRC) suggest significant advantages of these combination therapies. These findings underscore the potential of selective KRAS G12V inhibitors to address critical unmet needs and enhance patient outcomes in RAS-driven cancers.

As such, compounds of this invention were invented to target and inhibit KRAS G12V selectively for the treatment of KRAS G12V mutant driven cancers.

### SUMMARY OF THE INVENTION

The present invention relates to novel compounds of formula (I),

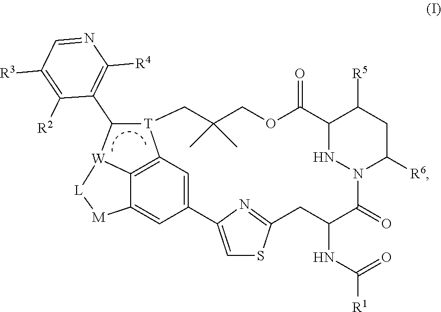

wherein
R¹ is 3-oxabicyclo[4.1.0]heptanyl,
   3-oxabicyclo[3.1.0]hexanyl,
   norcaranyl unsubstituted or substituted by one or two substituents independently selected from a group consisting of halogen and C₁₋₆alkyl, or
   C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is H, halogen, C₁₋₆alkyl, haloC₁₋₆alkyl or C₃₋₇cycloalkyl;
R³ is C₂₋₆alkynyl or 4-12 membered monocyclic or bicyclic heterocyclyl, wherein said C₂₋₆alkynyl and said heterocyclyl are unsubstituted or substituted by one or two substituents independently selected from a group consisting of halogen, hydroxy, C₁₋₆alkyl, haloC₁₋₆alkyl, C₁₋₆alkoxy, C₃₋₇cycloalkyl, morpholino and piperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

The invention also relates to their manufacture in medicaments based on a compound in accordance with the invention and their production as well as the use of compounds of formula (I), (Ia), (Ia-1) or (Ia-2) thereof as inhibitor of KRAS.

The compounds of formula (I), (Ia), (Ia-1) or (Ia-2) showed excellent KRAS G12V inhibition, significantly improved selectivity, SDPK and safety profiles in human cells or organoids over reference compounds.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₁₋₆alkylene” denotes a linear saturated divalent hydrocarbon group of 1 to 6 carbon atoms or a divalent branched saturated hydrocarbon group of 3 to 6 carbon atoms. Examples of C₁₋₆alkylene groups include methylene, ethylene, propylene, 2-methylpropylene, butylene, 2-ethylbutylene, pentylene, hexylene.

The term “C₂₋₆alkynyl” denotes a monovalent linear or branched hydrocarbon group of 2 to 6 carbon atoms with at least one triple bond. In particular embodiments, alkynyl has 2 to 4 carbon atoms with at least one triple bond. Examples of C₂₋₆alkynyl include ethynyl (—C≡CH), prop-1-ynyl (—C≡CCH₃), prop-2-ynyl (propargyl, —CH₂C≡CH), but-1-ynyl, but-2-ynyl, and but-3-ynyl.

The term “halogen” and “halo” are used interchangeably herein and denote fluoro, chloro, bromo, or iodo.

The term “halopyridyl” denotes a pyridyl group wherein at least one of the hydrogen atoms of the pyridyl group has been replaced by same or different halogen atoms.

The term “haloC₁₋₆alkyl” refers to a C₁₋₆alkyl group wherein at least one of the hydrogen atoms of the C₁₋₆alkyl group has been replaced by same or different halogen atoms, particularly fluoro atoms. Examples of haloC₁₋₆alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, or trifluoromethyl.

The term “C₃₋₇cycloalkyl” denotes a monovalent saturated monocyclic or bicyclic hydrocarbon group of 3 to 7 ring carbon atoms. Bicyclic means consisting of two saturated carbocycles having one or more carbon atoms in common. Examples of monocyclic cycloalkyl are cyclopropyl, cyclobutyl, cyclopentyl, cyclohexyl or cycloheptyl. Examples for bicyclic cycloalkyl are bicyclo[1.1.0]butyl, bicyclo[2.2.1]heptanyl, bicyclo[1.1.1]pentanyl, or bicyclo[2.2.2]octanyl.

The term “heterocycle” or “heterocyclyl” refers to a 3, 4, 5, 6, 7, 8, 9, 10-membered monocyclic, 7, 8, 9 and 10-membered bicyclic (including fused, bridged and spiro bicyclic), or 10, 11, 12, 13, 14 and 15-membered bicyclic (including fused, bridged and spiro bicyclic) or tricyclic heterocyclic moiety that is saturated or partially unsaturated, and has one or more (e.g., 1, 2, 3 or 4) heteroatoms selected from oxygen, nitrogen and sulfur in the ring with the remaining ring atoms being carbon. In some aspects, the heterocycle is a heterocycloalkyl. In particular aspects heterocycle or heterocyclyl refers to a 4, 5, 6 or 7-membered Heterocycle. When used in reference to the ring atom of a heterocycle, a nitrogen or sulfur may also be in an oxidized form, and a nitrogen may be substituted with one or more C₁₋₆alkyl or groups. The heterocycle can be attached to its pendant group at any heteroatom or carbon atom that results in a stable structure. Any of the heterocycle ring atoms can be optionally substituted with one or more substituents described herein. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Examples of such saturated or partially unsaturated heterocycles include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, pyrrolidine 1-oxide, N-hydroxypiperidine, 1-methylpyrrolidine N-oxide, diazirinyl and quinuclidinyl. The term heterocycle also includes groups in which a heterocycle is fused to one or more aryl, heteroaryl, or cycloalkyl rings, such as indolinyl, 3H-indolyl, chromanyl, azabicyclo[2.2.1]heptanyl, azabicyclo[3.1.0]hexanyl, azabicyclo[3.1.1]heptanyl, octahydroindolyl, or tetrahydroquinolinyl. Particular heterocyclyl includes 2-oxooxazolyl, 2-oxo-imidazolyl, 7-oxo-pyrrolo[2,3-c]pyridinyl, 6-oxo-4H-pyrrolo[3,4-b]pyrrolyl, 3,4,6,7,9,9a-hexahydro-1H-pyrazino[2,1-c][1,4]oxazinyl and 5-oxo-6H-pyrrolo[3,2-b]pyrrolyl.

The term “cis” and “trans” denote the relative stereochemistry of the molecule or moiety. For example: intermediate compound O4-b,

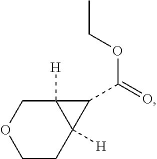

as the trans-isomer, refers to a mixture of

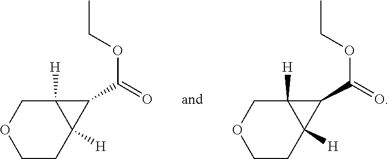

The way of showing relative stereochemistry also applies to the final compounds.

The skilled of the art would understand that the following structures of compounds of formula (Ia) and (Ia′) are equal especially for the chiral centers:

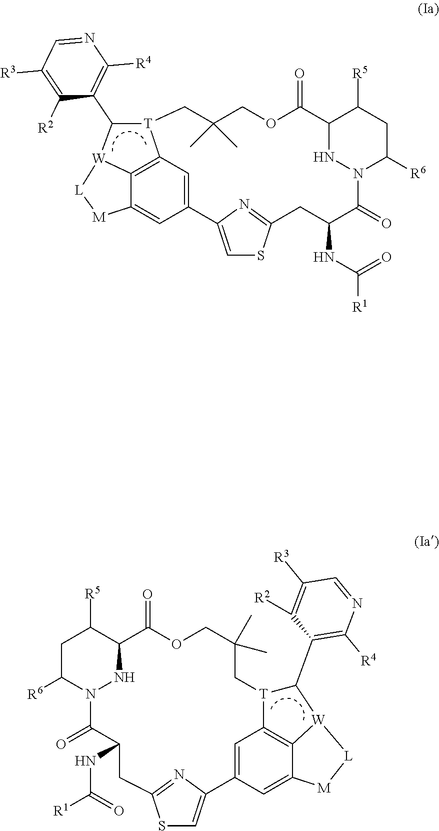

The skilled of the art would understand that the structure of

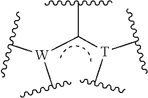

represents the alternative bonds of single bond and double bond as or

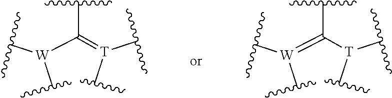

whenever applicable depending on the definition of W and T.

The term “deuterio” or “deuterium” refers to the isotope of hydrogen that has a single neutron as well as a proton in the nucleus.

Examples of isotopes suitable for inclusion in the compounds of formula (I) may include isotopes of hydrogen, such as ²H (D, deuterium) and 3H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

In one embodiment, at least one of the hydrogens of compound of formula (I) can be replaced with deuterium. A person with ordinary skill may make additional deuterated analogs of the compounds of this invention with different combinations of deuterium replacing hydrogen. Such additional deuterated analogs may provide similar or better therapeutic advantages, similar or better physical properties, or similar or better pharmacokinetic properties.

In one embodiment, atoms specified in the compounds described herein may optionally be replaced by other atoms from the same group of the Periodic Table of Elements, wherein such replacements may or may not improve the compounds' properties or functionality, such as physical, toxicity, or pharmacokinetic properties. For instance, carbon (C) may be replaced with silicon (Si), sulfur (S) may be replaced with selenium (Se) or tellurium (Te), while nitrogen (N) may be replaced with phosphorus (P) or arsenic (As). Such modifications are well within the skill of a person of ordinary skill in the art and are intended to fall within the scope of the present invention.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Inhibitor OF KRAS

The present invention relates to (i″) a compound of formula (I),

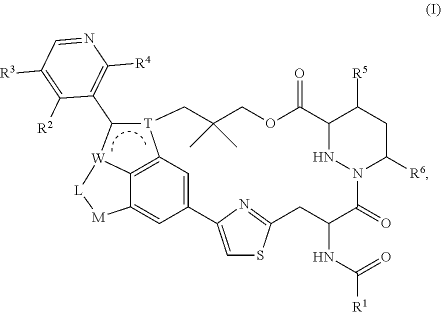

wherein
R¹ is 3-oxabicyclo[4.1.0]heptanyl,
   3-oxabicyclo[3.1.0]hexanyl,
   norcaranyl unsubstituted or substituted by one or two substituents independently selected from a group consisting of halogen and C₁₋₆alkyl, or
   C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is H, halogen, C₁₋₆alkyl, haloC₁₋₆alkyl or C₃₋₇cycloalkyl;
R³ is C₂₋₆alkynyl or 4-12 membered monocyclic or bicyclic heterocyclyl, wherein said C₂₋₆alkynyl and said heterocyclyl are unsubstituted or substituted by one or two substituents independently selected from a group consisting of halogen, hydroxy, C₁₋₆alkyl, haloC₁₋₆alkyl, C₁₋₆alkoxy, C₃₋₇cycloalkyl, morpholino and piperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

A further embodiment of present invention is (ii″) a compound of formula (I) according to (i″), or a pharmaceutically acceptable salt, enantiomers and diastereomers thereof, wherein R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl.

Another embodiment of present invention is (iii″) a compound of formula (Ia),

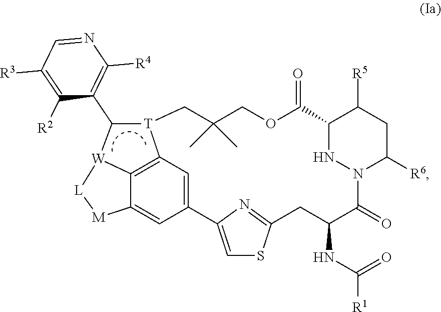

wherein
R¹ is 3-oxabicyclo[4.1.0]heptanyl,
   3-oxabicyclo[3.1.0]hexanyl,
   norcaranyl unsubstituted or substituted by one or two substituents independently selected from a group consisting of halogen and C₁₋₆alkyl, or
   C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is H, halogen, C₁₋₆alkyl, haloC₁₋₆alkyl or C₃₋₇cycloalkyl;
R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

In one embodiment, R¹ is 2-(2-fluoro-3-pyridyl)cyclopropyl, 2-(2-methyl-3-pyridyl)cyclopropyl, 2-(3-pyridyl)cyclopropyl, 2-(difluoromethyl)cyclopropyl, 2,2-difluoronorcaran-7-yl, 2,3-dimethylcyclopropyl, 2-methylcyclopropyl, 2-tetrahydropyran-4-ylcyclopropyl, 2-tetrahydropyran-4-yl-cyclopropyl, 3-methylnorcaran-7-yl, 3-oxabicyclo[3.1.0]hexan-6-yl, 3-oxabicyclo[4.1.0]heptan-7-yl or norcaran-7-yl.

In one embodiment, R² is H, fluoro, chloro, methyl, ethyl, difluoromethyl, trifluoromethyl or cyclopropyl. In another embodiment, R² is fluoro, chloro, methyl, ethyl, difluoromethyl, trifluoromethyl or cyclopropyl.

In one embodiment, R³ is 4-cyclopropylpiperazin-1-yl, 3,4,6,7,9,9a-hexahydro-1H-pyrazino[2,1-c][1,4]oxazin-8-yl, 3-morpholinoprop-1-ynyl or 4-methylpiperazin-1-yl.

A further embodiment of present invention is (iv″) a compound of formula (I) or (Ia), according to any one of (i″) to (iii″), or a pharmaceutically acceptable salt, enantiomers and diastereomers thereof, wherein R² is halogen or C₁₋₆alkyl.

A further embodiment of present invention is (v″) a compound of formula (I) or (Ia) according to any one of (i″) to (iv″), or a pharmaceutically acceptable salt, enantiomers and diastereomers thereof, wherein R² is fluoro or methyl.

A further embodiment of present invention is (vi″) a compound of formula (I) or (Ia), or a pharmaceutically acceptable salt, enantiomers and diastereomers thereof, according to any one of (i″) to (v″), wherein R¹ is C₃₋₇cycloalkyl substituted by one or two substituents independently selected from C₁₋₆alkyl and halopyridyl.

A further embodiment of present invention is (vii″) a compound of formula (I) or (Ia), according to any one of (i″) to (vi″), or a pharmaceutically acceptable salt, enantiomers and diastereomers thereof, wherein R¹ is 2-(2-fluoro-3-pyridyl)cyclopropyl or 2,3-dimethylcyclopropyl.

A further embodiment of present invention is (viii″) a compound of formula (I) or (Ia), according to any one of (i″) to (vii″), or a pharmaceutically acceptable salt, enantiomers and diastereomers thereof, wherein R³ is morpholinoC₂₋₆alkynyl or C₁₋₆alkylpiperazinyl.

A further embodiment of present invention is (ix″) a compound of formula (I) or (Ia), according to any one of (i″) to (viii″), or a pharmaceutically acceptable salt, enantiomers and diastereomers thereof, wherein R³ is 3-morpholinoprop-1-ynyl or 4-methylpiperazin-1-yl.

A further embodiment of present invention is (x″) a compound of formula (I) or (Ia), according to any one of (i″) to (ix″), or a pharmaceutically acceptable salt, enantiomers and diastereomers thereof, wherein R⁴ is 1-methoxyethyl.

A further embodiment of present invention is (xi″) a compound of formula (I) or (Ia), according to any one of (i″) to (x″), or a pharmaceutically acceptable salt, enantiomers and diastereomers thereof, wherein R⁵ is H and R⁶ is H; or R⁵ and R⁶ together with the atoms to which they attached to form a cyclobutyl ring.

A further embodiment of present invention is (xii″) a compound of formula (I) or (Ia), according to any one of (i″) to (xi″), or a pharmaceutically acceptable salt, enantiomers and diastereomers thereof, wherein M is C₁₋₆alkylene.

A further embodiment of present invention is (xiii″) a compound of formula (I) or (Ia), according to any one of (i″) to (xii″), or a pharmaceutically acceptable salt, enantiomers and diastereomers thereof, wherein M is methylene.

A further embodiment of present invention is (xiv″) a compound of formula (I) or (Ia), according to any one of (i″) to (xiii″), or a pharmaceutically acceptable salt, enantiomers and diastereomers thereof, wherein L is ethylene.

A further embodiment of present invention is (xv″) a compound of formula (I) or (Ia), according to any one of (i″) to (iii″), wherein
R¹ is C₃₋₇cycloalkyl substituted by one or two substituents independently selected from C₁₋₆alkyl and halopyridyl;
R² is halogen or C₁₋₆alkyl;
R³ is morpholinoC₂₋₆alkynyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
W is N and T is C; or W is C and T is N;
or a pharmaceutically acceptable salt thereof.

A further embodiment of present invention is (xvi″) a compound of formula (I) or (Ia), according to (xv″), wherein
R¹ is 2-(2-fluoro-3-pyridyl)cyclopropyl or 2,3-dimethylcyclopropyl;
R² is fluoro or methyl;
R³ is 3-morpholinoprop-1-ynyl or 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
W is N and T is C; or W is C and T is N;
or a pharmaceutically acceptable salt thereof.

Another embodiment of present invention is (xvii″) a compound of formula (Ia-1),

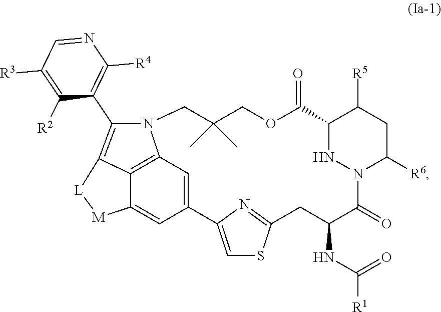

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is morpholinoC₂₋₆alkynyl, C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
or a pharmaceutically acceptable salt thereof.

A further embodiment of present invention is (xviii″) a compound of formula (Ia-1), according to (xvii″), or a pharmaceutically acceptable salt, enantiomers and diastereomers thereof, wherein R¹ is C₃₋₇cycloalkyl substituted by halopyridyl or di-substituted by C₁₋₆alkyl.

A further embodiment of present invention is (xix″) a compound of formula (Ia-1), according to (xvii″) or (xviii″), or a pharmaceutically acceptable salt, enantiomers and diastereomers thereof, wherein R² is halogen or C₁₋₆alkyl.

A further embodiment of present invention is (xx″) a compound of formula (Ia-1), according to any one of (xvii″) to (xix″), or a pharmaceutically acceptable salt, enantiomers and diastereomers thereof, wherein R³ is morpholinoC₂₋₆alkynyl or C₁₋₆alkylpiperazinyl.

A further embodiment of present invention is (xxi″) a compound of formula (Ia-1), according to any one of (xvii″) to (xx″), or a pharmaceutically acceptable salt, enantiomers and diastereomers thereof, wherein M is C₁₋₆alkylene.

A further embodiment of present invention is (xxii″) a compound of formula (Ia-1), according to any one of (xvii″) to (xxi″), wherein
R¹ is C₃₋₇cycloalkyl substituted by halopyridyl or di-substituted by C₁₋₆alkyl;
R² is halogen or C₁₋₆alkyl;
R³ is morpholinoC₂₋₆alkynyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
or a pharmaceutically acceptable salt thereof.

A further embodiment of present invention is (xxiii″) a compound of formula (Ia-1), according to any one of (xvii″) to (xxii″), wherein
R¹ is 2-(2-fluoro-3-pyridyl)cyclopropyl or 2,3-dimethylcyclopropyl;
R² is fluoro or methyl;
R³ is 3-morpholinoprop-1-ynyl or 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
or a pharmaceutically acceptable salt thereof.

Another embodiment of present invention is (xxiv″) a compound of formula (Ia-2),

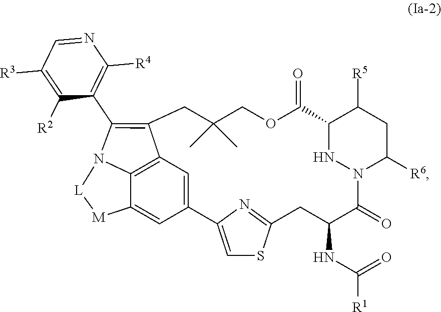

wherein
R¹ is 3-oxabicyclo[3.1.0]heptanyl, norcaranyl substituted by one or two substituents independently selected from a group consisting of halogen and C₁₋₆alkyl, or
   C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is halogen, C₁₋₆alkyl, haloC₁₋₆alkyl or C₃₋₇cycloalkyl;
R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
or a pharmaceutically acceptable salt thereof.

A further embodiment of present invention is (xxv″) a compound of formula (Ia-2), according to (xxiv″), wherein
R¹ is C₃₋₇cycloalkyl di-substituted by C₁₋₆alkyl;
R² is C₁₋₆alkyl;
R³ is C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
or a pharmaceutically acceptable salt thereof.

A further embodiment of present invention is (xxvi″) a compound of formula (Ia-2), according to (xxiv″) or (xxv″), wherein
R¹ is 2,3-dimethylcyclopropyl;
R² is methyl;
R³ is 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
or a pharmaceutically acceptable salt thereof.

The present invention relates to (i′) a compound of formula (I),

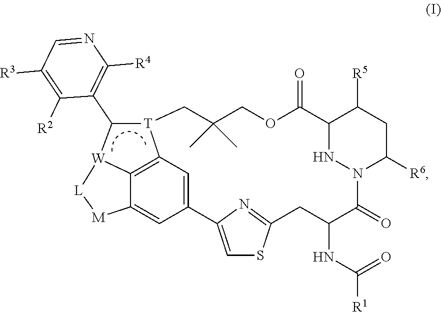

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is morpholinoC₂₋₆alkynyl, C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

Another embodiment of present invention is (ii′) a compound of formula (Ia),

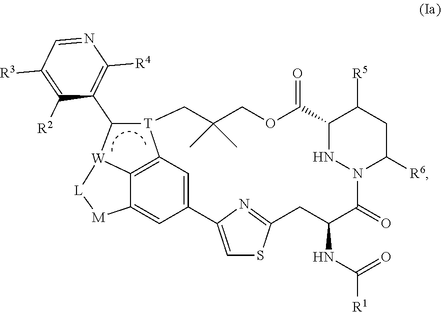

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is morpholinoC₂₋₆alkynyl, C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

A further embodiment of present invention is (iii′) a compound of formula (I) or (Ia) according to (i′) or (ii′), or a pharmaceutically acceptable salt, enantiomers and diastereomers thereof, wherein R¹ is C₃₋₇cycloalkyl substituted by one or two substituents independently selected from C₁₋₆alkyl and halopyridyl.

A further embodiment of present invention is (iv′) a compound of formula (I) or (Ia), according to any one of (i′) to (iii′), or a pharmaceutically acceptable salt, enantiomers and diastereomers thereof, wherein R¹ is 2-(2-fluoro-3-pyridyl)cyclopropyl or 2,3-dimethylcyclopropyl.

A further embodiment of present invention is (v′) a compound of formula (I) or (Ia) according to any one of (i′) to (iv′), or a pharmaceutically acceptable salt, enantiomers and diastereomers thereof, wherein R² is halogen or C₁₋₆alkyl.

A further embodiment of present invention is (vi′) a compound of formula (I) or (Ia), or a pharmaceutically acceptable salt, enantiomers and diastereomers thereof, according to any one of (i′) to (v′), wherein R² is fluoro or methyl.

A further embodiment of present invention is (vii′) a compound of formula (I) or (Ia), according to any one of (i′) to (vi′), or a pharmaceutically acceptable salt, enantiomers and diastereomers thereof, wherein R³ is morpholinoC₂₋₆alkynyl or C₁₋₆alkylpiperazinyl.

A further embodiment of present invention is (viii′) a compound of formula (I) or (Ia), according to any one of (i′) to (vii′), or a pharmaceutically acceptable salt, enantiomers and diastereomers thereof, wherein R³ is 3-morpholinoprop-1-ynyl or 4-methylpiperazin-1-yl.

A further embodiment of present invention is (ix′) a compound of formula (I) or (Ia), according to any one of (i′) to (viii′), or a pharmaceutically acceptable salt, enantiomers and diastereomers thereof, wherein R⁴ is 1-methoxyethyl.

A further embodiment of present invention is (x′) a compound of formula (I) or (Ia), according to any one of (i′) to (ix′), or a pharmaceutically acceptable salt, enantiomers and diastereomers thereof, wherein R⁵ is H and R⁶ is H; or R⁵ and R⁶ together with the atoms to which they attached to form a cyclobutyl ring.

A further embodiment of present invention is (xi′) a compound of formula (I) or (Ia), according to any one of (i′) to (x′), or a pharmaceutically acceptable salt, enantiomers and diastereomers thereof, wherein M is C₁₋₆alkylene.

A further embodiment of present invention is (xii′) a compound of formula (I) or (Ia), according to any one of (i′) to (xi′), or a pharmaceutically acceptable salt, enantiomers and diastereomers thereof, wherein M is methylene.

A further embodiment of present invention is (xiii′) a compound of formula (I) or (Ia), according to any one of (i′) to (xii′), or a pharmaceutically acceptable salt, enantiomers and diastereomers thereof, wherein L is ethylene.

A further embodiment of present invention is (xiv′) a compound of formula (I) or (Ia), according to (i′) or (ii′), wherein
R¹ is C₃₋₇cycloalkyl substituted by one or two substituents independently selected from C₁₋₆alkyl and halopyridyl;
R² is halogen or C₁₋₆alkyl;
R³ is morpholinoC₂₋₆alkynyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
W is N and T is C; or W is C and T is N;
or a pharmaceutically acceptable salt thereof.

A further embodiment of present invention is (xv′) a compound of formula (I) or (Ia), according to (xiv′), wherein
R¹ is 2-(2-fluoro-3-pyridyl)cyclopropyl or 2,3-dimethylcyclopropyl;
R² is fluoro or methyl;
R³ is 3-morpholinoprop-1-ynyl or 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
W is N and T is C; or W is C and T is N;
or a pharmaceutically acceptable salt thereof.

Another embodiment of present invention is (xvi′) a compound of formula (Ia-1),

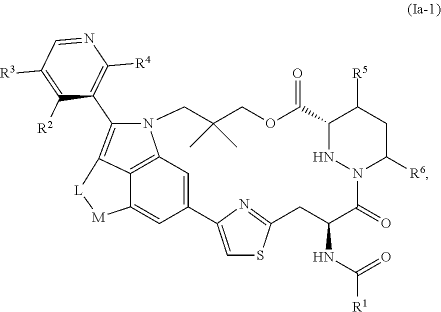

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is morpholinoC₂₋₆alkynyl, C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
or a pharmaceutically acceptable salt thereof.

A further embodiment of present invention is (xvii′) a compound of formula (Ia-1), according to (xvi′), wherein
R¹ is C₃₋₇cycloalkyl substituted by one or two substituents independently selected from C₁₋₆alkyl and halopyridyl;
R² is halogen or C₁₋₆alkyl;
R³ is morpholinoC₂₋₆alkynyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
or a pharmaceutically acceptable salt thereof.

A further embodiment of present invention is (xviii′) a compound of formula (Ia-1), according to (xvi′) or (xvii′), wherein
R¹ is 2-(2-fluoro-3-pyridyl)cyclopropyl or 2,3-dimethylcyclopropyl;
R² is fluoro or methyl;
R³ is 3-morpholinoprop-1-ynyl or 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
or a pharmaceutically acceptable salt thereof.

A further embodiment of present invention is (xvii′) a compound of formula (Ia-1), according to (xvi′), wherein
R¹ is C₃₋₇cycloalkyl substituted by halopyridyl;
R² is H;
R³ is C₃₋₇cycloalkylpiperazinyl;
R⁴ is C₁₋₆alkoxyC₁₋₆alkyl;
R⁵ and R⁶ together with the atoms to which they attached to form a C₃₋₇cycloalkyl ring;
M is O;
L is C₁₋₆alkylene;
or a pharmaceutically acceptable salt thereof.

A further embodiment of present invention is (xviii′) a compound of formula (Ia-1), according to (xvi′) or (xvii′), wherein
R¹ is 2-(2-fluoro-3-pyridyl)cyclopropyl;
R² is H;
R³ is 4-cyclopropylpiperazin-1-yl;
R⁴ is 1-methoxyethyl;
R⁵ and R⁶ together with the atoms to which they attached to form a cyclobutyl ring;
M is O;
L is ethylene;
or a pharmaceutically acceptable salt thereof.

The present invention relates to (i) a compound of formula (I),

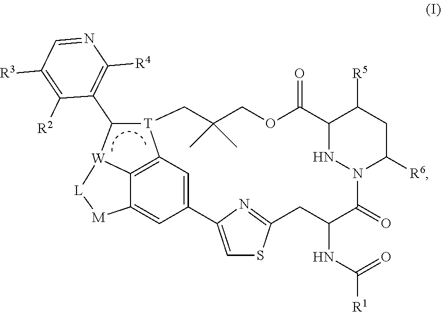

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

Another embodiment of present invention is (ii) a compound of formula (Ia),

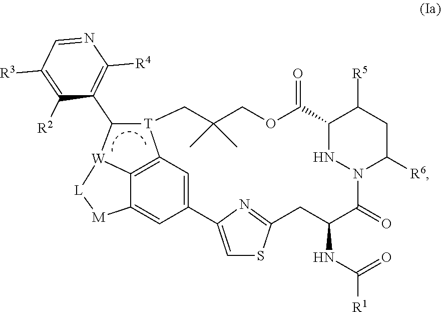

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.

A further embodiment of present invention is (iii) a compound of formula (I) or (Ia) according to (i) or (ii), or a pharmaceutically acceptable salt, enantiomers and diastereomers thereof, wherein R¹ is C₃₋₇cycloalkyl substituted by one or two substituents independently selected from C₁₋₆alkyl and halopyridyl.

A further embodiment of present invention is (iv) a compound of formula (I) or (Ia), according to any one of (i) to (iii), or a pharmaceutically acceptable salt, enantiomers and diastereomers thereof, wherein R¹ is 2-(2-fluoro-3-pyridyl)cyclopropyl or 2,3-dimethylcyclopropyl.

A further embodiment of present invention is (v) a compound of formula (I) or (Ia) according to any one of (i) to (iv), or a pharmaceutically acceptable salt, enantiomers and diastereomers thereof, wherein R² is halogen or C₁₋₆alkyl.

A further embodiment of present invention is (vi) a compound of formula (I) or (Ia), or a pharmaceutically acceptable salt, enantiomers and diastereomers thereof, according to any one of (i) to (v), wherein R² is fluoro or methyl.

A further embodiment of present invention is (vii) a compound of formula (I) or (Ia), according to any one of (i) to (vi), or a pharmaceutically acceptable salt, enantiomers and diastereomers thereof, wherein R³ is C₁₋₆alkylpiperazinyl.

A further embodiment of present invention is (viii) a compound of formula (I) or (Ia), according to any one of (i) to (vii), or a pharmaceutically acceptable salt, enantiomers and diastereomers thereof, wherein R³ is 4-methylpiperazin-1-yl.

A further embodiment of present invention is (ix) a compound of formula (I) or (Ia), according to any one of (i) to (viii), or a pharmaceutically acceptable salt, enantiomers and diastereomers thereof, wherein R⁴ is 1-methoxyethyl.

A further embodiment of present invention is (x) a compound of formula (I) or (Ia), according to any one of (i) to (ix), or a pharmaceutically acceptable salt, enantiomers and diastereomers thereof, wherein R⁵ is H and R⁶ is H; or R⁵ and R⁶ together with the atoms to which they attached to form a cyclobutyl ring.

A further embodiment of present invention is (xi) a compound of formula (I) or (Ia), according to any one of (i) to (x), or a pharmaceutically acceptable salt, enantiomers and diastereomers thereof, wherein M is C₁₋₆alkylene.

A further embodiment of present invention is (xii) a compound of formula (I) or (Ia), according to any one of (i) to (xi), or a pharmaceutically acceptable salt, enantiomers and diastereomers thereof, wherein M is methylene.

A further embodiment of present invention is (xiii) a compound of formula (I) or (Ia), according to any one of (i) to (xii), or a pharmaceutically acceptable salt, enantiomers and diastereomers thereof, wherein L is ethylene.

A further embodiment of present invention is (xiv) a compound of formula (I) or (Ia), according to (i) or (ii), wherein
R¹ is C₃₋₇cycloalkyl substituted by one or two substituents independently selected from C₁₋₆alkyl and halopyridyl;
R² is halogen or C₁₋₆alkyl;
R³ is C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
W is N and T is C; or W is C and T is N;
or a pharmaceutically acceptable salt thereof.

A further embodiment of present invention is (xv) a compound of formula (I) or (Ia), according to (xiv), wherein
R¹ is 2-(2-fluoro-3-pyridyl)cyclopropyl or 2,3-dimethylcyclopropyl;
R² is fluoro or methyl;
R³ is 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
W is N and T is C; or W is C and T is N;
or a pharmaceutically acceptable salt thereof.

Another embodiment of present invention is (xvi) a compound of formula (Ia-1),

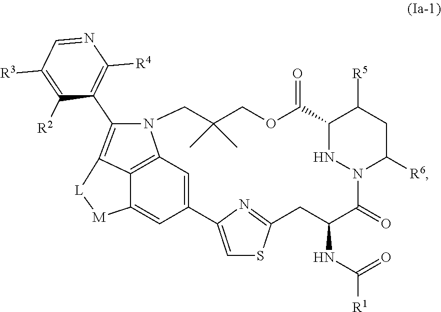

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
or a pharmaceutically acceptable salt thereof.

A further embodiment of present invention is (xvii) a compound of formula (Ia-1), according to (xvi), wherein
R¹ is C₃₋₇cycloalkyl substituted by one or two substituents independently selected from C₁₋₆alkyl and halopyridyl;
R² is halogen or C₁₋₆alkyl;
R³ is C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
or a pharmaceutically acceptable salt thereof.

A further embodiment of present invention is (xviii) a compound of formula (Ia-1), according to (xvi) or (xvii), wherein
R¹ is 2-(2-fluoro-3-pyridyl)cyclopropyl or 2,3-dimethylcyclopropyl;
R² is fluoro or methyl;
R³ is 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
or a pharmaceutically acceptable salt thereof.

Another embodiment of present invention is (xix) a compound of formula (Ia-2),

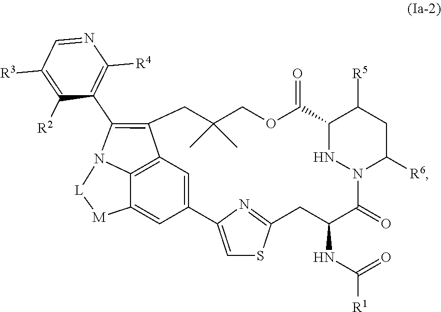

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
or a pharmaceutically acceptable salt thereof.

A further embodiment of present invention is (xx) a compound of formula (Ia-2), according to (xix), wherein
R¹ is C₃₋₇cycloalkyl di-substituted by C₁₋₆alkyl;
R² is C₁₋₆alkyl;
R³ is C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene;
L is C₁₋₆alkylene;
or a pharmaceutically acceptable salt thereof.

A further embodiment of present invention is (xxi) a compound of formula (Ia-2), according to (xix) or (xx), wherein
R¹ is 2,3-dimethylcyclopropyl;
R² is methyl;
R³ is 4-methylpiperazin-1-yl;
R⁴ is 1-methoxyethyl;
R⁵ is H and R⁶ is H; or R⁵ and R⁶ together with the atoms to which they attached to form a cyclobutyl ring;
M is methylene;
L is ethylene;
or a pharmaceutically acceptable salt thereof.

Another embodiment of present invention is (xxii) a compound selected from the following:
(1r,2R,3S)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1r,2R,3S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide;
(1S,6R,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]norcarane-7-carboxamide;
(1R,6S,7R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide;
(1r,2S,3R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide;
(1S,2S)-2-(2-fluoro-3-pyridyl)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]cyclopropanecarboxamide;
(1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(3-pyridyl)cyclopropanecarboxamide;
(1r,2S,3R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2,3-dimethyl-cyclopropanecarboxamide;
(1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-methyl-3-pyridyl)cyclopropanecarboxamide;
(1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-(2-fluoro-3-pyridyl)cyclopropanecarboxamide;
(1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-fluoro-3-pyridyl)cyclopropanecarboxamide;
(1r,2R,3S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide;
(1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1S,2S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2-(2-fluoro-3-pyridyl)cyclopropanecarboxamide;
(1R,6S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide;
(1R,6S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide;
(1R,6S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide;
(1R,6S,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]norcarane-7-carboxamide;
(1S,6R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-3-oxabicyclo[4.1.0]heptane-7-carboxamide;
(1R,6S,7r)-N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide;
(1R,6S,7R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-3-oxabicyclo[4.1.0]heptane-7-carboxamide;
(1R,6S,7R)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-3-oxabicyclo[4.1.0]heptane-7-carboxamide;
(1R,6S,7r)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide;
(1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2-(2-fluoro-3-pyridyl)cyclopropanecarboxamide;
(1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide;
(1S,2R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-tetrahydropyran-4-yl-cyclopropanecarboxamide;
(1r,2R,3S)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2S,3R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide;
(1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide;
cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide;
cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]cyclopropanecarboxamide;
cis-(1S,2S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-(2-fluoro-3-pyridyl)cyclopropanecarboxamide;
(1r,2R,3S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
cis-(1S,2R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide;
cis-(1r,2S,3R)—N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide;
cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide;
trans-(1S,2S)-2-(difluoromethyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide;
(1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-tetrahydropyran-4-yl-cyclopropanecarboxamide;
(1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[4.1.0]heptane-7-carboxamide;
(1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12} 0^{19,27}.0^{21,26}]dotriaconta-1(28),2,5(32),19,25(29),26-hexaen-7-yl]-2,3-dimethyl-cyclopropanecarboxamide;
(1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide;
(1S,5R)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide;
(1R,5S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide;
(1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[4.1.0]heptane-7-carboxamide;
(1r,2S,3R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-methyl-norcarane-7-carboxamide;
(1R,6R,7S)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide;
(1R,6R,7R)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide;
(1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2R,3S)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2R,3S)—N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2R,3S)—N-[(7S,13S)-(20P)-20-[4-chloro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide; and
(1r, 2R,3S)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
   or a pharmaceutically acceptable salt thereof.

A further embodiment of present invention is (xxiii) a compound selected from:

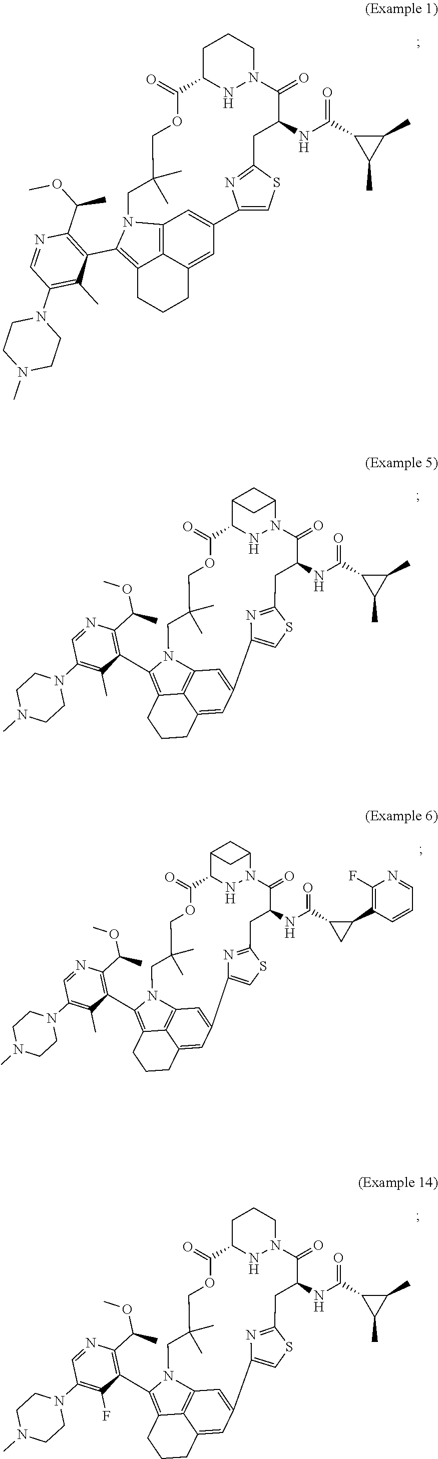

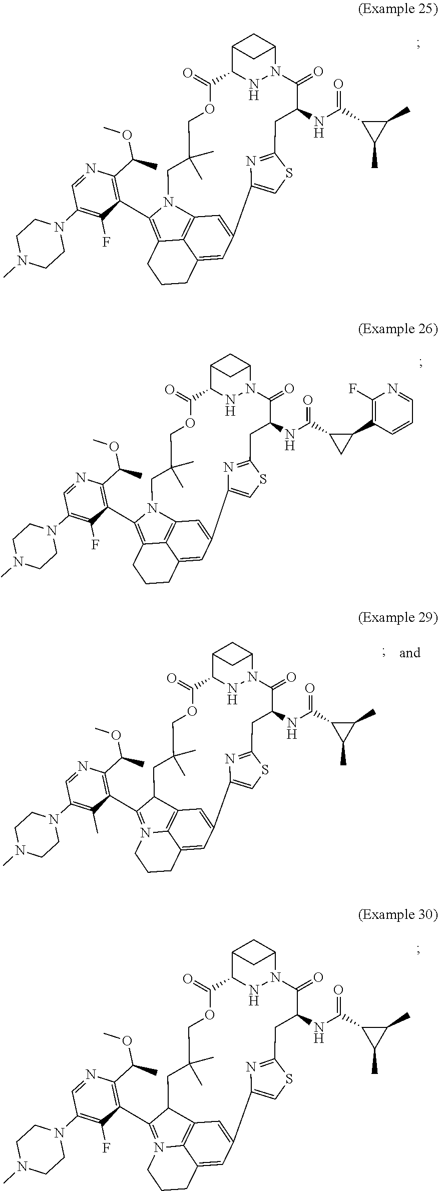

or a pharmaceutically acceptable salt, enantiomers and diastereomers thereof.

A further embodiment of present invention is (xxiii′) a compound selected from:

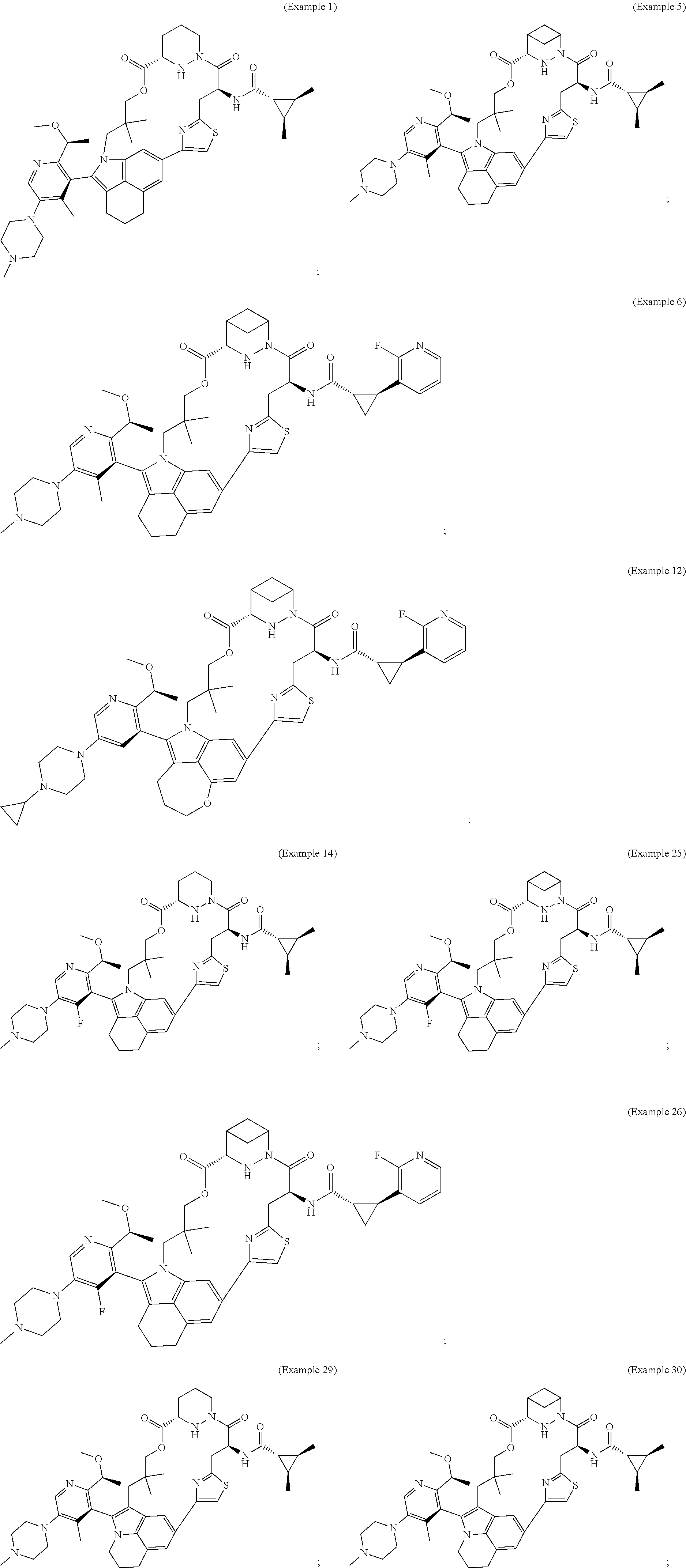

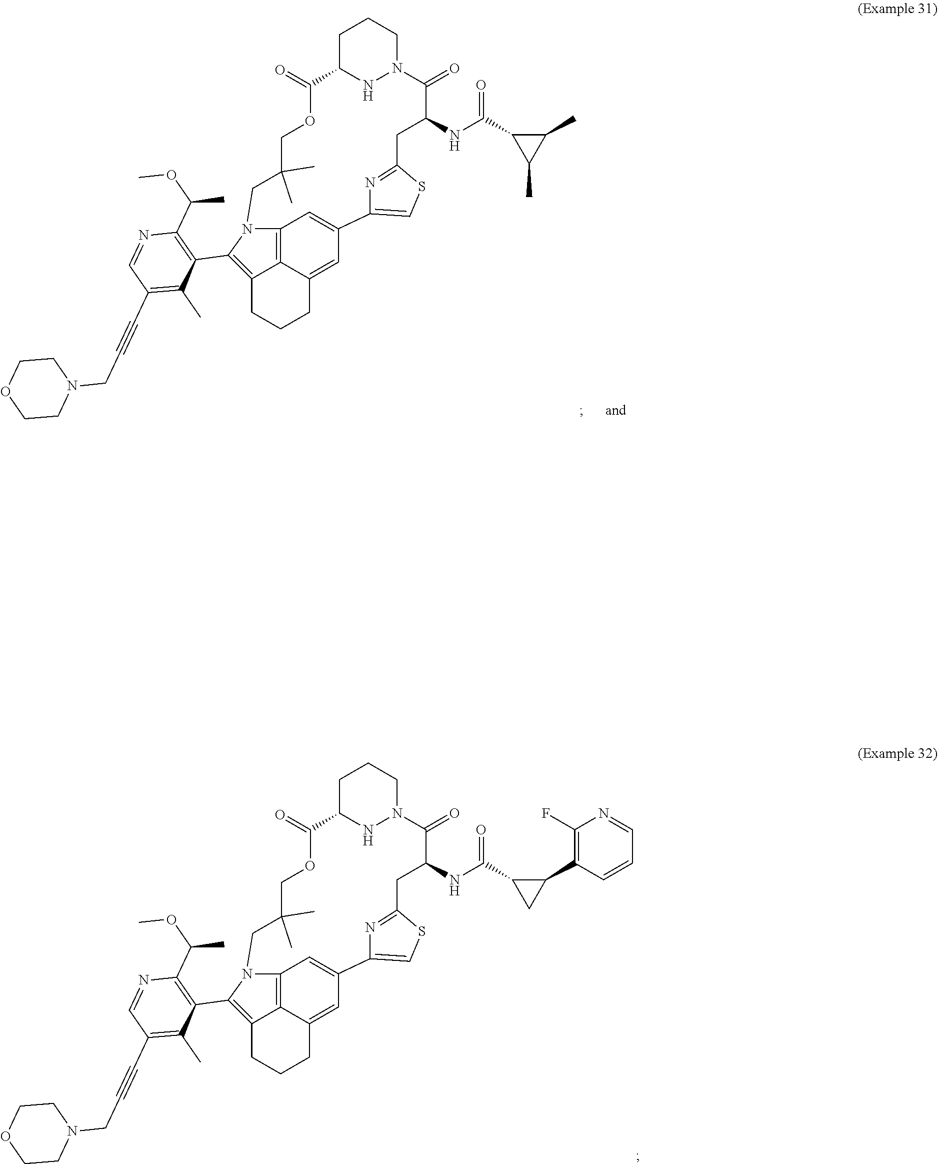

or a pharmaceutically acceptable salt, enantiomers and diastereomers thereof.

Another embodiment of present invention is related to (xxiv) a process for the preparation of a compound according to any one of (i) to (xxiii) comprising the following step:
a) coupling reaction between compound of formula (II),

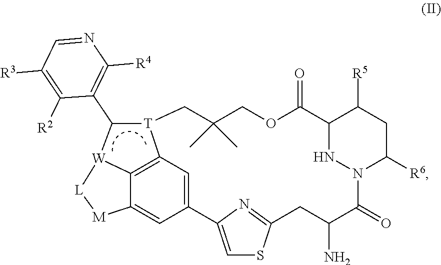

and acid (III),

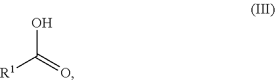

in the presence of a coupling reagent and a base to form the compound of formula (I); wherein R¹ to R⁶, M, L, T and W are defined as in any one of (i) to (xxii); the coupling reagent is T₃P, HATU, PyBOP or EDCI/HOBt; the base is TEA, DIEPA or DMAP.

Another embodiment of present invention is (xxv) a compound or pharmaceutically acceptable salt according to any one of (i) to (xxiii), (i′) to (xviii′) or (i″) to (xxvi″) for use as therapeutically active substance.

Another embodiment of present invention is (xxvi) a pharmaceutical composition comprising a compound in accordance with any one of (i) to (xxiii), (i′) to (xviii′) or (i″) to (xxvi″) and a pharmaceutically acceptable excipient.

Another embodiment of present invention is (xxvii) the use of a compound according to any one of (i) to (xxiii), (i′) to (xviii′) or (i″) to (xxvi″) for treating a KRAS G12V protein-related disease.

Another embodiment of present invention is (xxviii) the use of a compound according to any one of (i) to (xxiii), (i′) to (xviii′) or (i″) to (xxvi″) for inhibiting RAS interaction with downstream effectors, wherein the downstream effectors are RAF and PI3K.

Another embodiment of present invention is (xxix) the use of a compound according to any one of (i) to (xxiii), (i′) to (xviii′) or (i″) to (xxvi″) for inhibiting the propagating oncogenic MAPK and PI3K signaling.

Another embodiment of present invention is the use of a compound according to any one of (i) to (xxiii), (i′) to (xviii′) or (i″) to (xxvi″) for the treatment or prophylaxis of cancers in a subject in need thereof, wherein the cancer comprises a first RAS mutation that is G12V and a second RAS mutation at a position selected from the group consisting of V8A, V9Y, S17E, A59T, T58I, D69P, M72I, S65W, R68S, D92R, H95N, Y96D, Q99W and F156L.

Another embodiment of present invention is the use of a compound according to any one of (i) to (xxiii), (i′) to (xviii′) or (i″) to (xxvi″) for the preparation of a medicament for the treatment or prophylaxis of cancers in a subject in need thereof, wherein the cancer comprises a first RAS mutation that is G12V and a second RAS mutation at a position selected from the group consisting of V8A, V9Y, S17E, A59T, T58I, D69P, M72I, S65W, R68S, D92R, H95N, Y96D, Q99W and F156L.

Another embodiment of present invention is (xxx) the use of a compound according to any one of (i) to (xxiii), (i′) to (xviii′) or (i″) to (xxvi″) for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.

Another embodiment of present invention is (xxxi) the use of a compound according to any one of (i) to (xxiii), (i′) to (xviii′) or (i″) to (xxvi″) for the treatment or prophylaxis of KRAS mutation driven cancers, wherein the cancer is selected from pancreatic adenocarcinoma, colorectal cancer and non-small cell lung cancer.

Another embodiment of present invention is the use of a compound according to any one of (i) to (xxiii), (i′) to (xviii′) or (i″) to (xxvi″) for the treatment or prophylaxis of primary central nervous system (CNS) tumors harboring RAS mutations or RAS driven cancers with brain metastases; wherein the CNS tumor is primary melanocytic tumors of the CNS harboring NRAS mutation; wherein the cancer is selected from pancreatic adenocarcinoma, colorectal cancer, non-small cell lung cancer.

Another embodiment of present invention is the use of a compound according to any one of (i) to (xxiii), (i′) to (xviii′) or (i″) to (xxvi″) for the treatment or prophylaxis of primary central nervous system (CNS) tumors harboring RAS mutations or RAS driven cancers with brain metastases; wherein the CNS tumor is primary melanocytic tumors of the CNS harboring NRAS mutation; wherein the cancer is non-small cell lung cancer.

Another embodiment of present invention is a compound or pharmaceutically acceptable salt according to any one of (i) to (xxiii), (i′) to (xviii′) or (i″) to (xxvi″) for the treatment or prophylaxis of KRAS mutation driven cancers, wherein the cancer is selected from pancreatic adenocarcinoma, colorectal cancer and non-small cell lung cancer.

Another embodiment of present invention is (xxxii) the use of a compound according to any one of (i) to (xxiii), (i′) to (xviii′) or (i″) to (xxvi″) for the preparation of a medicament for the treatment or prophylaxis of KRAS mutation driven cancers, wherein the cancer is selected from pancreatic adenocarcinoma, colorectal cancer and non-small cell lung cancer.

Another embodiment of present invention is (xxxiii) a method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of (i) to (xxiii), (i′) to (xviii′) or (i″) to (xxvi″).

Another embodiment of present invention is (xxxiv) a compound or pharmaceutically acceptable salt according to any one of (i) to (xxiii), (i′) to (xviii′) or (i″) to (xxvi″), when manufactured according to a process of (xxiv).

Another embodiment of present invention is (xxxv) a compound selected from:
(1r,2R,3S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2,3-dimethyl-cyclopropanecarboxamide;
(1r,2R,3S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,29-tetrazahexacyclo[17.5.2.1^{2,5}.1^{9,13}.1^{10,12}.0^{22,26}]nonacosa-1(25),2,5(29),19,22(26),23-hexaen-7-yl]-2,3-dimethyl-cyclopropanecarboxamide;
(1S,2S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
   or a pharmaceutically acceptable salt thereof.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations and is the minimum amount necessary to inhibit mutant RAS (e.g. KRAS G12C) interaction with RAF, blocking the oncogenic MAPK signaling. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 0.1 to 1000 mg/kg of patient body weight per day, with the typical initial range of compound used being 0.3 to 15 mg/kg/day. In another embodiment, oral unit dosage forms, such as tablets and capsules, preferably contain from about 1 to about 1000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 1 to 1000 mg of the compound of the invention compounded with about 1 to 1000 mg anhydrous lactose, about 1 to 1000 mg sodium croscarmellose, about 1 to 1000 mg polyvinylpyrrolidone (PVP) K30, and about 1 to 1000 mg magnesium stearate. The powdered ingredients are first mixed together and then mixed with a solution of the PVP. The resulting composition can be dried, granulated, mixed with the magnesium stearate and compressed into tablet form using conventional equipment. An example of an aerosol formulation can be prepared by dissolving the compound, for example 5 to 400 mg, of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment of mutant KRAS-driven cancers. Another embodiment includes a pharmaceutical composition comprising a compound of Formula (I) for use in the treatment of mutant KRAS-driven cancers.

The following composition A and B illustrate typical compositions of the present invention but serve merely as representative thereof.

### Composition A

A compound of the present invention can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Composition B

A compound of the present invention can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Indications and Methods of Treatment

The compounds of the invention induce a new binding pocket in KRAS by driving formation of a high affinity tri-complex between KRAS protein and the widely expressed cyclophilin A (CYPA), which inhibit KRAS interaction with downstream effectors, such as RAF and PI3K. Accordingly, the compounds of the invention are useful for inhibiting the propagating oncogenic MAPK and PI3K signaling, reducing cell proliferation, in particular cancer cells. Compounds of the invention are useful for termination of RAS signaling in cells that express RAS mutant, e.g. KRAS mutation driven pancreatic cancer, colorectal cancer, lung cancer, esophageal cancer, gallbladder cancer, melanoma ovarian cancer, endometrial cancer, etc. Alternatively, compounds of the invention are useful for termination of RAS signaling in malignant solid tumor where the oncogenic role of KRAS mutation is reinforced by dysregulation or mutation of effector pathways as MAPK, PI3K-AKT-mTOR (Mammalian target of rapamycin) driven signaling, for targeted therapy in pancreatic adenocarcinoma, colorectal cancer, non-small cell lung cancer, etc.

Another embodiment includes a method of treating or preventing cancer in a mammal in need of such treatment, wherein the method comprises administering to said mammal a therapeutically effective amount of a compound of formula (I), a stereoisomer, tautomer or pharmaceutically acceptable salt thereof.

### Synthesis

The compounds of the present invention can be prepared by any conventional means. Suitable processes for synthesizing these compounds as well as their starting materials are provided in the schemes below and in the examples. All substituents, in particular, R¹ to R⁶, M, L and W and T are defined as above unless otherwise indicated. Furthermore, and unless explicitly otherwise stated, all reactions, reaction conditions, abbreviations and symbols have the meanings well known to a person of ordinary skill in organic chemistry.

General synthetic routes for preparing the compound of formula (I) are shown below.

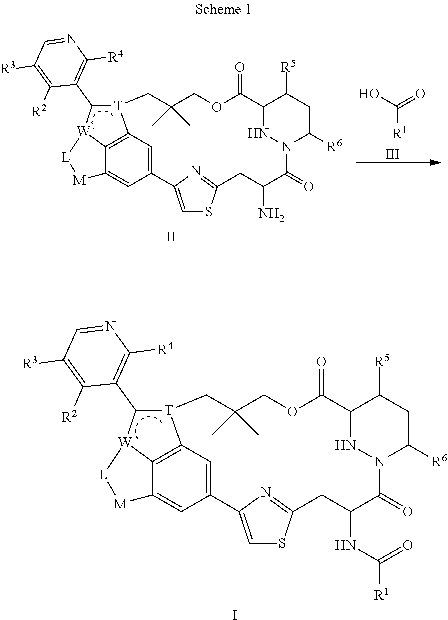

Compound of formula II was synthesized according to the procedure described in Intermediate A to Intermediate N2. Compound of formula (I) can be obtained by a coupling reaction between acid (III) and compound of formula (II) with coupling reagent(s), such as T₃P, HATU, PyBOP and EDCI/HOBt, in the presence of a base, such as TEA, DIEPA and DMAP.

Compounds of this invention can be obtained as mixtures of diastereomers or enantiomers, which can be separated by methods well known in the art, e.g. (chiral) HPLC or SFC. In another embodiment, compound of formula (I) can be obtained according to above scheme by using corresponding chiral starting materials.

This invention also relates to a process for the preparation of a compound of formula (I) comprising following step:
a) coupling reaction between compound of formula (II),

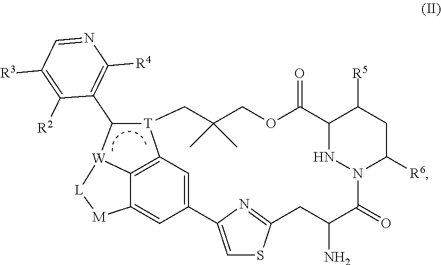

and acid (III),

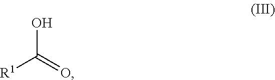

in the presence of a coupling reagent and a base to form the compound of formula (I);
wherein
in step a) the coupling reagent can be, for example, T₃P, HATU, PyBOP or EDCI/HOBt; the base can be, for example, TEA, DIEPA or DMAP.

A compound of formula (I) or (Ia) when manufactured according to the above process is also an object of the invention.

### EXAMPLES

The invention will be more fully understood by reference to the following examples. They should not, however, be construed as limiting the scope of the invention.

### Abbreviations

The invention will be more fully understood by reference to the following examples. They should not, however, be construed as limiting the scope of the invention.

Abbreviations used herein are as follows:
ACN acetonitrile
AgOTf Silver trifluoromethanesulfonate
AlCl₃ Aluminium Chloride
aq. Aqueous
(Boc)₂O Di-tert-butyldicarbonate
(R)-binap (R)-(+)-2,2′-Bis(diphenylphosphino)-1,1′-binaphthyl
cataCXium A Pd G3 Mesylate[(di(1-adamantyl)-n-butylphosphine)-2-(2′-amino-1,1′-biphenyl)]palladium(II)
CDCl₃: deuterated chloroform
CD₃OD deuterated methanol
CMPI 2-Chloro-1-methylpyridinium iodide
CMBP (Tributylphosphoranylidene)acetonitrile
COMU (1-Cyano-2-ethoxy-2-oxoethylidenaminooxy)dimethylamino-morpholino-carbenium hexafluorophosphate
conc. Concentrated
Cs₂CO₃ Cesium carbonate
DBU 1,8-Diazabicyclo[5.4.0]undec-7-ene
DIEPA N, N-diethylpropylamine
DMAP 4-Dimethylaminopyridine
DMF dimethyl formamide
DMSO dimethyl sulfoxide
dppf 1,1′-Bis(diphenylphosphino)ferrocene
EDCI·HCl 1-(3-Dimethylaminopropyl)-3-ethylcarbodiimide hydrochloride
EtOAc or EA ethyl acetate
Et₃SiH Triethylsilane
FRET fluorescence resonance energy transfer
HATU (1-[Bis(dimethylamino)methylene]-1H-1,2,3-triazolo[4,5-b]pyridinium 3-oxid hexafluorophosphate)
hr(s) hour(s)
HPLC high performance liquid chromatography
IPA Isopropyl alcohol
[Ir(OMe)(COD)]₂ (1,5-Cyclooctadiene)(methoxy)iridium(I) dimer
KOAc Potassium acetate
K₂CO₃ Potassium carbonate
K₃PO₄ Potassium phosphate
LDA Lithium diisopropylamide
LiOH Lithium hydroxide
LiBH₄ Lithium borohydride
MS: (ESI) mass spectroscopy (electron spray ionization)
m-CPBA 3-Chloroperbenzoic acid
min(s) minute(s)
NMI 1-Methylimidazole
NMR nuclear magnetic resonance
obsd Observed
(R)-binap (R)-(+)-2,2′-Bis(diphenylphosphino)-1,1′-binaphthalene
Pd-PEPPSI-IPent^{Ct} [1,3-bis[2,6-bis(1-ethylpropyl)phenyl]-4,5-dichloro-imidazol-2-ylidene]-dichloro-(3-chloropyridin-1-ium-1-yl)palladium
Pd(dppf)Cl₂ [1,1′-Bis(diphenylphosphino)ferrocene]dichloropalladium(II)
Pd(dtbpf)Cl₂ [1,1′-Bis(di-tert-butylphosphino)ferrocene]dichloropalladium(II)
prep-HPLC preparative high performance liquid chromatography
Rh₂(OAc)₄ Rhodium(II) acetate
RT or rt room temperature
PyBOP Benzotriazole-1-yl-oxytripyrrolidinophosphonium hexafluorophosphate
PyFluor 2-Pyridinesulfonyl Fluoride
sat. saturated
SFC supercritical fluid chromatography
Sphos 2-Dicyclohexylphosphino-2′,6′-dimethoxybiphenyl
TBAF Tetrabutylammonium fluoride
TBSCl tert-butyldimethylsilyl chloride
TCFH N,N,N′,N′-Tetramethylchloroformamidinium Hexafluorophosphate
TEA triethylamine
TFA trifluoroacetic acid
TFAA Trifluoroacetic anhydride
THF tetrahydrofuran
TMSCN Trimethylsilyl cyanide
T3P propylphosphonic anhydride
T4P butylphosphate anhydride
Vilsmeier reagent 1-Chloro-N,N-dimethylmethaniminium chloride
Xphos-G4 Methanesulfonato(2-dicyclohexylphosphino-2′,4′,6′-tri-1-propyl-1,1′-biphenyl)(2′-methylamino-1,1′-biphenyl-2-yl)palladium(II)

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 A, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: acetonitrile and 0.1% ammonium hydroxide in water; acetonitrile and 0.1% FA in water or acetonitrile and 0.1% TFA in water). Or Gilson-281 purification System (Pump 322, Detector: UV 156, solvent system: acetonitrile and 0.05% ammonium hydroxide in water; acetonitrile and 0.225% FA in water; acetonitrile and 0.05% HCl in water; acetonitrile and 0.075% TFA in water; or acetonitrile and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃·H₂O in MeOH), back pressure 100 bar, detection UV@254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):
Acidic condition I: A: 0.1% TFA in H₂O; B: 0.1% TFA in acetonitrile;
Acidic condition II: A: 0.0375% TFA in H₂O; B: 0.01875% TFA in acetonitrile;
Basic condition I: A: 0.1% NH₃·H₂O in H₂O; B: acetonitrile;
Basic condition II: A: 0.025% NH₃·H₂O in H₂O; B: acetonitrile;
Neutral condition: A: H₂O; B: acetonitrile.
Mass spectra (MS): generally only ions which indicate the parent mass are reported, and unless otherwise stated the mass ion quoted is the positive mass ion (MH)⁺.
NMR Spectra were obtained using Bruker Avance 400 MHz or 500 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Preparation for Intermediate

### Intermediate A1

### Benzyl 4-[5-chloro-6-[(1S)-1-methoxyethyl]-4-methyl-3-pyridyl]piperazine-1-carboxylate

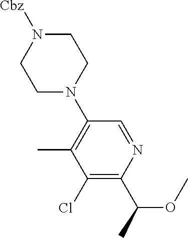 [SMILES: CO[C@@H](C)c1ncc(N2CCN(C(=O)OCc3ccccc3)CC2)c(C)c1Cl]

The title intermediate A1 was prepared according to the following scheme:

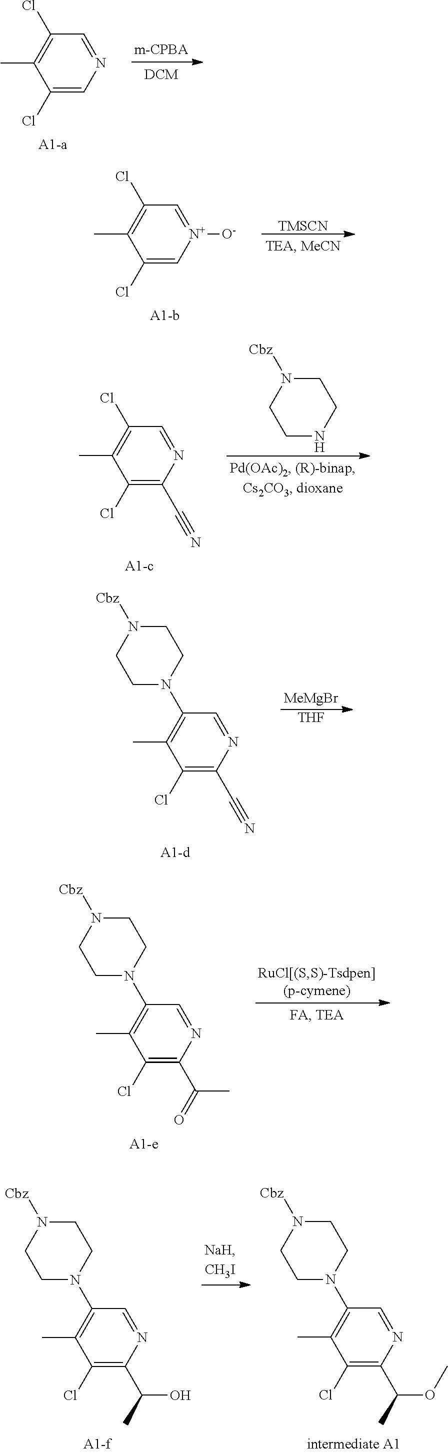

### Step 1: Preparation of 3,5-dichloro-4-methyl-1-oxido-pyridin-1-ium (compound A1-b)

To a solution of 3,5-dichloro-4-methyl-pyridine (compound A1-a, 24.0 g, 148.14 mmol) in DCM (500 mL) was added m-CPBA (30.1 g, 148.14 mmol) slowly at 0° C. The reaction mixture was then stirred at 20° C. for 24 hrs. After the reaction was completed, K₂CO₃ (23 g, 166 mmol) was added at 0° C. and the mixture was stirred at 20° C. for 1 h. The resulting suspension was then filtered, and the filtrate was washed with sat. Na₂SO₃ aq. (400 mL), dried over Na₂SO₄, filtered and concentrated under vacuum to give a crude 3,5-dichloro-4-methyl-1-oxido-pyridin-1-ium (compound A1-b, 24.0 g) as a white solid. MS calc'd 178 (MH⁺), measured 178 (MH⁺).

### Step 2: Preparation of 3,5-dichloro-4-methyl-pyridine-2-carbonitrile (compound A1-c)

To a solution of 3,5-dichloro-4-methyl-1-oxido-pyridin-1-ium (compound A1-b, 14.0 g, 78.65 mmol) in ACN (400 mL), TMSCN (24.0 g, 241.94 mmol) and TEA (54.8 mL, 393.24 mmol) were added at 20° C. The mixture was stirred at 85° C. for 12 hrs under N2 atmosphere. After the reaction was completed, water (500 mL) was added with, and the mixture was extracted with EtOAc (300 mL, three times). The combined organic layer was washed with brine (400 mL), dried over Na₂SO₄, filtered and concentrated under vacuum to give a residue. The residue was purified by column chromatography to afford 3,5-dichloro-4-methyl-pyridine-2-carbonitrile (compound A1-c, 13.5 g) as brown oil. MS calc'd 186.0 (MH⁺), measured 187.2 (MH⁺).

### Step 3: Preparation of benzyl 4-(5-chloro-6-cyano-4-methyl-3-pyridyl)piperazine-1-carboxylate (compound A1-d)

To a solution of 3,5-dichloro-4-methyl-pyridine-2-carbonitrile (compound A1-c, 13.5 g, 72.18 mmol) and benzyl piperazine-1-carboxylate (15.9 g, 72.18 mmol) in 1,4-dioxane (500 mL), Cs₂CO₃ (70.6 g, 216.55 mmol), (R)-BINAP (1.1 g, 1.8 mmol) and palladium (II) acetate (0.8 g, 3.61 mmol) were added. The mixture was degassed and purged with N2 three times and then stirred at 80° C. for 12 hrs. The reaction mixture was subsequently filtered, and the filtrate was concentrated under vacuum to yield a residue. The residue was purified via column chromatography to afford benzyl 4-(5-chloro-6-cyano-4-methyl-3-pyridyl)piperazine-1-carboxylate (compound A1-d, 7.0 g) as a yellow solid. MS calc'd 371.1 (MH⁺), measured 371.1 (MH⁺). ¹H NMR (400 MHz, CHLOROFORM-d) δ=8.19 (s, 1H), 7.40-7.33 (m, 5H), 5.18 (s, 2H), 3.74-3.67 (m, 4H), 3.04 (s, 4H), 2.40 (s, 3H).

### Step 4: Preparation of benzyl 4-(6-acetyl-5-chloro-4-methyl-3-pyridyl)piperazine-1-carboxylate (compound A1-e)

To a solution of benzyl 4-(5-chloro-6-cyano-4-methyl-3-pyridyl)piperazine-1-carboxylate (compound A1-d, 6.5 g, 17.53 mmol) in THF (200 mL), methyl magnesium bromide (14.6 mL, 43.82 mmol) was added dropwise at −5° C. under nitrogen atmosphere. The reaction mixture was stirred at −5° C. for 1 h. After the reaction was completed, it was quenched with HCl aq. (3M, 500 mL) at 0° C. and extracted with EtOAc (250 mL, three times). The combined organic layer was washed with brine (400 mL), dried over Na₂SO₄, filtered and concentrated under vacuum to give a residue. The residue was purified by column chromatography to afford benzyl 4-(6-acetyl-5-chloro-4-methyl-3-pyridyl)piperazine-1-carboxylate (compound A1-e, 4.9 g) as a white solid, MS calc'd 388.1 (MH⁺), measured 388.1 (MH⁺).

### Step 5: Preparation of benzyl 4-[5-chloro-6-[(1S)-1-hydroxyethyl]-4-methyl-3-pyridyl]piperazine-1-carboxylate (compound A1-f)

Formic acid (2.91 g, 63.17 mmol) was slowly added into TEA (81.7 mL, 585.93 mmol) at 0° C. After being stirred at 0° C. for 0.3 h, the reaction mixture was added with [(S,S)—N-(2-Amino-1,2-diphenylethyl)-p-toluenesulfonamide]chloro(p-cymene)ruthenium(II) (803.7 mg, 1.26 mmol), followed by addition of a solution of benzyl 4-(6-acetyl-5-chloro-4-methyl-3-pyridyl)piperazine-1-carboxylate (4.9 g, 12.63 mmol) in THF (10 mL) at 20° C. Then the reaction mixture was stirred at 45° C. for 12 hrs. The reaction mixture was concentrated under vacuum to give a residue. The residue was purified by column chromatography to afford benzyl 4-[5-chloro-6-[(1S)-1-hydroxyethyl]-4-methyl-3-pyridyl]piperazine-1-carboxylate (compound A1-f, 2.7 g) as pink oil. MS calc'd 390.2 (MH⁺), measured 390.2 (MH⁺).

### Step 6: Preparation of benzyl 4-[5-chloro-6-[(1S)-1-methoxyethyl]-4-methyl-3-pyridyl]piperazine-1-carboxylate (intermediate A1)

To a solution of benzyl 4-[5-chloro-6-[(1S)-1-hydroxyethyl]-4-methyl-3-pyridyl]piperazine-1-carboxylate (compound A1-f, 2.7 g, 6.87 mmol) in THF (50 mL), sodium hydride (824.8 mg, 20.62 mmol, 60% in oil) was slowly added at 0° C. After being stirred for 0.5 h under N2 atmosphere, iodomethane (1.9 g, 13.75 mmol) was added with at 0° C. and the reaction mixture was stirred for another 3 hrs. The reaction mixture was added into ice water (500 mL), extracted with EtOAc (200 mL, three times), The combined organic layer was washed with brine (300 mL), dried over Na₂SO₄, filtered and concentrated under vacuum to give a residue. The residue was purified by column chromatography to afford benzyl 4-[5-chloro-6-[(1S)-1-methoxyethyl]-4-methyl-3-pyridyl]piperazine-1-carboxylate (intermediate A1, 2.3 g) as pink oil. MS calc'd 404.2 (MH⁺), measured 404.2 (MH⁺).

### Intermediate A2

### Benzyl 4-[4-fluoro-5-iodo-6-[(1S)-1-methoxyethyl]-3-pyridyl]piperazine-1-carboxylate

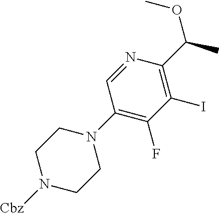 [SMILES: CO[C@@H](C)c1ncc(N2CCN(C(=O)OCc3ccccc3)CC2)c(F)c1I]

The title intermediate A2 was prepared according to the following scheme:

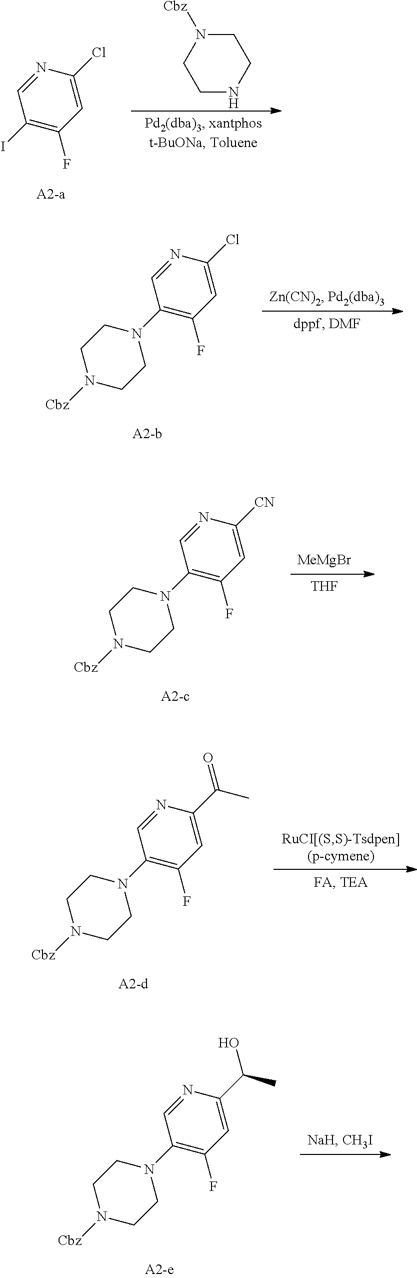

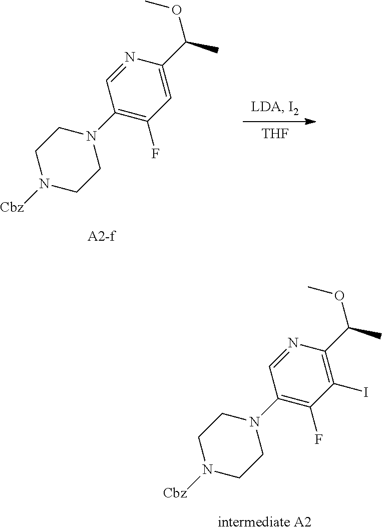 [SMILES: CO[C@@H](C)c1cc(F)c(N2CCN(C(=O)OCc3ccccc3)CC2)cn1 ; CO[C@@H](C)c1ncc(N2CCN(C(=O)OCc3ccccc3)CC2)c(F)c1I ; "THF"]

### Step 1: Preparation of benzyl 4-(6-chloro-4-fluoro-3-pyridyl)piperazine-1-carboxylate (compound A2-b)

To a solution of benzyl piperazine-1-carboxylate (16.5 g, 74.91 mmol) in toluene (300 mL), 2-chloro-4-fluoro-5-iodo-pyridine (compound A2-a, 13.7 g, 53.3 mmol), Xantphos (6.2 g, 10.66 mmol), sodium t-butoxide (7.7 g, 79.94 mmol) and Pd₂(dba)₃ (4.9 g, 5.33 mmol) were added. The mixture was degassed and purged with N2 for 3 times, and the mixture was stirred at 80° C. for 15 hrs under N2 atmosphere. The crude mixture was filtered through a pad of celite. The filtrate was concentrated under vacuum to give a residue which was purified via column chromatography to afford benzyl 4-(6-chloro-4-fluoro-3-pyridyl)piperazine-1-carboxylate (compound A2-b, 9.2 g) as a brown solid. MS calc'd 350.1 (MH⁺), measured 350.0 (MH⁺).

### Step 2: Preparation of benzyl 4-(6-cyano-4-fluoro-3-pyridyl)piperazine-1-carboxylate (compound A2-c)

To a solution of benzyl 4-(6-chloro-4-fluoro-3-pyridyl)piperazine-1-carboxylate (compound A2-b, 9.1 g, 26.13 mmol) in DMF (250 mL), Zn(CN)₂ (9.8 g, 26.67 mmol), dppf (2.9 g, 5.23 mmol) and Pd₂(dba)₃ (2.1 g, 2.61 mmol) were added. The mixture was degassed and purged with N₂ 3 times, and the mixture was stirred at 100° C. for 15 hrs under N2. The reaction mixture was filtered through a pad of celite. The filtrate was poured into water (1 L) and extracted with EtOAc (500 mL, three times). The combined organic layer was washed with brine (500 mL, three times), dried over Na₂SO₄, filtered and concentrated under vacuum to give a residue which was purified by column chromatography to afford benzyl 4-(6-cyano-4-fluoro-3-pyridyl)piperazine-1-carboxylate (compound A2-c, 7.0 g) as a yellow solid. MS calc'd 341.1 (MH⁺), measured 341.0 (MH⁺).

### Step 3: Preparation of benzyl 4-(6-acetyl-4-fluoro-3-pyridyl)piperazine-1-carboxylate (compound A2-d)

To a solution of benzyl 4-(6-cyano-4-fluoro-3-pyridyl)piperazine-1-carboxylate (compound A2-c, 6.0 g, 17.63 mmol) in THF (120 mL), methyl magnesium bromide (18.0 mL, 54.0 mmol) was added dropwise at −5° C. under N₂. The reaction mixture was stirred at −5° C. for 1 h. The reaction mixture was quenched with 1N HCl (20 mL) slowly at 0° C. and extracted with EtOAc (30 mL, three times). The combined organic phase was washed with brine (35 mL), dried over anhydrous Na₂SO₄, filtered and concentrated under vacuum to give a residue which was purified by column chromatography to afford benzyl 4-(6-acetyl-4-fluoro-3-pyridyl)piperazine-1-carboxylate (compound A2-d, 5.5 g) as a yellow solid. MS calc'd 358.1 (MH⁺), measured 358.3 (MH⁺).

### Step 4: Preparation of benzyl 4-[4-fluoro-6-[(1S)-1-hydroxyethyl]-3-pyridyl]piperazine-1-carboxylate (compound A2-e)

To a solution of benzyl 4-(6-acetyl-4-fluoro-3-pyridyl)piperazine-1-carboxylate (compound A2-d, 5.5 g, 15.25 mmol) in THF (31 mL), TEA (136.0 mL, 975.75 mmol) and formic acid (3.5 g, 76.25 mmol) were added at 20° C., then followed by (S,S)—N-(p-toluenesulfonyl)-1,2-diphenylethanediamine (chloro)(p-cymene)ruthenium (970.2 mg, 1.52 mmol). The mixture was stirred at 45° C. for 12 hrs under N2 atmosphere. The reaction mixture was concentrated under vacuum to give a residue. The residue was purified via column chromatography to afford benzyl 4-[4-fluoro-6-[(1S)-1-hydroxyethyl]-3-pyridyl]piperazine-1-carboxylate (compound A2-e, 5.2 g) as brown oil. MS calc'd 360.2 (MH⁺), measured 360.1 (MH⁺).

### Step 5: Preparation of benzyl 4-[4-fluoro-6-[(1S)-1-methoxyethyl]-3-pyridyl]piperazine-1-carboxylate (compound A2-f)

To a solution of benzyl 4-[4-fluoro-6-[(1S)-1-hydroxyethyl]-3-pyridyl]piperazine-1-carboxylate (compound A2-e, 5.2 g, 14.33 mmol) in THF (100 mL), sodium hydride (1.7 g, 43.78 mmol, 60% in oil) was slowly added at 0° C. After being stirred for 0.5 h under N2 atmosphere, the reaction mixture was added with iodomethane (4.1 g, 28.66 mmol) at 0° C. and stirred for another 1 h. The reaction mixture was added into ice water (100 mL), extracted with EtOAc (50 mL, three times). The combined organic phase was washed with brine (80 mL), dried over Na₂SO₄, filtered and concentrated under vacuum to give a residue. The residue was purified via column chromatography to afford benzyl 4-[4-fluoro-6-[(1S)-1-methoxyethyl]-3-pyridyl]piperazine-1-carboxylate (compound A2-f, 4.5 g) as brown oil. MS calc'd 374.2 (MH⁺), measured 374.1 (MH⁺).

### Step 6: Preparation of benzyl 4-[4-fluoro-5-iodo-6-[(1S)-1-methoxyethyl]-3-pyridyl]piperazine-1-carboxylate (intermediate A2)

To a solution of benzyl 4-[4-fluoro-6-[(1S)-1-methoxyethyl]-3-pyridyl]piperazine-1-carboxylate (compound A2-f, 3.4 g, 9.13 mmol) in THF (80 mL) at −78° C., LDA (8.2 mL, 16.44 mmol) was added dropwise via syringe under a nitrogen atmosphere. After being stirred at −78° C. for 30 min, the reaction mixture was added with a solution of 12 (4.2 g, 16.44 mmol) in THF (80 mL) at −78° C. and stirred for another 1 h. The mixture was quenched with sat. Na₂SO₃ aq. (100 mL) at 0° C. and extracted with EtOAc (50 mL, three times). The combined organic layer was washed with brine (30 mL), dried over Na₂SO₄, filtered and concentrated under vacuum to give a residue which was purified via column chromatography to afford benzyl 4-[4-fluoro-5-iodo-6-[(1S)-1-methoxyethyl]-3-pyridyl]piperazine-1-carboxylate (intermediate A2, 1.7 g). MS calc'd 500.1 (MH⁺), measured 500.1 (MH⁺).

### Intermediate A3

### 4-[3-[5-chloro-6-[(1S)-1-methoxyethyl]-4-methyl-3-pyridyl]prop-2-ynyl]morpholine

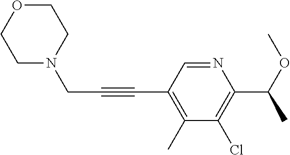 [SMILES: CO[C@@H](C)c1ncc(C#CCN2CCOCC2)c(C)c1Cl]

The intermediate A3 was prepared in analogy to the preparation of intermediate A1 by using 3-chloro-4-methyl-5-(3-morpholinoprop-1-ynyl)pyridine-2-carbonitrile (compound A3-d) instead of benzyl 4-(5-chloro-6-cyano-4-methyl-3-pyridyl)piperazine-1-carboxylate (compound A1-d).

The title compound A3-d was prepared according to the following scheme:

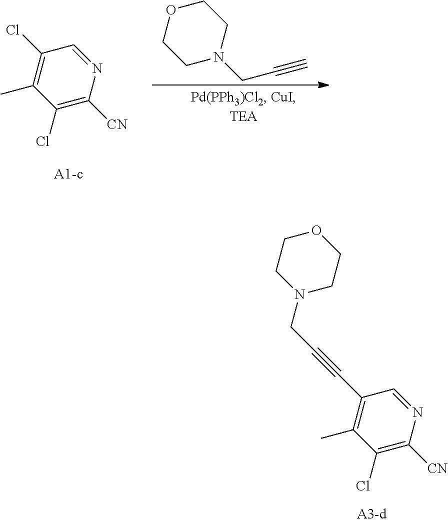

### Preparation of 3-chloro-4-methyl-5-(3-morpholinoprop-1-ynyl)pyridine-2-carbonitrile (compound A3-d)

To a solution of 3,5-dichloro-4-methyl-pyridine-2-carbonitrile (compound A1-c, 20.0 g, 106.94 mmol) in DMF (360 mL), 4-prop-2-ynylmorpholine (16.1 g, 128.33 mmol), CuI (4.1 g, 21.39 mmol), Pd(PPh₃)₂Cl₂ (7.5 g, 10.69 mmol) and TEA (74.5 mL, 534.7 mmol) were added. The mixture was evacuated and backfilled with N2 balloon (this sequence was repeated three times) and stirred at 35° C. for 16 hrs. EtOAc (500 mL) and water (1000 mL) were added, and the layers were separated. The aqueous phase was extracted with EtOAc (500 mL, twice). The combined organic layer was washed with brine (including NH₃·H₂O) (1000 mL, three times), dried over Na₂SO₄, filtered, and concentrated under vacuum to give a residue. The residue was purified via flash chromatography to obtain the crude product. The precipitate was triturated in EA and collected by filtration to give 3-chloro-4-methyl-5-(3-morpholinoprop-1-ynyl)pyridine-2-carbonitrile (compound A3-d, 5.6 g) as brown solid. MS calc'd 276.1 (MH⁺), measured 276.1 (MH); ¹H NMR (400 MHz, CHLOROFORM-d) δ=8.53 (s, 1H), 3.79 (t, J=4.4 Hz, 4H), 3.65 (s, 2H), 2.68-2.63 (m, 4H), 2.58 (s, 3H).

### Intermediate A4

### benzyl 4-[5-bromo-6-[(1S)-1-methoxyethyl]-4-methyl-3-pyridyl]piperazine-1-carboxylate

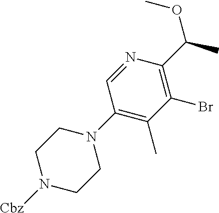 [SMILES: CO[C@@H](C)c1ncc(N2CCN(C(=O)OCc3ccccc3)CC2)c(C)c1Br]

The intermediate A4 was prepared in analogy to the preparation of intermediate A1 by using 3,5-dibromo-4-methyl-pyridine instead of 3,5-dichloro-4-methyl-pyridine (compound A1-a).

### Intermediate A5

### 4-[3-[5-bromo-6-[(1S)-1-methoxyethyl]-4-methyl-3-pyridyl]prop-2-ynyl]morpholine

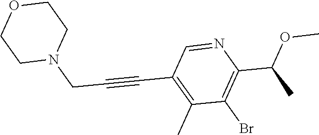 [SMILES: CO[C@@H](C)c1ncc(C#CCN2CCOCC2)c(C)c1Br]

The intermediate A5 was prepared in analogy to the preparation of intermediate A3 by using 3,5-dibromo-4-methyl-pyridine instead of 3,5-dichloro-4-methyl-pyridine (compound A1-a).

### Intermediate A6

### (9aS)-8-[5-bromo-6-[(1S)-1-methoxyethyl]-4-methyl-3-pyridyl]-3,4,6,7,9,9a-hexahydro-1H-pyrazino[2,1-c][1,4]oxazine

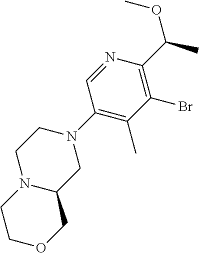 [SMILES: CO[C@@H](C)c1ncc(N2CCN3CCOC[C@@H]3C2)c(C)c1Br]

The intermediate A6 was prepared in analogy to the preparation of intermediate A1 by using 5-[(9aS)-3,4,6,7,9,9a-hexahydro-1H-pyrazino[2,1-c][1,4]oxazin-8-yl]-3-bromo-4-methyl-pyridine-2-carbonitrile (compound A6-d) instead of benzyl 4-(5-chloro-6-cyano-4-methyl-3-pyridyl)piperazine-1-carboxylate (compound A1-d).

The compound A6-d was prepared according to the following scheme:

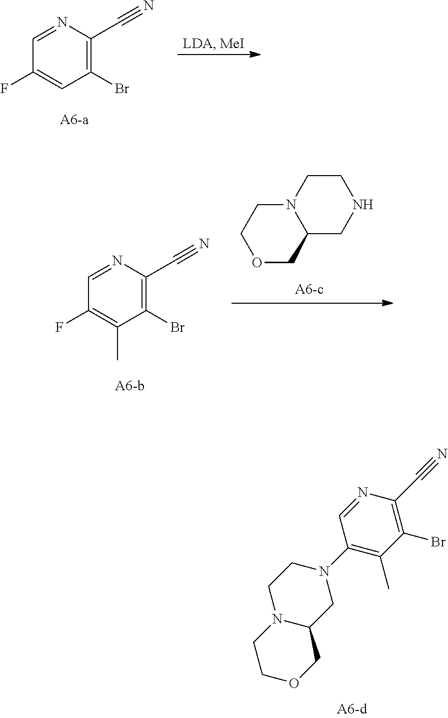

### Step 1: Preparation of 3-bromo-5-fluoro-4-methyl-pyridine-2-carbonitrile (compound A6-b)

A mixture of LDA (3.0 mL, 6.0 mmol) in THF (10 mL) was cooled to −65° C. under a nitrogen atmosphere. A solution of 3-bromo-5-fluoropyridine-2-carbonitrile (1.0 g, 4.98 mmol) in THF (3 mL) was added dropwise under the same nitrogen atmosphere. The reaction mixture was stirred at −65° C. for 10 minutes, after which iodomethane (1.3 g, 8.81 mmol) in THF (3 mL) was added dropwise. The resulting reaction mixture was stirred at −65° C. for a further 15 minutes. The reaction mixture was directly quenched with water (100 mL) and extracted with EtOAc (30 mL, twice). The combined organic phase was washed with brine (100 mL), dried over sodium sulfate (Na₂SO₄), filtered, and concentrated under reduced pressure to yield a residue. The residue was purified by silica gel column chromatography and reversed-phase chromatography to afford 3-bromo-5-fluoro-4-methyl-pyridine-2-carbonitrile (compound A6-b, 900.0 mg,) as a yellow solid. MS calc'd 214.9 (MH⁺), measured 214.9 (MH⁺).

### Step 2: Preparation of 5-[(9aS)-3,4,6,7,9,9a-hexahydro-1H-pyrazino[2,1-c][1,4]oxazin-8-yl]-3-bromo-4-methyl-pyridine-2-carbonitrile (compound A6-d)

To a solution of 3-bromo-5-fluoro-4-methyl-pyridine-2-carbonitrile (compound A6-b, 10.0 g, 46.51 mmol) in DMSO (150 mL) was added DIEA (24.3 mL, 139.52 mmol), (9aS)-1,3,4,6,7,8,9,9a-octahydropyrazino[2,1-c][1,4]oxazine;hydrochloride (compound A6-c, 12.5 g, 69.76 mmol) and stirred at 80° C. for 12 hrs. The reaction mixture was added to water (300 mL) and extracted with EtOAc (50 mL, three times). The combined organic layer was washed by brine (200 mL, three times), dried over Na₂SO₄, filtered and concentrated in vacuum to give a residue, which was purified via column chromatography to afford 5-[(9aS)-3,4,6,7,9,9a-hexahydro-1H-pyrazino[2,1-c][1,4]oxazin-8-yl]-3-bromo-4-methyl-pyridine-2-carbonitrile (5-[(9aS)-3,4,6,7,9,9a-hexahydro-1H-pyrazino[2,1-c][1,4]oxazin-8-yl]-3-bromo-4-methyl-pyridine-2-carbonitrile (compound A6-d, 15.0 g,) as a yellow solid, MS calc'd 337.1 (MH⁺), measured 337.0 (MH⁺).

### Intermediate A7

### benzyl 4-[5-chloro-4-ethyl-6-[(1S)-1-methoxyethyl]-3-pyridyl]piperazine-1-carboxylate

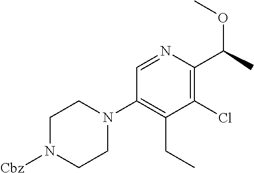 [SMILES: CCc1c(N2CCN(C(=O)OCc3ccccc3)CC2)cnc([C@H](C)OC)c1Cl]

The intermediate A7 was prepared in analogy to the preparation of intermediate A1 by using 3,5-dichloro-4-ethyl-pyridine (compound A7-a) instead of 3,5-dichloro-4-methyl-pyridine (compound A1-a).

The compound A7-a was prepared according to the following progress.

To a solution of 3,5-dichloro-4-pyridinecarbonitrile (50.0 g, 289.02 mmol) in THF (500 mL), the mixture was purged and degassed with nitrogen three times and cooled to −78° C. Ethylmagnesium bromide (100.0 mL, 300.0 mmol, 3.0 M) was added dropwise to the solution over 30 minutes at −78° C. under a nitrogen atmosphere. The reaction mixture was stirred at −78° C. for an additional 30 minutes under nitrogen. The reaction was quenched by the addition of sat. NH₄Cl aq. (300 mL) and extracted with EtOAc (200 mL, three times). The combined organic layer was washed with brine (200 mL, twice), dried over anhydrous sodium sulfate, filtered, and concentrated under reduced pressure to yield a residue. The residue was purified by flash column chromatography to afford 3,5-dichloro-4-ethylpyridine (Compound A7-a, 27 g) as a colorless oil. MS calc'd 176.0 (MH⁺), measured 176.0 (MH⁺).

### Intermediate A8

### Benzyl 4-[5-chloro-4-cyclopropyl-6-[(1S)-1-methoxyethyl]-3-pyridyl]piperazine-1-carboxylate

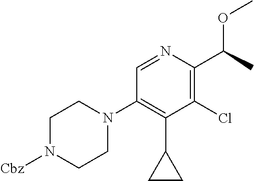 [SMILES: CO[C@@H](C)c1ncc(N2CCN(C(=O)OCc3ccccc3)CC2)c(C2CC2)c1Cl]

The intermediate A8 was prepared in analogy to the preparation of intermediate A1 by using 3,5-dichloro-4-cyclopropyl-pyridine (compound A8-a) instead of 3,5-dichloro-4-methyl-pyridine (compound A1-a).

The compound A8-a was prepared according to the following progress.

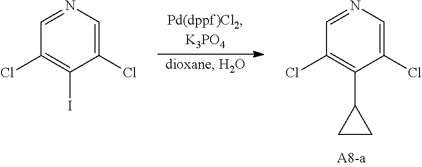 [SMILES: Clc1cncc(Cl)c1I ; Clc1cncc(Cl)c1C1CC1]

To a solution of 3,5-dichloro-4-iodopyridine (CAS #343781-41-9, BD216921-25g, 9.0 g, 32.86 mmol) in 1,4-dioxane (160 mL) and water (40 mL) was added cyclopropylboronic acid (3.7 g, 42.72 mmol), K₃PO₄ (15.6 g, 115.01 mmol), and Pd(dppf)Cl₂ (2.4 g, 3.29 mmol). The mixture was degassed and purged with nitrogen three times, then stirred at 100° C. for 4 hours under a nitrogen atmosphere. After reaction completion, the mixture was allowed to stand for layer separation. The organic layer was concentrated under reduced pressure to yield a residue, which was purified by silica gel column to afford 3,5-dichloro-4-cyclopropylpyridine (compound A8-a, 5.89 g) as a white solid. MS calc'd 187.0 (MH⁺), measured 187.9 (MH).

### Intermediate A9

### 1-[5-bromo-4-(difluoromethyl)-6-[(1S)-1-methoxyethyl]-3-pyridyl]-4-methyl-piperazine

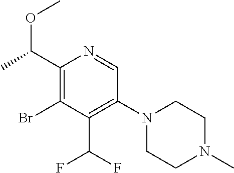 [SMILES: CO[C@@H](C)c1ncc(N2CCN(C)CC2)c(C(F)F)c1Br]

The title intermediate A9 was prepared according to the following scheme:

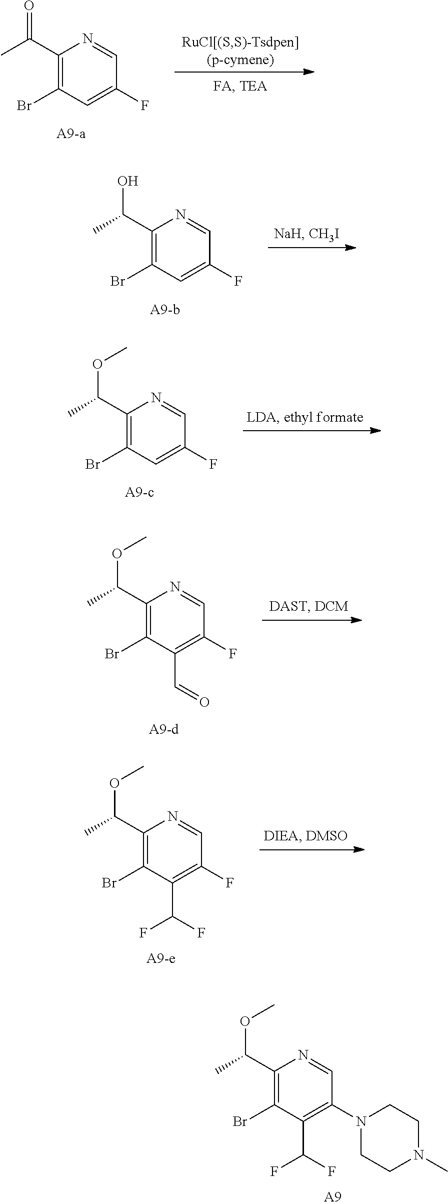

### Step 1: Preparation of (1S)-1-(3-bromo-5-fluoro-2-pyridyl)ethanol (compound A9-b)

To a solution of 1-(3-bromo-5-fluoro-2-pyridyl)ethanone (compound A9-a, CAS #1820641-72-2, BD561603-1g, 3.5 g, 7.54 mmol) in THF (18.3 mL), TEA (65.2 mL, 467.78 mmol) and formic acid (1.74 mL, 45.27 mmol) were added at 20° C. To this mixture was added (S,S)—N-(p-toluenesulfonyl)-1,2-diphenylethanediamine(chloro)(p-cymene)ruthenium(II) (960.0 mg, 1.51 mmol). The mixture was stirred at 45° C. for 3 hours under a nitrogen atmosphere. The reaction mixture was concentrated under vacuum to yield a residue, which was purified by column chromatography and reversed-phase chromatography to afford (1S)-1-(3-bromo-5-fluoro-2-pyridyl)ethanol (compound A9-b, 1.4 g) as a light yellow oil. MS calc'd 219.9 (MH⁺), measured 219.8 (MH⁺).

### Step 2: Preparation of 3-bromo-5-fluoro-2-[(1S)-1-methoxyethyl]pyridine (compound A9-c)

To a solution of (1S)-1-(3-bromo-5-fluoro-2-pyridyl)ethanol (compound A9-b, 1.4 g, 6.36 mmol) in THF (14 mL) was slowly added sodium hydride (198.5 mg, 8.27 mmol, 60% in oil) at 0° C. under a nitrogen atmosphere. The mixture was stirred for 0.5 hours, after which iodomethane (1.8 g, 12.72 mmol) was added at 0° C. The reaction was stirred for an additional 1 hour. After completion, the reaction mixture was poured into ice water (10 mL) and extracted with ethyl acetate (20 mL, three times). The combined organic layer was washed with brine (20 mL), dried over anhydrous sodium sulfate, filtered, and concentrated under reduced pressure to yield a residue. The residue was purified by column chromatography to afford 3-bromo-5-fluoro-2-[(1S)-1-methoxyethyl]pyridine (compound A9-c, 1.3 g) as a light yellow oil. MS calc'd 234.0 (MH⁺), measured 233.9 (MH⁺).

### Step 3: Preparation of 3-bromo-5-fluoro-2-[(1S)-1-methoxyethyl]pyridine-4-carbaldehyde (compound A9-d)

To a solution of 3-bromo-5-fluoro-2-[(1S)-1-methoxyethyl]pyridine (compound A9-c, 2.3 g, 9.83 mmol) in THF (34.5 mL) was added 1 M lithium diisopropylamide (11.8 mL, 11.8 mmol) dropwise at −78° C. under a nitrogen atmosphere. The mixture was stirred at −78° C. for 1 hour, after which ethyl formate (1.6 mL, 19.65 mmol) was added slowly. The mixture was stirred at room temperature for an additional 1 hour. The reaction was quenched by the addition of sat. ammonium chloride aq. (10 mL). The resulting mixture was extracted with ethyl acetate (50 mL, three times). The combined organic layer was washed with brine, dried over anhydrous sodium sulfate, and concentrated under reduced pressure to yield a residue. The residue was purified by silica gel column chromatography to afford 3-bromo-5-fluoro-2-[(15)-1-methoxyethyl]pyridine-4-carbaldehyde (compound A9-d, 1.5 g) as light yellow oil. MS calc'd 280.0 (M+H₂O+H⁺), measured 279.8 (M+H₂O+H⁺).

### Step 4: Preparation of 3-bromo-4-(difluoromethyl)-5-fluoro-2-[(1S)-1-methoxyethyl]pyridine (compound A9-e)

To a solution of 3-bromo-5-fluoro-2-[(15)-1-methoxyethyl]pyridine-4-carbaldehyde (compound A9-d, 1.8 g, 6.87 mmol) in DCM (21.0 mL) was added diethylaminosulfur trifluoride (1.36 mL, 10.3 mmol) under an ice bath. The mixture was stirred at 0° C. for 30 minutes, then warmed to room temperature and stirred for an additional 1 hour. The reaction was quenched with water. The organic layer was separated, dried over anhydrous magnesium sulfate, filtered, and concentrated under reduced pressure to yield a residue. The residue was purified by silica gel column chromatography to afford 3-bromo-4-(difluoromethyl)-5-fluoro-2-[(15)-1-methoxyethyl]pyridine (compound A9-e, 1.5 g) as a light-yellow oil. MS calc'd 284.0 (MH⁺), measured 283.8 (MH⁺).

### Step 5: Preparation of 1-[5-bromo-4-(difluoromethyl)-6-[(1S)-1-methoxyethyl]-3-pyridyl]-4-methyl-piperazine (intermediate A9)

A solution of 1-methylpiperazine (940.2 μL, 8.45 mmol), 3-bromo-4-(difluoromethyl)-5-fluoro-2-[(15)-1-methoxyethyl]pyridine (compound A9-e, 1.2 g, 4.22 mmol), and DIEA (1.5 mL, 8.45 mmol) in DMSO (0.5 mL) was stirred at 120° C. for 24 hours. After completion of the reaction, the mixture was dissolved in ethyl acetate (EA, 80 mL) and washed with water (30 mL, three times) and brine (20 mL). The organic layer was dried over anhydrous sodium sulfate, filtered, and concentrated under reduced pressure to obtain a residue. The residue was purified by silica gel column chromatography to afford 1-[5-bromo-4-(difluoromethyl)-6-[(1S)-1-methoxyethyl]-3-pyridyl]-4-methyl-piperazine (intermediate A9, 1.2 g) as a light yellow solid. MS calc'd 364.1 (MH⁺), measured 263.9 (MH⁺).

### Intermediate A10

### 1-[5-chloro-6-[(1S)-1-methoxyethyl]-4-(trifluoromethyl)-3-pyridyl]-4-methyl-piperazine

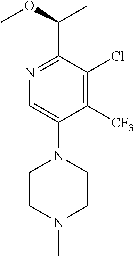 [SMILES: CO[C@@H](C)c1ncc(N2CCN(C)CC2)c(C(F)(F)F)c1Cl]

The title intermediate A10 was prepared according to the following scheme:

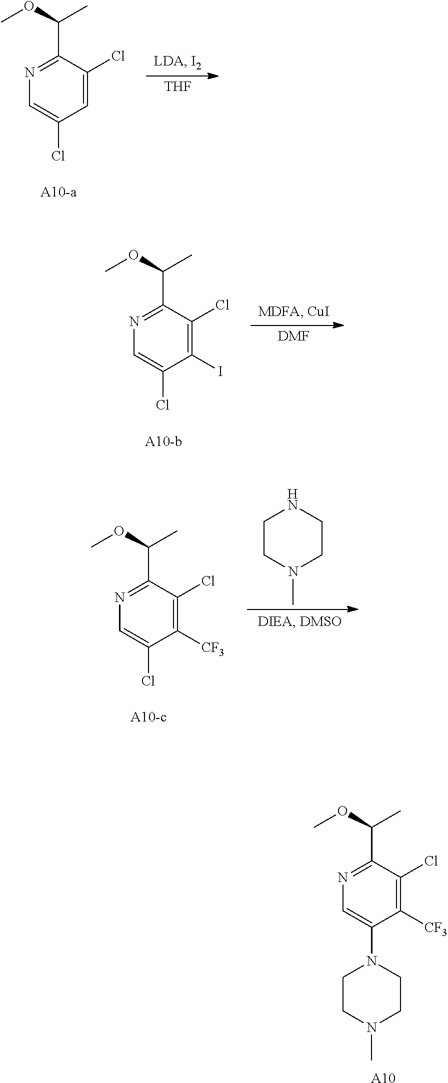

### Step 1: Preparation of 3,5-dichloro-4-iodo-2-[(1S)-1-methoxyethyl]pyridine (compound A10-b)

1. Solution 1: 3,5-dichloro-2-[(1S)-1-methoxyethyl]pyridine (compound A10-a, 14.9 g, 72.31 mmol) dissolved in THF (298 mL).
2. Solution 2: LDA (54.2 mL, 108.46 mmol).
3. Solution 3: Iodine (22.0 g, 86.77 mmol) dissolved in THF (298 mL).

Flow Reactor 1 (FLR1): Solution 1 was pumped (Pump 1, Flow Rate=9.46 mL/min) and Solution 2 was pumped (Pump 2, Flow Rate=3.26 mL/min) into Flow Reactor 1 (FLR1, PFA coil reactor, 1.588 mm ( 1/16″) tubing, internal volume=0.636 mL) at −20.0° C. The residence time in FLR1 was set to 0.05 min.

Flow Reactor 2 (FLR2): The output of FLR1 was combined with Solution 3, which was pumped by Pump 3 (Flow Rate=9.7 mL/min) into Flow Reactor 2 (FLR2, PFA coil reactor, 1.588 mm ( 1/16″) tubing, internal volume=0.636 mL). The residence time in FLR2 was set to 0.028 min.

The reaction mixture was collected in a bottle, then the reaction was quenched with NH₄Cl aq. (300 mL) and the mixture was extracted with EtOAc (150 mL, three times). The combined organic phase was concentrated under reduced pressure to afford the crude product. The crude product was purified by flash silica gel chromatography to yield 3,5-dichloro-4-iodo-2-[(1S)-1-methoxyethyl]pyridine (compound A10-b, 13.3 g) as a white solid. MS calc'd 331.9 (MH⁺), measured 331.9 (MH⁺). ¹H NMR (400 MHz, CHLOROFORM-d) 6=8.49 (s, 1H), 4.93 (q, J=6.4 Hz, 1H), 3.32 (s, 3H), 1.48 (d, J=6.4 Hz, 3H).

### Step 2: Preparation of 3,5-dichloro-2-[(1S)-1-methoxyethyl]-4-(trifluoromethyl)pyridine (compound A10-c)

To a solution of 3,5-dichloro-4-iodo-2-[(15)-1-methoxyethyl]pyridine (compound A10-b, 12.3 g, 37.05 mmol) and CuI (14.1 g, 74.13 mmol) in DMF (120 mL) was slowly added methyl 2,2-difluoro-2-(fluorosulfonyl)acetate (28.5 g, 148.21 mmol) at 20° C. The mixture was stirred at 80° C. for 14 hours under a nitrogen atmosphere. The reaction mixture was poured into ethyl acetate (500 mL) and washed with brine (800 mL, three times). The combined organic phase was dried over anhydrous sodium sulfate and concentrated under reduced pressure to afford the crude residue. The crude product was purified by flash silica gel chromatography to afford 3,5-dichloro-2-[(1S)-1-methoxyethyl]-4-(trifluoromethyl)pyridine (compound A10-c, 9.8 g) as a yellow oil. MS calc'd 274.0 (MH⁺), measured 274.0 (MH⁺).

### Step 3: Preparation of 1-[5-chloro-6-[(1S)-1-methoxyethyl]-4-(trifluoromethyl)-3-pyridyl]-4-methyl-piperazine (intermediate A10)

To a solution of 3,5-dichloro-2-[(15)-1-methoxyethyl]-4-(trifluoromethyl)pyridine (compound A10-c, 9.8 g, 35.76 mmol) in DMSO (98 mL) was added DIEA (12.5 mL, 71.52 mmol) and 1-methylpiperazine (14.3 g, 143.03 mmol). The reaction mixture was stirred at 100° C. for 48 hours under a nitrogen atmosphere. The reaction mixture was poured into ethyl acetate (500 mL) and washed with brine (500 mL, three times). The combined organic layer was concentrated under reduced pressure to obtain the crude product. The crude product was purified by flash silica gel chromatography to afford 1-[5-chloro-6-[(1S)-1-methoxyethyl]-4-(trifluoromethyl)-3-pyridyl]-4-methyl-piperazine (intermediate A10, 9.7 g) as a brown oil. MS calc'd 338.1 (MH⁺), measured 338.1 (MH⁺). ¹H NMR (400 MHz, CDCl₃): δ=8.51 (s, 1H), 5.02 (d, J=6.5 Hz, 1H), 3.33 (s, 3H), 3.13 (t, J=4.7 Hz, 4H), 2.62 (s, 1H), 2.58 (br s, 3H), 2.37 (s, 3H), 1.49 (d, J=6.4 Hz, 3H).

### Intermediate B1

### 7-chloro-2-(4,4,5,5-tetramethyl-1,3,2-dioxaborolan-2-yl)-1,3,4,5-tetrahydrobenzo[cd]indole

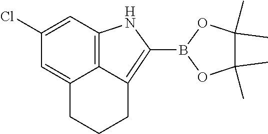 [SMILES: CC1(C)OB(c2[nH]c3cc(Cl)cc4c3c2CCC4)OC1(C)C]

The title intermediate B1 was prepared according to the following scheme:

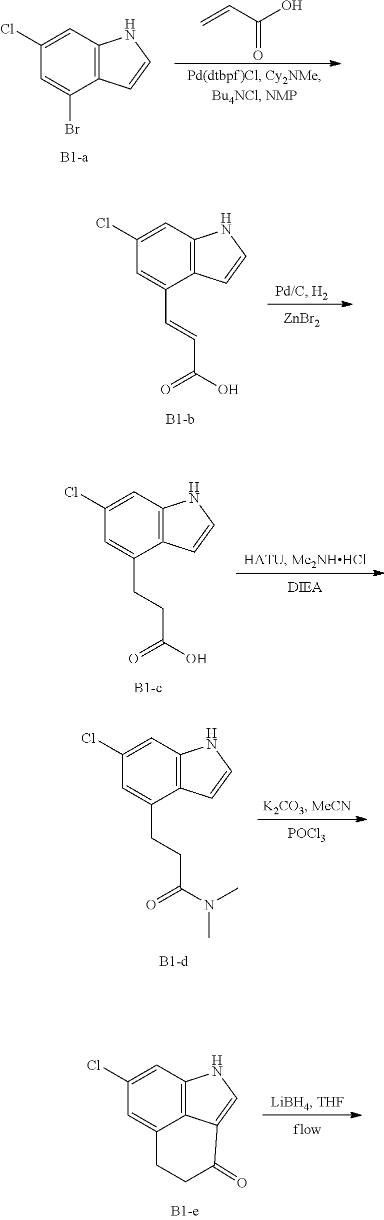

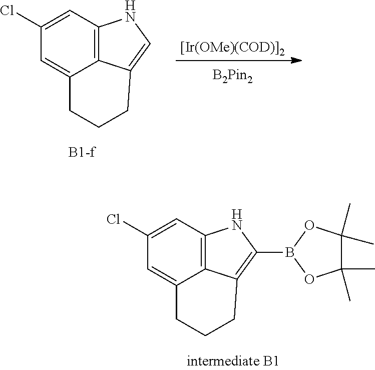

### Step 1: Preparation of (E)-3-(6-chloro-1H-indol-4-yl)prop-2-enoic acid (compound B1-b)

To a mixture of 4-bromo-6-chloro-1H-indole (compound B1-a, 50.0 g, 210.69 mmol) and acrylic acid (23.4 g, 325.4 mmol) in NMP (500 mL), N,N-dicyclohexylmethylamine (203.0 g, 1.0 mol), tetrabutylammonium chloride (6.0 g, 21.7 mmol) and Pd(dtbpf)Cl₂ (14.11 g, 21.7 mmol) were added in one portion. The mixture was stirred at 100° C. for 16 hrs under nitrogen atmosphere. After the reaction was completed, it cooled to room temperature. The reaction mixture was poured into water (2 L) and EtOAc (500 mL). The mixture was filtered through a pad of celite. The filter cake was washed with sat. NaHCO₃ (500 mL) and EtOAc (500 mL). Then layer of the filtrate was separated, the aqueous phase was washed with EtOAc (500 mL, three times). The pH of aqueous phase was adjusted to 2-3 using 6N HCl aq. and extracted with EtOAc (500 mL, three times). The organic phase was dried over Na₂SO₄, filtered and concentrated under vacuum to afford (E)-3-(6-chloro-1H-indol-4-yl)prop-2-enoic acid (compound B1-b, 32.0 g,) as a light yellow solid. ¹H NMR (400 MHz, METHANOL-d₄) δ=7.99 (d, J=16.0 Hz, 1H), 7.46 (s, 1H), 7.40 (d, J=3.2 Hz, 1H), 7.32 (d, J=1.6 Hz, 1H), 6.76-6.71 (m, 1H), 6.60 (d, J=16.0 Hz, 1H).

### Step 2: Preparation of 3-(6-chloro-1H-indol-4-yl)propanoic acid (compound B1-c)

To the mixture of Pd on activated carbon (28.8 g) in methanol (1 L) and THF (125 mL), (E)-3-(6-chloro-1H-indol-4-yl)prop-2-enoic acid (compound B1-b, 30.0 g, 135.4 mmol) and zinc bromide (6.1 g, 27.1 mmol) were added under nitrogen atmosphere, the mixture was degassed under vacuum and purged with H2 for 3 times. The mixture was stirred at 25° C. for 24 hrs under H2 (15 psi) atmosphere. The mixture was filtered; the filtrate was concentrated under vacuum to afford 3-(6-chloro-1H-indol-4-yl)propanoic acid (compound B1-c, 42.0 g, crude) as dark brown oil. MS calc'd 222.0 (M−H)⁻, measured 222.0 (M−H)⁻.

### Step 3: Preparation of 3-(6-chloro-1H-indol-4-yl)-N,N-dimethyl-propanamide (compound B1-d)

To a solution of 3-(6-chloro-1H-indol-4-yl)propanoic acid (compound B1-c, 42.0 g) in DMF (800 mL), DIEA (163.5 mL, 938.93 mmol) and HATU (107.1 g, 281.68 mmol) were added at 0° C. After being stirred at 0° C. for 1 h, the reaction was added with dimethylamine hydrochloride (30.6 g, 375.57 mmol) and stirred at 25° C. for another 2 hrs. The reaction mixture was poured into water (1.5 L), extracted with EtOAc (700 mL, three times). The combined organic phase was washed with brine (1000 mL, twice), dried over Na₂SO₄, filtered and concentrated under vacuum to give a residue. The residue was purified via column chromatography to afford 3-(6-chloro-1H-indol-4-yl)-N,N-dimethyl-propanamide (compound B1-d, 22.0 g) as a brown solid. MS calc'd 251.1 (MH⁺), measured 251.1 (MH⁺).

### Step 4: Preparation of 7-chloro-4,5-dihydro-1H-benzo[cd]indol-3-one (compound B1-e)

To the mixture of afford 3-(6-chloro-1H-indol-4-yl)-N,N-dimethyl-propanamide (compound B1-d, 22.0 g, 87.74 mmol) and K₂CO₃ (32.7 g, 236.91 mmol) in ACN (500 mL), phosphorus oxychloride (29.4 mL, 315.88 mmol) was added slowly at 25° C. The mixture was stirred at 65° C. for 3 hrs. The mixture was cooled to 25° C., poured into water (1 L) and EtOAc (400 mL) slowly and stirred at 25° C. for 30 min. Sat. Na₂CO₃ aq. (1 L) was added slowly, the resulting mixture was stirred at 25° C. for 16 h and extracted with EtOAc (800 mL, three times). The combined organic phase was washed with brine (1 L), dried over anhydrous Na₂SO₄, filtered and concentrated under vacuum, the residue was purified by column to afford 7-chloro-4,5-dihydro-1H-benzo[cd]indol-3-one (compound B1-e, 8.9 g) as a yellow solid. MS calc'd 204.0 (M−H)⁻, measured 204.1 (M−H)⁻.

### Step 5: Preparation of 7-chloro-1,3,4,5-tetrahydrobenzo[cd]indole (compound B1-f)

Solution 1: 7-chloro-4,5-dihydro-1H-benzo[cd]indol-3-one (compound B1-e, 8.9 g, 43.52 mmol) was diluted to 100 mL with methoxycyclopentane (100.0 mL).

Solution 2: LiBH₄ (43.5 mL, 87.05 mmol). The whole reaction process was protected by nitrogen.

The solution 1 was pumped by Pump 1 (S1, P1, 6.888 mL/min) through flow reactor 1 (FLR1, GL, CSTRs, 100 mL, 90° C.), flow reactor 2 (FLR2, GL, CSTRs, 100 mL, 90° C.) and flow reactor 3(FLR3, GL, CSTRs, 100 mL, 90° C.). The solution 2 was pumped by Pump 2 (S2, P2, 3.112 mL/min) through flow reactor 1 (FLR1, GL, CSTRs, 100 mL, 90° C.), flow reactor 2 (FLR2, GL, CSTRs, 100 mL, 90° C.) and flow reactor 3(FLR3, GL, CSTRs, 100 mL, 90° C.).

The residence time of flow reactor 1 was (FLR1, 10 min). The residence time of flow reactor 2 was (FLR2, 10 min). The residence time of flow reactor 3 was (FLR3, 10 min). The mixture was collected in a bottle after Pump 1 and Pump 2 were started.

The mixture was quenched with NH₄Cl aq. at 0° C. The mixture was extracted with EtOAc (150 mL, three times). The combined organic phase was washed with brine (300 mL), dried over anhydrous Na₂SO₄, filtered and concentrated under vacuum to get a residue, which was purified via column chromatography to afford 7-chloro-1,3,4,5-tetrahydrobenzo[cd]indole (compound B1-f, 8.26 g) as pink oil. MS calc'd 190.1 (M−H)⁻, measured 190.1 (M−H)⁻.

### Step 6: Preparation of 7-chloro-2-(4,4,5,5-tetramethyl-1,3,2-dioxaborolan-2-yl)-1,3,4,5-tetrahydrobenzo[cd]indole (intermediate B1)

To a solution of 7-chloro-1,3,4,5-tetrahydrobenzo[cd]indole (compound B1-f, 3.1 g, 19.31 mmol) and bis(pinacolato)diboron (4.9 g, 19.31 mmol) in THF (100 mL), pinacolborane (691.8 mg, 5.41 mmol), 4-tert-butyl-2-(4-tert-butylpyridin-2-yl)pyridine (155.4 mg, 0.58 mmol) and [Ir(OMe)(COD)]2 (209.4 mg, 0.29 mmol) were added. The mixture was stirred at 60° C. under nitrogen atmosphere for 0.75 h. The reaction mixture was concentrated under vacuum to give a residue. The residue was purified via column chromatography to afford 7-chloro-2-(4,4,5,5-tetramethyl-1,3,2-dioxaborolan-2-yl)-1,3,4,5-tetrahydrobenzo[cd]indole (intermediate B1, 6.7 g) as yellow oil. MS calc'd 318.1 (MH⁺), measured 318.1 (MH⁺).

### Intermediate B2

### 6-bromo-2-(4,4,5,5-tetramethyl-1,3,2-dioxaborolan-2-yl)-9-oxa-3-azatricyclo[6.4.1.0^{4,13}]trideca-1,4(13),5,7-tetraene

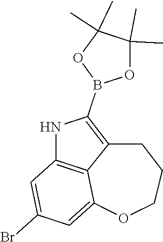 [SMILES: CC1(C)OB(c2[nH]c3cc(Br)cc4c3c2CCCO4)OC1(C)C]

The title intermediate B2 was prepared according to the following scheme:

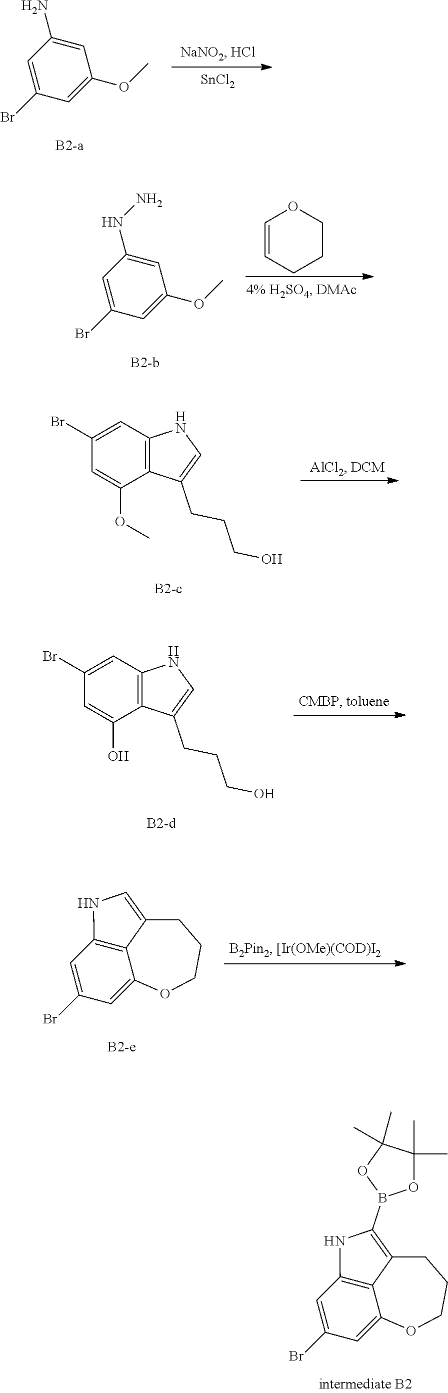

### Step 1: Preparation of (3-bromo-5-methoxy-phenyl)hydrazine (compound B2-b)

To a solution of 3-bromo-5-methoxyaniline (compound B2-a, 25.0 g, 123.73 mmol) in conc. HCl (200.0 mL, 2.4 mol), a solution of NaNO₂ (8.8 g, 126.83 mmol) in water (36 mL) was added at −10° C., and the mixture was stirred at −10° C. for 30 min. A solution of SnCl₂·H₂O (106.3 g, 470.86 mmol) in conc. HCl (600.0 mL, 7.2 mol) was added dropwise to the reaction mixture at −10° C. and stirred for 15 min, then the reaction mixture was stirred at 20° C. for 30 min. The reaction mixture was cooled to 0° C. The mixture was basified by 40% NaOH solution until pH reached 9-10, and extracted by EtOAc (1 L, four times). The organic phase was dried over Na₂SO₄, filtered and concentrated under reduced pressure to give a residue which was purified via column chromatography to afford (3-bromo-5-methoxy-phenyl)hydrazine (compound B2-b, 22.2 g) as a brown oil. MS calc'd 217.0 (MH⁺), measured 217.1 (MH⁺).

### Step 2: Preparation of 3-(6-bromo-4-methoxy-1H-indol-3-yl)propan-1-ol (compound B2-c)

To a solution of (3-bromo-5-methoxy-phenyl)hydrazine (compound B2-b, 18.0 g, 82.92 mmol) in 4% H₂SO₄ (1.8 L) and DMA (15 mL), dihydropyran (7.6 mL, 82.91 mmol) was added dropwise, and the mixture was stirred at 100° C. for 12 hrs. The reaction mixture was poured into water (200 mL), and the mixture was extracted with EtOAc (100 mL, three times). The combined organic layer was washed with brine (100 mL), dried over Na₂SO₄, filtered and concentrated under vacuum to give a residue which was purified via column chromatography (EtOAc in PE: 10%-50%) to give a crude product. The crude product was purified by reversed flash chromatography to afford 3-(6-bromo-4-methoxy-1H-indol-3-yl)propan-1-ol (compound B2-c, 1.8 g) as a yellow solid. The compound was confirmed by 2H NMR. MS calc'd 282.0 (M−H)⁻, measured 282.0 (M−H)⁻; ¹H NMR (400 MHz, DMSO) δ=10.84 (br s, 1H), 7.09 (d, J=1.6 Hz, 1H), 6.94 (d, J=2.0 Hz, 1H), 6.54 (d, J=1.2 Hz, 1H), 4.38 (t, J=5.2 Hz, 1H), 3.83 (s, 3H), 3.45-3.41 (m, 2H), 2.75 (t, J=7.6 Hz, 2H), 1.79-1.69 (m, 2H).

### Step 3: Preparation of 6-bromo-3-(3-hydroxypropyl)-1H-indol-4-ol (compound B2-d)

To a solution of 3-(6-bromo-4-methoxy-1H-indol-3-yl)propan-1-ol (compound B2-c, 1.5 g, 5.28 mmol) in DCM (30 mL), AlCl₃ (2.1 g, 15.86 mmol) was added at 0° C., and the mixture was stirred at 20° C. for 15 hrs. The reaction mixture was added with MeOH (50 mL) and concentrated under vacuum to give a residue which was purified by reversed flash chromatography to afford 6-bromo-3-(3-hydroxypropyl)-1H-indol-4-ol (compound B2-d, 1.2 g) as colorless oil. MS calc'd 270.0 (MH⁺), measured 269.9 (MH⁺).

### Step 4: Preparation of 6-bromo-9-oxa-3-azatricyclo[6.4.1.0^{4,13}]trideca-1,4(13),5,7-tetraene (compound B2-e)

To a solution of 6-bromo-3-(3-hydroxypropyl)-1H-indol-4-ol (compound B2-d, 1.2 g, 4.44 mmol) in toluene (120 mL), CMBP (5.4 g, 22.31 mmol) was added at 0° C. The mixture was degassed and purged with N2 three times, and it was stirred at 100° C. for 15 hrs under N2. The reaction mixture was concentrated under vacuum to give a residue which was purified via column chromatography to afford 6-bromo-9-oxa-3-azatricyclo[6.4.1.0^{4,13}]trideca-1,4(13),5,7-tetraene (compound B2-e, 840.0 mg) as yellow oil. MS calc'd 250.0 (M−H)⁻, measured 250.1 (M−H)⁻.

### Step 5: Preparation of 6-bromo-2-(4,4,5,5-tetramethyl-1,3,2-dioxaborolan-2-yl)-9-oxa-3-azatricyclo[6.4.1.0^{4,13}]trideca-1,4(13),5,7-tetraene (intermediate B2)

To a mixture of 6-bromo-9-oxa-3-azatricyclo[6.4.1.0^{4,13}]trideca-1,4(13),5,7-tetraene (compound B2-e, 600.0 mg, 2.38 mmol) in THF, bis(pinacolato)diboron (906.5 mg, 3.57 mmol) (15 mL), 4,4′-di-tert-butyl-2,2′-bipyridin (95.81 mg, 0.36 mmol) and [Ir(OMe)(COD)]2 (236.6 mg, 0.36 mmol) were added. The mixture was degassed and purged with N2 three times, and the mixture was stirred at 75° C. for 15 hrs under N2 atmosphere. The reaction mixture was concentrated under vacuum to give the residue which was purified via column chromatography to afford 6-bromo-2-(4,4,5,5-tetramethyl-1,3,2-dioxaborolan-2-yl)-9-oxa-3-azatricyclo[6.4.1.0^{4,13}]trideca-1,4(13),5,7-tetraene (intermediate B2, 748.0 mg) as an off-white gum. MS calc'd 378.1 (MH⁺), measured 378.1 (MH⁺).

### Intermediate B3

### 6-chloro-2-(4,4,5,5-tetramethyl-1,3,2-dioxaborolan-2-yl)-1-azatricyclo[6.3.1.0^{4,12}]dodeca-2,4(12),5,7-tetraene

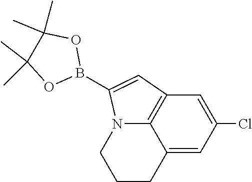 [SMILES: CC1(C)OB(c2cc3cc(Cl)cc4c3n2CCC4)OC1(C)C]

The intermediate B3 was prepared in analogy to the preparation of intermediate B1 by using 6-chloro-1-azatricyclo[6.3.1.0^{4,12}]dodeca-2,4(12),5,7-tetraene (compound B3-a) instead of 7-chloro-1,3,4,5-tetrahydrobenzo[cd]indole (compound B1-f). The synthesis of compound B3-a was disclosed in WO 03/076442 A1, page 25, as Prep. 8.

### Intermediate C1

### Methyl (3S)-1-[(2S)-3-(4-bromothiazol-2-yl)-2-(tert-butoxycarbonylamino)propanoyl]hexahydropyridazine-3-carboxylate

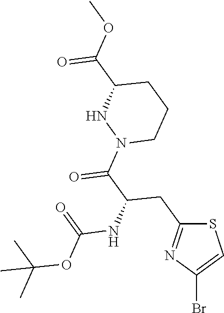 [SMILES: COC(=O)[C@@H]1CCCN(C(=O)[C@H](Cc2nc(Br)cs2)NC(=O)OC(C)(C)C)N1]

The intermediate C1 was prepared according to the following scheme:

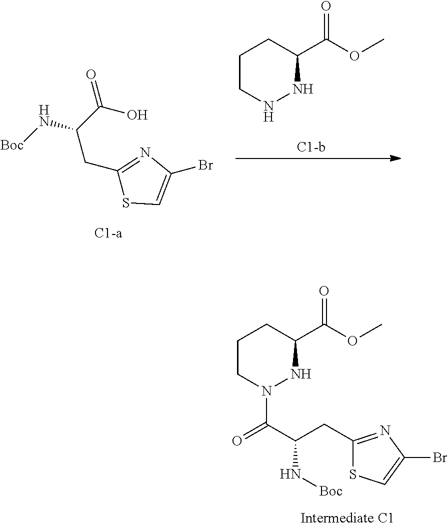

To a solution of (2S)-3-(4-bromothiazol-2-yl)-2-(tert-butoxycarbonylamino)propanoic acid (synthesis of compound C1-a refers to WO 2024/169914 compound B8, 3.1 g, 8.83 mmol) in DCM (50 mL), methyl (3S)-hexahydropyridazine-3-carboxylate;hydrochloride (compound C1-b, 2.4 g, 13.24 mmol), EDCI HCl (3.4 g, 17.65 mmol), 1-hydroxybenzotriazole (238.5 mg, 1.77 mmol) and NMM (9.92 mL, 88.26 mmol) were added at 0° C. After being stirred at 25° C. for 1 hour, the reaction mixture was diluted with water (60 mL) and extracted with EtOAc (60 mL, three times). The combined organic layer was washed with brine (100 mL), dried over anhydrous sodium sulfate, filtered and the filtrate was concentrated under vacuum. The residue was purified by silica gel column and eluted with ethyl acetate in petroleum ether (10˜30%) to afford methyl (3S)-1-[(2S)-3-(4-bromothiazol-2-yl)-2-(tert-butoxycarbonylamino)propanoyl]hexahydropyridazine-3-carboxylate (intermediate C1, 2.4 g). MS calc'd 477(MH⁺), measured 476.9 (MH⁺).

### Intermediate C2

### Methyl (4S)-2-[(2S)-3-(4-bromothiazol-2-yl)-2-(tert-butoxycarbonylamino)propanoyl]-2,3-diazabicyclo[3.1.1]heptane-4-carboxylate

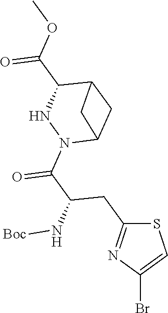 [SMILES: COC(=O)[C@H]1NN(C(=O)[C@H](Cc2nc(Br)cs2)NC(=O)OC(C)(C)C)C2CC1C2]

The intermediate C2 was prepared according to the following scheme:

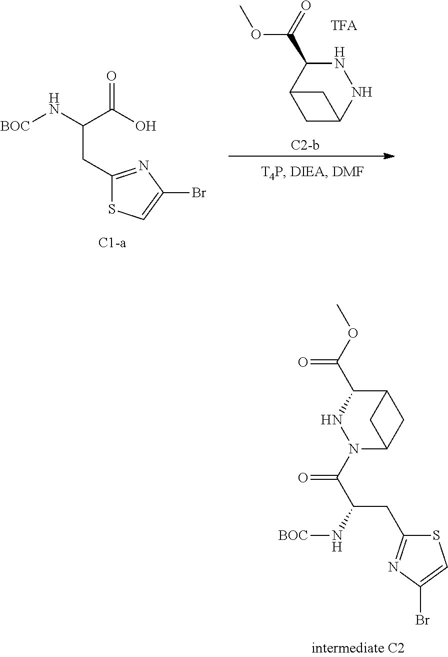 [SMILES: CC(C)(C)OC(=O)NC(Cc1nc(Br)cs1)C(=O)O ; COC(=O)[C@H]1NNC2CC1C2 ; COC(=O)[C@H]1NN(C(=O)[C@H](Cc2nc(Br)cs2)NC(=O)OC(C)(C)C)C2CC1C2]

To a solution of methyl (4S)-2,3-diazabicyclo[3.1.1]heptane-4-carboxylate;2,2,2-trifluoroacetic acid (synthesis of compound C2-b refers to WO 2024/067857 compound M9, 2.9 g, 8.53 mmol) and (2S)-3-(4-bromothiazol-2-yl)-2-(tert-butoxycarbonylamino)propanoic acid (compound C1-a, 3.0 g, 8.53 mmol) in DMF (30 mL) was added DIEA (22.04 g, 170.53 mmol) at 0° C. The reaction was stirred at 0° C. for 5 min. T4P (12.3 g, 17.05 mmol) was then added to the mixture at 0° C. The reaction was stirred at 20° C. for 1 h. The reaction was quenched with water (300 mL), and then extracted with EA (100 mL, three times). The combined organic layer was washed with brine (100 mL), dried over Na₂SO₄ and concentrated to give crude product. The crude product was purified by flash chromatography column to give methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2, 4.0 g) as light yellow oil. MS calc'd 489.1 (MH⁺), measured 489.1 (MH⁺).

### Intermediate C3

### Methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate

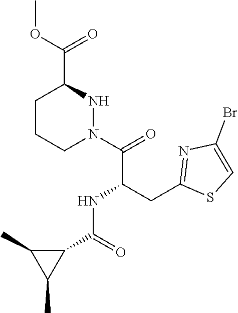 [SMILES: COC(=O)[C@@H]1CCCN(C(=O)[C@H](Cc2nc(Br)cs2)NC(=O)[C@H]2[C@H](C)[C@@H]2C)N1]

The title intermediate C3 was prepared according to the following scheme:

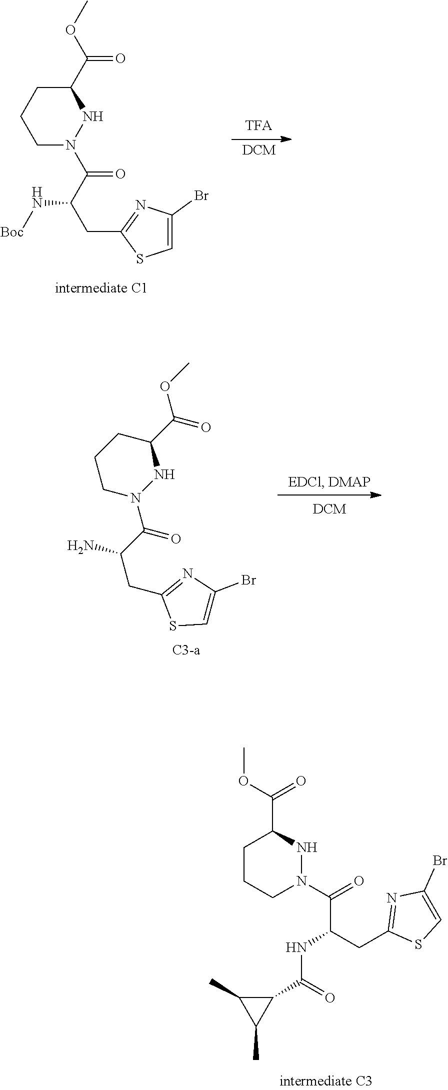 [SMILES: COC(=O)[C@@H]1CCCN(C(=O)[C@H](Cc2nc(Br)cs2)NC(=O)OC(C)(C)C)N1 ; COC(=O)[C@@H]1CCCN(C(=O)[C@@H](N)Cc2nc(Br)cs2)N1 ; COC(=O)[C@@H]1CCCN(C(=O)[C@H](Cc2nc(Br)cs2)NC(=O)[C@H]2[C@H](C)[C@@H]2C)N1]

### Step 1: Preparation of methyl (3S)-1-[(2S)-2-amino-3-(4-bromothiazol-2-yl)propanoyl]hexahydropyridazine-3-carboxylate (compound C3-a)

To a solution of methyl (3S)-1-[(2S)-3-(4-bromothiazol-2-yl)-2-(tert-butoxycarbonylamino)propanoyl]hexahydropyridazine-3-carboxylate (intermediate C1, 47.0 g, 98.46 mmol) in DCM (600 mL) was added TFA (200.0 mL, 2595.97 mmol) at 0° C. The reaction mixture was stirred at 20° C. for 2 hrs. The reaction mixture was concentrated under vacuum to remove DCM and co-evaporated with DCM (200 mL) three times to give a residue. The residue was dissolved in DCM (150 mL) and pH was adjusted to 8 with sat. NaHCO₃ aq., then the mixture was extracted with DCM:MeOH (10:1, 110 mL, four times), washed with brine (300 mL), dried over Na₂SO₄, filtered and concentrated under vacuum to give a residue. The residue was dissolved in EtOAc (200 mL) and then concentrated under vacuum again to give methyl (3S)-1-[(2S)-2-amino-3-(4-bromothiazol-2-yl)propanoyl]hexahydropyridazine-3-carboxylate (compound C3-a, 33.0 g) as yellow gum. MS calc'd 377.0 (MH⁺), measured 377.0 (MH⁺).

### Step 2: Preparation of methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3)

To a solution of methyl (3S)-1-[(2S)-2-amino-3-(4-bromothiazol-2-yl)propanoyl]hexahydropyridazine-3-carboxylate (compound C3-a, 33.0 g, 87.47 mmol,) and (1r,2S,3R)-2,3-dimethylcyclopropanecarboxylic acid (15.0 g, 131.42 mmol) in DCM (400 mL), DMAP (32.0 g, 261.93 mmol) and EDCI HCl (20.2 g, 105.11 mmol) were added at 0° C. The reaction mixture was stirred at 20° C. for 2 hrs. The reaction mixture was washed with sat. citric acid aq. (300 mL, twice) and sat. NaHCO₃ aq. (300 mL, twice), brine (300 mL, twice), dried over Na₂SO₄, filtered then concentrated under vacuum and co-evaporated with ACN (150 mL) to give methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3, 37.0 g) as a white solid. MS calc'd 473.1 (MH⁺), measured 473.1 (MH⁺).

### Intermediate C4

### [2-[(2S)-2-(tert-butoxycarbonylamino)-3-[(3S)-3-methoxycarbonylhexahydropyridazin-1-yl]-3-oxo-propyl]thiazol-4-yl]boronic acid

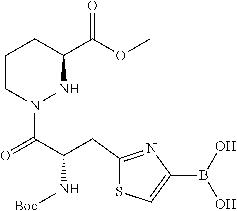 [SMILES: COC(=O)[C@@H]1CCCN(C(=O)[C@H](Cc2nc(B(O)O)cs2)NC(=O)OC(C)(C)C)N1]

The title intermediate C4 was prepared according to the following scheme:

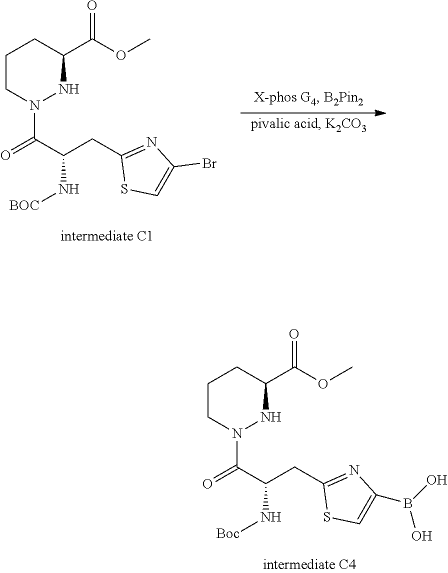 [SMILES: COC(=O)[C@@H]1CCCN(C(=O)[C@H](Cc2nc(Br)cs2)NC(=O)OC(C)(C)C)N1 ; COC(=O)[C@@H]1CCCN(C(=O)[C@H](Cc2nc(B(O)O)cs2)NC(=O)OC(C)(C)C)N1]

To a solution of methyl (3S)-1-[(2S)-3-(4-bromothiazol-2-yl)-2-(tert-butoxycarbonylamino)propanoyl]hexahydropyridazine-3-carboxylate (intermediate C1, 3.0 g, 6.28 mmol) and bis(pinacolato)diboron (6.4 g, 25.14 mmol) in 2-methyltetrahydrofuran (63 mL), pivalic acid (320.9 mg, 3.14 mmol), XPhos Pd G4 (540.5 mg, 0.63 mmol) and K₂CO₃ (2.6 g, 18.85 mmol) were added. Then the mixture was degassed with N2 (this sequence was repeated three times), stirred at 60° C. for 3 hrs under N2, then dried under vacuum and purified via reversed-phase flash chromatography to afford [2-[(2S)-2-(tert-butoxycarbonylamino)-3-[(3S)-3-methoxycarbonylhexahydropyridazin-1-yl]-3-oxo-propyl]thiazol-4-yl]boronic acid (intermediate C4, 2.0 g) as a white solid. MS calc'd 443.2 (MH⁺), measured 443.2 (MH⁺).

### Intermediate C5

### [2-[(2S)-2-(tert-butoxycarbonylamino)-3-[(4S)-4-methoxycarbonyl-2,3-diazabicyclo[3.1.1]heptan-2-yl]-3-oxo-propyl]thiazol-4-yl]boronic acid

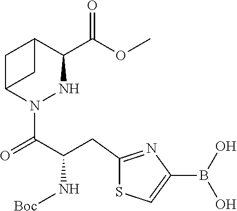 [SMILES: COC(=O)[C@H]1NN(C(=O)[C@H](Cc2nc(B(O)O)cs2)NC(=O)OC(C)(C)C)C2CC1C2]

The intermediate C5 was prepared in analogy to the preparation of intermediate C4 by using methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of methyl (3S)-1-[(2S)-3-(4-bromothiazol-2-yl)-2-(tert-butoxycarbonylamino)propanoyl]hexahydropyridazine-3-carboxylate (intermediate C1).

### Intermediate C6

### Methyl (4S)-2-[(2S)-3-(4-bromothiazol-2-yl)-2-[[(1r,2S,3R)-2,3-dimethylcyclopropanecarbonyl]amino]propanoyl]-2,3-diazabicyclo[3.1.1]heptane-4-carboxylate

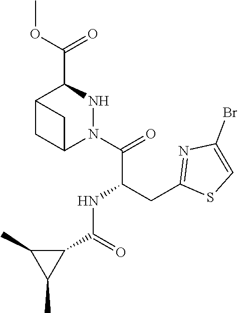 [SMILES: COC(=O)[C@H]1NN(C(=O)[C@H](Cc2nc(Br)cs2)NC(=O)[C@H]2[C@H](C)[C@@H]2C)C2CC1C2]

The intermediate C6 was prepared in analogy to the preparation of intermediate C3 by using methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of methyl (3S)-1-[(2S)-3-(4-bromothiazol-2-yl)-2-(tert-butoxycarbonylamino)propanoyl]hexahydropyridazine-3-carboxylate (intermediate C1).

### Intermediate D1

### Benzyl 4-[(5P)-5-[1l-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate

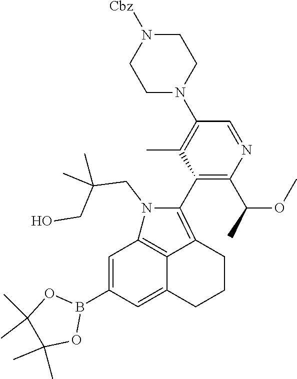

The title intermediate D1 was prepared according to the following scheme:

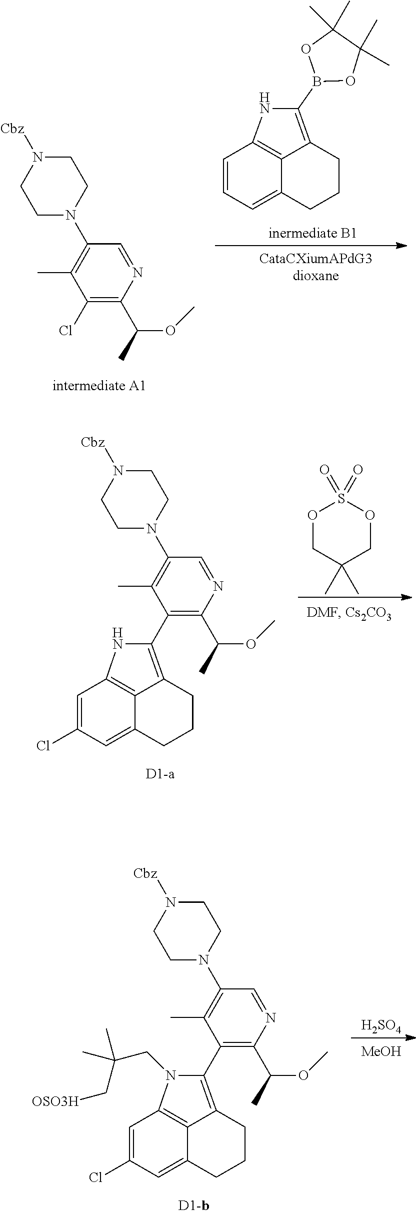

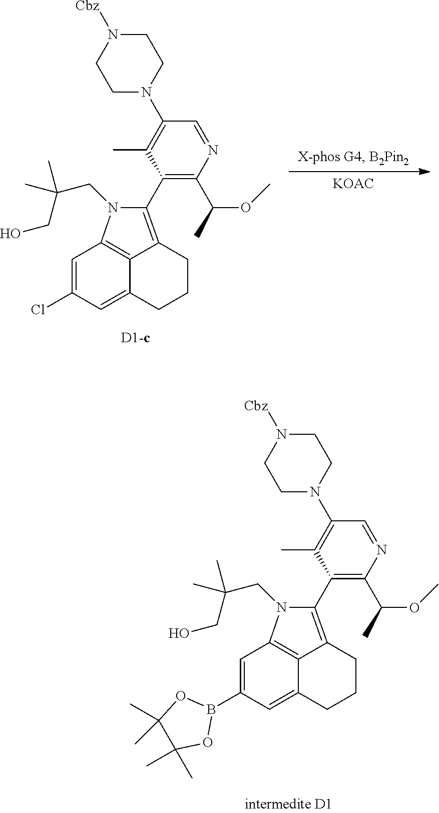

### Step 1: Preparation of benzyl 4-[5-(7-chloro-1,3,4,5-tetrahydrobenzo[cd]indol-2-yl)-6-[(1S)-1-methoxyethyl]-4-methyl-3-pyridyl]piperazine-1-carboxylate (Compound D1-a)

To a solution of 7-chloro-2-(4,4,5,5-tetramethyl-1,3,2-dioxaborolan-2-yl)-1,3,4,5-tetrahydrobenzo[cd]indole (intermediate B1, 3.2 g, 5.99 mmol) and benzyl 4-[5-chloro-6-[(1S)-1-methoxyethyl]-4-methyl-3-pyridyl]piperazine-1-carboxylate (intermediate A1, 2.2 g, 5.45 mmol) in 1,4-dioxane (80 mL) and water (16 mL), K₃PO₄ (3.5 g, 16.34 mmol) and cataCXiumA-Pd-G3 (198.3 mg, 0.27 mmol) were added. The mixture was degassed and purged with nitrogen three times and stirred at 60° C. for 0.5 h. The reaction mixture was filtered, and the filtrate was concentrated under vacuum to give a residue. The residue was purified via column chromatography to afford benzyl 4-[5-(7-chloro-1,3,4,5-tetrahydrobenzo[cd]indol-2-yl)-6-[(1S)-1-methoxyethyl]-4-methyl-3-pyridyl]piperazine-1-carboxylate (compound D1-a, 900.0 mg) as a light brown solid. MS calc'd 559.2 (MH⁺), measured 559.2 (MH⁺).

### Step 2: Preparation of benzyl 4-[5-[7-chloro-1-(2,2-dimethyl-3-sulfooxy-propyl)-4,5-dihydro-3H-benzo[cd]indol-2-yl]-6-[(1S)-1-methoxyethyl]-4-methyl-3-pyridyl]piperazine-1-carboxylate (compound D1-b)

To a solution of benzyl 4-[5-(7-chloro-1,3,4,5-tetrahydrobenzo[cd]indol-2-yl)-6-[(1S)-1-methoxyethyl]-4-methyl-3-pyridyl]piperazine-1-carboxylate (compound D1-a, 800.0 mg, 1.43 mmol) in DMF (3 mL), Cs₂CO₃ (932.4 mg, 2.86 mmol) and 5,5-dimethyl-1,3,2-dioxathiane 2,2-dioxide (285.4 mg, 1.72 mmol) were added. The reaction mixture was stirred at 125° C. for 1 h. The reaction mixture was filtered and the filtrate was concentrated under vacuum to get a residue which was purified by reversed phase chromatography to afford benzyl 4-[5-[7-chloro-1-(2,2-dimethyl-3-sulfooxy-propyl)-4,5-dihydro-3H-benzo[cd]indol-2-yl]-6-[(15)-1-methoxyethyl]-4-methyl-3-pyridyl]piperazine-1-carboxylate (compound D1-b, 800.0 mg) as an off-white solid. MS calc'd 725.3 (MH⁺), measured 725.4 (MH⁺).

### Step 3: Preparation of benzyl 4-[(5P)-5-[7-chloro-1-(3-hydroxy-2,2-dimethyl-propyl)-4,5-dihydro-3H-benzo[cd]indol-2-yl]-6-[(1S)-1-methoxyethyl]-4-methyl-3-pyridyl]piperazine-1-carboxylate (compound D1-c)

To a solution of benzyl 4-[5-[7-chloro-1-(2,2-dimethyl-3-sulfooxy-propyl)-4,5-dihydro-3H-benzo[cd]indol-2-yl]-6-[(15)-1-methoxyethyl]-4-methyl-3-pyridyl]piperazine-1-carboxylate (compound D1-b, 800.0 mg, 1.1 mmol) in methanol (0.8 mL), H₂SO₄ (800.0 mg, 8.16 mmol) was added dropwise. The mixture was stirred at 40° C. for 2 hrs. The reaction mixture was added into sat. NaHCO₃ aq. (300 mL), extracted with EtOAc (100 mL, four times). The combined organic layer was washed with brine (200 mL), dried over Na₂SO₄, filtered and concentrated under vacuum to give a residue. The residue was purified via column chromatography to afford benzyl 4-[(5P)-5-[7-chloro-1-(3-hydroxy-2,2-dimethyl-propyl)-4,5-dihydro-3H-benzo[cd]indol-2-yl]-6-[(1S)-1-methoxyethyl]-4-methyl-3-pyridyl]piperazine-1-carboxylate (compound D1-c, 440.0 mg, faster eluted) as a yellow solid. MS calc'd 645.3 (MH⁺), measured 645.4 (MH⁺).

### Step 4: Preparation of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1)

To a solution of benzyl 4-[(5P)-5-[7-chloro-1-(3-hydroxy-2,2-dimethyl-propyl)-4,5-dihydro-3H-benzo[cd]indol-2-yl]-6-[(1S)-1-methoxyethyl]-4-methyl-3-pyridyl]piperazine-1-carboxylate (compound D1-c, 440.0 mg, 0.68 mmol) and bis(pinacolato)diboron (865.8 mg, 3.41 mmol) in toluene (10 mL), KOAc (307 mg, 3.41 mmol) and Xphos-G4 (117.36 mg, 0.14 mmol) were added. The reaction mixture was degassed and purged with N2 three times, and then the mixture was stirred at 110° C. for 0.25 h. The reaction mixture was filtered and the filtrate was concentrated under vacuum to give a residue, the residue was purified by reversed phase chromatography to afford benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1, 284.0 mg) as a white solid. MS calc'd 737.4, (MH⁺), measured 737.5, (MH⁺).

### Intermediate D2

### Benzyl 4-[(5P)-5-[3-(3-hydroxy-2,2-dimethyl-propyl)-6-(4,4,5,5-tetramethyl-1,3,2-dioxaborolan-2-yl)-9-oxa-3-azatricyclo[6.4.1.0^{4,13}]trideca-1,4(13),5,7-tetraen-2-yl]-6-[(1S)-1-methoxyethyl]-3-pyridyl]piperazine-1-carboxylate

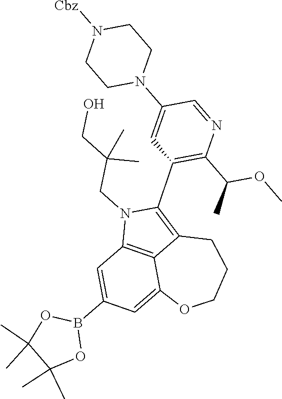

The intermediate D2 was prepared in analogy to the preparation of intermediate D1 by using 6-bromo-2-(4,4,5,5-tetramethyl-1,3,2-dioxaborolan-2-yl)-9-oxa-3-azatricyclo[6.4.1.0^{4,13}]trideca-1,4(13),5,7-tetraene (intermediate B2) and benzyl 4-[5-bromo-6-[(1S)-1-methoxyethyl]-3-pyridyl]piperazine-1-carboxylate (CAS #2641451-78-5, see WO 2024/169914 compound A5) instead of 7-chloro-2-(4,4,5,5-tetramethyl-1,3,2-dioxaborolan-2-yl)-1,3,4,5-tetrahydrobenzo[cd]indole (intermediate B1) and benzyl 4-[5-chloro-6-[(1S)-1-methoxyethyl]-4-methyl-3-pyridyl]piperazine-1-carboxylate (intermediate A1).

### Intermediate D3

### Benzyl 4-[(5M)-5-[1-[3-[tert-butyl(dimethyl)silyl]oxy-2,2-dimethyl-propyl]-7-(4,4,5,5-tetramethyl-1,3,2-dioxaborolan-2-yl)-4,5-dihydro-3H-benzo[cd]indol-2-yl]-4-fluoro-6-[(1S)-1-methoxyethyl]-3-pyridyl]piperazine-1-carboxylate

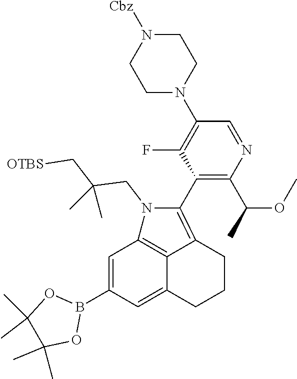

The title intermediate D3 was prepared according to the following scheme:

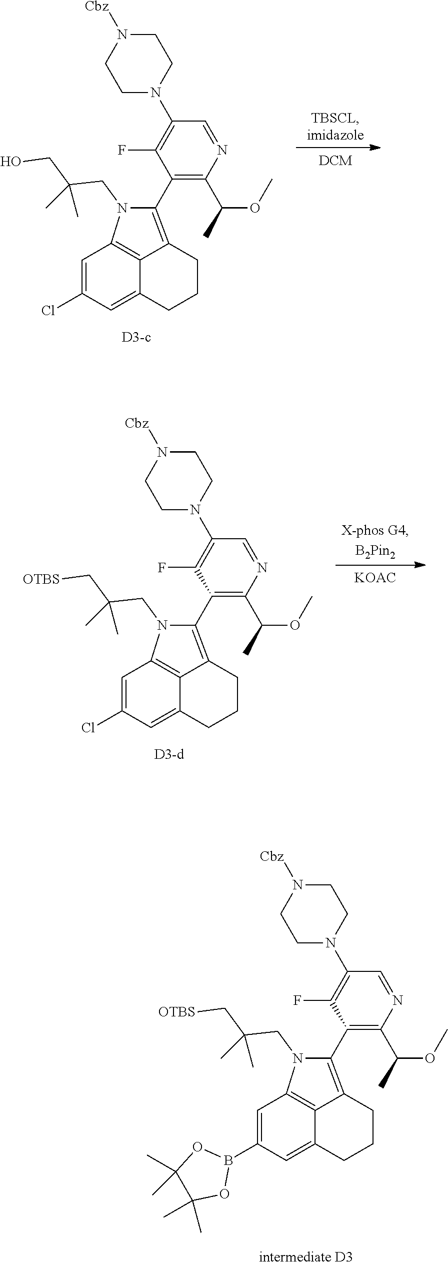

### Step 1: Preparation of benzyl 4-[(5M)-5-[1-[3-[tert-butyl(dimethyl)silyl]oxy-2,2-dimethyl-propyl]-7-chloro-4,5-dihydro-3H-benzo[cd]indol-2-yl]-4-fluoro-6-[(1S)-1-methoxyethyl]-3-pyridyl]piperazine-1-carboxylate (compound D3-d)

To a solution of benzyl 4-[(5M)-5-[7-chloro-1-(3-hydroxy-2,2-dimethyl-propyl)-4,5-dihydro-3H-benzo[cd]indol-2-yl]-4-fluoro-6-[(1S)-1-methoxyethyl]-3-pyridyl]piperazine-1-carboxylate (compound D3-c, 430 mg, 0.56 mmol) and imidazole (225.5 mg, 3.31 mmol) in DCM (10 mL), TBSCl (300 mg, 2 mmol) was added at 0° C. The mixture was stirred at 25° C. for 4 hrs. The reaction mixture was poured into water (40 mL) and extracted with EtOAc (20 mL, three times). The combined organic layer was washed with brine (30 mL), dried over Na₂SO₄, filtered and concentrated under vacuum to give a residue which was purified via column chromatography to afford benzyl 4-[(5M)-5-[1-[3-[tert-butyl(dimethyl)silyl]oxy-2,2-dimethyl-propyl]-7-chloro-4,5-dihydro-3H-benzo[cd]indol-2-yl]-4-fluoro-6-[(1S)-1-methoxyethyl]-3-pyridyl]piperazine-1-carboxylate (compound D3-d, 500.0 mg) as colorless oil. MS calc'd 763.4 (MH⁺), measured 763.4 (MH⁺).

The compound D3-c was prepared in analogy to the preparation of compound D1-c by using benzyl 4-[4-fluoro-5-iodo-6-[(1S)-1-methoxyethyl]-3-pyridyl]piperazine-1-carboxylate (intermediate A2) instead of benzyl 4-[5-chloro-6-[(1S)-1-methoxyethyl]-4-methyl-3-pyridyl]piperazine-1-carboxylate (intermediate A1).

### Step 2: Preparation of benzyl 4-[(5M)-5-[1-[3-[tert-butyl(dimethyl)silyl]oxy-2,2-dimethyl-propyl]-7-(4,4,5,5-tetramethyl-1,3,2-dioxaborolan-2-yl)-4,5-dihydro-3H-benzo[cd]indol-2-yl]-4-fluoro-6-[(1S)-1-methoxyethyl]-3-pyridyl]piperazine-1-carboxylate (intermediate D3)

To a solution of benzyl 4-[(5M)-5-[1-[3-[tert-butyl(dimethyl)silyl]oxy-2,2-dimethyl-propyl]-7-chloro-4,5-dihydro-3H-benzo[cd]indol-2-yl]-4-fluoro-6-[(15)-1-methoxyethyl]-3-pyridyl]piperazine-1-carboxylate (compound D3-c, 400.0 mg, 0.52 mmol) and bis(pinacolato)diboron (665.2 mg, 2.62 mmol) in toluene (8 mL), KOAc (256.8 mg, 2.62 mmol) and XPhos-Pd-G4 (90.2 mg, 0.1 mmol) were added. The mixture was degassed and purged with N2 for three times, and the mixture was stirred at 110° C. for 1.5 hrs under N2 atmosphere. The reaction mixture was concentrated under vacuum to give a residue which was purified via column chromatography to afford benzyl 4-[(5M)-5-[1-[3-[tert-butyl(dimethyl)silyl]oxy-2,2-dimethyl-propyl]-7-(4,4,5,5-tetramethyl-1,3,2-dioxaborolan-2-yl)-4,5-dihydro-3H-benzo[cd]indol-2-yl]-4-fluoro-6-[(15)-1-methoxyethyl]-3-pyridyl]piperazine-1-carboxylate (intermediate D3, 500.0 mg) as a yellow gum. MS calc'd 855.5 (MH⁺), measured 855.6 (MH⁺).

### Intermediate D4

### benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate

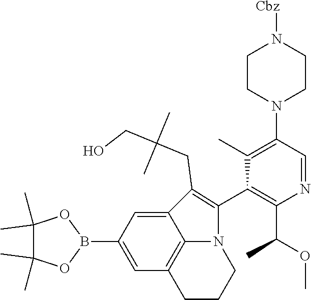

The title intermediate D4 was prepared according to the following scheme:

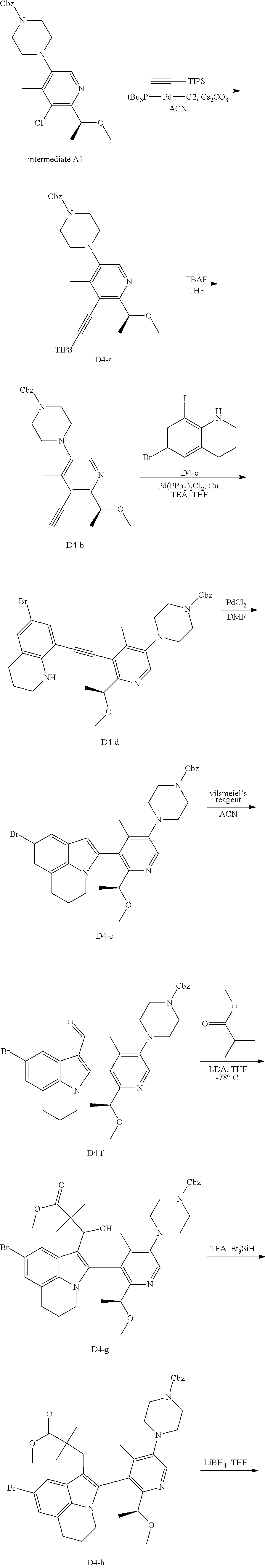

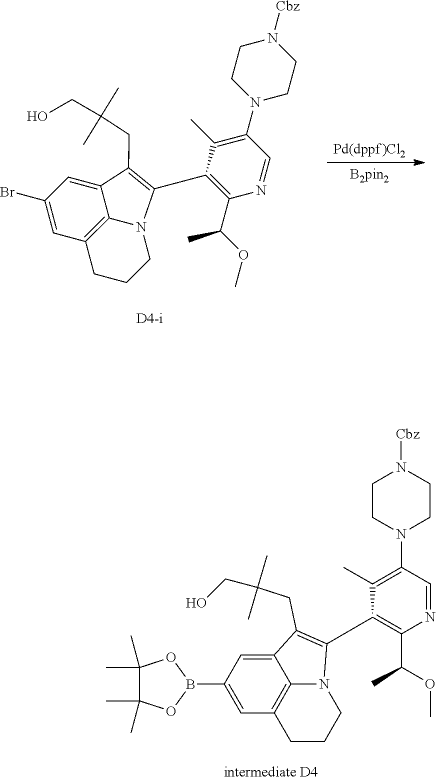

### Step 1: Preparation of benzyl 4-[6-[(1S)-1-methoxyethyl]-4-methyl-5-(2-triisopropylsilylethynyl)-3-pyridyl]piperazine-1-carboxylate (compound D4-a)

To a solution of benzyl 4-[5-chloro-6-[(1S)-1-methoxyethyl]-4-methyl-3-pyridyl]piperazine-1-carboxylate (intermediate A1, 5.0 g, 12.38 mmol) and (triisopropylsilyl)acetylene (3.4 g, 18.57 mmol) in ACN (75 mL), Cs₂CO₃ (10.5 g, 32.19 mmol) and t-Bu₃P-Pd-G2 (634.3 mg, 1.24 mmol) were added in one portion. The mixture was degassed under vacuum and purged with N2 for 3 times before stirred at 90° C. for 3 hrs under N2 atmosphere. The mixture was filtered, and the filtrate was concentrated under vacuum, the residue was purified via column chromatography to afford benzyl 4-[6-[(1S)-1-methoxyethyl]-4-methyl-5-(2-triisopropylsilylethynyl)-3-pyridyl]piperazine-1-carboxylate (compound D4-a, 6.0 g) as brown oil. MS calc'd 550.3 (MH⁺), measured 550.4 (MH⁺).

### Step 2: Preparation of benzyl 4-[5-ethynyl-6-[(1S)-1-methoxyethyl]-4-methyl-3-pyridyl]piperazine-1-carboxylate (compound D4-b)

To the mixture of benzyl 4-[6-[(1S)-1-methoxyethyl]-4-methyl-5-(2-triisopropylsilylethynyl)-3-pyridyl]piperazine-1-carboxylate (compound D4-a, 4.5 g, 8.22 mmol) in THF (50 mL), TBAF (12.3 mL, 12.33 mmol, 1 M in THF) was added dropwise at 0° C. The mixture was stirred at 0° C. for 1 h. The mixture was poured into water (200 mL) and extracted with EtOAc (50 mL, three times). The combined organic phase was washed with brine (100 mL), dried over anhydrous Na₂SO₄, filtered and concentrated under vacuum, the residue was purified via column chromatography to afford benzyl 4-[5-ethynyl-6-[(1S)-1-methoxyethyl]-4-methyl-3-pyridyl]piperazine-1-carboxylate (compound D4-b, 3.1 g) as yellow oil. MS calc'd 394.2 (MH⁺), measured 394.2 (MH⁺).

### Step 3: Preparation of benzyl 4-[5-[2-(6-bromo-1,2,3,4-tetrahydroquinolin-8-yl)ethynyl]-6-[(1S)-1-methoxyethyl]-4-methyl-3-pyridyl]piperazine-1-carboxylate (compound D4-d)

To a solution of 6-bromo-8-iodo-1,2,3,4-tetrahydroquinoline (synthesis of the compound D4-c refers to WO2024169914 intermediate D1, 3.5 g, 10.29 mmol), Pd(PPh₃)₂Cl₂ (482.8 mg, 0.69 mmol), CuI (130.9 mg, 0.69 mmol) and triethylamine (4.8 mL, 34.31 mmol) in DMF (20 mL), benzyl 4-[5-ethynyl-6-[(15)-1-methoxyethyl]-4-methyl-3-pyridyl]piperazine-1-carboxylate (compound D4-b, 2.7 g, 6.86 mmol) in DMF (30 mL) was added dropwise under N2 atmosphere at 25° C. over 1 h. The mixture was stirred at 25° C. for 1 h. The reaction mixture was poured into water (200 mL) and extracted with EtOAc (50 mL, three times). The combined organic phase was washed with brine (50 mL, three times), dried over anhydrous Na₂SO₄, filtered and concentrated under vacuum. The residue was purified via column chromatography to afford benzyl 4-[5-[2-(6-bromo-1,2,3,4-tetrahydroquinolin-8-yl)ethynyl]-6-[(1S)-1-methoxyethyl]-4-methyl-3-pyridyl]piperazine-1-carboxylate (compound D4-d, 3.3 g) as yellow gum. MS calc'd 603.2 (MH⁺), measured 603.2 (MH⁺).

### Step 4: Preparation of benzyl 4-[5-(6-bromo-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-e)

To a mixture of benzyl 4-[5-[2-(6-bromo-1,2,3,4-tetrahydroquinolin-8-yl)ethynyl]-6-[(1S)-1-methoxyethyl]-4-methyl-3-pyridyl]piperazine-1-carboxylate (compound D4-d, 3.2 g, 5.3 mmol) in DMF (50 mL), PdCl₂ (188.03 mg, 1.06 mmol) was added. The mixture was degassed under vacuum and purged with N₂ for three times before stirring at 65° C. for 16 h under N₂ atmosphere. The mixture was filtered; the filtrate was added into water (150 mL) and extracted with EtOAc (50 mL, three times). The combined organic phase was washed with brine (50 mL, three times), dried over anhydrous Na₂SO₄, filtered and concentrated under vacuum to give a residue, which was purified via column chromatography to afford benzyl 4-[5-(6-bromo-1-azatricyclo[6.3.1.0^{4,12}]dodeca-2,4,6,8(12)-tetraen-2-yl)-6-[(15)-1-methoxyethyl]-4-methyl-3-pyridyl]piperazine-1-carboxylate (compound D4-e, 1.0 g) as yellow gum. MS calc'd 603.2 (MH⁺), measured 603.2 (MH⁺).

### Step 5: Preparation of benzyl 4-[5-(6-bromo-3-formyl-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-f)

To the mixture of benzyl 4-[5-(6-bromo-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-e, 1.0 g, 1.66 mmol) in ACN (15 mL), vilsmeier reagent (1.1 g, 8.28 mmol) was added in one portion. The mixture was stirred at 70° C. for 4 hrs. The mixture was poured into sat. NaHCO₃ aq. (50 mL) slowly and stirred at 25° C. for 10 min, the mixture was extracted with EtOAc (20 mL, three times). The combined organic phase was washed with brine (30 mL), dried over anhydrous Na₂SO₄, filtered and concentrated under vacuum, the residue was purified via column chromatography to afford benzyl 4-[5-(6-bromo-3-formyl-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-f, 662.0 mg) as a yellow gum. MS calc'd 631.2 (MH⁺), measured 631.2 (MH⁺).

### Step 6: Preparation of benzyl 4-[5-[6-bromo-3-(1-hydroxy-3-methoxy-2,2-dimethyl-3-oxo-propyl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (compound D4-g)

To a solution of methyl isobutyrate (429.8 mg, 4.21 mmol) in THF (9 mL), LDA (2.1 mL, 4.21 mmol) was added dropwise at −70° C. under N2. After being stirred for 0.5 h, benzyl 4-[5-(6-bromo-3-formyl-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-f, 886.0 mg, 1.4 mmol) in THF (9 mL) was added dropwise at −70° C. Then the mixture was stirred at −70° C. for 1 h. The mixture was quenched with sat. NH₄Cl aq. (50 mL) and extracted with EtOAc (30 mL, three times). The organic phase was washed with brine (50 mL), dried over anhydrous Na₂SO₄, filtered and concentrated under vacuum. The residue was purified via column chromatography to afford benzyl 4-[5-[6-bromo-3-(1-hydroxy-3-methoxy-2,2-dimethyl-3-oxo-propyl)-1-azatricyclo[6.3.1.0^{4,12}]dodeca-2,4,6,8(12)-tetraen-2-yl]-6-[(15)-1-methoxyethyl]-4-methyl-3-pyridyl]piperazine-1-carboxylate (compound D4-g, 823.0 mg) as a yellow solid. MS calc'd 733.3 (MH⁺), measured 733.3 (MH⁺).

### Step 7: Preparation of benzyl 4-[5-[6-bromo-3-(3-methoxy-2,2-dimethyl-3-oxo-propyl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (compound D4-h)

To a solution of benzyl 4-[5-[6-bromo-3-(1-hydroxy-3-methoxy-2,2-dimethyl-3-oxo-propyl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (compound D4-g, 821.0 mg, 1.12 mmol) in DCM (16 mL), triethylsilane (1.0 g, 8.95 mmol) was added, followed by addition of TFA (3.3 mL, 44.76 mmol) dropwise at 0° C. The mixture was stirred at 0° C. for 1 h. The mixture was poured into water (50 mL) and extracted with EtOAc (30 mL, three times). The combined organic phase was washed with brine (30 mL, three times), dried over anhydrous Na₂SO₄, filtered and concentrated under vacuum to give a residue, which was purified by silica gel column to afford benzyl 4-[(5-[6-bromo-3-(3-methoxy-2,2-dimethyl-3-oxo-propyl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (compound D4-h, 751.0 mg) as a yellow solid. MS calc'd 717.3 (MH⁺), measured 717.3 (MH⁺).

### Step 8: Preparation of benzyl 4-[(5M)-5-[6-bromo-3-(3-hydroxy-2,2-dimethyl-propyl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (compound D4-i)

To a solution of benzyl 4-[5-[6-bromo-3-(3-methoxy-2,2-dimethyl-3-oxo-propyl)-1-azatricyclo[6.3.1.0^{4,12}]dodeca-2,4,6,8(12)-tetraen-2-yl]-6-[(15)-1-methoxyethyl]-4-methyl-3-pyridyl]piperazine-1-carboxylate (compound D4-h, 635.0 mg, 0.88 mmol) in THF (10 mL), LiBH₄ (5.3 mL, 10.62 mmol, 2 M in THF) was added dropwise under N2 at 0° C. The mixture was stirred at 20° C. for 16 hrs under N2, quenched with sat. NH₄Cl aq. (50 mL) slowly at 0° C. and stirred at 20° C. for another 20 min, and then extracted with EtOAc (20 mL, three times). The combined organic phase was washed with brine (50 mL), dried over anhydrous Na₂SO₄, filtered and concentrated under vacuum. The residue was purified via column chromatography to afford benzyl 4-[(5M)-5-[6-bromo-3-(3-hydroxy-2,2-dimethyl-propyl)-1-azatricyclo[6.3.1.0^{4,12}]dodeca-2,4,6,8(12)-tetraen-2-yl]-6-[(15)-1-methoxyethyl]-4-methyl-3-pyridyl]piperazine-1-carboxylate (compound D4-i, 401.0 mg, slower eluted) as a yellow solid. MS calc'd 689.3 (MH⁺), measured 689.3 (MH⁺).

### Step 9: Preparation of benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (intermediate D4)

To a solution of benzyl 4-[(5M)-5-[6-bromo-3-(3-hydroxy-2,2-dimethyl-propyl)-1-azatricyclo[6.3.1.0^{4,12}]dodeca-2,4,6,8(12)-tetraen-2-yl]-6-[(15)-1-methoxyethyl]-4-methyl-3-pyridyl]piperazine-1-carboxylate (compound D4-i, 296.0 mg, 0.43 mmol) in 2-methyltetrahydrofuran (6 mL), bis(pinacolato)diboron (326.9 mg, 1.29 mmol), K₂CO₃ (177.9 mg, 1.29 mmol), pivalic acid (21.9 mg, 0.21 mmol) and Pd(dppf)Cl₂ (31.4 mg, 0.04 mmol) were added. The mixture was degassed by bubbling nitrogen for 2 min before stirred at 75° C. for 4 hrs under N2 atmosphere. The mixture was filtered, and the filtrate was concentrated under vacuum. The residue was purified via column chromatography to afford benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (intermediate D4, 311.0 mg) as a yellow solid. MS calc'd 737.4 (MH⁺), measured 737.5 (MH⁺).

### Intermediate D5

### 3-[7-chloro-(2P)-2-[2-[(1S)-1-methoxyethyl]-4-methyl-5-(3-morpholinoprop-1-ynyl)-3-pyridyl]-4,5-dihydro-3H-benzo[cd]indol-1-yl]-2,2-dimethyl-propan-1-ol

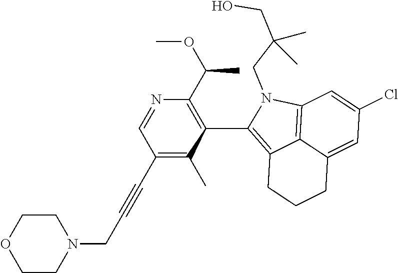

The intermediate D5 was prepare in analogy to the preparation of compound D1-C by using 4-[3-[5-chloro-6-[(1S)-1-methoxyethyl]-4-methyl-3-pyridyl]prop-2-ynyl]morpholine (intermediate A3) instead of benzyl 4-[5-chloro-6-[(1S)-1-methoxyethyl]-4-methyl-3-pyridyl]piperazine-1-carboxylate (intermediate A1).

### Intermediate D6

### Benzyl 4-[(5M)-5-[1-ethyl-3-(3-hydroxy-2,2-dimethyl-propyl)-5-(4,4,5,5-tetramethyl-1,3,2-dioxaborolan-2-yl)indol-2-yl]-6-[(1S)-1-methoxyethyl]-4-methyl-3-pyridyl]piperazine-1-carboxylate

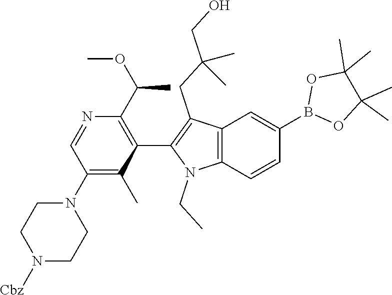

The intermediate D6 was prepared in analogy to the preparation of intermediate D4 by using benzyl 4-[5-(5-chloro-1-ethyl-3-formyl-indol-2-yl)-6-[(1S)-1-methoxyethyl]-4-methyl-3-pyridyl]piperazine-1-carboxylate (compound D6-e) instead of benzyl 4-[5-(6-bromo-3-formyl-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-f).

The compound D6-e was prepared according to the following scheme:

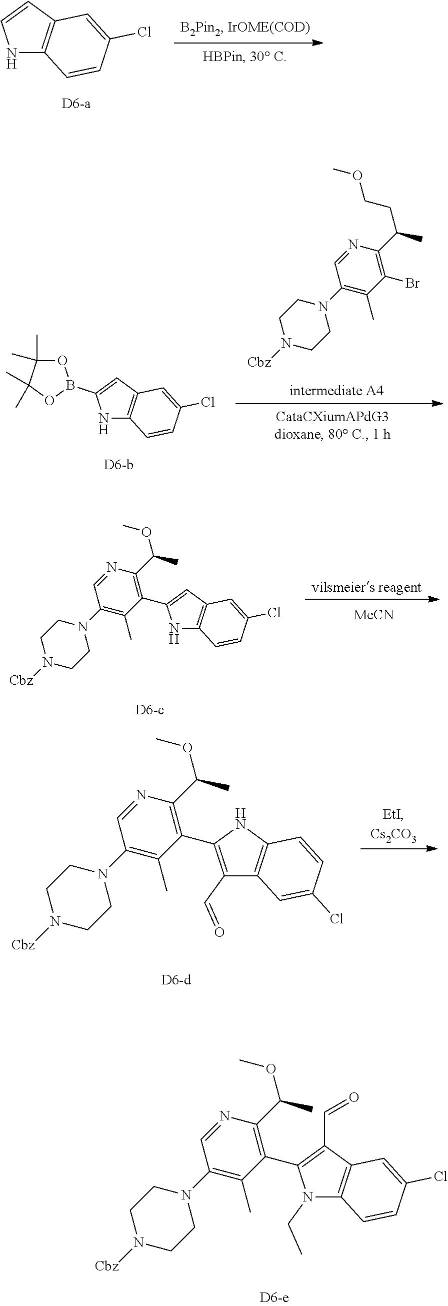

### Step 1: Preparation of 5-chloro-2-(4,4,5,5-tetramethyl-1,3,2-dioxaborolan-2-yl)-1H-indole (compound D6-b)

To a solution of 5-chloro-1H-indole (10.0 g, 65.97 mmol) and bis(pinacolato)diboron (8.4 g, 32.98 mmol) in THF (120 mL) were added HBpin (2.4 g, 18.47 mmol), 4,4′-Di-tert-butyl-2,2′-dipyridyl (531.2 mg, 1.98 mmol) and [Ir(OMe)(COD)]2 (715.4 mg, 0.99 mmol). The mixture was degassed and purged with N2 for 3 times and stirred at 30° C. for 0.75 hr. The reaction mixture was then added to water (300 mL) and extracted with EtOAc (50 mL, three times). The combined organic layer was washed with brine (200 mL, three times), dried over Na₂SO₄, filtered and concentrated under vacuum to give a residue, which was purified by prep-HPLC to afford 5-chloro-2-(4,4,5,5-tetramethyl-1,3,2-dioxaborolan-2-yl)-1H-indole (compound D6-b, 10.2 g) as a yellow solid. MS calc'd 278.1 (MH⁺), measured 278.1 (MH⁺).

### Step 2: Preparation of benzyl 4-[5-(5-chloro-1H-indol-2-yl)-6-[(1S)-1-methoxyethyl]-4-methyl-3-pyridyl]piperazine-1-carboxylate (compound D6-c)

To a solution of 5-chloro-2-(4,4,5,5-tetramethyl-1,3,2-dioxaborolan-2-yl)-1H-indole (compound D6-b, 9.2 g, 16.52 mmol) and benzyl 4-[5-bromo-6-[(1S)-1-methoxyethyl]-4-methyl-3-pyridyl]piperazine-1-carboxylate (intermediate A4, 7.78 g, 17.35 mmol) in 1,4-dioxane (200 mL) and water (40 mL) were added K₃PO₄ (10.5 g, 49.56 mmol) and cataCXiumA-Pd-G3 (3.6 g, 4.96 mmol). The mixture was degassed and purged with N2 for three times, then stirred at 80° C. for 1 h.

The reaction mixture was filtered and concentrated under vacuum to give a residue, which was purified by reversed phased chromatography to afford benzyl 4-[5-(5-chloro-1H-indol-2-yl)-6-[(1S)-1-methoxyethyl]-4-methyl-3-pyridyl]piperazine-1-carboxylate (compound D6-c, 4.2 g) as a yellow solid. MS calc'd 519.2 (MH⁺), measured 519.2 (MH⁺).

### Step 3: Preparation of benzyl 4-[5-(5-chloro-3-formyl-1H-indol-2-yl)-6-[(1S)-1-methoxyethyl]-4-methyl-3-pyridyl]piperazine-1-carboxylate (compound D6-d)

A mixture of benzyl 4-[5-(5-chloro-1H-indol-2-yl)-6-[(1S)-1-methoxyethyl]-4-methyl-3-pyridyl]piperazine-1-carboxylate (compound D6-c, 3.8 g, 7.32 mmol) in ACN (80 mL) was prepared. Vilsmeier reagent (9.37 g, 73.21 mmol) was added in one portion to the mixture, and the reaction was stirred at 70° C. for 4 hrs. The reaction mixture was slowly poured into sat. NaHCO₃ aq. (200 mL) and stirred at 25° C. for 20 min. The mixture was extracted with EtOAc (50 mL, three times). The combined organic phase was washed with brine (300 mL), dried over anhydrous Na₂SO₄, filtered and concentrated under vacuum, the residue was purified by column chromatography to afford benzyl 4-[5-(5-chloro-3-formyl-1H-indol-2-yl)-6-[(15)-1-methoxyethyl]-4-methyl-3-pyridyl]piperazine-1-carboxylate (compound D6-d, 3.0 g) as a yellow gum. MS calc'd 547.2 (MH⁺), measured 547.3 (MH⁺).

### Step 4: Preparation of benzyl 4-[5-(5-chloro-1-ethyl-3-formyl-indol-2-yl)-6-[(1S)-1-methoxyethyl]-4-methyl-3-pyridyl]piperazine-1-carboxylate (compound D6-e)

To a solution of benzyl 4-[5-(5-chloro-3-formyl-1H-indol-2-yl)-6-[(1S)-1-methoxyethyl]-4-methyl-3-pyridyl]piperazine-1-carboxylate (compound D6-d, 2.7 g, 4.94 mmol) in DMF (50 mL) were added Cs₂CO₃ (4.8 g, 14.81 mmol) and ethyl iodide (1.5 g, 9.87 mmol) at 0° C. The mixture was stirred at 25° C. for 12 hrs. Water (200 mL) was added to the reaction mixture and it was extracted with EtOAc (50 mL, three times). The combined organic layer was washed with brine (200 mL, three times), dried over Na₂SO₄, filtered and concentrated under vacuum to give a residue, which was purified by column chromatography to afford benzyl 4-[5-(5-chloro-1-ethyl-3-formyl-indol-2-yl)-6-[(1S)-1-methoxyethyl]-4-methyl-3-pyridyl]piperazine-1-carboxylate (compound D6-e, 3.0 g) as a yellow gum. MS calc'd 575.2 (MH⁺), measured 575.1 (MW).

### Intermediate D7

### Benzyl 4-[(5P)-5-[3-[3-[tert-butyl(dimethyl)silyl]oxy-2,2-dimethyl-propyl]-6-(4,4,5,5-tetramethyl-1,3,2-dioxaborolan-2-yl)-1-azatricyclo[6.3.1.0^{4,12}]dodeca-2,4(12),5,7-tetraen-2-yl]-4-fluoro-6-[(1S)-1-methoxyethyl]-3-pyridyl]piperazine-1-carboxylate

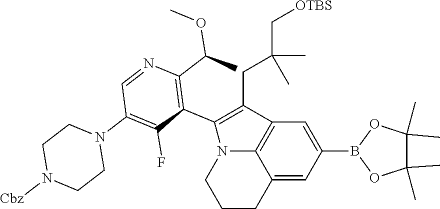

The intermediate D7 was prepared in analogy to the preparation of intermediate D3 by using benzyl 4-[(5P)-5-[6-bromo-3-(3-hydroxy-2,2-dimethyl-propyl)-1-azatricyclo[6.3.1.0^{4,12}]dodeca-2,4(12),5,7-tetraen-2-yl]-4-fluoro-6-[(1S)-1-methoxyethyl]-3-pyridyl]piperazine-1-carboxylate (compound D7-i) instead of benzyl 4-[(5M)-5-[7-chloro-1-(3-hydroxy-2,2-dimethyl-propyl)-4,5-dihydro-3H-benzo[cd]indol-2-yl]-4-fluoro-6-[(1S)-1-methoxyethyl]-3-pyridyl]piperazine-1-carboxylate (compound D3-c).

The compound D7-i was prepared in analogy to the preparation of compound D4-i by using benzyl 4-[4-fluoro-5-iodo-6-[(1S)-1-methoxyethyl]-3-pyridyl]piperazine-1-carboxylate (intermediate A2) instead of benzyl 4-[5-chloro-6-[(1S)-1-methoxyethyl]-4-methyl-3-pyridyl]piperazine-1-carboxylate (intermediate A1).

### Intermediate D8

### 3-[(2M)-2-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol

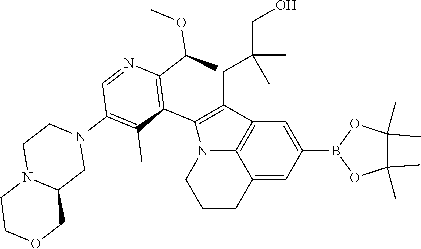

The intermediate was prepare in analogy to the preparation o intermediate D4 by using (9aS)-8-[5-(6-chloro-1-azatricyclo[6.3.1.0^{4,12}]dodeca-2,4(12),5,7-tetraen-2-yl)-6-[(1S)-1-methoxyethyl]-4-methyl-3-pyridyl]-3,4,6,7,9,9a-hexahydro-1H-pyrazino[2,1-c][1,4]oxazine (compound D8-a) instead of benzyl 4-[5-(6-bromo-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-e).

The compound D8-a was prepared according to the following scheme.

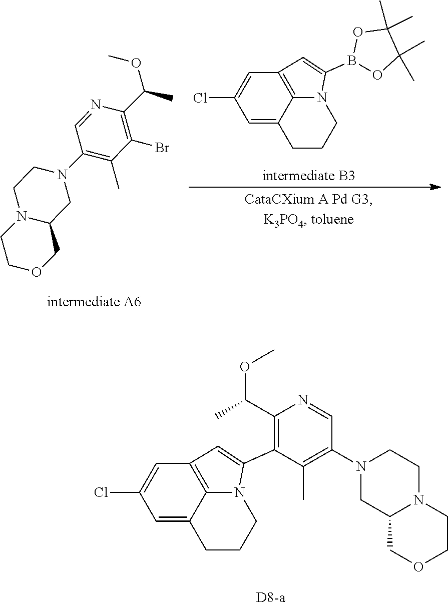

To a solution of (9aS)-8-[5-bromo-6-[(1S)-1-methoxyethyl]-4-methyl-3-pyridyl]-3,4,6,7,9,9a-hexahydro-1H-pyrazino[2,1-c][1,4]oxazine (intermediate A6, 3.0 g, 8.1 mmol) and 6-chloro-2-(4,4,5,5-tetramethyl-1,3,2-dioxaborolan-2-yl)-1-azatricyclo[6.3.1.0^{4,12}]dodeca-2,4(12),5,7-tetraene (intermediate B3, 4.8 g, 12.15 mmol) in 1,4-dioxane (60 mL) and water (12 mL) was added K₃PO₄ (5.2 g, 24.31 mmol) and cataCXium A Pd G3 (1.7 g, 2.43 mmol). The mixture was degassed and purged with nitrogen three times and stirred at 80° C. for 1 h. The reaction mixture was adjusted pH to 3 by HCl (1M), extracted with EtOAc (50 mL, three times). The combined water phase was adjusted pH=8 by sat. NaHCO₃ aqueous solution, extracted with EtOAc (50 mL, three times). The combined organic layer was washed by brine (100 mL), dried over anhydrous Na₂SO₄, filtered and concentrated under vacuum to afford (9aS)-8-[5-(6-chloro-1-azatricyclo[6.3.1.04,12]dodeca-2,4,6,8(12)-tetraen-2-yl)-6-[(1S)-1-methoxyethyl]-4-methyl-3-pyridyl]-3,4,6,7,9,9a-hexahydro-1H-pyrazino[2,1-c][1,4]oxazine (compound D8-a, 3.0 g) as a yellow gum. MS calc'd 481.2 (MH⁺), measured 481.2 (MH⁺)

### Intermediate D9

### Benzyl 4-[4-ethyl-(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate

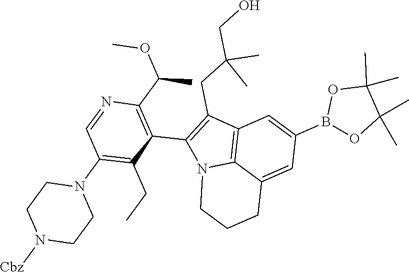

The intermediate D9 was prepared in analogy to the preparation of intermediate D8 by using benzyl 4-[5-chloro-4-ethyl-6-[(1S)-1-methoxyethyl]-3-pyridyl]piperazine-1-carboxylate (intermediate A7) instead of (9aS)-8-[5-bromo-6-[(1S)-1-methoxyethyl]-4-methyl-3-pyridyl]-3,4,6,7,9,9a-hexahydro-1H-pyrazino[2,1-c][1,4]oxazine (intermediate A6).

### Intermediate D10

### Benzyl 4-[4-cyclopropyl-(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate

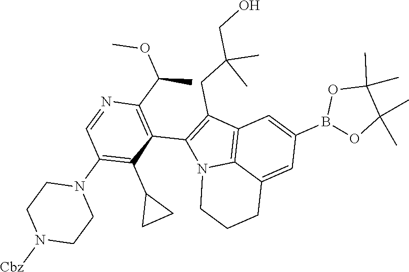

The intermediate D10 was prepared in analogy to the preparation of intermediate D8 by using benzyl 4-[5-chloro-4-cyclopropyl-6-[(1S)-1-methoxyethyl]-3-pyridyl]piperazine-1-carboxylate (intermediate A8) instead of (9aS)-8-[5-bromo-6-[(1S)-1-methoxyethyl]-4-methyl-3-pyridyl]-3,4,6,7,9,9a-hexahydro-1H-pyrazino[2,1-c][1,4]oxazine (intermediate A6).

### Intermediate D11

### 3-[2-[4-(difluoromethyl)-2-[(1S)-1-methoxyethyl]-5-(4-methylpiperazin-1-yl)-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol

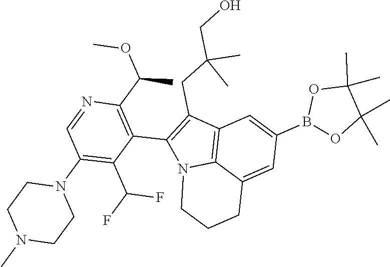

The intermediate D11 was prepared in analogy to the preparation of intermediate D8 by using 1-[5-bromo-4-(difluoromethyl)-6-[(15)-1-methoxyethyl]-3-pyridyl]-4-methyl-piperazine (intermediate A9) instead of (9aS)-8-[5-bromo-6-[(15)-1-methoxyethyl]-4-methyl-3-pyridyl]-3,4,6,7,9,9a-hexahydro-1H-pyrazino[2,1-c][1,4]oxazine (intermediate A6).

### Intermediate D12

### Benzyl 4-[4-chloro-(5P)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate

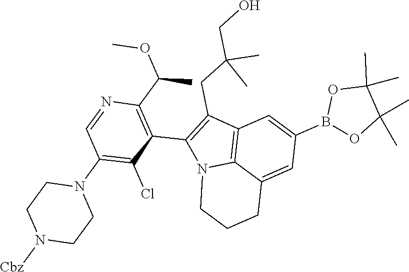

The intermediate D12 was prepared in analogy to the preparation of intermediate D4 by using benzyl 4-[4-chloro-5-(6-chloro-1-azatricyclo[6.3.1.0^{4,12}]dodeca-2,4(12),5,7-tetraen-2-yl)-6-[(1S)-1-methoxyethyl]-3-pyridyl]piperazine-1-carboxylate (compound D12-e) instead of benzyl 4-[5-(6-bromo-1-azatricyclo[6.3.1.0^{4,12}]dodeca-2,4,6,8(12)-tetraen-2-yl)-6-[(1S)-1-methoxyethyl]-4-methyl-3-pyridyl]piperazine-1-carboxylate (compound D4-e).

The compound D12-e was prepared according to the following scheme.

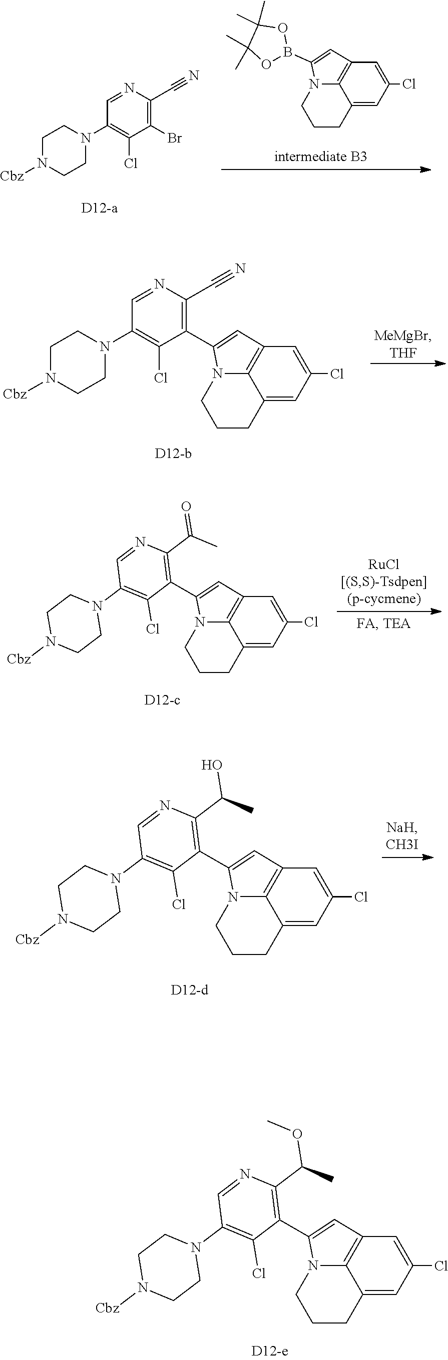

### Step 1: Preparation of benzyl 4-[4-chloro-5-(6-chloro-1-azatricyclo[6.3.1.0^{4,12}]dodeca-2,4,6,8(12)-tetraen-2-yl)-6-cyano-3-pyridyl]piperazine-1-carboxylate (compound D12-b)

To a solution of benzyl 4-(5-bromo-4-chloro-6-cyano-3-pyridyl)piperazine-1-carboxylate (compound D12-a, 5.0 g, 11.48 mmol) and 6-chloro-2-(4,4,5,5-tetramethyl-1,3,2-dioxaborolan-2-yl)-1-azatricyclo[6.3.1.0^{4,12}]dodeca-2,4(12),5,7-tetraene (intermediate B3, 4.7 g, 14.92 mmol) in toluene (100 mL) was added sodium sulfate decahydrate (5.0 g, 15.52 mmol), potassium phosphate (7.3 g, 34.43 mmol), and cataCXium® A Pd G3 (4.2 g, 5.74 mmol). The mixture was bubbled with nitrogen gas for 5 minutes and stirred at 45° C. for 12 hours. The reaction mixture was filtered, and the filtrate was concentrated under reduced pressure to yield a residue. The residue was purified by silica gel column chromatography to afford benzyl 4-[4-chloro-5-(6-chloro-1-azatricyclo[6.3.1.0^{4,12}]dodeca-2,4,6,8(12)-tetraen-2-yl)-6-cyano-3-pyridyl]piperazine-1-carboxylate (compound D12-b, 5.2 g). MS calc'd 546.2 (MH⁺), measured 546.2 (MH⁺).

The compound D12-a was prepared in analogy to the preparation of compound A1-d by using 3,5-dibromo-4-chloro-pyridine instead of 3,5-dichloro-4-methyl-pyridine (compound A1-a).

### Step 2: Preparation of benzyl 4-[6-acetyl-4-chloro-5-(6-chloro-1-azatricyclo[6.3.1.0^{4,12}]dodeca-2,4(12),5,7-tetraen-2-yl)-3-pyridyl]piperazine-1-carboxylate (compound D12-c)

To a solution of benzyl 4-[4-chloro-5-(6-chloro-1-azatricyclo[6.3.1.0^{4,12}]dodeca-2,4,6,8(12)-tetraen-2-yl)-6-cyano-3-pyridyl]piperazine-1-carboxylate (compound D12-b, 2.0 g, 3.66 mmol) in THF (40 mL) was added dropwise methylmagnesium bromide (6.1 mL, 18.3 mmol, 3.0 M) at −5° C. under a nitrogen atmosphere. The mixture was stirred at 0° C. for 3 hours under nitrogen. The reaction mixture was poured into an ice-cold 3 M hydrochloric acid (HCl, 300 mL) aqueous solution and extracted with ethyl acetate (80 mL, three times). The combined organic layer was washed with brine, dried over anhydrous sodium sulfate, filtered, and concentrated under reduced pressure to yield a residue. The residue was purified by silica gel column chromatography to afford benzyl 4-[6-acetyl-4-chloro-5-(6-chloro-1-azatricyclo[6.3.1.0^{4,12}]dodeca-2,4(12),5,7-tetraen-2-yl)-3-pyridyl]piperazine-1-carboxylate (compound D12-c, 900.0 mg). MS calc'd 563.2 (MH⁺), measured 563.2 (MH⁺).

### Step 3: Preparation of benzyl 4-[4-chloro-5-(6-chloro-1-azatricyclo[6.3.1.0^{4,12}]dodeca-2,4(12),5,7-tetraen-2-yl)-6-[(1S)-1-hydroxyethyl]-3-pyridyl]piperazine-1-carboxylate (compound D12-d)

Formic acid (571.7 mg, 12.42 mmol) was added slowly to TEA (20.0 mL, 143.49 mmol) at 0° C. The mixture was stirred at 0° C. for 10 minutes, after which (S,S)—N-(p-toluenesulfonyl)-1,2-diphenylethanediamine(chloro)(p-cymene)ruthenium (47.4 mg, 0.07 mmol) was added at 25° C., followed by a solution of benzyl 4-[6-acetyl-4-chloro-5-(6-chloro-1-azatricyclo[6.3.1.0^{4,12}]dodeca-2,4(12),5,7-tetraen-2-yl)-3-pyridyl]piperazine-1-carboxylate (compound D12-c, 700.0 mg, 1.24 mmol) in THF (5 mL). The reaction mixture was stirred at 45° C. for 4 hours. The reaction mixture was concentrated under reduced pressure to give a residue. The residue was purified by silica gel column chromatography to afford benzyl 4-[4-chloro-5-(6-chloro-1-azatricyclo[6.3.1.04,12]dodeca-2,4(12),5,7-tetraen-2-yl)-6-[(1S)-1-hydroxyethyl]-3-pyridyl]piperazine-1-carboxylate (compound D12-d, 430.0 mg, faster eluted). MS calc'd 565.2 (MH⁺), measured 565.2 (MH⁺).

### Step 4: Preparation of benzyl 4-[4-chloro-5-(6-chloro-1-azatricyclo[6.3.1.0^{4,12}]dodeca-2,4(12),5,7-tetraen-2-yl)-6-[(1S)-1-methoxyethyl]-3-pyridyl]piperazine-1-carboxylate (compound D12-e)

To a solution of benzyl 4-[4-chloro-(5P)-5-(6-chloro-1-azatricyclo[6.3.1.0^{4,12}]dodeca-2,4(12),5,7-tetraen-2-yl)-6-[(1S)-1-hydroxyethyl]-3-pyridyl]piperazine-1-carboxylate (compound D12-d, 390.0 mg, 0.69 mmol) in THF (6 mL) was added sodium hydride (60% in oil, 41.4 mg, 1.03 mmol) slowly at 0° C. The mixture was stirred at 0° C. for 10 minutes, after which iodomethane (117.5 mg, 0.83 mmol) was added dropwise at 0° C. The mixture was stirred at 25° C. for 1 hour. The reaction mixture was poured into ice water (30 mL) and extracted with EtOAc (20 mL, three times). The combined organic layer was washed with brine (30 mL), dried over Na₂SO₄, filtered, and concentrated under reduced pressure to yield a residue. The residue was purified by silica gel column chromatography to afford benzyl 4-[4-chloro-5-(6-chloro-1-azatricyclo[6.3.1.0^{4,12}]dodeca-2,4(12),5,7-tetraen-2-yl)-6-[(1S)-1-methoxyethyl]-3-pyridyl]piperazine-1-carboxylate (compound D12-e, 390.0 mg). MS calc'd 579.2 (MH⁺), measured 579.2 (MH⁺).

### Intermediate D13

### 3-[(2M)-2-[2-[(1S)-1-methoxyethyl]-5-(4-methylpiperazin-1-yl)-4-(trifluoromethyl)-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol

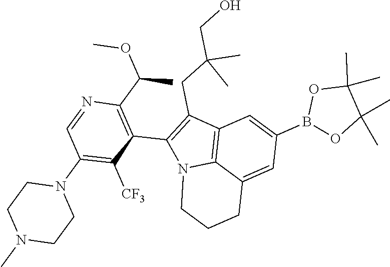

The intermediate D13 was prepared in analogy to the preparation of intermediate D4 by using 3,5-dichloro-2-[(1S)-1-methoxyethyl]-4-(trifluoromethyl)pyridine (intermediate A10) instead of benzyl 4-[5-chloro-6-[(15)-1-methoxyethyl]-4-methyl-3-pyridyl]piperazine-1-carboxylate (intermediate A1).

### Intermediate E

### (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione

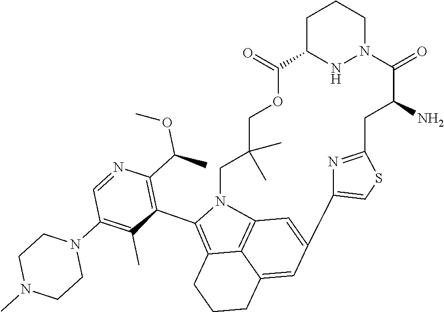

The title intermediate E was prepared according to the following scheme:

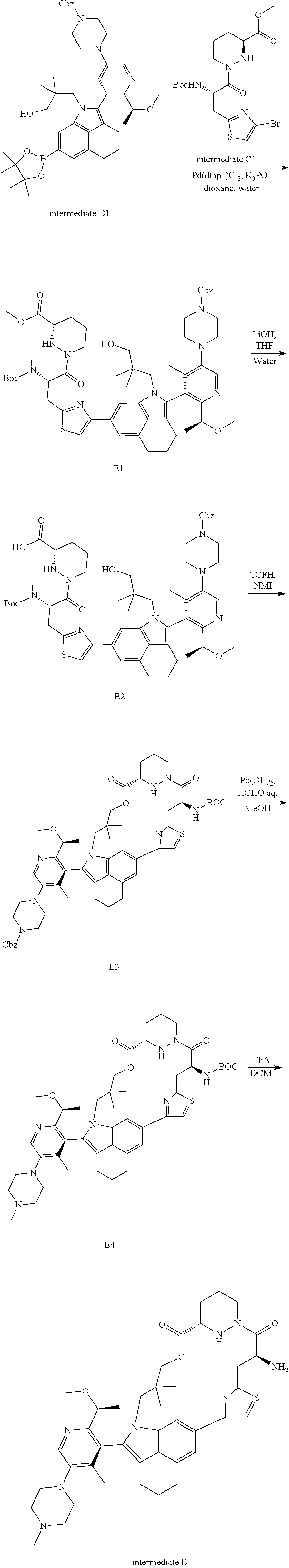

### Step 1: Preparation of methyl (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylate (compound E1)

To a solution of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1, 900.0 mg, 1.2 mmol) and methyl (3S)-1-[(2S)-3-(4-bromothiazol-2-yl)-2-(tert-butoxycarbonylamino)propanoyl]hexahydropyridazine-3-carboxylate (intermediate C1, 612.3 mg, 1.28 mmol) in 1,4-dioxane (30 mL) and water (6 mL), K₃PO₄ (777.9 mg, 3.66 mmol) and Pd(dtbpf)Cl₂ (79.6 mg, 0.12 mmol) were added. The mixture was degassed and purged with N2 for three times and stirred at 60° C. for 1 h under nitrogen atmosphere. The reaction mixture was concentrated under vacuum to give a residue, the residue was purified via column chromatography to afford methyl (35)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylate (compound E1, 918.0 mg,) as a yellow solid. MS calc'd 1007.5 (MH⁺), measured 1007.5 (MH⁺).

### Step 2: Preparation of (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylic acid (compound E2)

To a solution of methyl (35)-1-[(25)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylate (compound E1, 818.0 mg, 0.81 mmol) in THF (15 mL), a solution of LiOH (68.2 mg, 1.62 mmol) in water (3 mL) was added dropwise at 0° C. The mixture was stirred at 0° C. for 0.5 h, then poured into water (150 mL), pH was adjusted to 3 with HCl aq. (1 M), and then the mixture was extracted with EtOAc (80 mL, three times). The combined organic layer was washed with brine (100 mL), dried over Na₂SO₄, filtered and concentrated under vacuum to afford (35)-1-[(25)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylic acid (compound E2, 910.0 mg) as a yellow solid. MS calc'd 991.5 (M−H)⁻, measured 991.5, (M−H)⁻.

### Step 3: Preparation of benzyl 4-[(5P)-5-[(7S,13S)-7-(tert-butoxycarbonylamino)-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound E3)

To a solution of TCFH (960.4 mg, 3.42 mmol) and NMI (0.7 mL, 8.56 mmol) in ACN (50 mL), a solution of (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylic acid (compound E2, 850.0 mg, 0.86 mmol) in ACN (100 mL) was added dropwise over 1 h, and the mixture was stirred at 25° C. for 0.5 h. The reaction mixture was concentrated under vacuum to give a residue. To the residue, water (100 mL) and EtOAc (100 mL) were added. The aqueous phase was extracted with EtOAc (80 mL, three times). The combined organic layer was washed with brine (200 mL), dried over Na₂SO₄, filtered and concentrated under vacuum to give a residue. The residue was purified by reversed phase chromatography to afford benzyl 4-[(5P)-5-[(7S,13S)-7-(tert-butoxycarbonylamino)-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound E3, 700.0 mg) as a yellow solid. MS calc'd 975.5 (MW), measured 975.5 (MH⁺).

### Step 4: Preparation of tert-butyl N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]carbamate (compound E4)

To a solution of Pd(OH)₂ on activated carbon (700.0 mg) in methanol (10 mL), HCHO aq. (37%, 293.9 mg, 3.59 mmol) and benzyl 4-[(5P)-5-[(7S,13S)-7-(tert-butoxycarbonylamino)-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound E3, 700 mg, 0.72 mmol) were added. The mixture was degassed and purged with Ar for three times, then the mixture was degassed and purged with H2 for three times and stirred at 30° C. for 12 hrs under H2 atmosphere (50 psi). The reaction mixture was filtered, and the filtrate was concentrated under vacuum to give a residue. The residue was dissolved in EtOAc (80 mL), washed with brine (50 mL), dried over Na₂SO₄, filtered and concentrated under vacuum to give a residue. The residue was purified by reversed phase chromatography to afford tert-butyl N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]carbamate (compound E4, 610.0 mg) as a yellow solid. MS calc'd 855.5 (MH⁺), measured 855.5 (MW).

### Step 5: Preparation of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E)

To a solution of tert-butyl N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]carbamate (compound E4, 600.0 mg, 0.7 mmol) in DCM (12 mL), TFA (6.0 mL, 77.88 mmol) was added dropwise at 0° C. The mixture was stirred at 25° C. for 0.5 h. The reaction mixture was concentrated under vacuum to give a crude. To the crude, sat. NaHCO₃ aq. (180 mL) and EtOAc (100 mL) were added. The mixture was separated, and the aqueous phase was extracted with EtOAc (60 mL, three times). The combined organic layer was washed with brine (100 mL), dried over Na₂SO₄, filtered and concentrated under vacuum to afford (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E, 400.0 mg) as a yellow solid. MS calc'd 755.4 (MW), measured 755.4 (MH⁺).

### Intermediate F

### (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione

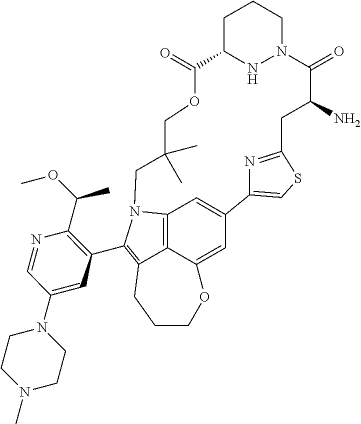

The title intermediate F was prepared in analogy to the preparation of intermediate E by using benzyl 4-[(5P)-5-[3-(3-hydroxy-2,2-dimethyl-propyl)-6-(4,4,5,5-tetramethyl-1,3,2-dioxaborolan-2-yl)-9-oxa-3-azatricyclo[6.4.1.0^{4,13}]trideca-1,4(13),5,7-tetraen-2-yl]-6-[(1S)-1-methoxyethyl]-3-pyridyl]piperazine-1-carboxylate (intermediate D2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Intermediate G

### (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione

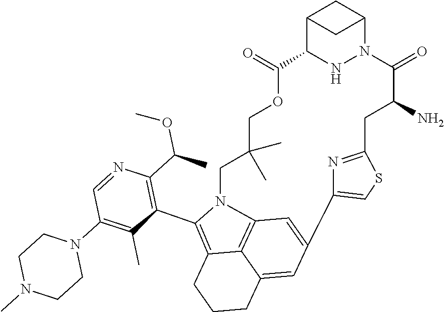

The title intermediate G was prepared in analogy to the preparation of intermediate E by using methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of methyl (3S)-1-[(2S)-3-(4-bromothiazol-2-yl)-2-(tert-butoxycarbonylamino)propanoyl]hexahydropyridazine-3-carboxylate (intermediate C1).

### Intermediate H

### (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione

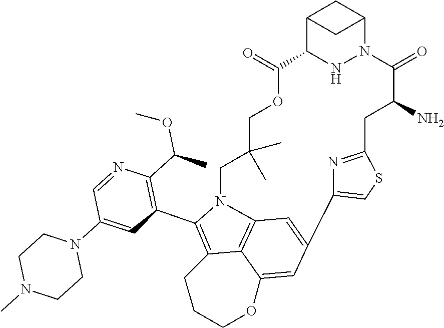

The title intermediate H was prepared in analogy to the preparation of intermediate E by using benzyl 4-[(5P)-5-[3-(3-hydroxy-2,2-dimethyl-propyl)-6-(4,4,5,5-tetramethyl-1,3,2-dioxaborolan-2-yl)-9-oxa-3-azatricyclo[6.4.1.0^{4,13}]trideca-1,4(13),5,7-tetraen-2-yl]-6-[(1S)-1-methoxyethyl]-3-pyridyl]piperazine-1-carboxylate (intermediate D2) and methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (35)-1-[(2S)-3-(4-bromothiazol-2-yl)-2-(tert-butoxycarbonylamino)propanoyl]hexahydropyridazine-3-carboxylate (intermediate C1).

### Intermediate I

### (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione

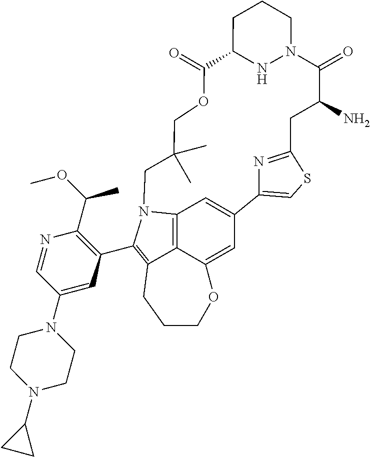

The title intermediate I was prepared according to the following scheme:

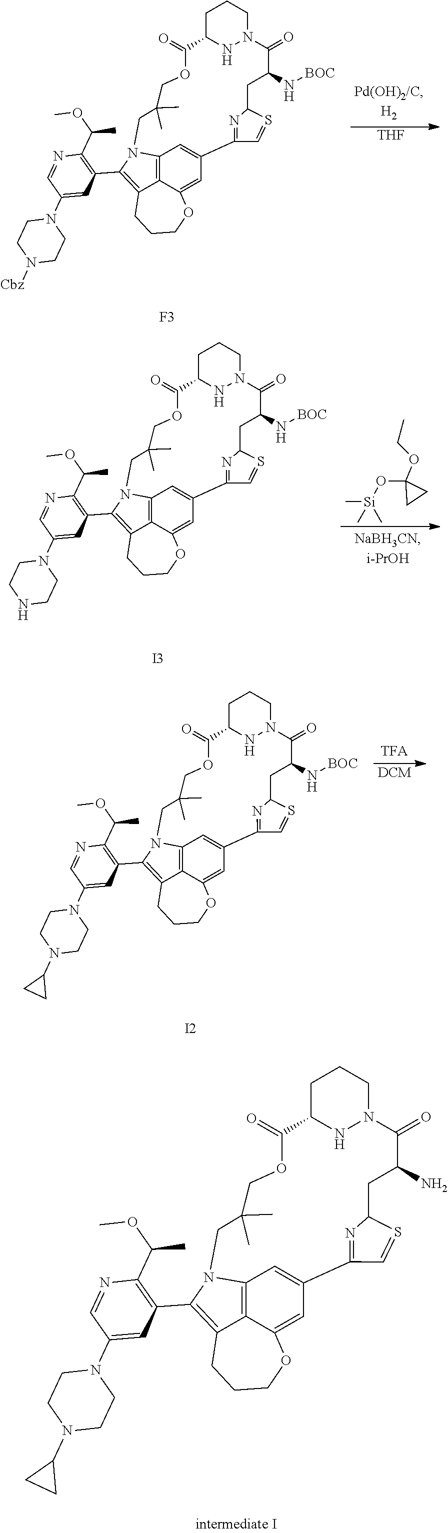

### Step 1: Preparation of tert-butyl N-[(7S,13S)-(20P)-20-[2-[(1S)-1-methoxyethyl]-5-piperazin-1-yl-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]carbamate (compound I1)

To the mixture of dry Pd(OH)₂ on activated carbon (467.1 mg, 0.67 mmol) in THF (15 mL), benzyl 4-[(5P)-5-[(7S,13S)-7-(tert-butoxycarbonylamino)-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-20-yl]-6-[(1S)-1-methoxyethyl]-3-pyridyl]piperazine-1-carboxylate (compound F3, 500.0 mg, 0.51 mmol) was added under N2 atmosphere in one portion. The mixture was degassed under vacuum and purged with N2 three times before degassed under vacuum and purged with H2 three times. The mixture was stirred at 25° C. for 4 hrs under H2 atmosphere (15 psi). The mixture was filtered, the filtrate was concentrated under vacuum to afford tert-butyl N-[(7S,13S)-(20P)-20-[2-[(1S)-1-methoxyethyl]-5-piperazin-1-yl-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]carbamate (compound I1, 420.0 mg) as an off-white solid. MS calc'd 843.4 (MH⁺), measured 843.4 (MH⁺).

The compound F3 was prepared in analogy to the preparation of compound E3 by using benzyl 4-[(5P)-5-[3-(3-hydroxy-2,2-dimethyl-propyl)-6-(4,4,5,5-tetramethyl-1,3,2-dioxaborolan-2-yl)-9-oxa-3-azatricyclo[6.4.1.0^{4,13}]trideca-1,4(13),5,7-tetraen-2-yl]-6-[(1S)-1-methoxyethyl]-3-pyridyl]piperazine-1-carboxylate (intermediate D2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Step 2: Preparation of tert-butyl N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]carbamate (compound I2)

To the mixture of tert-butyl N-[(7S,13S)-(20P)-20-[2-[(1S)-1-methoxyethyl]-5-piperazin-1-yl-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]carbamate (compound I1, 420.0 mg, 0.5 mmol), Molecular sieves, 4A (420.0 mg) and acetic acid (149.6 mg, 2.49 mmol) in IPA (10 mL), (1-ethoxycyclopropoxy)trimethylsilane (173.7 mg, 1.0 mmol) was added in one portion. After being stirred at 25° C. for 0.5 h, the mixture was added with sodium cyanoborohydride (93.9 mg, 1.49 mmol). The mixture was stirred at 60° C. for 16 hrs under N2 atmosphere. The mixture was filtered; the filtrate was poured into water (40 mL) and extracted with EtOAc (20 mL, three times). The combined organic layer was washed with brine (30 mL), dried over anhydrous Na₂SO₄, filtered and concentrated under vacuum to get a residue. The residue was purified by reversed phase chromatography to afford tert-butyl N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]carbamate (compound 12, 305.0 mg) as a white solid. MS calc'd 883.4 (MH⁺), measured 883.4 (MH⁺).

### Step 3: Preparation of (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate I)

To the mixture of tert-butyl N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]carbamate (compound 12, 300.0 mg, 0.34 mmol) in DCM (3 mL), TFA (1.5 mL, 39.26 mmol) was added dropwise at 0° C. The mixture was stirred at 25° C. for 1 h. The mixture was poured into sat. NaHCO₃ aq. (20 mL) slowly and extracted with DCM (10 mL, three times). The combined organic layer was washed with brine (10 mL), dried over anhydrous Na₂SO₄, filtered and concentrated under vacuum to afford (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate I, 270.0 mg) as an off-white solid. MS calc'd 783.4 (MH⁺), measured 783.5 (MH⁺).

### Intermediate J

### (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione

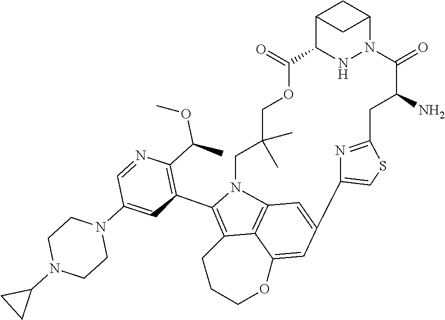

The title intermediate J was prepared in analogy to the preparation of intermediate I by using benzyl 4-[(5P)-5-[(7S,13S)-7-(tert-butoxycarbonylamino)-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-20-yl]-6-[(1S)-1-methoxyethyl]-3-pyridyl]piperazine-1-carboxylate (compound 113) instead of benzyl 4-[(5P)-5-[(7S,13S)-7-(tert-butoxycarbonylamino)-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-20-yl]-6-[(1S)-1-methoxyethyl]-3-pyridyl]piperazine-1-carboxylate (compound F3).

The compound H3 was prepared in analogy to the preparation of compound E3 by using benzyl 4-[(5P)-5-[3-(3-hydroxy-2,2-dimethyl-propyl)-6-(4,4,5,5-tetramethyl-1,3,2-dioxaborolan-2-yl)-9-oxa-3-azatricyclo[6.4.1.0^{4,13}]trideca-1,4(13),5,7-tetraen-2-yl]-6-[(1S)-1-methoxyethyl]-3-pyridyl]piperazine-1-carboxylate (intermediate D2) and methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-(tert-butoxycarbonylamino)propanoyl]hexahydropyridazine-3-carboxylate (intermediate C1).

### Intermediate K

### (7S,13S)-7-amino-(20M)-20-[4-fluoro-2-[(1S)-1-methoxyethyl]-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione

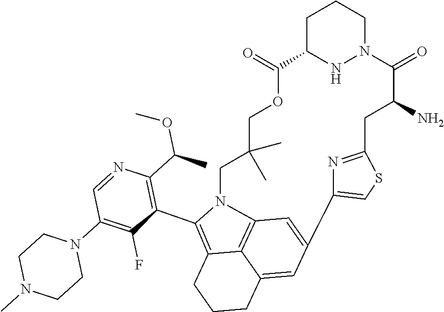

The title intermediate K was prepared in analogy to the preparation of intermediate E by using (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylic acid (compound K3) instead of (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylic acid (compound E2).

The compound K3 was prepared according to the following scheme:

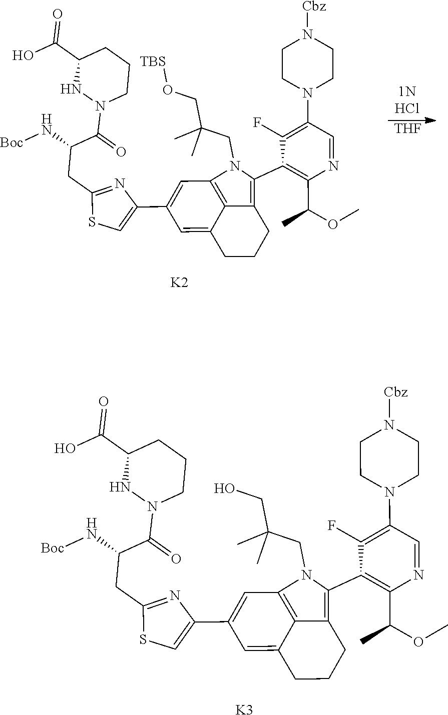

To a solution of (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-(tert-butoxycarbonylamino)propanoyl]hexahydropyridazine-3-carboxylic acid (compound K2, 400.0 mg, 0.36 mmol) in THF (1.5 mL), HCl (1N in THF, 4.04 mL, 4.04 mmol) was added at 0° C., and the mixture was stirred at 0° C. for 0.2 h. The reaction mixture (combined with other batch) was poured into water (20 mL), and extracted with EtOAc (15 mL, three times). The combined organic layer was washed with brine (20 mL), dried over Na₂SO₄, filtered and concentrated under vacuum to afford (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-(3-hydroxy-2,2-dimethyl-propyl)-4,5-dihydro-3H-benzo[cd]indol-7-yl]thiazol-2-yl]-2-(tert-butoxycarbonylamino)propanoyl]hexahydropyridazine-3-carboxylic acid (compound K3, 450.0 mg) as a yellow solid which was used in the next step without further purification. MS calc'd 997.5 (MH⁺), measured 997.5 (MH⁺).

The compound K2 was prepared in analogy to the preparation of compound E2 by using benzyl 4-[(5M)-5-[1-[3-[tert-butyl(dimethyl)silyl]oxy-2,2-dimethyl-propyl]-7-(4,4,5,5-tetramethyl-1,3,2-dioxaborolan-2-yl)-4,5-dihydro-3H-benzo[cd]indol-2-yl]-4-fluoro-6-[(1S)-1-methoxyethyl]-3-pyridyl]piperazine-1-carboxylate (intermediate D3) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Intermediate L

### (7S,13S)-7-amino-(20P)-20-[5-(4-cyclopropylpiperazin-1-yl)-2-[(1S)-1-methoxyethyl]-4-methyl-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione

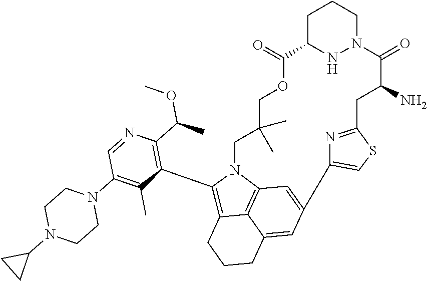

The title intermediate L was prepared in analogy to the preparation of intermediate I by using benzyl 4-[(5P)-5-[(7S,13S)-7-(tert-butoxycarbonylamino)-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound E3) instead of benzyl 4-[(5P)-5-[(7S,13S)-7-(tert-butoxycarbonylamino)-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-20-yl]-6-[(1S)-1-methoxyethyl]-3-pyridyl]piperazine-1-carboxylate (compound F3).

### Intermediate M

### (7S,13S)-7-amino-(20M)-20-[4-fluoro-2-[(1S)-1-methoxyethyl]-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione

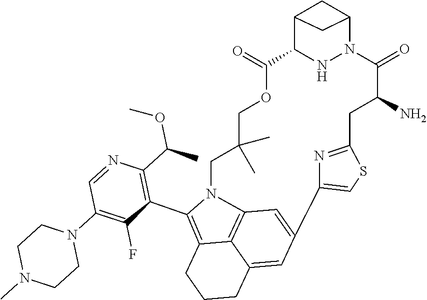

The title intermediate M was prepared in analogy to the preparation of intermediate K by using methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of methyl (3S)-1-[(2S)-3-(4-bromothiazol-2-yl)-2-(tert-butoxycarbonylamino)propanoyl]hexahydropyridazine-3-carboxylate (intermediate C1).

### Intermediate N1

### (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione

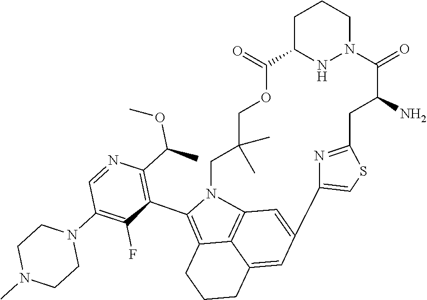

The title intermediate N1 was prepared in analogy to the preparation of intermediate E by using benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (intermediate D4) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Intermediate N2

### (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

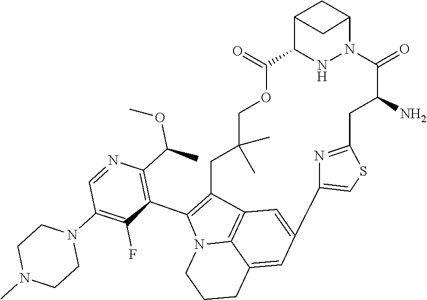

The title intermediate N2 was prepared in analogy to the preparation of Intermediate E by using benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (intermediate D4) and methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-(tert-butoxycarbonylamino)propanoyl]hexahydropyridazine-3-carboxylate (intermediate C1).

### Intermediate N3

### (7S,13S)-7-amino-21-ethyl-(20M)-20-[2-[(1S)-1-methoxyethyl]-4-methyl-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione

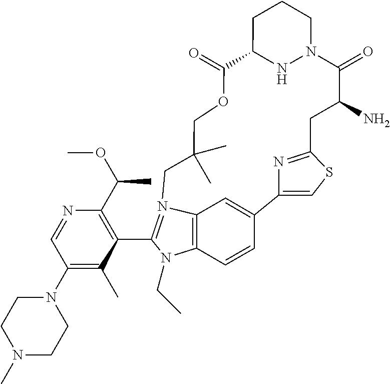

The title intermediate N3 was prepared in analogy to the preparation of intermediate E by using Benzyl 4-[(5M)-5-[1-ethyl-3-(3-hydroxy-2,2-dimethyl-propyl)-5-(4,4,5,5-tetramethyl-1,3,2-dioxaborolan-2-yl)indol-2-yl]-6-[(1S)-1-methoxyethyl]-4-methyl-3-pyridyl]piperazine-1-carboxylate (intermediate D6) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Intermediate N4

### (7S,13S)-7-amino-21-ethyl-(20M)-20-[2-[(1S)-1-methoxyethyl]-4-methyl-5-(4-methylpiperazin-1-yl)-3-pyridyl]-17,17-dimethyl-15-oxa-4-thia-9,21,27,29-tetrazahexacyclo[17.5.2.1^{2,5}.1^{9,13}.1^{10,12}.0^{22,26}]nonacosa-1(25),2,5(29),19,22(26),23-hexaene-8,14-dione

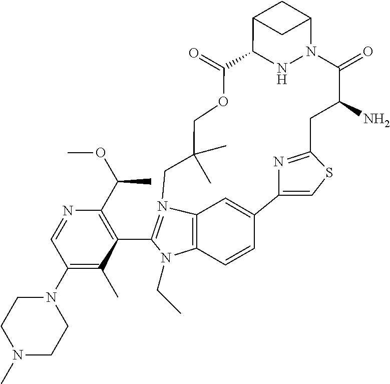

The title intermediate N4 was prepared in analogy to the preparation of intermediate E by using Benzyl 4-[(5M)-5-[1-ethyl-3-(3-hydroxy-2,2-dimethyl-propyl)-5-(4,4,5,5-tetramethyl-1,3,2-dioxaborolan-2-yl)indol-2-yl]-6-[(1S)-1-methoxyethyl]-4-methyl-3-pyridyl]piperazine-1-carboxylate (intermediate D6) and methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-(tert-butoxycarbonylamino)propanoyl]hexahydropyridazine-3-carboxylate (intermediate C1).

### Intermediate N5

### (7S,13S)-7-amino-(20M)-20-[5-(4-cyclopropylpiperazin-1-yl)-2-[(1S)-1-methoxyethyl]-4-methyl-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

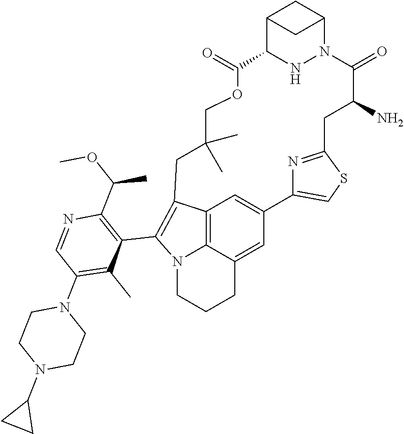

The title intermediate N5 was prepared in analogy to the preparation of intermediate I by using benzyl 4-[(5M)-5-[(7S,13S)-7-(tert-butoxycarbonylamino)-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-20-yl]-6-[(1S)-1-methoxyethyl]-4-methyl-3-pyridyl]piperazine-1-carboxylate (compound N5-a) instead of benzyl 4-[(5P)-5-[(7S,13S)-7-(tert-butoxycarbonylamino)-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-20-yl]-6-[(1S)-1-methoxyethyl]-3-pyridyl]piperazine-1-carboxylate (compound F3).

The compound N5-a was prepared in analogy to the preparation of compound E3 by using benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (intermediate D4) and methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (35)-1-[(25)-3-(4-bromothiazol-2-yl)-2-(tert-butoxycarbonylamino)propanoyl]hexahydropyridazine-3-carboxylate (intermediate C1).

### Intermediate N6

### (7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-7-amino-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

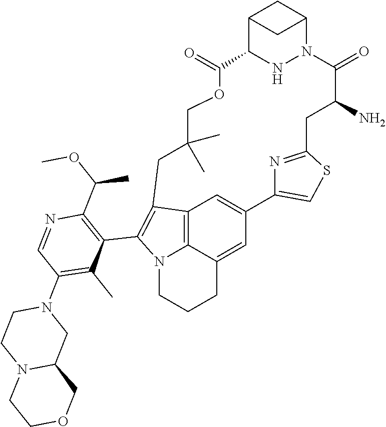

The title intermediate N6 was prepared in analogy to the preparation of intermediate E by using 3-[(2M)-2-[5-[(9aS)-3,4,6,7,9,9a-hexahydro-1H-pyrazino[2,1-c][1,4]oxazin-8-yl]-2-[(15)-1-methoxyethyl]-4-methyl-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol (intermediate D8) and methyl (45)-2-[(25)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-(tert-butoxycarbonylamino)propanoyl]hexahydropyridazine-3-carboxylate (intermediate C1).

### Intermediate N7

### (7S,13S)-7-amino-(20M)-20-[5-(4-cyclopropylpiperazin-1-yl)-2-[(1S)-1-methoxyethyl]-4-methyl-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N7)

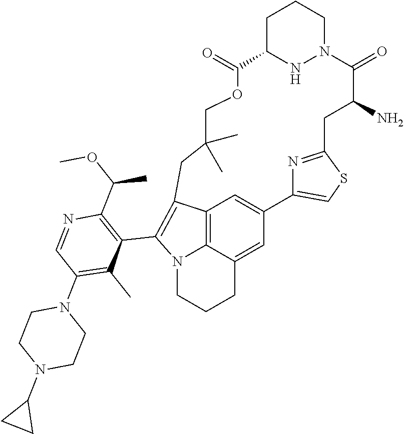

The title intermediate N7 was prepared in analogy to the preparation of intermediate I by using benzyl 4-[(5M)-5-[(7S,13S)-7-(tert-butoxycarbonylamino)-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-20-yl]-6-[(1S)-1-methoxyethyl]-4-methyl-3-pyridyl]piperazine-1-carboxylate (compound N7-a) instead of benzyl 4-[(5P)-5-[(7S,13S)-7-(tert-butoxycarbonylamino)-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-20-yl]-6-[(1S)-1-methoxyethyl]-3-pyridyl]piperazine-1-carboxylate (compound F3).

The compound N7-a was prepared in analogy to the preparation of compound E3 by using benzyl 4-[(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4,6,8(12)-tetraen-2-yl]-6-[(1S)-1-methoxyethyl]-4-methyl-3-pyridyl]piperazine-1-carboxylate (intermediate D4) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Intermediate N8

### (7S,13S)-7-amino-(20M)-20-[4-(difluoromethyl)-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione

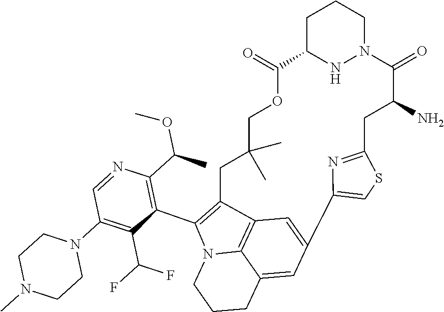

The intermediate N8 was prepared according to the following scheme:

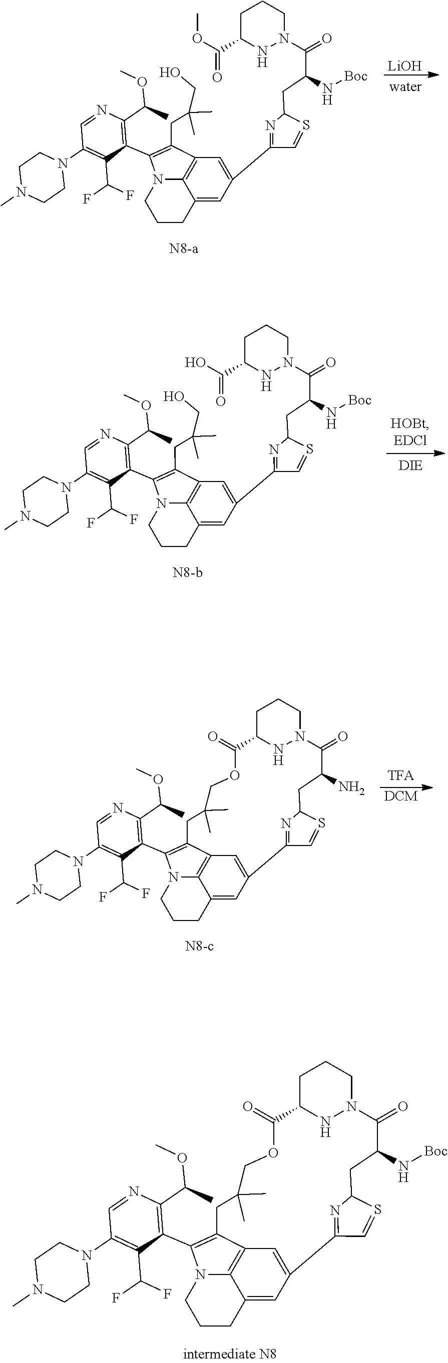

### Step 1: Preparation of (3S)-1-[(2S)-2-(tert-butoxycarbonylamino)-3-[4-[(2M)-2-[4-(difluoromethyl)-2-[(1S)-1-methoxyethyl]-5-(4-methylpiperazin-1-yl)-3-pyridyl]-3-(3-hydroxy-2,2-dimethyl-propyl)-1-azatricyclo[6.3.1.0^{4,12}]dodeca-2,4(12),5,7-tetraen-6-yl]thiazol-2-yl]propanoyl]hexahydropyridazine-3-carboxylic acid (compound N8-b)

A solution of methyl (3S)-1-[(2S)-2-(tert-butoxycarbonylamino)-3-[4-[(2M)-2-[4-(difluoromethyl)-2-[(1S)-1-methoxyethyl]-5-(4-methylpiperazin-1-yl)-3-pyridyl]-3-(3-hydroxy-2,2-dimethyl-propyl)-1-azatricyclo[6.3.1.0^{4,12}]dodeca-2,4(12),5,7-tetraen-6-yl]thiazol-2-yl]propanoyl]hexahydropyridazine-3-carboxylate (compound N8-a, 250 mg, 0.271 mmol) and LiOH·H₂O (13.0 mg, 0.542 mmol) in MeOH/THF/water (2 mL/2 mL/2 mL) was stirred at rt for 2 hours. The reaction mixture was concentrated under reduced pressure. The pH of the resulting solution was adjusted to 5 with 1 N hydrochloric acid (HCl). The mixture was purified by reversed-phase chromatography to afford (3S)-1-[(2S)-2-(tert-butoxycarbonylamino)-3-[4-[(2M)-2-[4-(difluoromethyl)-2-[(1S)-1-methoxyethyl]-5-(4-methylpiperazin-1-yl)-3-pyridyl]-3-(3-hydroxy-2,2-dimethyl-propyl)-1-azatricyclo[6.3.1.0^{4,12}]dodeca-2,4(12),5,7-tetraen-6-yl]thiazol-2-yl]propanoyl]hexahydropyridazine-3-carboxylic acid (compound N8-b, 177 mg) as a light yellow lyophilized powder. MS calc'd 909.4 (MH⁺), measured 909.4 (MH⁺).

### Step 2: Preparation of tert-butyl N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]carbamate (compound N8-c)

A solution of (3S)-1-[(2S)-2-(tert-butoxycarbonylamino)-3-[4-[(2M)-2-[4-(difluoromethyl)-2-[(1S)-1-methoxyethyl]-5-(4-methylpiperazin-1-yl)-3-pyridyl]-3-(3-hydroxy-2,2-dimethyl-propyl)-1-azatricyclo[6.3.1.0^{4,12}]dodeca-2,4(12),5,7-tetraen-6-yl]thiazol-2-yl]propanoyl]hexahydropyridazine-3-carboxylic acid (compound N8-b, 144 mg, 158.4 μmol,), HOBt (145.54 mg, 950.38 μmol), and DIEA (1.1 mL, 6.34 mmol) in DCM (15 mL) was cooled to O ° C. and added EDC HCl (1.2 g, 6.34 mmol). The reaction mixture was stirred at rt for 12 hours. After completion, the reaction mixture was dissolved in DCM (50 mL). The organic layer was washed with sat. NH₄Cl aq. (10 mL, three times), washed with brine (10 mL), dried over Na₂SO₄, and concentrated under reduced pressure. The residue was purified by silica gel chromatography to afford tert-butyl N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]carbamate (compound N8-c, 82 mg, faster eluted) as a light yellow powder. MS calc'd 891.4 (MH⁺), measured 891.4 (MH⁺).

### Step 3: Preparation of (7S,13S)-7-amino-(20M)-20-[4-(difluoromethyl)-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N8)

A solution of tert-butyl N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]carbamate (compound N8-c, 82 mg, 0.092 mmol) and TFA (1 mL) in DCM (2 mL) was stirred at rt for 2 hrs. The solvent was removed under reduced pressure to afford a residue. The residue was dissolved in DCM (10 mL), and the pH was adjusted to 8 using sat. NaHCO₃ aqueous sodium. The mixture was extracted with DCM (10 mL, three times). The combined organic layers were washed with brine (5 mL), dried over Na₂SO₄, filtered, and concentrated under vacuum to afford (7S,13S)-7-amino-(20M)-20-[4-(difluoromethyl)-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N8, 15 mg) as a light yellow oil. MS calc'd 791.4 (MH⁺), measured 791.4 (MH⁺).

The compound N8-a was prepared in analogy to the preparation of compound E1 by using 3-[2-[4-(difluoromethyl)-2-[(1S)-1-methoxyethyl]-5-(4-methylpiperazin-1-yl)-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol (intermediate D11) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and purified via silica gel chromatography (faster eluted).

### Intermediate O1

### (1S,2S)-2-(2-fluoro-3-pyridyl)cyclopropanecarboxylic acid

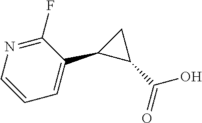 [SMILES: O=C(O)[C@H]1C[C@@H]1c1cccnc1F]

The intermediate O1 was prepared according to the following scheme:

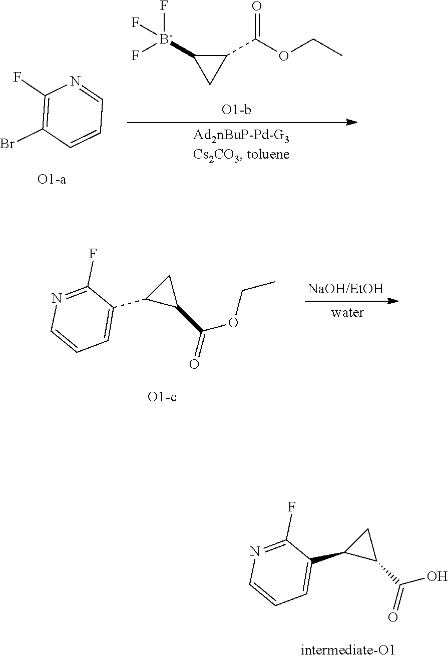

### Step 1: Preparation of trans-ethyl (1R,2R)-2-(2-fluoro-3-pyridyl)cyclopropanecarboxylate (compound O1-c)

To a solution of 3-bromo-2-fluoro-pyridine (compound O1-a, 600 mg, 3.41 mmol), potassium (trans-2-(ethoxycarbonyl)cyclopropyl)trifluoroborate (750.2 mg, 3.41 mmol) in toluene/water (6 mL/0.6 mL) were added with cataCXiumA-Pd-G3 (248.3 mg, 340.93 μmol) and Cs₂CO₃ (2.2 g, 6.82 mmol, 2.0 eq). After being stirred at 90° C. under nitrogen atmosphere for 12 hrs, the suspension was filtered and then the filtrate was concentrated under vacuum to get a residue. The resulting residue was purified by reversed-phase chromatography to afford trans-ethyl (1R,2R)-2-(2-fluoro-3-pyridyl)cyclopropanecarboxylate (compound O1-c, 215 mg) as light yellow oil. MS calc'd 210.1 (MH⁺), measured 210.1 (MH⁺).

### Step 2: Preparation of (1S,2S)-2-(2-fluoro-3-pyridyl)cyclopropanecarboxylic acid (intermediate O1)

A solution of trans-ethyl (1R,2R)-2-(2-fluoro-3-pyridyl)cyclopropanecarboxylate (compound O1-c, 215 mg, 1.03 mmol) and NaOH (82.2 mg, 2.06 mmol) in ethanol/water (1 mL/1 mL) was stirred at rt for 12 hrs. The solvent was removed under vacuum to get a crude. The crude was purified by Prep-HPLC and SFC to afford (1S,2S)-2-(2-fluoro-3-pyridyl)cyclopropanecarboxylic acid (intermediate O1, 33 mg, faster eluted). MS calc'd 182.1 (MH⁺), measured 182.1 (MH⁺).

SFC conditions: Instrument: SFC 150 Mgm; Column: IG, 250×30 mm I.D., 5 μm; Mobile phase: A for CO₂ and B for ethanol (0.1% NH₃H₂O); Gradient: B 15%; Flow rate: 80 mL/min; Back pressure: 100 bar; Column temperature: 35° C.

### Intermediate O2

### (1S,2S)-2-(2-methyl-3-pyridyl)cyclopropanecarboxylic acid

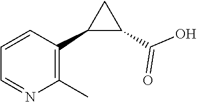

The title intermediate O2 was prepared in analogy to the preparation of intermediate O1 by using 3-bromo-2-methyl-pyridine instead of 3-bromo-2-fluoro-pyridine (compound O1-a).

### Intermediate O3

### (1S,6R,7r)-norcarane-7-carboxylic acid

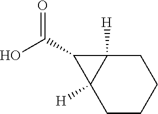

The title intermediate O3 was prepared according to the following scheme:

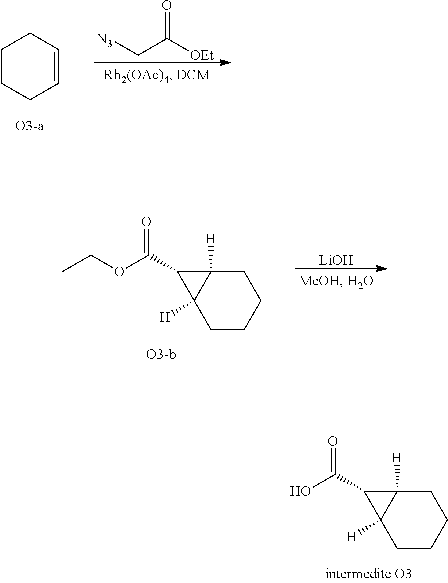

### Step 1: Preparation of ethyl (1S,6R,7r)-norcarane-7-carboxylate (compound O3-b)

To a solution of cyclohexene (compound O3-a, 10 g, 121.75 mmol) in DCM (200 mL) was added Rh₂(OAc)₄ (340.9 mg, 1.2 mmol). The resulting suspension was allowed to stir vigorously at 0° C. while a solution of ethyl diazoacetate (10.0 g, 87.65 mmol) in DCM (200 mL) was added dropwise over a period of 5 hrs. The mixture was stirred at 20° C. for 15 hrs. The reaction mixture was poured into water (150 mL) and extracted with DCM (50 mL, twice). The combined organic layer was washed with brine (100 mL), dried over Na₂SO₄, filtered and concentrated under vacuum to afford ethyl (1S,6R,7r)-norcarane-7-carboxylate (compound O3-b, 12.0 g, crude) as yellow oil which was used in the next step directly without further purification.

### Step 2: Preparation of (1S,6R,7r)-norcarane-7-carboxylic acid (intermediate O3)

To a solution of ethyl (1S,6R,7r)-norcarane-7-carboxylate (compound O3-b, 10.0 g, 59.44 mmol) in methanol (80 mL) and water (20 mL) was added LiOH (12.5 g, 297.19 mmol) portion wise. The mixture was stirred at 25° C. for 16 hrs. EtOAc (200 mL) and water (200 mL) were added, and layers were separated. The aqueous phase was washed with EtOAc (100 mL, twice). The aqueous phase was acidified with 1N HCl to pH=1-2 and extracted by EtOAc (100 mL, twice). The organic phase was dried over Na₂SO₄, filtered and concentrated under vacuum to give a residue which was purified via column chromatography to afford (1S,6R,7r)-norcarane-7-carboxylic acid (intermediate O3, 4.4 g) as a white solid. MS calc'd 139.1 (M−H)⁻, measured 139.2 (M−H)⁻.

### Intermediate O4

### (1R,6S,7R)-3-oxabicyclo[4.1.0]heptane-7-carboxylic acid

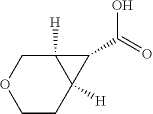

The title intermediate O4 was prepared according to the following scheme:

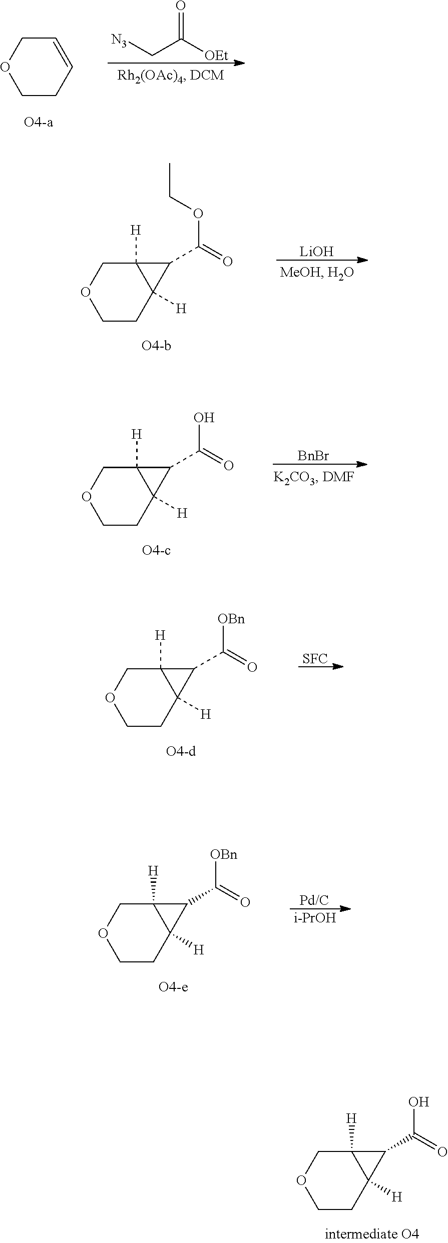

### Step 1: Preparation of trans-ethyl (1R,6S,7R)-3-oxabicyclo[4.1.0]heptane-7-carboxylate (compound O4-b)

To a mixture of Rh₂(OAc)₄ (525.4 mg, 1.19 mmol) in DCM (20 mL), 3,6-dihydro-2H-pyran (compound O4-a, 2.0 g, 23.78 mmol) was added under N2 atmosphere. Then the mixture was stirred to 40° C., and a solution of ethyl diazoacetate (2.9 g, 26.15 mmol) in DCM (20 mL) was added dropwise over 2 hrs. Then the mixture was stirred at 40° C. for another 2 hrs. The mixture was filtered and concentrated under vacuum to give a residue. The residue was purified via column chromatography to afford trans-ethyl (1R,6S,7R)-3-oxabicyclo[4.1.0]heptane-7-carboxylate (compound O4-b, 1.0 g crude) as colorless oil. ¹H NMR (400 MHz, CHLOROFORM-d) δ=4.18-4.09 (m, 2H), 4.00-3.91 (m, 1H), 3.90-3.80 (m, 1H), 3.67-3.50 (m, 1H), 3.33-3.18 (m, 1H), 1.99-1.90 (m, 1H), 1.90-1.82 (m, 1H), 1.82-1.74 (m, 1H), 1.73-1.68 (m, 1H), 1.63-1.55 (m, 1H), 1.28 (s, 3H).

### Step 2: Preparation of trans-(1R,6S,7R)-3-oxabicyclo[4.1.0]heptane-7-carboxylic acid (compound O4-c)

To a solution of trans-ethyl (1R,6S,7R)-3-oxabicyclo[4.1.0]heptane-7-carboxylate (compound O4-b, 1 g, 5.88 mmol) in THF (10 mL) and water (1 mL), LiOH (295.8 mg, 7.05 mmol) was added at 25° C., then the solution was stirred at 25° C. for 16 hrs. The solution was poured into 10 mL of ice-water and then extracted with EtOAc (10 mL). The organic layer was removed, and the pH of water layer was adjusted to 4 with 1 N aq. HCl. Then the water phase was extracted with EtOAc (10 mL, twice). The organic layer was dried over Na₂SO₄ and concentrated to give trans-(1R,6S,7R)-3-oxabicyclo[4.1.0]heptane-7-carboxylic acid (compound O4-c, 300.0 mg) as a light brown solid. ¹H NMR (400 MHz, CHLOROFORM-d) δ=4.05-3.93 (m, 1H), 3.90-3.82 (m, 1H), 3.67-3.56 (m, 1H), 3.35-3.18 (m, 1H), 2.02-1.81 (m, 3H), 1.73 (s, 2H).

### Step 3: Preparation of trans-benzyl(1R,6S,7R)-3-oxabicyclo[4.1.0]heptane-7-carboxylate (compound O4-d)

To a mixture of trans-(1R,6S,7R)-3-oxabicyclo[4.1.0]heptane-7-carboxylic acid (compound O4-c, 300.0 mg, 2.11 mmol) in DMF (1 mL), K₂CO₃ (875.0 mg, 6.33 mmol), BnBr (541.3 mg, 3.17 mmol) were added. The mixture was stirred at 25° C. for 16 hrs. The reaction mixture was poured into ice-water (10 mL), and the mixture was extracted with EtOAc (10 mL, three times). The combined organic layer was washed with brine (10 mL, three times), dried over Na₂SO₄ and concentrated under vacuum to give a residue. The residue was purified via column chromatography to give trans-benzyl (1R,6S,7R)-3-oxabicyclo[4.1.0]heptane-7-carboxylate (compound O4-d, 150.0 mg) as a colorless oil. MS calc'd 233.1 (MH⁺), measured 233.2 (MH⁺).

### Step 4: Preparation of benzyl (1R,6S,7R)-3-oxabicyclo[4.1.0]heptane-7-carboxylate (compound O4-e)

The racemic product trans-benzyl (1R,6S,7R)-3-oxabicyclo[4.1.0]heptane-7-carboxylate (compound O4-d, 150.0 mg, 0.65 mmol) was purified by preparative SFC (Instrument: Waters 80Q, Column: DAICEL CHIRALPAK AY-H (250 mm×30 mm×10 μm), Mobile phase: 20% IPA(BASE) in Supercritical CO₂, Gradient: B %=20.00% isocratic elution mode, Flow rate: 50.00 g/min, Monitor wavelength: 220&254 nm, Column temperature: 35° C., System back pressure: 100 bar and concentrated under vacuum to give benzyl (1R,6S,7R)-3-oxabicyclo[4.1.0]heptane-7-carboxylate (compound O4-e, 67.0 mg, faster eluted) as colorless oil. MS calc'd 233.2 (MH⁺), measured 233.2 (MH⁺).

### Step 5: Preparation of (1R,6S,7R)-3-oxabicyclo[4.1.0]heptane-7-carboxylic acid (intermediate O4)

To a mixture of Pd on activated carbon (20.0 mg, 10% W/W) in IPA (2 mL), benzyl (1R,6S,7R)-3-oxabicyclo[4.1.0]heptane-7-carboxylate (compound O4-e, 17.0 mg, 0.07 mmol) was added under N₂ atmosphere, and the mixture was degassed under vacuum and purged with N2 atmosphere for three times, then the mixture was stirred at 25° C. for 1 h under H2 atmosphere (hydrogen balloon). The mixture was filtered off and the filtrate was concentrated under vacuum to give (1R,6S,7R)-3-oxabicyclo[4.1.0]heptane-7-carboxylic acid (intermediate O4, 16.0 mg) as a colorless solid, which was used in the next step directly without any further purification.

### Intermediate O5

### (1S,2R)-2-tetrahydropyran-4-ylcyclopropanecarboxylic acid

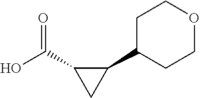 [SMILES: O=C(O)[C@H]1C[C@@H]1C1CCOCC1]

The title intermediate O5 was prepared according to the following scheme:

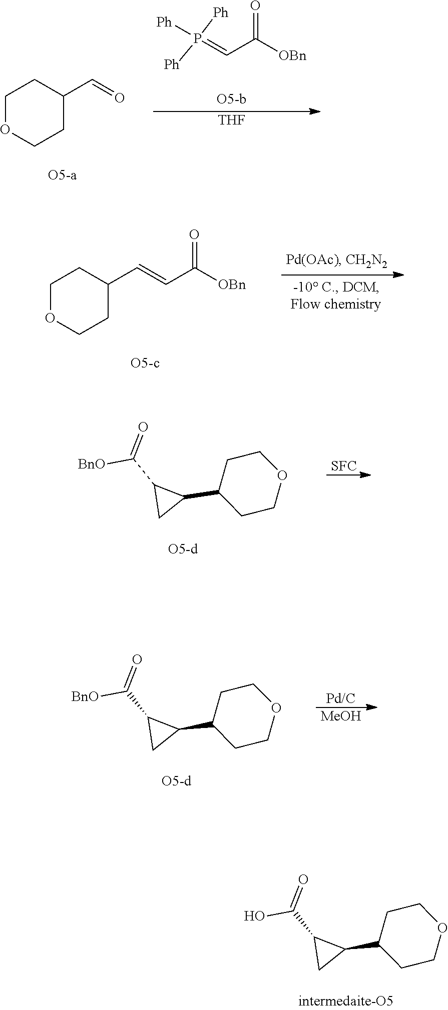

### Step 1: Preparation of benzyl (E)-3-tetrahydropyran-4-ylprop-2-enoate (compound O5-c)

To a solution of tetrahydropyran-4-carbaldehyde (compound O5-a, 5.0 g, 43.81 mmol) in THF (200 mL), benzyl 2-(triphenyl-λ⁵-phosphanylidene)acetate (compound O5-b, 19.8 g, 48.19 mmol) was added. The reaction mixture was stirred at 20° C. for 6 hrs. The reaction mixture was concentrated under vacuum to give a residue, which was purified via column chromatography to afford benzyl (E)-3-tetrahydropyran-4-ylprop-2-enoate (intermediate O5-c, 10.0 g, 40.6 mmol, 92.68% yield) as a colorless oil.

### Step 2: Preparation of benzyl (1S,2R)-2-tetrahydropyran-4-ylcyclopropanecarboxylate (compound O5-e)

Solution 1: (benzyl (E)-3-tetrahydropyran-4-ylprop-2-enoate, 5 g) in (DCM, 100 mL).
Solution 2: (diazomethane, 5.1 g, 0.75 M, 162.4 mL).
Solution 3: (Pd(OAc)₂, 0.03 g, 0.004 M, 28.9 mL).

The solution 1 was pumped by Pump 1 (S1, P1, 34.7 mL/min) to flow reactor 1 (FLR1, PFA, Coils reactor, 3.2 (⅛″) mm, 5.0 mL, −10° C.). The solution 2 was pumped by Pump 2 (S2, P2, 36.6 mL/min) to flow reactor 1 (FLR1, PFA, Coils reactor, 3.1 (⅛″) mm, 5.0 mL, −10° C.). The solution 3 was pumped by Pump 3 (S3, P3, 6.5 mL/min) to flow reactor 2 (FLR2, PFA, Coils reactor, 3.1 (⅛″) mm, 10.0 mL, −10° C.). The residence time of flow reactor 1 was (FLR1, 0.07 min). The residence time of flow reactor 2 was (FLR2, 0.13 min).

The mixture was collected with a bottle at 20° C. The Pump 1 and Pump 2 started at the same time. After 0.07 min, the Pump 3 started. The reaction mixture was collected after running 0.2 min. The reaction mixture was filtered and concentrated under vacuum to give the residue. The residue was purified via column chromatography to give the crude trans-benzyl (1S,2R)-2-tetrahydropyran-4-cyclopropanecarboxylate (intermediate O5-d). The crude product was purified again by prep-HPLC and SFC to afford benzyl (1S,2R)-2-tetrahydropyran-4-cyclopropanecarboxylate (compound O5-e, 260 mg). ¹H NMR (400 MHz, CHLOROFORM-d) δ=7.43-7.30 (m, 5H), 5.20-5.05 (m, 2H), 4.06-3.91 (m, 2H), 3.40-3.26 (m, 2H), 1.70-1.61 (m, 2H), 1.54-1.42 (m, 3H), 1.39-1.30 (m, 1H), 1.25-1.17 (m, 1H), 1.04-0.92 (m, 1H), 0.86-0.76 (m, 1H).

SFC conditions: column: DAICEL CHIRALPAK AD (250 mm×50 mm, 10 μm); mobile phase: [A: CO₂; B: MeOH]; B %: 25.00%-25.00%, 9.20 min; flow rate: 150.00 g/min.

### Step 3: Preparation of (1S,2R)-2-tetrahydropyran-4-ylcyclopropanecarboxylic acid (intermediate O5)

To a solution of benzyl (1S,2R)-2-tetrahydropyran-4-ylcyclopropanecarboxylate (compound O5-e, 260.0 mg, 1.0 mmol) in methanol (8 mL), Pd on activated carbon (200.0 mg) was added. The reaction mixture was purged and degassed with H2 three times then stirred under H2 atmosphere at 25° C. for another 2 hrs. The reaction mixture was filtered and the filtrate was co-evaporated with ACN for three times to afford (1S,2R)-2-tetrahydropyran-4-ylcyclopropanecarboxylic acid (intermediate O5, 180.0 mg) as a white solid. ¹H NMR (400 MHz, CHLOROFORM-d) δ=4.00-3.94 (m, 2H), 3.37-3.28 (m, 2H), 1.71-1.60 (m, 2H), 1.54-1.41 (m, 3H), 1.39-1.31 (m, 1H), 1.25-1.16 (m, 1H), 1.03-0.93 (m, 1H), 0.88-0.80 (m, 1H).

### Intermediate O6

### (1R,6S,7R)-3-methylnorcarane-7-carboxylic acid

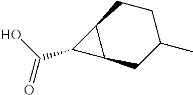

The title intermediate O6 was prepared in analogy to the preparation of intermediate O3 by using 4-methylcyclohexene instead of cyclohexene (compound O3-a).

### Intermediate P

### (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione

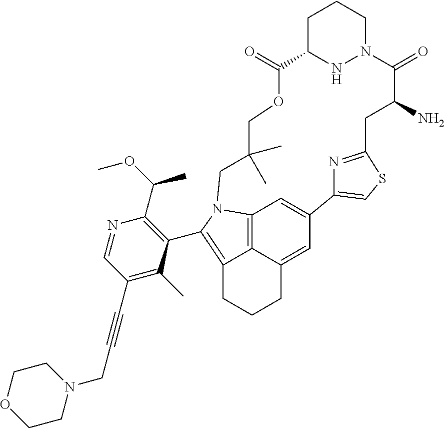

The title intermediate P was prepared according to the following scheme:

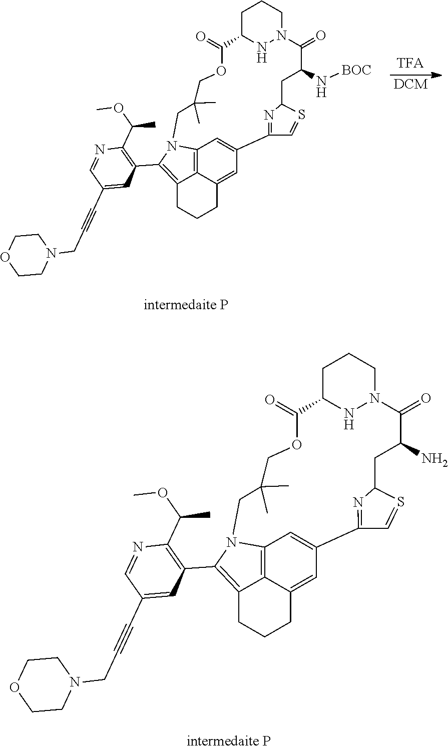

To a solution of tert-butyl N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]carbamate (compound P-4, 60.0 mg, 0.07 mmol) in DCM (2 mL), TFA (0.5 mL, 6.73 mmol) was added. The mixture was stirred at 25° C. for 0.5 h. The reaction mixture was concentrated under vacuum to give a crude. To the crude, sat. NaHCO₃ aq. (180 mL) and EtOAc (100 mL) were added. The mixture was separated, and the aqueous phase was extracted with EtOAc (60 mL, three times). The combined organic layer was washed with brine (100 mL), dried over Na₂SO₄, filtered and concentrated under vacuum to afford (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate P, 40.0 mg) as a brown solid. MS calc'd 780.4 (MH⁺), measured 780.9 (MH⁺).

The compound P-4 was prepared in analogy to the preparation of compound E4 by using 3-[7-chloro-(2P)-2-[2-[(1S)-1-methoxyethyl]-4-methyl-5-(3-morpholinoprop-1-ynyl)-3-pyridyl]-4,5-dihydro-3H-benzo[cd]indol-1-yl]-2,2-dimethyl-propan-1-ol (intermediate D5) and [2-[(2S)-2-(tert-butoxycarbonylamino)-3-[(3S)-3-methoxycarbonylhexahydropyridazin-1-yl]-3-oxo-propyl]thiazol-4-yl]boronic acid (intermediate C4) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-(tert-butoxycarbonylamino)propanoyl]hexahydropyridazine-3-carboxylate (intermediate C1).

### Intermediate Q

### (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,32-tetrazaheptacyclo[23.3.1.1^{2,5}1^{9,13}.1^{10,12}.0^{19,27}.0^{21,26}]dotriaconta-1(28),2,5(32),20,25(29),26-hexaene-8,14-dione

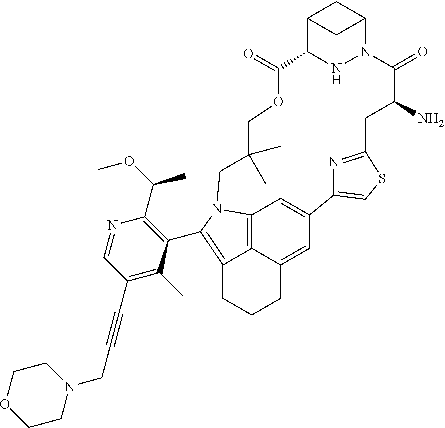

The title intermediate Q was prepared in analogy to the preparation of Intermediate P by using [2-[(2S)-2-(tert-butoxycarbonylamino)-3-[(4S)-4-methoxycarbonyl-2,3-diazabicyclo[3.1.1]heptan-2-yl]-3-oxo-propyl]thiazol-4-yl]boronic acid (intermediate C5) instead of [2-[(2S)-2-(tert-butoxycarbonylamino)-3-[(3S)-3-methoxycarbonylhexahydropyridazin-1-yl]-3-oxo-propyl]thiazol-4-yl]boronic acid (intermediate C4).

### Intermediate R1

### (7S,13S)-7-amino-(20P)-20-[4-fluoro-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione

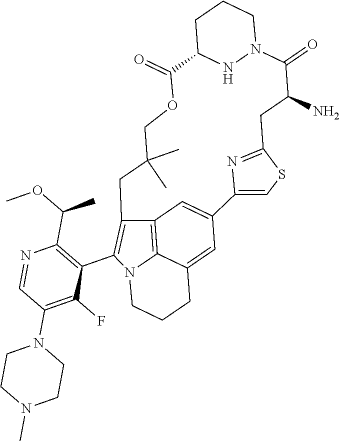

The title intermediate R1 was prepared in analogy to the preparation of intermediate E by using benzyl 4-[(5P)-5-[3-[3-[tert-butyl(dimethyl)silyl]oxy-2,2-dimethyl-propyl]-6-(4,4,5,5-tetramethyl-1,3,2-dioxaborolan-2-yl)-1-azatricyclo[6.3.1.0^{4,12}]dodeca-2,4(12),5,7-tetraen-2-yl]-4-fluoro-6-[(1S)-1-methoxyethyl]-3-pyridyl]piperazine-1-carboxylate (intermediate D7) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

### Intermediate R2

### (7S,13S)-7-amino-(20P)-20-[4-fluoro-2-[(1S)-1-methoxyethyl]-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione intermediate R2)

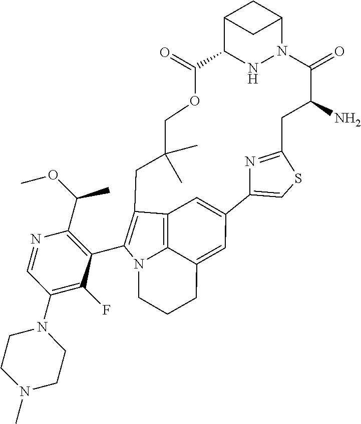

The title intermediate R2 was prepared in analogy to the preparation of intermediate E by using benzyl 4-[(5P)-5-[3-[3-[tert-butyl(dimethyl)silyl]oxy-2,2-dimethyl-propyl]-6-(4,4,5,5-tetramethyl-1,3,2-dioxaborolan-2-yl)-1-azatricyclo[6.3.1.0^{4,12}]dodeca-2,4(12),5,7-tetraen-2-yl]-4-fluoro-6-[(1S)-1-methoxyethyl]-3-pyridyl]piperazine-1-carboxylate (intermediate D7) and methyl (4S)-2-[(2S)-3-(4-bromothiazol-2-yl)-2-(tert-butoxycarbonylamino) propanoyl]-2,3-diazabicyclo[3.1.1]heptane-4-carboxylate (intermediate C2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(15)-1-methoxyethyl]-4-methyl-3-pyridyl]piperazine-1-carboxylate (intermediate D1) and methyl (35)-1-[(2S)-3-(4-bromothiazol-2-yl)-2-(tert-butoxycarbonylamino)propanoyl]hexahydropyridazine-3-carboxylate (intermediate C1).

### Intermediate S1

### (7S,13S)-7-amino-(20M)-20-[2-[(1S)-1-methoxyethyl]-4-methyl-5-(3-morpholinoprop-1-ynyl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione

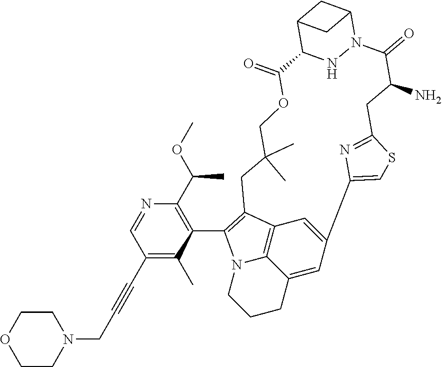

The title intermediate S1 was prepared according to the following scheme:

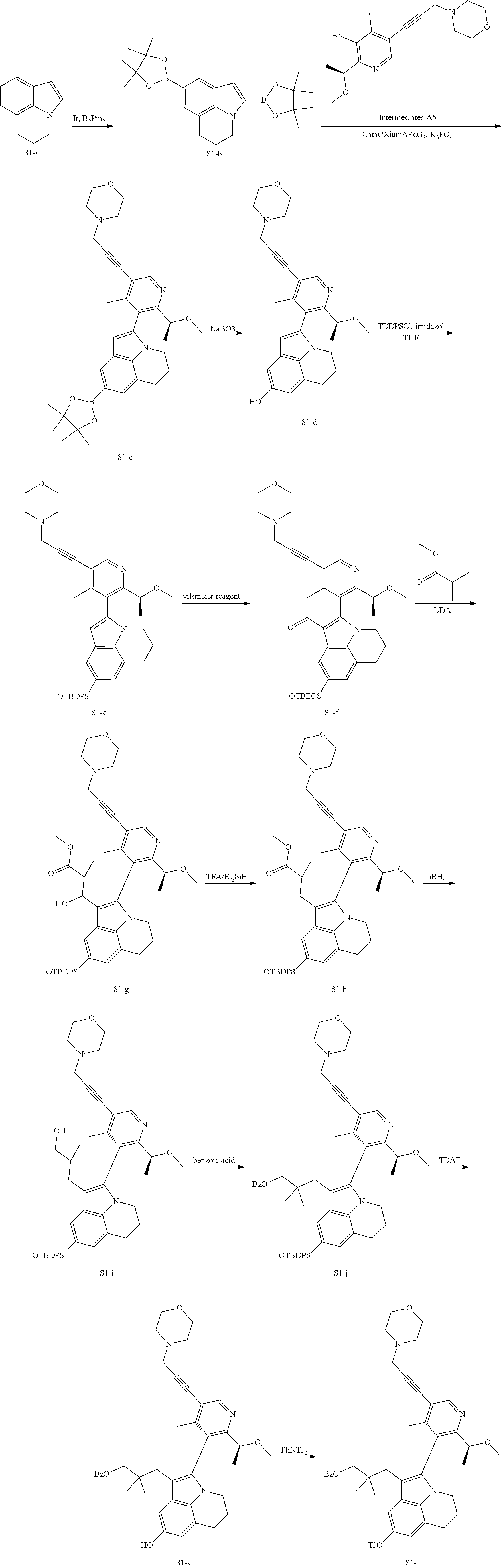

### Step 1: Preparation of 2,6-bis(4,4,5,5-tetramethyl-1,3,2-dioxaborolan-2-yl)-1-azatricyclo[6.3.1.0^{4,12}]dodeca-2,4,6,8(12)-tetraene (compound S1-b)

A mixture of 3,4,7,8-tetramethyl-1,10-phenanthroline (902 mg, 3.82 mmol), bis(pinacolato)diboron (32 g, 127 mmol) and [Ir(OMe)(COD)]2 (1.3 mg, 1.91 mmol) in THF (100 mL) was stirred at 25° C. for 5 min. Then 1-azatricyclo[6.3.1.0^{4,12}]dodeca-2,4,6,8(12)-tetraene (compound S1-a, 10.0 g, 63.6 mmol) in THF (100 mL) was added to the mixture and the mixture was degassed under vacuum and purged with nitrogen for three times. After being stirred at 70° C. for 15 hrs, the reaction mixture was concentrated under vacuum to give a residue. The residue was purified by reversed phase chromatography to afford 2,6-bis(4,4,5,5-tetramethyl-1,3,2-dioxaborolan-2-yl)-1-azatricyclo[6.3.1.0^{4,12}]dodeca-2,4,6,8(12)-tetraene (compound S1-b, 15.9 g) as brown oil. MS calc'd 410.3 (MH⁺), measured 410.3 (MH⁺)

### Step 2: Preparation of 4-[3-[6-[(1S)-1-methoxyethyl]-4-methyl-5-[6-(4,4,5,5-tetramethyl-1,3,2-dioxaborolan-2-yl)-1-azatricyclo[6.3.1.0^{4,12}]dodeca-2,4(12),5,7-tetraen-2-yl]-3-pyridyl]prop-2-ynyl]morpholine (compound S1-c)

To a solution of 2,6-bis(4,4,5,5-tetramethyl-1,3,2-dioxaborolan-2-yl)-1-azatricyclo[6.3.1.0^{4,12}]dodeca-2,4,6,8(12)-tetraene (compound S1-b, 8.9 g, 21.7 mmol) and 4-[3-[5-bromo-6-[(1S)-1-methoxyethyl]-4-methyl-3-pyridyl]prop-2-ynyl]morpholine (intermediate A5, 4.8 g, 13.6 mmol) in 1,4-dioxane (200 mL) and Water (40 mL) were added K₃PO₄ (8.6 g, 40.8 mmol) and cataCXium A Pd G3 (3.0 g, 4.08 mmol) at 25° C., the mixture was degassed and purged with nitrogen for three times and stirred at 60° C. for 1 hr. The reaction mixture was poured into water (100 mL), and EtOAc (20 mL) was added. Then the solution was extracted with EtOAc (50 mL, three times). The combined organic layer was washed with brine (50 mL), dried over Na₂SO₄ and concentrated under reduced pressure to give a residue. The residue was purified via flash chromatography to afford 4-[3-[6-[(1S)-1-methoxyethyl]-4-methyl-5-[6-(4,4,5,5-tetramethyl-1,3,2-dioxaborolan-2-yl)-1-azatricyclo[6.3.1.0^{4,12}]dodeca-2,4(12),5,7-tetraen-2-yl]-3-pyridyl]prop-2-ynyl]morpholine (compound S1-c, 1.3 g) as off-white solid. MS calc'd 556.3 (MH⁺), measured 556.5 (MH⁺).

### Step 3: Preparation of 2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-ol (compound S1-d)

To a solution of 4-[3-[6-[(15)-1-methoxyethyl]-4-methyl-5-[6-(4,4,5,5-tetramethyl-1,3,2-dioxaborolan-2-yl)-1-azatricyclo[6.3.1.0^{4,12}]dodeca-2,4(12),5,7-tetraen-2-yl]-3-pyridyl]prop-2-ynyl]morpholine (compound S1-c, 7.0 g, 12.6 mmol) in THF (70 mL), a solution of sodium perborate (5.8 g, 37.8 mmol) in water (70 mL) was added at 25° C. The mixture was stirred at 25° C. for 1 hr. The reaction mixture was poured into water (50 mL), and then extracted with EtOAc (70 mL, three times). The combined organic layer was washed with brine (100 mL), dried over Na₂SO₄ and concentrated under reduced pressure to give a residue. The residue was purified by Prep-HPLC to afford 2-[2-[(15)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-ol (compound S1-d, 900 mg) as a yellow solid. MS calc'd 446.2 (MH⁺), measured 446.2 (MH⁺).

### Step 4: Preparation of tert-butyl-[[2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]oxy]-diphenyl-silane (compound S1-e)

To a solution of 2-[2-[(15)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-ol (compound S1-d, 1.0 g, 2.24 mmol) in THF (20 mL) was added imidazole (458 mg, 6.73 mmol) and tert-butylchlorodiphenylsilane (1.8 g, 6.73 mmol) at 25° C. The reaction mixture was stirred at 25° C. for 16 hrs. The reaction mixture was poured into water (70 mL) and extracted with EtOAc (50 mL, three times). The combined organic layer was washed with brine (100 mL), dried over Na₂SO₄ and concentrated under reduced pressure to give a residue. The residue was purified by column chromatography to afford tert-butyl-[[2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]oxy]-diphenyl-silane (compound S1-e, 1.30 g) as a yellow solid. MS calc'd 684.4 (MH⁺), measured 684.5 (MW).

### Step 5: Preparation of 6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraene-3-carbaldehyde (compound S1-f)

To the mixture of tert-butyl-[[2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]oxy]-diphenyl-silane (compound S1-e, 1.3 g, 1.90 mmol) in DMF (30 mL), vilsmeier reagent (1.2 g, 9.50 mmol) was added in one portion. The mixture was stirred at 50° C. for 2 hrs. The reaction was poured to sat. NaHCO₃ (100 mL) aqueous solution and stirred at 25° C. for 0.5 hr. Then the reaction mixture was separated, and the aqueous phase was extracted with EtOAc (50 mL, twice) and washed with brine (50 mL, twice). The combined organic layer was concentrated under vacuum to give the crude product. The crude was purified via column to afford 6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraene-3-carbaldehyde (compound S1-f, 250 mg) as brown oil. MS calc'd 712.4 (MH⁺), measured 712.4 (MH⁺).

### Step 6: Preparation of methyl 3-[6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-3-hydroxy-2,2-dimethyl-propanoate (compound S1-g)

To a solution of methyl isobutyrate (170 mg, 1.66 mmol) in THF (4 mL) was added LDA (0.8 mL, 1.66 mmol) dropwise at −70° C. under nitrogen. After being stirred for 0.5 hr, the reaction mixture was added with a solution of 6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraene-3-carbaldehyde (compound S1-f, 395 mg, 0.55 mmol) in THF (4 mL) dropwise at −70° C. Then the mixture was stirred at −70° C. for 1 hr. under nitrogen. The mixture was quenched with sat. NH₄Cl (10 mL) aqueous solution and extracted with EtOAc (15 mL, three times). The organic phase was washed with brine (20 mL) and concentrated under vacuum. The crude was purified via column chromatography to give methyl 3-[6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-3-hydroxy-2,2-dimethyl-propanoate (compound S1-g, 446 mg) as brown oil. MS calc'd 814.4 (MH⁺), measured 814.4 (MH⁺).

### Step 7: methyl 3-[6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propanoate (compound S1-h)

To a solution of methyl 3-[6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-3-hydroxy-2,2-dimethyl-propanoate (compound S1-g, 446 mg, 0.55 mmol) in DCM (15 mL) was added TFA (0.8 mL, 11.0 mmol) and Et₃SiH (510 mg, 4.38 mmol) at 0° C. The mixture was stirred at 25° C. for 5 hrs. The reaction was concentrated under vacuum to give a residue. The residue was dissolved in EtOAc and quenched with sat. NaHCO₃ (10 mL) aqueous solution. The mixture was extracted with EtOAc (30 mL, three times), washed with brine (20 mL) and concentrated under vacuum to give crude product. The crude was purified via column chromatography to give methyl 3-[6-[tert-butyl(diphenyl)silyl]oxy-2-[2-[(15)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propanoate (compound S1-h, 389 mg) as yellow oil. MS calc'd 798.4 (MH⁺), measured 798.5 (MH⁺).

### Step 8: 3-[6-[tert-butyl(diphenyl)silyl]oxy-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propan-1-ol (compound S1-i)

To a solution of methyl 3-[6-[tert-butyl(diphenyl)silyl]oxy-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propanoate (compound S1-h, 389 mg, 0.49 mmol) in THF (1.5 mL), lithium aluminum hydride (28 mg, 0.73 mmol) was added dropwise at 0° C. under a nitrogen atmosphere. The mixture was stirred for 10 min at 0° C. Subsequently, the reaction was quenched by the slow addition of aqueous hydrochloric acid solution (1 M) until the pH was less than 5. The resulting mixture was extracted with ethyl acetate (25 mL, three times). The combined organic layer was dried over anhydrous sodium sulfate, filtered, and concentrated under reduced pressure to get a residue. The residue was further purified by silica gel column chromatography to afford 3-[6-[tert-butyl(diphenyl)silyl]oxy-(2M)-2-[2-[(15)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propan-1-ol (compound S1-i, 248 mg, faster eluted) as yellow oil. MS calc'd 770.4 (MH⁺), measured 770.3 (MH⁺).

### Step 9: Preparation of [3-[6-[tert-butyl(diphenyl)silyl]oxy-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propyl]benzoate (compound S1-j)

To a solution of 3-[6-[tert-butyl(diphenyl)silyl]oxy-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propan-1-ol (compound S1-i, 307 mg, 0.40 mmol) and benzoic acid (48.7 mg, 0.40 mmol) in ACN (3 mL) was added NMI (115 mg, 1.40 mmol) and TCFH (224 mg, 0.48 mmol) at 20° C. The resulting mixture was stirred at 20° C. for 1 hr. The reaction mixture was concentrated under vacuum to give a residue. The residue was purified via column chromatography to afford [3-[6-[tert-butyl(diphenyl)silyl]oxy-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propyl]benzoate (compound S1-j, 327 mg) as brown oil. MS calc'd 874.5 (MH⁺), measured 874.5 (MH⁺).

### Step 10: Preparation of [3-[6-hydroxy-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propyl]benzoate (compound S1-k)

To a solution of [3-[6-[tert-butyl(diphenyl)silyl]oxy-(2A4)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propyl]benzoate (compound S1-j, 327 mg, 0.37 mmol) in THF (1.5 mL), a solution of TBAF in THF (0.56 mL, 0.56 mmol) was added at 0° C. for 10 min. After being stirred for 1 hr, the reaction mixture was poured into sat. NH₄Cl aq. (10 mL), extracted with EtOAc (15 mL, twice), dried over Na₂SO₄ and concentrated under vacuum to give crude product. The crude was purified via column chromatography to afford [3-[6-hydroxy-(2A4)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propyl]benzoate (compound S1-k, 173 mg) as brown oil. MS calc'd 636.6 (MH⁺), measured 636.5 (MH⁺).

### Step 11: Preparation of [3-[(2M)-2-[2-[(1S)-1-methoxyethyl]-4-methyl-5-(3-morpholinoprop-1-ynyl)-3-pyridyl]-6-(trifluoromethylsulfonyloxy)-1-azatricyclo[6.3.1.0^{4,12}]dodeca-2,4(12),5,7-tetraen-3-yl]-2,2-dimethyl-propyl]benzoate (compound S1-1)

To a solution of [3-[6-hydroxy-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-3-yl]-2,2-dimethyl-propyl]benzoate (compound S1-k, 168 mg, 0.26 mmol) in DCM (3 mL), was added TEA (53.5 mg, 0.53 mmol), DMAP (2.9 mg, 0.03 mmol) and PhNTf₂ (142 mg, 0.40 mmol) at 20° C. The mixture was stirred at 20° C. for 1 hr. The reaction was concentrated under vacuum to give the crude product. The crude was purified via column chromatography to give [3-[(2M)-2-[2-[(1S)-1-methoxyethyl]-4-methyl-5-(3-morpholinoprop-1-ynyl)-3-pyridyl]-6-(trifluoromethylsulfonyloxy)-1-azatricyclo[6.3.1.0^{4,12}]dodeca-2,4(12),5,7-tetraen-3-yl]-2,2-dimethyl-propyl]benzoate (compound S1-1, 246 mg) as brown oil. MS calc'd 768.3 (MH⁺), measured 768.2 (MH⁺).

### Step 12: Preparation of methyl (4S)-2-[(2S)-3-[4-[3-(3-benzoyloxy-2,2-dimethyl-propyl)-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]thiazol-2-yl]-2-(tert-butoxycarbonylamino)propanoyl]-2,3-diazabicyclo[3.1.1]heptane-4-carboxylate (compound S1-m)

To a mixture of [3-[(2M)-2-[2-[(1S)-1-methoxyethyl]-4-methyl-5-(3-morpholinoprop-1-ynyl)-3-pyridyl]-6-(trifluoromethylsulfonyloxy)-1-azatricyclo[6.3.1.0^{4,12}]dodeca-2,4(12),5,7-tetraen-3-yl]-2,2-dimethyl-propyl]benzoate (compound S1-1, 158 mg, 0.21 mmol) and [2-[(2S)-2-(tert-butoxycarbonylamino)-3-[(4S)-4-methoxycarbonyl-2,3-diazabicyclo[3.1.1]heptan-2-yl]-3-oxo-propyl]thiazol-4-yl]boronic acid (intermediate C5, 117 mg, 0.21 mmol) in toluene (3 mL) was added K₃PO₄ in water (0.4 mL, 0.62 mmol, 1.5 mol/L), followed by XPhos G4 Pd (17.7 mg, 0.02 mmol). The mixture was degassed by bubbling nitrogen gas through the solution for 2 minutes. The mixture was stirred at 110° C. for 10 min under nitrogen atmosphere. The mixture was purified via column chromatography to afford methyl (4S)-2-[(2S)-3-[4-[3-(3-benzoyloxy-2,2-dimethyl-propyl)-(2A4)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]thiazol-2-yl]-2-(tert-butoxycarbonylamino)propanoyl]-2,3-diazabicyclo[3.1.1]heptane-4-carboxylate (compound S1-m, 101 mg) as yellow oil. MS calc'd 1028.5 (MH⁺), measured 1028.5 (MH⁺).

### Step 13: Preparation of (4S)-2-[(2S)-2-(tert-butoxycarbonylamino)-3-[4-[3-(3-hydroxy-2,2-dimethyl-propyl)-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]thiazol-2-yl]propanoyl]-2,3-diazabicyclo[3.1.1]heptane-4-carboxylic acid (compound S1-n)

To a solution of methyl (4S)-2-[(2S)-3-[4-[3-(3-benzoyloxy-2,2-dimethyl-propyl)-(2M f)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]thiazol-2-yl]-2-(tert-butoxycarbonylamino)propanoyl]-2,3-diazabicyclo[3.1.1]heptane-4-carboxylate (compound S1-m, 101 mg, 0.10 mmol) in Methanol (1 mL)/THF (1 mL)/water (0.300 mL) was added NaOH (9.82 mg, 0.25 mmol). The mixture was stirred at 20° C. for 2 hrs. The pH was adjusted to around 3 by 1 M HCl. The aqueous phase was extracted with 10 mL DCM/MeOH (10:1) three times. The organic layer was dried over Na₂SO₄ and concentrated under vacuum to (4S)-2-[(2S)-2-(tert-butoxycarbonylamino)-3-[4-[3-(3-hydroxy-2,2-dimethyl-propyl)-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]thiazol-2-yl]propanoyl]-2,3-diazabicyclo[3.1.1]heptane-4-carboxylic acid (compound S1-n, 55.0 mg) as yellow oil. MS calc'd 910.5 (MH⁺), measured 910.6 (MH⁺).

### Step 14: Preparation of tert-butyl N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (compound S1-o)

To a solution of NMI (50 μL, 0.60 mmol) and TCFH (84.8 mg, 0.30 mmol) in ACN (2 mL), a solution of (4S)-2-[(2S)-2-(tert-butoxycarbonylamino)-3-[4-[3-(3-hydroxy-2,2-dimethyl-propyl)-(2M)-2-[2-[(1S)-1-methoxyethyl]-4-methyl-5-(3-morpholinoprop-1-ynyl)-3-pyridyl]-1-azatricyclo[6.3.1.0^{4,12}]dodeca-2,4(12),5,7-tetraen-6-yl]thiazol-2-yl]propanoyl]-2,3-diazabicyclo[3.1.1]heptane-4-carboxylic acid (compound S1-n, 55.0 mg, 0.06 mmol) in ACN (3 mL)/DMF (0.5 mL) was added dropwise over 5 min. The mixture was stirred at 25° C. for 0.5 hr. The reaction was concentrated under vacuum to give a residue. The residue was purified by prep-TLC to afford tert-butyl N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (compound S1-o, 16.0 mg) as yellow oil. MS calc'd 892.4 (MH⁺), measured 892.5 (MH⁺).

### Step 15: Preparation of (7S,13S)-7-amino-(20M)-20-[2-[(1S)-1-methoxyethyl]-4-methyl-5-(3-morpholinoprop-1-ynyl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate S1)

A mixture of tert-butyl N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]carbamate (compound S1-o, 16.0 mg, 0.02 mmol) in DCM (1 mL) was added TFA (0.2 mL, 3.18 mmol). The mixture was stirred at 20° C. for 0.5 hr, quenched with NaHCO₃ aq. until pH >7 and extracted with DCM (2 mL, three times). The combined organic layer was washed with brine (2 mL), dried over Na₂SO₄ and concentrated under vacuum to give (7S,13S)-7-amino-(20M)-20-[2-[(1S)-1-methoxyethyl]-4-methyl-5-(3-morpholinoprop-1-ynyl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate S1, 10.0 mg) as yellow oil. MS calc'd 792.4 (MH⁺), measured 792.5 (MH⁺).

### Example 1

### (1r,2R,3S)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide

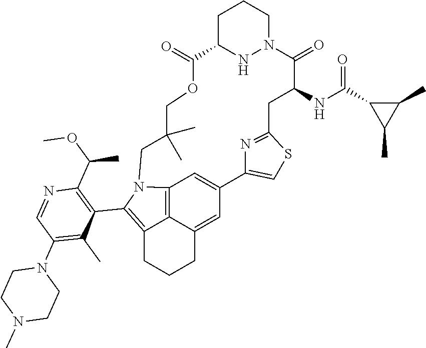

The title compound was prepared according to the following scheme:

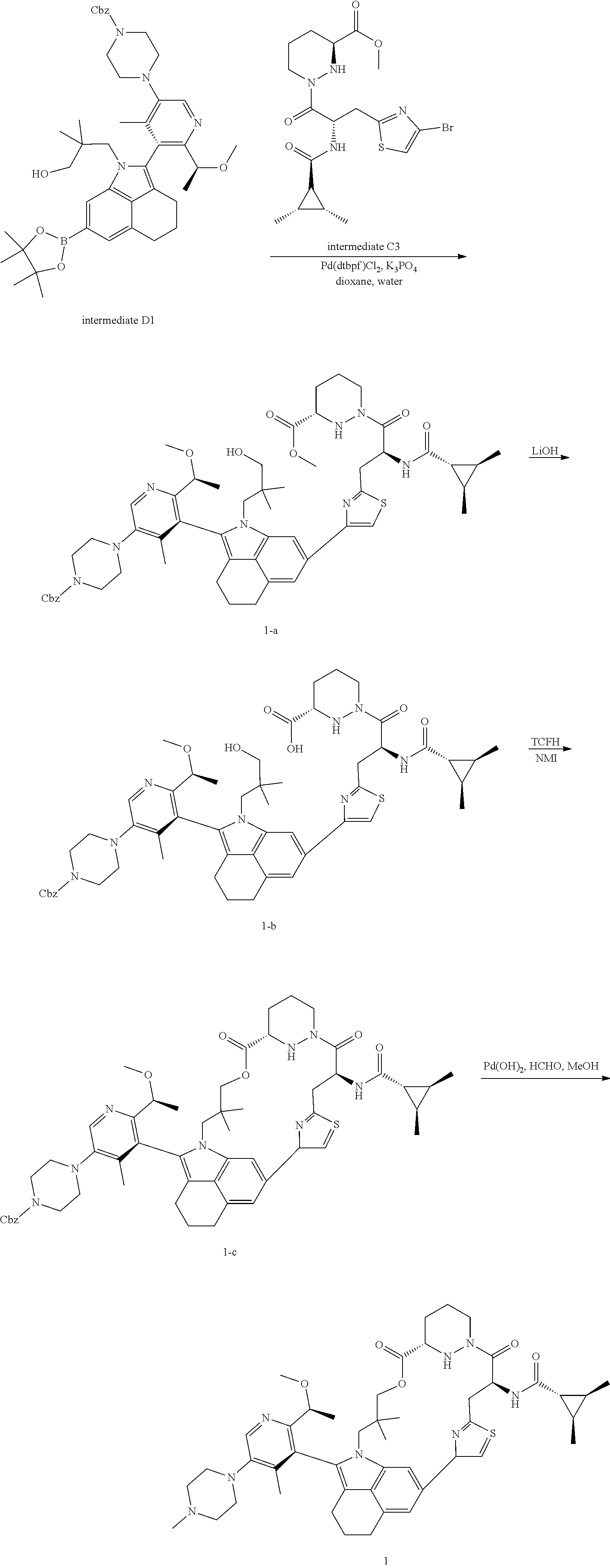

### Step 1: Preparation of methyl (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylate (compound 1-a)

To a solution of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1, 244.0 mg, 0.33 mmol) and methyl (3S)-1-[(2S)-3-(4-bromothiazol-2-yl)-2-[[(1r,2R,3S)-2,3-dimethylcyclopropanecarbonyl]amino]propanoyl]hexahydropyridazine-3-carboxylate (intermediate C3, 156.8 mg, 0.33 mmol) in 1,4-dioxane (10 mL) and water (2 mL), K₃PO₄ (210.9 mg, 0.99 mmol) and Pd(dtbpf)Cl₂ (32.4 mg, 0.05 mmol) were added. The mixture was degassed and purged with N2 three times then stirred at 60° C. for 1 h. The reaction mixture was filtered, and the filtrate was concentrated under vacuum to give a residue. The residue was purified by reversed phase chromatography to afford methyl (35)-1-[(2S)-3-[4-[(2P)-2-[5-(4-benzyloxycarbonylpiperazin-1-yl)-2-[(15)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylate (compound 1-a, 320.0 mg) as a yellow solid. MS calc'd 1003.5, (MH⁺), measured 1003.6, (MH⁺).

### Step 2: Preparation of (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylic acid (compound 1-b)

To a solution of methyl (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-[[(2R,3S)-2,3-dimethylcyclopropanecarbonyl]amino]propanoyl]hexahydropyridazine-3-carboxylate (compound 1-a, 290.0 mg, 0.26 mmol) in THF (5 mL), a solution of LiOH·H₂O (32.7 mg, 0.78 mmol) in water (1 mL) was added dropwise at −5° C. The reaction mixture was stirred at −5° C. for 1 h. The reaction mixture was added into ice water (50 mL) and acidified until pH reached 4 with 1 M HCl aqueous solution. The mixture was then extracted with EtOAc (60 mL, three times). The combined organic layer was washed with brine (100 mL), dried over anhydrous Na₂SO₄, filtered and concentrated under vacuum to afford (35)-1-[(25)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylic acid (compound 1-b, 289.0 mg) as a yellow solid. MS calc'd 987.5 (M−H)⁻, measured 987.5 (M−H)⁻.

### Step 3: Preparation of benzyl 4-[(5P)-5-[(7S,13S)-7-[[(1r,2R,3S)-2,3-dimethylcyclopropanecarbonyl]amino]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound 1-c)

To a solution of TCFH (293.9 mg, 1.05 mmol) and NMI (0.2 mL, 2.62 mmol) in ACN (20 mL), a solution of (3S)-1-[(2S)-3-[4-[(2P)-2-[5-(4-benzyloxycarbonylpiperazin-1-yl)-2-[(1S)-1-methoxyethyl]-4-methyl-3-pyridyl]-1-(3-hydroxy-2,2-dimethyl-propyl)-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylic acid (compound 1-b, 259.0 mg, 0.26 mmol) in ACN (30 mL) was added dropwise over 1 hr. The mixture was stirred at 25° C. for 1 hr. The reaction mixture was concentrated under vacuum to remove the ACN. The suspension was quenched by addition of water (80 mL) and EtOAc (80 mL), the layers were separated and the aqueous was extracted with EtOAc (60 mL, three times). The combined organic phase was washed with brine (200 mL), dried over anhydrous Na₂SO₄, filtered and concentrated under vacuum to give a residue. The residue was purified via column chromatography to afford benzyl 4-[(5P)-5-[(7S,13S)-7-[[(1r,2R,3S)-2,3-dimethylcyclopropanecarbonyl]amino]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound 1-c, 190.0 mg) as a yellow solid. MS calc'd 971.5 (MW), measured 971.5 (MH⁺).

### Step 4: Preparation of (1r,2R,3S)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide (Example 1)

To a mixture of Pd(OH)₂ on activated carbon (136.9 mg, 0.2 mmol) in methanol (10 mL), benzyl 4-[(5P)-5-[(7S,13S)-7-[[(1r,2R,3S)-2,3-dimethylcyclopropanecarbonyl]amino]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-6-[(1S)-1-methoxyethyl]-4-methyl-3-pyridyl]piperazine-1-carboxylate (compound 1-c, 190.0 mg, 0.2 mmol) and HCHO aq. (158.7 mg, 1.96 mmol) were added. The mixture was degassed and purged with N2 for three times, then the mixture was degassed and purged with H2 for three times and stirred at 25° C. for 3 hrs under H2 atmosphere. The reaction mixture was filtered and the filtrate was concentrated under vacuum to give a residue, the residue was purified by prep-HPLC to afford (1r,2R,3S)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide (example 1, 79.12 mg). MS calc'd 851.5 (MH⁺), measured 851.4 (MH⁺). ¹H NMR (400 MHz, METHANOL-d₄) δ=8.39 (s, 1H), 8.18 (s, 1H), 7.57 (s, 1H), 7.31 (s, 1H), 5.87-5.77 (m, 1H), 4.41 (d, J=11.6 Hz, 2H), 4.20-4.06 (m, 2H), 3.85 (d, J=11.2 Hz, 1H), 3.78-3.71 (m, 1H), 3.59-3.49 (m, 1H), 3.46-3.35 (m, 2H), 3.19 (s, 4H), 3.08-2.96 (m, 4H), 2.82-2.75 (m, 2H), 2.73-2.61 (m, 4H), 2.49-2.43 (m, 1H), 2.40-2.36 (m, 3H), 2.12-2.03 (m, 3H), 1.98 (s, 3H), 1.91-1.88 (m, 1H), 1.72-1.53 (m, 3H), 1.50 (d, J=6.4 Hz, 3H), 1.45-1.30 (m, 3H), 1.16-1.13 (m, 5H), 1.12 (s, 1H), 0.78 (s, 3H), 0.57 (s, 3H).

### Example 2

### (1r,2R,3S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide

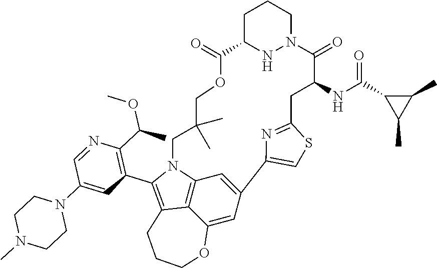

The title compound was prepared in analogy to the preparation of example 1 by using benzyl 4-[(5P)-5-[3-(3-hydroxy-2,2-dimethyl-propyl)-6-(4,4,5,5-tetramethyl-1,3,2-dioxaborolan-2-yl)-9-oxa-3-azatricyclo[6.4.1.0^{4,13}]trideca-1,4(13),5,7-tetraen-2-yl]-6-[(1S)-1-methoxyethyl]-3-pyridyl]piperazine-1-carboxylate (intermediate D2) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1).

Example 2 (12.2 mg) was obtained. MS calc'd 853.4 (MH⁺), measured 853.4 (MW). ¹H NMR (400 MHz, Methanol-d₄) δ=8.49 (d, J=2.4 Hz, 1H), 8.00 (s, 1H), 7.57 (s, 2H), 7.03 (s, 1H), 4.93-4.88 (m, 1H), 4.83-4.78 (m, 1H), 4.45-4.19 (m, 5H), 3.89-3.61 (m, 5H), 3.40-3.32 (m, 3H), 3.12-2.91 (m, 6H), 2.87-2.65 (m, 3H), 2.22-2.00 (m, 3H), 1.96-1.70 (m, 2H), 1.58-1.45 (m, 5H), 1.38-1.27 (m, 5H), 1.14-1.06 (m, 8H), 0.76-0.56 (m, 6H).

### Example 3

### (1S,6R,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]norcarane-7-carboxamide

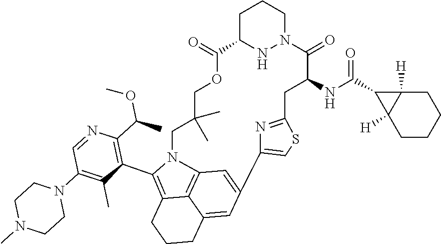

The title compound was prepared according to the following scheme:

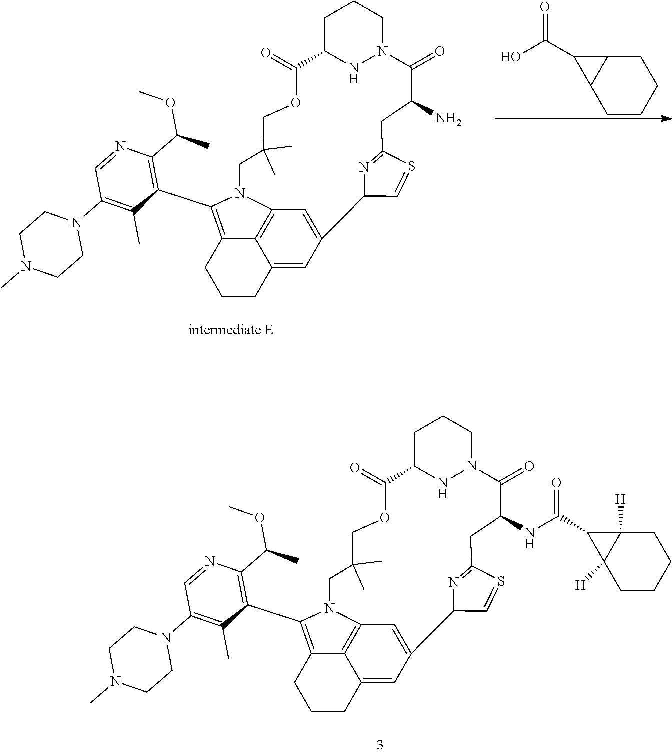

To a solution of norcarane-7-carboxylic acid (6.7 mg, 47.68 μmol) and DIEA (20.5 mg, 27.76 μL, 158.94 μmol) in THF (0.5 mL), COMU (20.4 mg, 47.68 μmol) at 0° C. was added. After being stirred at rt for 0.5 h, the reaction mixture was added with (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E, 30 mg, 39.74 μmol). The reaction mixture was stirred for 12 hrs, then concentrated under vacuum to get a residue, which was purified by prep-HPLC and SFC to afford (1S,6R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]norcarane-7-carboxamide (example 3, slower eluted, 2.9 mg). MS calc'd 877.5 (MH⁺), measured 877.6 (MH⁺). ¹H NMR (400 MHz, Methanol-d₄) δ=8.39 (s, 1H), 8.18 (s, 1H), 7.57 (s, 1H), 7.30 (s, 1H), 5.82 (br d, J=5.6 Hz, 1H), 4.40 (br d, J=11.8 Hz, 2H), 4.19-4.05 (m, 3H), 3.90-3.82 (m, 1H), 3.78-3.71 (m, 1H), 3.63 (s, 1H), 3.59-3.47 (m, 1H), 3.43-3.36 (m, 1H), 3.18 (br s, 5H), 3.09-2.92 (m, 4H), 2.84-2.60 (m, 6H), 2.51-2.43 (m, 1H), 2.38 (s, 3H), 2.04 (br s, 3H), 2.00-1.86 (m, 6H), 1.79-1.64 (m, 3H), 1.63-1.54 (m, 4H), 1.50 (br d, J=6.1 Hz, 4H), 1.16 (d, J=6.1 Hz, 2H), 0.77 (s, 3H), 0.57 (s, 3H).

SFC conditions: Instrument: SFC 150 Mgm; Column: IK, 250×30 mm I.D., 5 μm; Mobile phase: A for CO₂ and B for ethanol (0.1% NH₁₃H₂O); Gradient: B 50%; Flow rate: 80 mL/min; Back pressure: 100 bar; Column temperature: 35° C.

### Example 4

### (1R,6S,7R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide

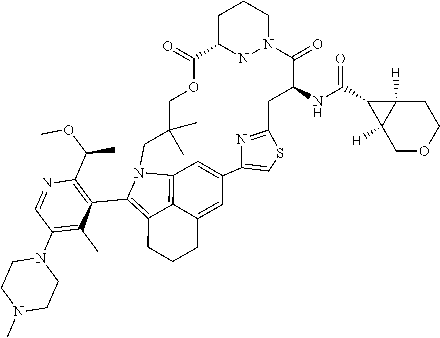

The title compound was prepared in analogy to the preparation of example 3 by using trans-(1R,6S,7R)-3-oxabicyclo[4.1.0]heptane-7-carboxylic acid (Enamine, CAS #2763584-94-5) instead of norcarane-7-carboxylic acid. Example 4 (5.5 mg, slower eluted) was obtained. MS calc'd 879.5 (MH⁺), measured 879.5 (MH⁺). H NMR (400 MHz, Methanol-d₄) δ=8.39 (s, 1H), 8.18 (s, 1H), 7.58 (s, 1H), 7.31 (s, 1H), 5.86-5.79 (m, 1H), 4.57 (s, 1H), 4.44-4.37 (m, 2H), 4.17-4.07 (m, 2H), 3.97-3.91 (m, 1H), 3.88-3.82 (m, 2H), 3.77-3.72 (m, 1H), 3.65-3.62 (m, 3H), 3.58 (td, J=2.8, 5.8 Hz, 2H), 3.49-3.44 (m, 1H), 3.41 (br dd, J=2.9, 14.6 Hz, 2H), 3.18 (br s, 4H), 3.10-3.04 (m, 2H), 3.01-2.95 (m, 2H), 2.40 (s, 3H), 2.09-2.03 (m, 3H), 1.98 (br s, 3H), 1.92-1.85 (m, 2H), 1.73-1.66 (m, 2H), 1.61-1.55 (m, 2H), 1.50 (d, J=6.3 Hz, 4H), 1.15-1.10 (m, 2H), 1.00-0.94 (m, 1H), 0.92-0.87 (m, 2H), 0.78 (s, 3H), 0.57 (s, 3H).

SFC conditions: Instrument: SFC 150 Mgm; Column: OJ, 250×30 mm I.D., 5 μm; Mobile phase: A for CO₂ and B for ethanol (0.1% NH₁₃H₂O); Gradient: B 15%; Flow rate: 80 mL/min; Back pressure: 100 bar; Column temperature: 35° C.

### Example 5

### (1r,2S,3R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide

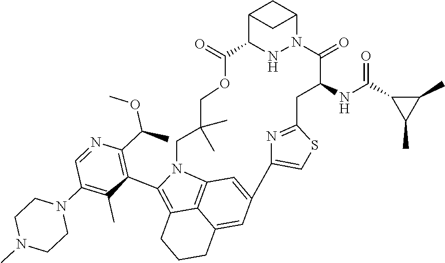

The title compound was prepared according to the following scheme:

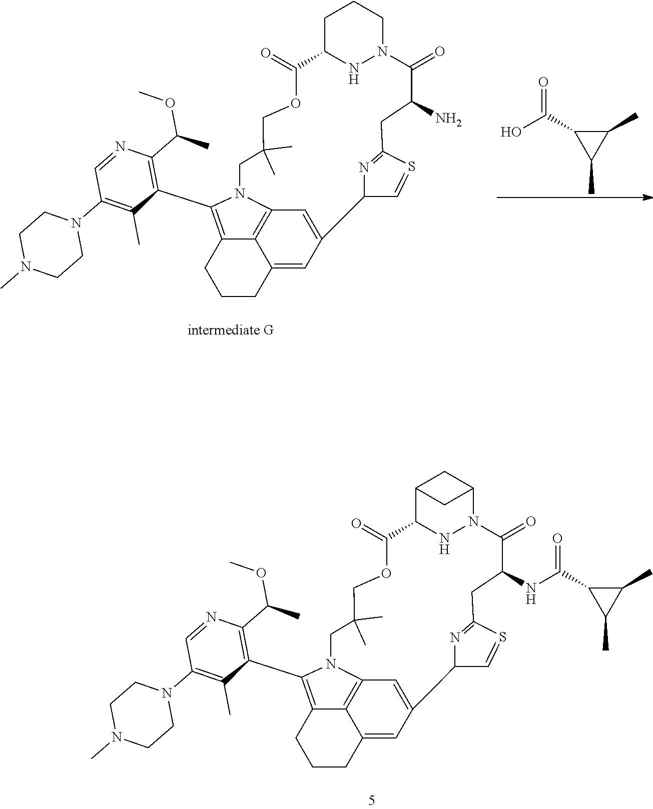

To a solution of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G, 30.0 mg, 0.04 mmol) in DMF (1.5 mL), (1r,2S,3R)-2,3-dimethylcyclopropanecarboxylic acid (9.0 mg, 0.08 mmol), DIPEA (0.1 mL, 0.23 mmol) and T4P (56.0 mg, 0.08 mmol) were added at 0° C. The mixture was stirred at 25° C. for 1 hr. After the reaction was completed, it was poured into water (20 mL) and extracted with ethyl acetate (10 mL, three times). The combined organic layer was concentrated under vacuum to give a residue. The residue was purified by prep-HPLC (column: Phenomenex Luna C18 150×25 mm×10 um;mobile phase: [A: H₂O(0.1% TFA);B: ACN];B %: 22.00%-52.0%,15.00 min;flow rate:25.00 ml/min) to afford (1r,2S,3R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide (example 5, 18.1 mg). MS calc'd 863.5 (MH⁺), measured 863.2 (MH⁺). ¹H NMR (400 MHz, METHANOL-d₄) δ=8.50 (s, 1H), 8.11 (s, 1H), 7.64 (s, 1H), 7.35 (s, 1H), 5.84-5.61 (m, 1H), 4.72-4.64 (m, 2H), 4.56-4.41 (m, 2H), 3.92-3.87 (m, 1H), 3.80 (s, 1H), 3.71-3.65 (m, 3H), 3.58-3.50 (m, 3H), 3.45-3.38 (m, 4H), 3.27-3.19 (m, 3H), 3.07-3.01 (m, 5H), 2.79-2.73 (m, 1H), 2.68-2.62 (m, 1H), 2.58-2.41 (m, 4H), 2.27-2.18 (m, 2H), 2.18-2.03 (m, 4H), 1.60-1.55 (m, 3H), 1.48-1.35 (m, 3H), 1.20-1.13 (m, 7H), 0.83-0.73 (m, 3H), 0.63 (s, 3H).

### Example 6

### (1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide

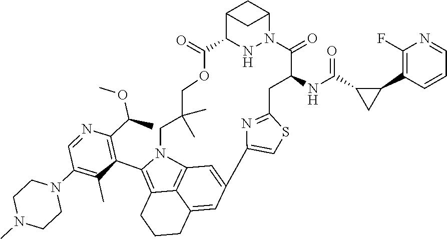

The title example 6 was prepared in analogy to the preparation of example 5 by using (1S,2S)-2-(2-fluoro-3-pyridyl)cyclopropanecarboxylic acid (intermediate O1) instead of (1r,2R,3S)-2,3-dimethylcyclopropanecarboxylic acid. Example 6 (14.9 mg) was obtained. MS calc'd 930.4 (MH⁺), measured 930.5 (MH⁺). ¹H NMR (400 MHz, Methanol-d₄) δ=8.39 (s, 1H), 8.08 (s, 1H), 8.03 (d, J=4.4 Hz, 1H), 7.71-7.64 (m, 1H), 7.59 (s, 1H), 7.31-7.29 (m, 1H), 7.28-7.25 (m, 1H), 6.04 (d, J=10.0 Hz, 1H), 4.65-4.61 (m, 2H), 3.96-3.84 (m, 2H), 3.68-3.59 (m, 2H), 3.46-3.39 (m, 2H), 3.19-3.13 (m, 4H), 3.10-2.97 (m, 5H), 2.72-2.62 (m, 5H), 2.56-2.40 (m, 5H), 2.38 (s, 3H), 2.29-2.14 (m, 3H), 2.05 (s, 4H), 1.65-1.48 (m, 5H), 1.48-1.34 (m, 2H), 0.74 (s, 3H), 0.59 (s, 3H).

### Example 7

### (1S,2S)-2-(2-fluoro-3-pyridyl)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]cyclopropanecarboxamide

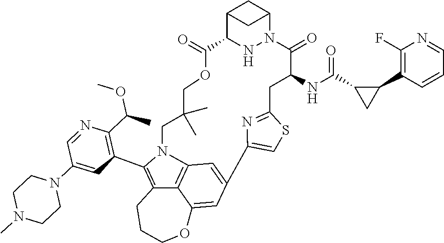

The title compound was prepared in analogy to the preparation of example 5 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate H) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 7 (18 mg) was obtained. MS calc'd 932.4 (MH⁺), measured 932.5 (MH⁺). ¹H NMR (400 MHz, Methanol-d₄) δ=8.49 (s, 1H), 8.03 (d, J=4.8 Hz, 1H), 7.92 (s, 1H), 7.71-7.61 (m, 2H), 7.56 (s, 1H), 7.30-7.22 (m, 1H), 7.00 (s, 1H), 4.62 (d, J=2.8 Hz, 1H), 4.43-4.37 (m, 1H), 4.35-4.27 (s, 1H), 4.05-3.94 (m, 1H), 3.82-3.75 (m, 1H), 3.49-3.47 (m, 1H), 3.45-3.41 (m, 2H), 2.98 (s, 3H), 2.87-2.83 (m, 1H), 2.51-2.46 (m, 1H), 2.43-2.34 (m, 2H), 2.22 (t, J=9.6 Hz, 2H), 2.14-2.04 (m, 2H), 1.85-1.74 (m, 4H), 1.68-1.65 (m, 1H), 1.64-1.61 (m, 2H), 1.61-1.59 (m, 1H), 1.58-1.52 (m, 3H), 1.45-1.43 (m, 2H), 1.42-1.37 (m, 4H), 1.29 (s, 1H), 0.95 (s, 1H), 0.94-0.93 (m, 4H), 0.91 (d, J=2.4 Hz, 3H), 0.75-0.66 (m, 2H), 0.56-0.52 (m, 1H).

### Example 8

### (1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(3-pyridyl)cyclopropanecarboxamide

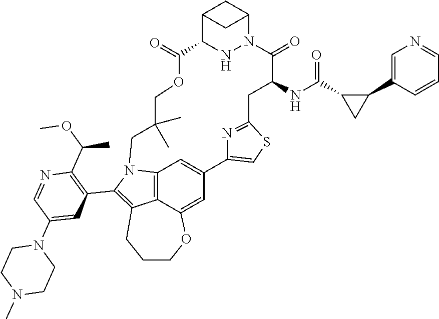

The title compound was prepared in analogy to the preparation of example 5 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate H) and (1S,2S)-2-(3-pyridyl)cyclopropanecarboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 8 (20.3 mg) was obtained. MS calc'd 914.4 (MH⁺), measured 914.4 (MH⁺). ¹H NMR (400 MHz, Methanol-d₄) δ=8.71-8.45 (m, 3H), 8.11-8.02 (m, 1H), 7.92 (s, 1H), 7.80-7.65 (m, 2H), 7.57 (s, 1H), 7.08-6.96 (m, 1H), 4.61 (s, 1H), 4.44-4.37 (m, 1H), 4.35-4.28 (m, 1H), 4.08-4.00 (m, 1H), 3.86-3.79 (m, 1H), 3.51-3.47 (m, 1H), 3.45-3.40 (m, 2H), 2.97 (s, 3H), 2.88-2.81 (m, 1H), 2.57-2.52 (m, 1H), 2.46-2.35 (m, 2H), 2.21 (d, J=9.6 Hz, 2H), 2.13-2.03 (m, 2H), 1.84-1.80 (m, 4H), 1.66 (s, 1H), 1.65-1.63 (m, 2H), 1.62-1.60 (m, 2H), 1.55-1.51 (m, 2H), 1.46-1.43 (m, 4H), 1.43-1.41 (m, 2H), 1.41-1.39 (m, 1H), 0.95 (s, 1H), 0.94-0.93 (m, 4H), 0.92 (d, J=4.4 Hz, 3H), 0.81-0.69 (m, 2H), 0.60-0.53 (m, 1H).

### Example 9

### (1r,2S,3R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2,3-dimethyl-cyclopropanecarboxamide

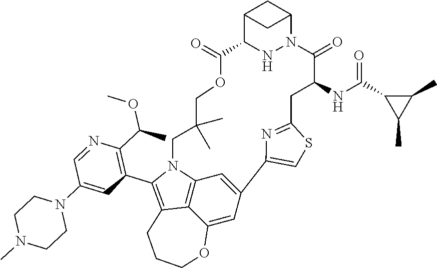

The title compound was prepared in analogy to the preparation of example 5 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate H) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 9 (17.7 mg) was obtained. MS calc'd 865.4 (MH⁺), measured 865.5 (MH⁺). ¹H NMR (400 MHz, Methanol-d₄) δ=8.54 (s, 1H), 7.93 (s, 2H), 7.59 (s, 1H), 7.03 (s, 1H), 4.63 (s, 2H), 4.47-4.28 (m, 3H), 4.19-3.91 (m, 3H), 3.56-3.40 (m, 6H), 3.08-2.98 (m, 4H), 2.96-2.83 (m, 2H), 2.68-2.31 (m, 4H), 2.30-2.02 (m, 4H), 1.65-1.49 (m, 4H), 1.48-1.31 (m, 3H), 1.22-1.02 (m, 10H), 0.96-0.50 (m, 8H).

### Example 10

### (1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-methyl-3-pyridyl)cyclopropanecarboxamide

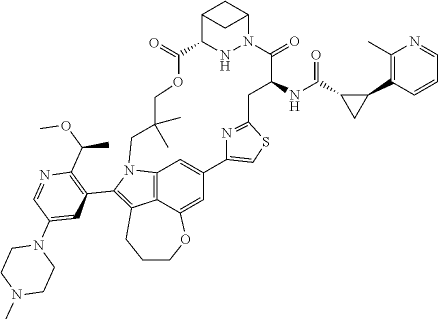

The title compound was prepared in analogy to the preparation of example 5 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate H) and (1S,2S)-2-(2-methyl-3-pyridyl)cyclopropanecarboxylic acid (intermediate O2) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 10 (20.7 mg) was obtained. MS calc'd 928.4 (MH⁺), measured 928.6 (MH⁺). ¹H NMR (400 MHz, Methanol-d₄) δ=8.58 (d, J=6.0 Hz, 1H), 8.53 (s, 1H), 8.29 (d, J=7.2 Hz, 1H), 7.96 (s, 1H), 7.87 (t, J=6.0 Hz, 2H), 7.61 (s, 1H), 7.05 (s, 1H), 4.76-4.49 (m, 3H), 4.48-4.24 (m, 3H), 4.20-3.76 (m, 4H), 3.74-3.35 (m, 8H), 3.01 (s, 4H), 2.87 (s, 4H), 2.66-2.32 (m, 5H), 2.29-1.99 (m, 5H), 1.70 (s, 1H), 1.65-1.37 (m, 6H), 0.96-0.42 (m, 8H).

### Example 11

### (1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-(2-fluoro-3-pyridyl)cyclopropanecarboxamide

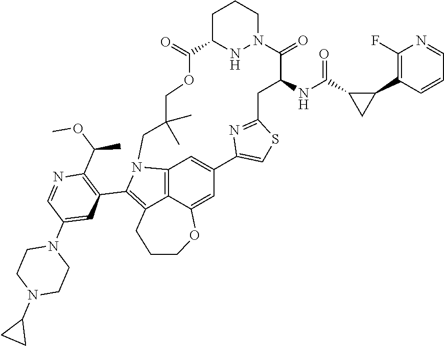

The title compound was prepared in analogy to the preparation of example 5 by using (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate I) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 11 (19 mg) was obtained. MS calc'd 946.4 (MH⁺), measured 946.5 (MH⁺). ¹H NMR (400 MHz, Methanol-d₄) δ=8.50 (d, J=2.8 Hz, 1H), 8.05-8.01 (m, 2H), 7.71-7.51 (m, 3H), 7.26 (t, J=6.4 Hz, 1H), 7.04 (s, 1H), 4.49-4.15 (m, 5H), 3.85 (d, J=11.2 Hz, 3H), 3.70 (d, J=11.6 Hz, 3H), 3.63 (s, 6H), 3.46-3.32 (m, 4H), 3.29-3.15 (m, 1H), 2.94 (d, J=5.2 Hz, 3H), 2.76 (t, J=11.6 Hz, 2H), 2.50-2.44 (m, 1H), 2.14 (s, 3H), 1.85 (s, 2H), 1.63-1.48 (m, 6H), 1.38 (s, 1H), 1.06-1.02 (m, 4H), 0.65 (s, 6H).

### Example 12

### (1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-fluoro-3-pyridyl)cyclopropanecarboxamide

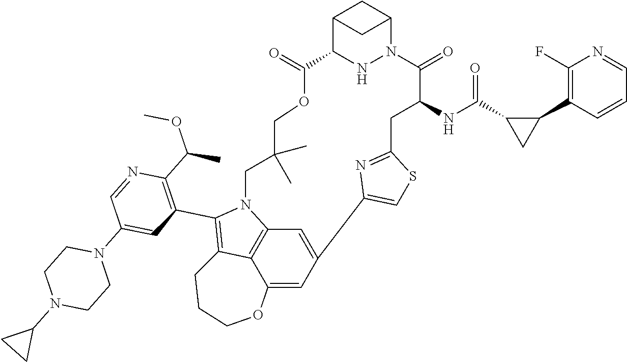

The title compound was prepared in analogy to the preparation of example 5 by using (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate J) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 12 (16 mg) was obtained. MS calc'd 958.4 (MH⁺), measured 958.5 (MH⁺). ¹H NMR (400 MHz, Methanol-d₄) δ=8.78-8.75 (s, 1H), 8.66 (d, J=5.2 Hz, 1H), 8.55-8.48 (s, 1H), 8.30 (d, J=7.6 Hz, 1H), 7.99-7.89 (m, 2H), 7.58 (s, 1H), 7.02 (s, 1H), 4.60 (s, 1H), 4.45-4.28 (m, 2H), 3.63 (s, 14H), 3.48 (dd, J=5.6 Hz, 14.8 Hz, 3H), 3.02-2.74 (m, 4H), 2.67-2.55 (m, 2H), 2.53-2.34 (m, 3H), 2.30-2.02 (m, 5H), 1.72 (s, 1H), 1.60-1.46 (m, 5H), 1.08 (s, 2H), 1.01 (d, J=5.6 Hz, 2H), 0.96-0.88 (m, 1H), 0.79-0.44 (m, 7H).

### Example 13

### (1r,2R,3S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide

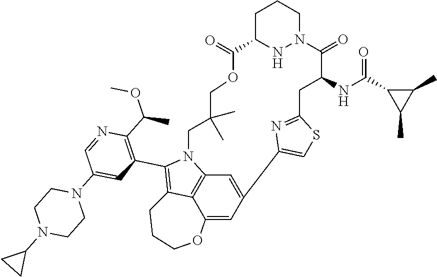

The title compound was prepared in analogy to the preparation of example 5 by using (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate I) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 13 (16.1 mg) was obtained. MS calc'd 879.5 (MH⁺), measured 879.6 (MH⁺). ¹H NMR (400 MHz, Methanol-d₄) δ=8.49 (d, J=2.4 Hz, 1H), 8.00 (s, 1H), 7.57 (s, 2H), 7.03 (s, 1H), 6.22-5.76 (m, 1H), 4.43-4.36 (m, 2H), 4.33-4.29 (m, 1H), 3.89-3.78 (m, 2H), 3.68 (d, J=11.6 Hz, 2H), 3.62 (s, 5H), 3.51-3.45 (m, 1H), 3.45-3.32 (m, 4H), 3.15-3.07 (m, 1H), 2.97-2.90 (m, 2H), 2.87-2.62 (m, 3H), 2.14 (s, 2H), 1.83 (s, 2H), 1.51 (d, J=6.0 Hz, 5H), 1.42-1.28 (m, 3H), 1.13-1.10 (m, 8H), 1.09-0.99 (m, 5H), 0.63 (s, 6H).

### Example 14

### (1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide

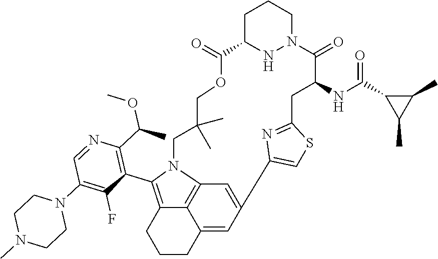

The title compound was prepared according to the following scheme:

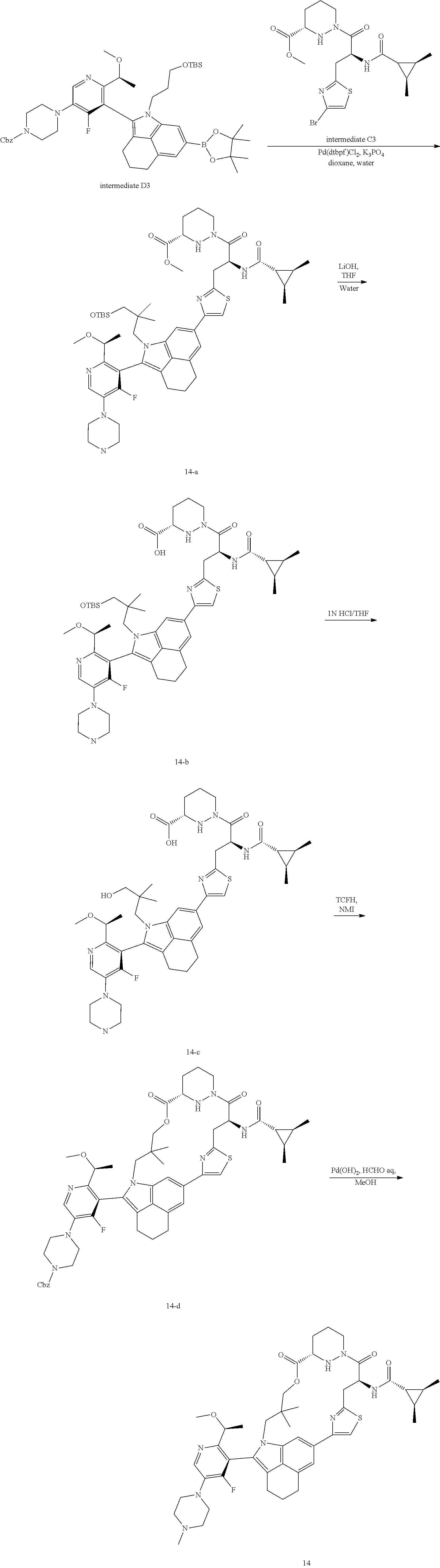

### Step 1: Preparation of methyl (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylate (compound 14-a)

To a solution of benzyl 4-[(5M)-5-[1-[3-[tert-butyl(dimethyl)silyl]oxy-2,2-dimethyl-propyl]-7-(4,4,5,5-tetramethyl-1,3,2-dioxaborolan-2-yl)-4,5-dihydro-3H-benzo[cd]indol-2-yl]-4-fluoro-6-[(1S)-1-methoxyethyl]-3-pyridyl]piperazine-1-carboxylate (intermediate D3, 200.0 mg, 0.23 mmol) and methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3, 110.74 mg, 0.23 mmol) in 1,4-dioxane (5 mL) and water (1 mL) was added K₃PO₄ (149.0 mg, 0.7 mmol) and Pd(dtbpf)Cl₂ (22.9 mg, 0.04 mmol). The mixture was degassed and purged with N₂ for 3 times then stirred at 70° C. for 1 h. The reaction mixture was filtered and the filtrate was concentrated under vacuum to give a residue which was purified via column chromatography to afford methyl (3S)-1-[(2S)-3-[4-[(2A)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylate (compound 14-a, 170.0 mg) as yellow solid. MS calc'd 1121.6 (MH⁺), measured 1121.4 (MW).

### Step 2: Preparation of (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-[[(1r,2S,3R)-2,3-dimethylcyclopropanecarbonyl]amino]propanoyl]hexahydropyridazine-3-carboxylic acid (compound 14-b)

To a solution of methyl (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-[[(1r,2R,3S)-2,3-dimethylcyclopropanecarbonyl]amino]propanoyl]hexahydropyridazine-3-carboxylate (compound 14-a, 170.0 mg, 0.15 mmol) in THF (2 mL) was added a solution of LiOH (12.7 mg, 0.3 mmol) in water (0.4 mL) at 0° C. The mixture was stirred at 0° C. for 1 h. After the reaction was completed, it was added with water (10 mL). The reaction mixture was acidified with 1N HCl aq. to a pH of 4. The mixture was extracted with EtOAc (20 mL, three times). The combined organic phases was washed with brine (20 mL), dried over Na₂SO₄, filtered and concentrated under vacuum to afford (3S)-1-[(2S)-3-[4-[(2A4)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-[[(1r,2S,3R)-2,3-dimethylcyclopropanecarbonyl]amino]propanoyl]hexahydropyridazine-3-carboxylic acid (compound 14-b, 170.0 mg) as a yellow solid which was used in the next step without further purification. MS calc'd 1107.6 (MH⁺), measured 1107.6 (MH⁺)

### Step 3: Preparation of (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-(3-hydroxy-2,2-dimethyl-propyl)-4,5-dihydro-3H-benzo[cd]indol-7-yl]thiazol-2-yl]-2-[[(1r,2S,3R)-2,3-dimethylcyclopropanecarbonyl]amino]propanoyl]hexahydropyridazine-3-carboxylic acid (compound 14-c)

A mixture of (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-[3-[tert-butyl(dimethyl)silyl]oxy-2,2-dimethyl-propyl]-4,5-dihydro-3H-benzo[cd]indol-7-yl]thiazol-2-yl]-2-[[(1r,2S,3R)-2,3-dimethylcyclopropanecarbonyl]amino]propanoyl]hexahydropyridazine-3-carboxylic acid (compound 14-b, 170.0 mg, 0.15 mmol) in a mixture solution of 2 M HCl aq. (1.7 mL)/THF (1.7 mL) was stirred at 0° C. for 0.2 h. The reaction mixture was poured into water (10 mL) and extracted with EtOAc (10 mL, three times). The combined organic layer was washed with brine (20 mL), dried over Na₂SO₄, filtered and concentrated under vacuum to afford (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-(3-hydroxy-2,2-dimethyl-propyl)-4,5-dihydro-3H-benzo[cd]indol-7-yl]thiazol-2-yl]-2-[[(1r,2S,3R)-2,3-dimethylcyclopropanecarbonyl]amino]propanoyl]hexahydropyridazine-3-carboxylic acid (compound 14-c, 150.0 mg) as a yellow solid. MS calc'd 993.5 (MH⁺), measured 993.5 (MH⁺).

### Step 4: Preparation of benzyl 4-[(5M)-5-[(7S,13S)-7-[[(1r,2S,3R)-2,3-dimethylcyclopropanecarbonyl]amino]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-4-fluoro-6-[(1S)-1-methoxyethyl]-3-pyridyl]piperazine-1-carboxylate (compound 14-d)

To a solution of TCFH (423.8 mg, 1.51 mmol) and NMI (0.2 mL, 2.27 mmol) in ACN (20 mL), (3S)-1-[(2S)-3-[4-[(2M)-2-[5-(4-benzyloxycarbonylpiperazin-1-yl)-4-fluoro-2-[(1S)-1-methoxyethyl]-3-pyridyl]-1-(3-hydroxy-2,2-dimethyl-propyl)-4,5-dihydro-3H-benzo[cd]indol-7-yl]thiazol-2-yl]-2-[[(1r,2S,3R)-2,3-dimethylcyclopropanecarbonyl]amino]propanoyl]hexahydropyridazine-3-carboxylic acid (compound 14-c, 150.0 mg, 0.15 mmol) and NMI (0.2 mL, 2.27 mmol) in ACN (20 mL) were added dropwise at 25° C. for 0.5 hr. The mixture was stirred at 25° C. for 0.5 hr. The reaction mixture was concentrated under vacuum to give a residue which was purified by reversed phased chromatography to afford benzyl 4-[(5M)-5-[(7S,13S)-7-[[(1r,2S,3R)-2,3-dimethylcyclopropanecarbonyl]amino]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-4-fluoro-6-[(1S)-1-methoxyethyl]-3-pyridyl]piperazine-1-carboxylate (compound 14-d, 111.0 mg) as a yellow solid. MS calc'd 975.5 (MH⁺), measured 975.4 (MH⁺).

### Step 5: Preparation of (1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide (example 14)

To a solution of Pd(OH)₂ on activated carbon (110 mg, 0.11 mmol) in methanol (8 mL), benzyl 4-[(5M)-5-[(7S,13S)-7-[[(1r,2S,3R)-2,3-dimethylcyclopropanecarbonyl]amino]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-20-yl]-4-fluoro-6-[(1S)-1-methoxyethyl]-3-pyridyl]piperazine-1-carboxylate (compound 14-d, 110.0 mg, 0.11 mmol) and formaldehyde aq. (16.9 mg, 0.56 mmol) were added under Ar atmosphere. The mixture was degassed under vacuum and purged with Ar three times before degassed under vacuum and purged with H2 for three times and then stirred at 25° C. for 16 hrs under H2 atmosphere (15 psi). The mixture was filtered through a pad of celite. The filtrate was concentrated under vacuum to give a residue which was purified by prep-HPLC to afford (1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide (example 14, 30.72 mg). MS calc'd 855.4 (MH⁺), measured 855.5 (MW). ¹H NMR (400 MHz, Methanol-d₄) δ=8.52-8.41 (m, 1H), 8.21 (s, 1H), 7.60 (s, 1H), 7.33 (s, 1H), 6.06-5.77 (m, 1H), 4.90-4.84 (m, 1H), 4.52-4.27 (m, 2H), 4.21-4.04 (m, 1H), 3.94-3.59 (m, 3H), 3.48-3.34 (m, 2H), 3.32-3.23 (m, 5H), 3.23-3.14 (m, 2H), 3.11-2.83 (m, 3H), 2.82-2.73 (m, 2H), 2.71-2.63 (m, 4H), 2.62-2.52 (m, 1H), 2.41-2.35 (m, 3H), 2.12-2.03 (m, 2H), 1.94-1.83 (m, 1H), 1.62-1.56 (m, 1H), 1.50 (d, J=6.4 Hz, 3H), 1.44-1.34 (m, 2H), 1.23-1.00 (m, 8H), 0.79 (s, 3H), 0.64-0.53 (m, 3H).

### Example 15

### (1S,2S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2-(2-fluoro-3-pyridyl)cyclopropanecarboxamide

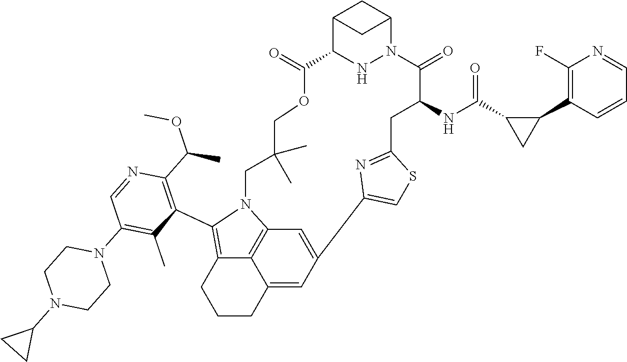

The title compound was prepared in analogy to the preparation of example 5 by using (7S,13S)-7-amino-(20P)-20-[5-(4-cyclopropylpiperazin-1-yl)-2-[(1S)-1-methoxyethyl]-4-methyl-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate L) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 15 (24.9 mg) was obtained. MS calc'd 944.5 (MH⁺), measured 944.5 (MH⁺). ¹H NMR (400 MHz, Methanol-d₄) δ=8.56-8.47 (m, 1H), 8.30-8.20 (m, 1H), 8.06-7.99 (m, 1H), 7.72-7.60 (m, 2H), 7.40-7.33 (m, 1H), 7.30-7.22 (m, 1H), 5.96-5.81 (m, 1H), 4.67-4.50 (m, 1H), 4.47-4.36 (m, 1H), 4.34-3.94 (m, 2H), 3.87-3.81 (m, 1H), 3.78-3.75 (m, 1H), 3.74-3.47 (m, 7H), 3.46-3.39 (m, 2H), 3.38-3.32 (m, 2H), 3.30-3.24 (m, 3H), 3.10-2.94 (m, 3H), 2.83-2.70 (m, 2H), 2.56-2.44 (m, 2H), 2.27-1.96 (m, 7H), 1.96-1.87 (m, 1H), 1.79-1.59 (m, 2H), 1.59-1.52 (m, 4H), 1.44-1.37 (m, 1H), 1.13-0.94 (m, 4H), 0.86-0.54 (m, 6H).

### Example 16

### (1R,6S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide

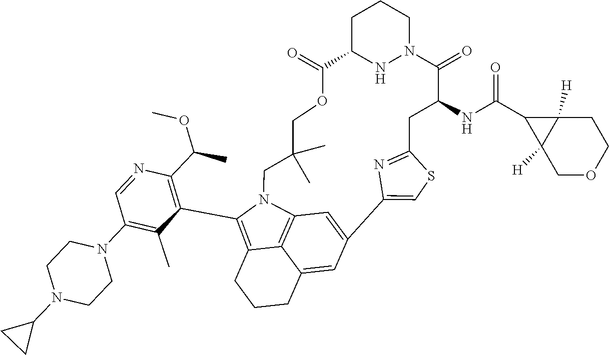

The title compound was prepared in analogy to the preparation of example 3 by using (7S,13S)-7-amino-(20P)-20-[5-(4-cyclopropylpiperazin-1-yl)-2-[(1S)-1-methoxyethyl]-4-methyl-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate L) and trans-(1R,6S,7R)-3-oxabicyclo[4.1.0]heptane-7-carboxylic acid (Enamine, CAS #2763584-94-5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 16 (8.4 mg) was obtained. MS calc'd 905.5 (MH⁺), measured 905.6 (MH⁺). ¹H NMR (400 MHz, Methanol-d₄) δ=8.36 (s, 1H), 8.18 (s, 1H), 7.57 (d, J=2.9 Hz, 1H), 7.31 (s, 1H), 5.82 (br d, J=4.8 Hz, 1H), 4.44-4.37 (m, 2H), 4.17-4.06 (m, 3H), 3.99-3.91 (m, 1H), 3.89-3.82 (m, 2H), 3.74 (br d, J=11.4 Hz, 1H), 3.64-3.56 (m, 3H), 3.43-3.37 (m, 2H), 3.18 (br s, 3H), 3.14-3.12 (m, 1H), 3.04-3.08 (m, 1H), 2.99-3.02 (m, 3H), 2.87-2.80 (m, 4H), 2.53-2.43 (m, 1H), 2.10-2.03 (m, 3H), 2.01-1.97 (m, 3H), 1.81-1.74 (m, 3H), 1.54-1.55 (m, 1H), 1.50 (d, J=6.1 Hz, 3H), 1.16 (d, J=6.3 Hz, 3H), 1.13-1.10 (m, 1H), 0.92-0.83 (m, 3H), 0.78 (s, 3H), 0.57 (s, 3H), 0.55-0.51 (m, 2H), 0.47-0.48 (m, 2H).

### Example 17

### (1R,6S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide

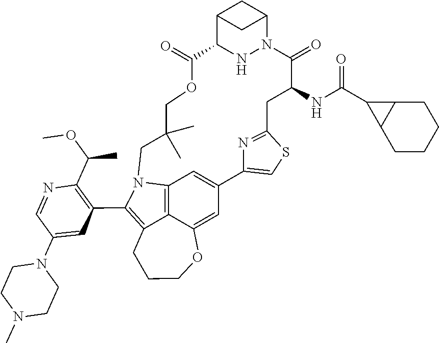

The title compound was prepared in analogy to the preparation of example 3 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate H) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E). Example 17 (7 mg) was obtained. MS calc'd 891.5 (MH⁺), measured 891.5 (MH⁺). ¹H NMR (400 MHz, Methanol-d₄) δ=8.42 (br s, 1H), 7.88 (s, 1H), 7.65-7.45 (m, 2H), 6.97 (br s, 1H), 6.39-5.86 (m, 1H), 4.60 (br d, J=1.9 Hz, 2H), 4.44-4.26 (m, 2H), 4.15-4.05 (m, 1H), 4.01-3.79 (m, 1H), 3.63 (s, 1H), 3.35 (br s, 8H), 2.87-2.76 (m, 1H), 2.63-2.65 (m, 5H), 2.36 (s, 3H), 2.20 (br t, J=9.8 Hz, 2H), 2.12-2.02 (m, 1H), 1.97-1.88 (m, 2H), 1.77-1.66 (m, 2H), 1.63-1.54 (m, 2H), 1.53-1.46 (m, 5H), 1.29 (br s, 4H), 1.16 (d, J=6.3 Hz, 3H), 0.93-0.81 (m, 3H), 0.72-0.61 (m, 3H), 0.58-0.44 (m, 3H).

### Example 18

### (1R,6S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide

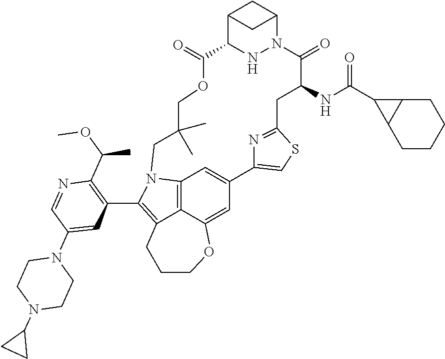

The title compound was prepared in analogy to the preparation of example 3 by using (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaene-8,14-dione (intermediate J) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E). Example 18 (5.1 mg) was obtained. MS calc'd 917.5 (MH⁺), measured 917.5 (MH⁺). ¹H NMR (400 MHz, Methanol-d₄) δ=8.48 (br d, J=1.4 Hz, 1H), 7.89 (br s, 1H), 7.76-7.49 (m, 2H), 6.99 (br s, 1H), 6.34-6.17 (m, 1H), 4.64-4.48 (m, 3H), 4.44-4.26 (m, 3H), 4.02-3.74 (m, 4H), 3.68-3.54 (m, 6H), 3.49-3.37 (m, 3H), 2.97-2.78 (m, 3H), 2.68-2.51 (m, 1H), 2.45-2.32 (m, 2H), 2.25-2.08 (m, 3H), 1.98-1.86 (m, 2H), 1.80-1.61 (m, 3H), 1.59-1.44 (m, 7H), 1.37-1.22 (m, 5H), 1.15-1.09 (m, 1H), 1.03 (d, J=5.5 Hz, 4H), 0.90-0.79 (m, 1H), 0.75-0.45 (m, 5H).

### Example 19

### (1R,6S,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]norcarane-7-carboxamide

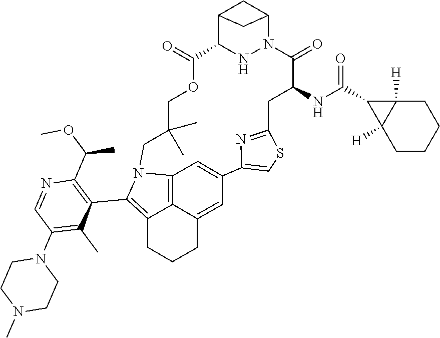

The title compound was prepared in analogy to the preparation of example 3 by using (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1R,6S,7r)-3-oxabicyclo[4.1.0]heptane-7-carboxylic acid (intermediate O4) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 19 (13.4 mg) was obtained. MS calc'd 889.5 (MH⁺), measured 889.5 (MH⁺). ¹H NMR (400 MHz, Methanol-d₄) δ=8.49 (s, 1H), 8.10 (s, 1H), 7.61 (s, 1H), 7.33 (s, 1H), 5.73 (s, 1H), 4.67-4.62 (m, 1H), 4.49 (s, 2H), 3.92-3.84 (m, 2H), 3.70-3.63 (m, 5H), 3.58-3.51 (m, 4H), 3.25-3.20 (m, 2H), 3.05-3.00 (m, 4H), 2.76-2.71 (m, 1H), 2.66-2.61 (m, 1H), 2.57-2.43 (m, 4H), 2.24-2.14 (m, 4H), 2.09-2.04 (m, 2H), 1.98-1.93 (m, 2H), 1.81-1.71 (m, 4H), 1.61-1.54 (m, 6H), 1.39-1.23 (m, 7H), 0.83-0.76 (m, 3H), 0.67-0.61 (m, 3H).

### Example 20

### (1S,6R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-3-oxabicyclo[4.1.0]heptane-7-carboxamide

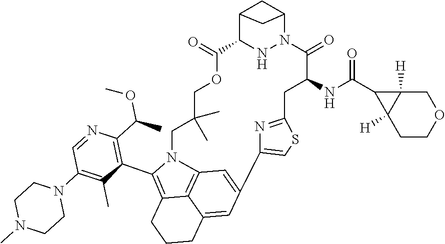

The title compound was prepared in analogy to the preparation of example 3 by using (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and trans-(1R,6S,7R)-3-oxabicyclo[4.1.0]heptane-7-carboxylic acid (Enamine, CAS #2763584-94-5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 20 (5.8 mg) was obtained. MS calc'd 891.5 (MH⁺), measured 891.5 (MH⁺). ¹H NMR (400 MHz, Methanol-d₄) δ=8.39 (s, 1H), 8.07 (s, 1H), 7.57 (d, J=2.1 Hz, 1H), 7.29 (s, 1H), 5.98 (br d, J=10.5 Hz, 1H), 4.63 (td, J=4.4, 8.6 Hz, 1H), 4.09 (ttd, J=3.2, 6.2, 12.3 Hz, 2H), 3.99-3.81 (m, 4H), 3.68-3.55 (m, 4H), 3.50-3.35 (m, 3H), 3.20-3.10 (m, 5H), 3.09-3.03 (m, 2H), 3.01-2.94 (m, 2H), 2.71-2.63 (m, 4H), 2.57-2.40 (m, 4H), 2.37 (s, 3H), 2.21 (br t, J=9.9 Hz, 1H), 2.10-1.98 (m, 5H), 1.53-1.46 (m, 5H), 0.93-0.84 (m, 3H), 0.76-0.69 (m, 3H), 0.58 (br s, 3H).

### Example 21

### (1R,6S,7r)-N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide

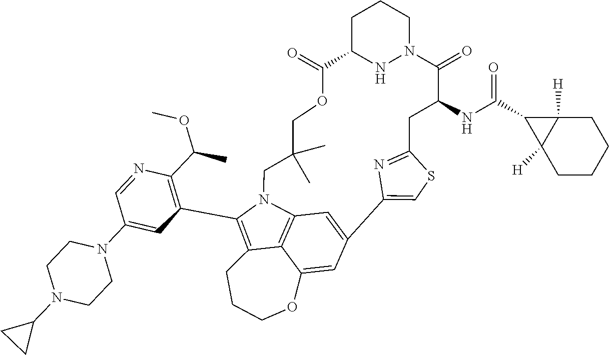

The title compound was prepared in analogy to the preparation of example 3 by using (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate I) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E). Example 21 (9.6 mg, slower eluted) was obtained. MS calc'd 905.5 (MH), measured 905.5 (MH⁺). ¹H NMR (400 MHz, Methanol-d₄) δ=8.41 (d, J=2.8 Hz, 1H), 7.98 (s, 1H), 7.55 (s, 1H), 7.41-7.26 (m, 1H), 7.00 (s, 1H), 6.26-5.87 (m, 1H), 4.43-4.25 (m, 4H), 4.14-4.05 (m, 1H), 3.84 (br d, J=10.8 Hz, 1H), 3.70-3.55 (m, 3H), 3.51-3.43 (m, 1H), 3.10-2.95 (m, 2H), 2.88-2.77 (m, 5H), 2.77-2.66 (m, 2H), 2.21-2.08 (m, 2H), 1.98-1.84 (m, 3H), 1.80-1.65 (m, 4H), 1.62-1.53 (m, 2H), 1.53-1.44 (m, 7H), 1.35-1.28 (m, 7H), 1.16 (d, J=6.3 Hz, 2H), 1.14-1.09 (m, 1H), 0.92-0.86 (m, 1H), 0.62 (br s, 4H), 0.56-0.51 (m, 2H), 0.49-0.44 (m, 2H).

SFC conditions: Instrument: SFC 150 Column: IK, 250×30 mm I.D., 5 μm; Mobile phase: A for CO₂ and B for EtOH (0.1% NH₃·H₂O); Gradient: B 50%; Flow rate: 80 mL/min; Back pressure: 100 bar; Column temperature: 35° C.

### Example 22

### (1R,6S,7R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}1.^{10,12}.0^{19,28}.0^{21,27}]tritriaconta-1(29),2,5(33),20,26(30),27-hexaen-7-yl]-3-oxabicyclo[4.1.0]heptane-7-carboxamide

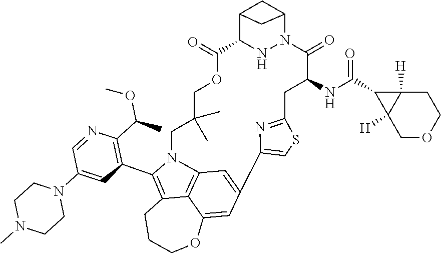

The title compound was prepared in analogy to the preparation of example 3 by using (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and trans-(1R,6S,7R)-3-oxabicyclo[4.1.0]heptane-7-carboxylic acid (Enamine, CAS #2763584-94-5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 22 (3.2 mg, slower eluted) was obtained. MS calc'd 893.4 (MH⁺), measured 893.4 (MH⁺). ¹H NMR (400 MHz, Methanol-d₄) δ=8.42 (br s, 1H), 7.88 (s, 1H), 7.54 (br s, 2H), 7.05-6.91 (m, 1H), 6.35-5.97 (m, 1H), 4.65-4.54 (m, 1H), 4.43-4.24 (m, 1H), 4.00 (d, J=5.6 Hz, 1H), 3.95-3.78 (m, 3H), 3.67-3.61 (m, 4H), 3.59-3.53 (m, 2H), 3.50-3.35 (m, 7H), 2.87-2.78 (m, 1H), 2.67-2.62 (m, 3H), 2.36 (s, 3H), 2.33-2.28 (m, 2H), 2.22-2.17 (m, 1H), 1.62-1.54 (m, 2H), 1.52-1.46 (m, 3H), 1.16-1.12 (m, 3H), 0.91 (br d, J=1.9 Hz, 3H), 0.89 (br d, J=2.5 Hz, 3H), 0.88 (br s, 3H), 0.71-0.63 (m, 3H), 0.57-0.47 (m, 3H).

SFC conditions: Instrument: SFC 150; Column: IK, 250×30 mm I.D., 5 μm; Mobile phase: A for CO₂ and B for IPA (0.1% NH₃·H₂O); Gradient: B 40%; Flow rate: 80 mL/min; Back pressure: 100 bar; Column temperature: 35° C.

### Example 23

### (1R,6S,7R)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-3-oxabicyclo[4.1.0]heptane-7-carboxamide

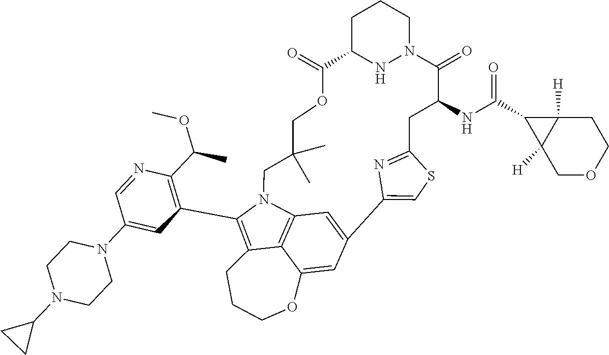

The title compound was prepared in analogy to the preparation of example 3 by using (7S,13S)-7-amino-(20P)-20-[5-(4-cyclopropylpiperazin-1-yl)-2-[(1S)-1-methoxyethyl]-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate I) and trans-(1R,6S,7R)-3-oxabicyclo[4.1.0]heptane-7-carboxylic acid (Enamine, CAS #2763584-94-5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 23 (4.1 mg, faster eluted) was obtained. MS calc'd 907.5 (MH⁺), measured 907.5 (MH⁺). ¹H NMR (400 MHz, Methanol-d₄) δ=8.44 (d, J=2.6 Hz, 1H), 7.99 (d, J=1.0 Hz, 1H), 7.55 (s, 1H), 7.45-7.34 (m, 1H), 7.01 (s, 1H), 6.14-5.93 (m, 1H), 4.42-4.27 (m, 4H), 4.00 (d, J=5.6 Hz, 1H), 3.96-3.90 (m, 1H), 3.88-3.81 (m, 2H), 3.68 (br d, J=11.0 Hz, 1H), 3.64-3.62 (m, 2H), 3.61-3.54 (m, 2H), 3.50-3.39 (m, 3H), 3.36-3.33 (m, 1H), 3.24-3.21 (m, 1H), 3.13 (br dd, J=1.6, 3.3 Hz, 1H), 2.79-2.69 (m, 2H), 2.31 (t, J=7.3 Hz, 1H), 2.19-2.11 (m, 2H), 1.98-1.89 (m, 2H), 1.85-1.78 (m, 2H), 1.77-1.73 (m, 1H), 1.66-1.59 (m, 2H), 1.50 (br d, J=6.1 Hz, 4H), 1.33-1.30 (m, 4H), 1.15-1.11 (m, 2H), 0.95-0.85 (m, 5H), 0.75-0.60 (m, 8H).

SFC conditions: Instrument: SFC 150; Column: IA, 250×30 mm I.D., 5 μm; Mobile phase: A for CO₂ and B for IPA (0.1% NH₃·H₂O); Gradient: B 40%; Flow rate: 80 mL/min; Back pressure: 100 bar; Column temperature: 35° C.

### Example 24

### (1R,6S,7r)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide

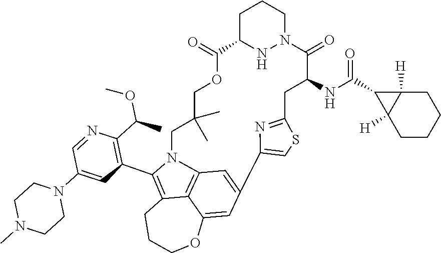

The title compound was prepared in analogy to the preparation of example 3 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate F) and (1S,6R,7r)-norcarane-7-carboxylic acid (intermediate O3) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 24 (14 mg) was obtained. MS calc'd 879.5 (MH⁺), measured 879.5 (MH⁺). ¹H NMR (400 MHz, Methanol-d₄) δ=8.49 (d, J=2.4 Hz, 1H), 8.00 (s, 1H), 7.76-7.49 (m, 2H), 7.03 (s, 1H), 6.22 (s, 1H), 4.54-4.18 (m, 5H), 4.15-3.94 (m, 2H), 3.88-3.65 (m, 4H), 3.57-3.34 (m, 5H), 3.23-3.10 (m, 2H), 3.04-2.95 (m, 4H), 2.90-2.64 (m, 3H), 2.23-2.08 (m, 2H), 2.01-1.86 (m, 3H), 1.86-1.79 (m, 1H), 1.79-1.65 (m, 3H), 1.56-1.40 (m, 8H), 1.39-1.15 (m, 6H), 0.80-0.53 (m, 6H).

### Example 25

### (1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide

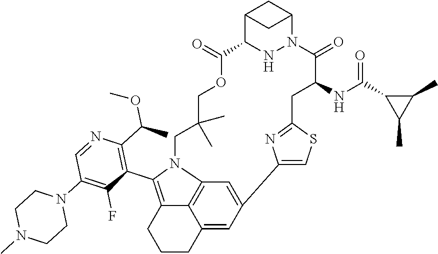

The title compound was prepared in analogy to the preparation of example 5 by using (7S,13S)-7-amino-(20M)-20-[4-fluoro-2-[(1S)-1-methoxyethyl]-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate M) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 25 (12.2 mg) was obtained. MS calc'd 867.4 (MH⁺), measured 867.5 (MH⁺). ¹H NMR (400 MHz, Methanol-d₄) δ=8.58-8.47 (m, 1H), 8.11-8.04 (m, 1H), 7.65-7.57 (m, 1H), 7.37-7.27 (m, 1H), 4.94-4.90 (m, 1H), 4.86-8.43 (m, 1H), 4.66-4.59 (m, 1H), 3.88-3.57 (m, 6H), 3.47-3.32 (m, 5H), 3.28-3.18 (m, 2H), 3.15-2.89 (m, 7H), 2.83-2.34 (m, 6H), 2.24-2.17 (m, 1H), 2.13-1.87 (m, 2H), 1.60-1.47 (m, 3H), 1.47-1.26 (m, 3H), 1.19-1.05 (m, 8H), 0.96-0.69 (m, 3H), 0.69-0.49 (m, 4H).

### Example 26

### (1S,2S)—N-[(7S,13S)-(20M1)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2-(2-fluoro-3-pyridyl)cyclopropanecarboxamide

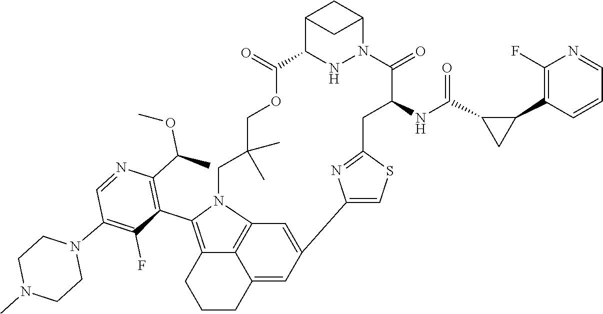

The title compound was prepared in analogy to the preparation of example 5 by using (7S,13S)-7-amino-(20M)-20-[4-fluoro-2-[(1S)-1-methoxyethyl]-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate M) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 26 (10.4 mg) was obtained. MS calc'd 934.4 (MH⁺), measured 934.6 (MH⁺). ¹H NMR (400 MHz, Methanol-d₄) δ=8.57-8.47 (m, 1H), 8.14-7.98 (m, 2H), 7.73-7.57 (m, 2H), 7.36-7.22 (m, 2H), 4.86-4.81 (m, 2H), 4.72-4.53 (m, 2H), 3.86-3.55 (m, 7H), 3.49-3.33 (m, 5H), 3.15-2.86 (m, 7H), 2.80-2.56 (m, 3H), 2.56-2.37 (m, 4H), 2.36-2.10 (m, 3H), 2.10-1.92 (m, 2H), 1.67-1.46 (m, 5H), 1.45-1.28 (m, 2H), 0.94-0.67 (m, 3H), 0.66-0.49 (m, 3H).

### Example 27

### (1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide

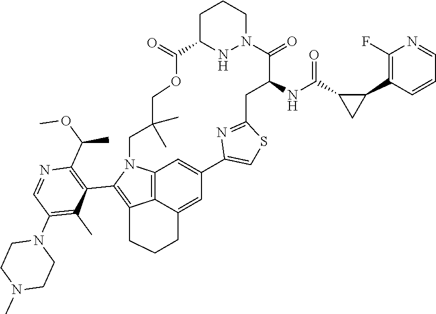

The title compound was prepared in analogy to the preparation of example 5 by using (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 27 (28.3 mg) was obtained. MS calc'd 918.4 (MH⁺), measured 918.5 (MH⁺). ¹H NMR (400 MHz, Methanol-d₄) δ=8.53-8.46 (m, 1H), 8.22 (s, 1H), 8.04 (s, 1H), 7.71-7.64 (m, 1H), 7.62 (s, 1H), 7.35 (s, 1H), 7.30-7.24 (m, 1H), 5.96-5.83 (m, 1H), 4.54-4.37 (m, 2H), 4.25-4.08 (m, 2H), 3.87-3.84 (m, 1H), 3.78-3.75 (m, 1H), 3.71-3.59 (m, 3H), 3.56-3.48 (m, 3H), 3.46-3.40 (m, 3H), 3.24 (s, 3H), 3.02 (s, 1H), 3.01 (s, 4H), 2.84-2.75 (m, 2H), 2.53-2.39 (m, 3H), 2.24-2.12 (m, 2H), 2.09 (s, 4H), 2.07-2.04 (m, 1H), 1.94-1.88 (m, 1H), 1.71-1.64 (m, 1H), 1.64-1.56 (m, 3H), 1.54 (d, J=6.4 Hz, 3H), 1.44-1.39 (m, 1H), 0.81 (s, 3H), 0.61 (s, 3H).

### Example 28

### (1S,2R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-tetrahydropyran-4-yl-cyclopropanecarboxamide

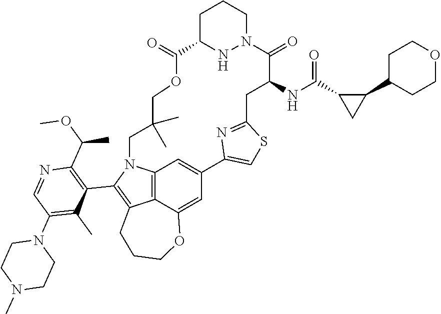

The title compound was prepared in analogy to the preparation of example 5 by using (7S,13S)-7-amino-(20P)-20-[2-[(1S)-1-methoxyethyl]-5-(4-methylpiperazin-1-yl)-3-pyridyl]-17,17-dimethyl-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaene-8,14-dione (intermediate F) and (1S,2R)-2-tetrahydropyran-4-ylcyclopropanecarboxylic acid (intermediate O5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 28 (15.6 mg) was obtained. MS calc'd 909.5 (MH⁺), measured 909.5 (MH⁺). ¹H NMR (400 MHz, Methanol-d₄) δ=8.50 (d, J=2.0 Hz, 1H), 8.01 (s, 1H), 7.57 (s, 2H), 7.03 (s, 1H), 4.42-4.36 (m, 3H), 4.34-4.29 (m, 2H), 3.95-3.91 (m, 3H), 3.86-3.81 (m, 2H), 3.72-3.66 (m, 2H), 3.40-3.33 (m, 6H), 3.18-3.12 (m, 4H), 3.00 (s, 4H), 2.77-2.72 (m, 3H), 2.14 (s, 2H), 1.84-1.80 (m, 1H), 1.74-1.62 (m, 3H), 1.59-1.55 (m, 2H), 1.52 (d, J=6.0 Hz, 4H), 1.46-1.38 (m, 4H), 1.15-1.12 (m, 1H), 1.06-1.01 (m, 3H), 0.76-0.69 (m, 4H), 0.64 (s, 4H).

### Example 29

### (1r,2R,3S)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

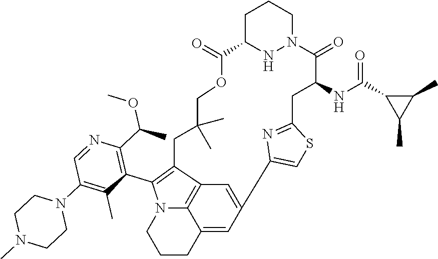

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 29 (19.9 mg) was obtained. MS calc'd 851.5 (MH⁺), measured 851.6 (MH⁺). ¹H NMR (400 MHz, Methanol-d₄) δ=8.50 (s, 1H), 8.39 (s, 1H), 7.51 (s, 1H), 7.43 (s, 1H), 5.80 (dd, J=1.6, 8.4 Hz, 1H), 4.47-4.39 (m, 2H), 4.22 (dd, J=3.2, 12.0 Hz, 1H), 4.11-4.03 (m, 1H), 3.88-3.79 (m, 2H), 3.64 (d, J=11.2 Hz, 2H), 3.52 (d, J=9.2 Hz, 3H), 3.45-3.33 (m, 5H), 3.29-3.20 (m, 2H), 3.13-3.02 (m, 3H), 3.01 (s, 3H), 2.82-2.73 (m, 1H), 2.39-2.14 (m, 5H), 2.14-2.11 (m, 3H), 1.93 (d, J=13.2 Hz, 1H), 1.74 (d, J=13.2 Hz, 1H), 1.66-1.56 (m, 1H), 1.52 (d, J=6.4 Hz, 3H), 1.42-1.32 (m, 2H), 1.14 (dd, J=6.0, 10.8 Hz, 8H), 0.93 (s, 3H), 0.62 (s, 3H).

### Example 30

### (1r,2S,3R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

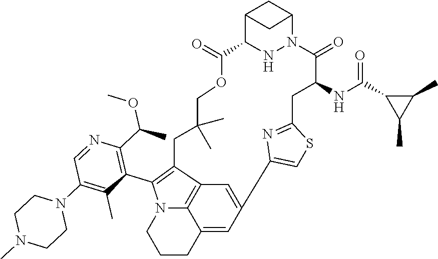

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate N2) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 30 (22.1 mg) was obtained. MS calc'd 863.5 (MH⁺), measured 863.6 (MH⁺). ¹H NMR (400 MHz, Methanol-d₄) δ=8.53-8.48 (m, 1H), 8.30 (s, 1H), 7.52 (s, 1H), 7.43 (s, 1H), 5.65 (s, 1H), 4.65 (d, J=4.8 Hz, 1H), 4.58 (s, 1H), 4.44 (d, J=5.6 Hz, 1H), 4.03 (s, 1H), 3.87 (d, J=10.8 Hz, 1H), 3.72-3.62 (m, 3H), 3.53 (s, 3H), 3.44-3.35 (m, 4H), 3.28-3.25 (m, 4H), 3.08 (s, 1H), 3.01 (s, 3H), 2.96 (s, 1H), 2.67 (d, J=5.6 Hz, 1H), 2.54 (d, J=4.4 Hz, 1H), 2.48-2.29 (m, 3H), 2.25-2.13 (m, 6H), 1.55-1.34 (m, 7H), 1.15 (dd, J=6.0, 12.4 Hz, 7H), 0.88 (s, 3H), 0.64 (s, 3H).

### Example 31

### (1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide

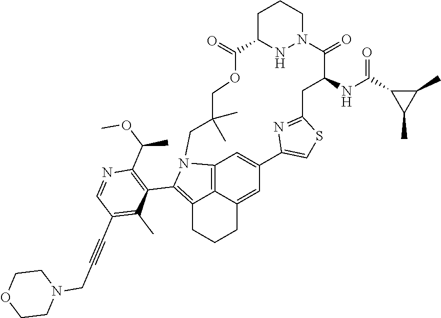

The title compound was prepared in analogy to the preparation of example 5 by using (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate P) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 31 (16.1 mg) was obtained. MS calc'd 876.4 (MH⁺), measured 876.7 (MH⁺). ¹H NMR (400 MHz, Methanol-d₄) δ=8.79 (s, 1H), 8.20 (s, 1H), 7.60 (s, 1H), 7.33 (s, 1H), 5.86 (d, J=6.0 Hz, 1H), 4.47 (s, 2H), 4.41 (d, J=13.2 Hz, 2H), 4.09-4.05 (m, 1H), 3.99-3.94 (m, 1H), 3.86 (d, J=11.2 Hz, 1H), 3.75 (d, J=11.6 Hz, 1H), 3.64-3.57 (m, 1H), 3.51-3.47 (m, 1H), 3.39 (dd, J=3.2, 14.8 Hz, 2H), 3.30-3.25 (m, 1H), 3.17 (s, 3H), 3.05-2.94 (m, 2H), 2.80-2.72 (m, 2H), 2.56-2.44 (m, 1H), 2.21 (s, 3H), 2.09-2.02 (m, 3H), 1.91-1.86 (m, 1H), 1.65-1.56 (m, 3H), 1.54 (d, J=6.4 Hz, 3H), 1.47-1.40 (m, 1H), 1.40-1.32 (m, 3H), 1.29 (s, 1H), 1.13 (dd, J=5.6, 9.2 Hz, 7H), 0.97-0.89 (m, 2H), 0.77 (s, 3H), 0.61 (s, 3H).

### Example 32

### (1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide

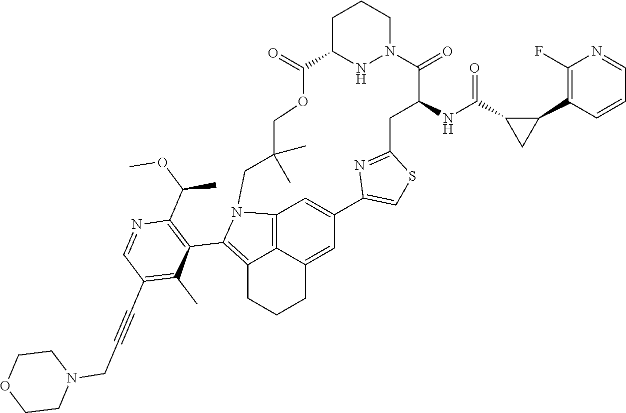

The title compound was prepared in analogy to the preparation of example 5 by using (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate P) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 32 (16.4 mg) was obtained. MS calc'd 943.4 (MH⁺), measured 943.3 (MH⁺). ¹H NMR (400 MHz, Methanol-d₄) δ=8.79 (s, 1H), 8.22 (s, 1H), 8.03 (d, J=4.8 Hz, 1H), 7.70-7.63 (m, 1H), 7.61 (s, 1H), 7.34 (s, 1H), 7.29-7.23 (m, 1H), 5.98-5.89 (m, 1H), 4.48 (s, 2H), 4.42 (d, J=13.1 Hz, 2H), 4.13-4.06 (m, 2H), 4.02-3.92 (m, 2H), 3.87 (d, J=11.6 Hz, 1H), 3.76 (d, J=11.2 Hz, 1H), 3.65-3.58 (m, 1H), 3.52-3.48 (m, 1H), 3.43 (dd, J=2.8, 14.4 Hz, 2H), 3.30-3.26 (m, 1H), 3.17 (s, 3H), 3.08-2.96 (m, 2H), 2.83-2.71 (m, 2H), 2.55-2.44 (m, 2H), 2.21 (s, 2H), 2.20-2.13 (m, 2H), 2.10-2.02 (m, 3H), 1.93-1.86 (m, 1H), 1.64-1.56 (m, 3H), 1.54 (d, J=6.0 Hz, 3H), 1.48-1.37 (m, 2H), 1.34-1.28 (m, 1H), 0.99-0.88 (m, 2H), 0.78 (s, 3H), 0.62 (s, 3H).

### Example 33

### (1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide

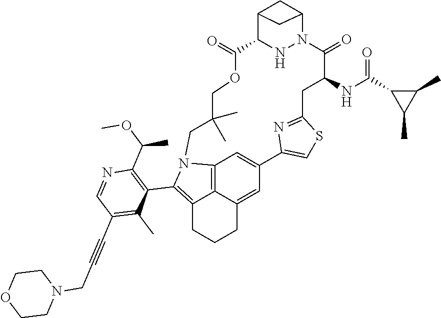

The title compound was prepared in analogy to the preparation of example 5 by using (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate Q) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 33 (11.2 mg) was obtained. MS calc'd 888.4 (MH⁺), measured 888.4 (MH⁺). ¹H NMR (400 MHz, Methanol-d₄) δ=8.81 (s, 1H), 8.11 (s, 1H), 7.61 (s, 1H), 7.34 (s, 1H), 4.69-4.62 (m, 1H), 4.50 (s, 3H), 4.10-3.86 (m, 5H), 3.68-3.60 (m, 1H), 3.59-3.47 (m, 3H), 3.44 (d, J=4.4 Hz, 1H), 3.41 (d, J=4.4 Hz, 1H), 3.31-3.27 (m, 2H), 3.20-3.08 (m, 2H), 3.05-2.99 (m, 2H), 2.89-2.68 (m, 2H), 2.66-2.59 (m, 1H), 2.57-2.49 (m, 2H), 2.47-2.41 (m, 1H), 2.31-2.18 (m, 3H), 2.11-2.03 (m, 2H), 1.62-1.54 (m, 4H), 1.47-1.30 (m, 4H), 1.16 (dd, J=6.4, 10.8 Hz, 8H), 0.96 (t, J=7.2 Hz, 1H), 0.78-0.69 (m, 3H), 0.64 (s, 3H).

### Example 34

### (1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide

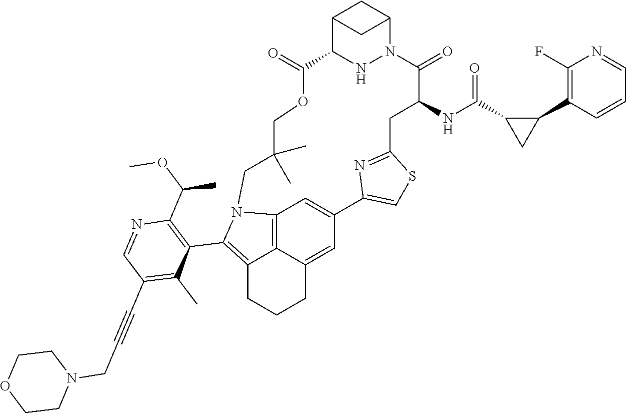

The title compound was prepared in analogy to the preparation of example 5 by using (7S,13S)-7-amino-(20P)-20-[2-[(1S)-1-methoxyethyl]-4-methyl-5-(3-morpholinoprop-1-ynyl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate Q) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 34 (11.2 mg) was obtained. MS calc'd 955.4 (MH⁺), measured 955.4 (MH⁺). ¹H NMR (400 MHz, Methanol-d₄) δ=8.90-8.77 (m, 2H), 8.13 (s, 1H), 8.05 (d, J=4.8 Hz, 1H), 7.72-7.65 (m, 1H), 7.63 (s, 1H), 7.34 (s, 1H), 7.31-7.26 (m, 1H), 4.70-4.63 (m, 1H), 4.56-4.47 (m, 3H), 4.06-3.87 (m, 5H), 3.72-3.61 (m, 2H), 3.52-3.49 (m, 1H), 3.48 (d, J=4.8 Hz, 1H), 3.44 (d, J=4.8 Hz, 1H), 3.31-3.28 (m, 1H), 3.18-3.12 (m, 1H), 3.05-2.99 (m, 2H), 2.84-2.71 (m, 2H), 2.66-2.60 (m, 1H), 2.59-2.48 (m, 4H), 2.48-2.42 (m, 1H), 2.34-2.15 (m, 5H), 2.13-2.03 (m, 2H), 1.96-1.80 (m, 1H), 1.65-1.59 (m, 2H), 1.57 (d, J=6.4 Hz, 3H), 1.50-1.40 (m, 2H), 1.38-1.29 (m, 1H), 0.96 (t, J=7.6 Hz, 1H), 0.81-0.70 (m, 3H), 0.65 (s, 3H).

### Example 35

### (1r,2R,3S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2,3-dimethyl-cyclopropanecarboxamide

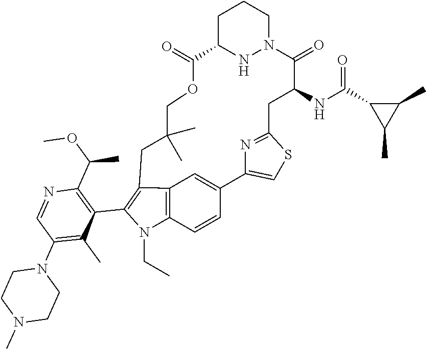

The title compound was prepared in analogy to the preparation of example 5 by using (7S,13S)-7-amino-21-ethyl-(20M)-20-[2-[(1S)-1-methoxyethyl]-4-methyl-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate N3) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 35 (13.3 mg) was obtained. MS calc'd 839.5 (MH⁺), measured 839.5 (MH⁺). ¹H NMR (400 MHz, Methanol-d₄) δ=8.55 (d, J=1.2 Hz, 1H), 8.41 (s, 1H), 7.70 (dd, J=1.6, 8.8 Hz, 1H), 7.56 (s, 1H), 7.49 (d, J=8.8 Hz, 1H), 5.75-5.69 (m, 1H), 4.85-4.80 (m, 1H), 4.48-4.39 (m, 1H), 4.29-4.17 (m, 3H), 4.16-4.07 (m, 1H), 3.79 (s, 2H), 3.50-3.38 (m, 1H), 3.29-3.24 (m, 4H), 3.21-3.11 (m, 3H), 3.09-3.03 (m, 2H), 2.81-2.74 (m, 1H), 2.71-2.61 (m, 3H), 2.38 (s, 3H), 2.22-2.16 (m, 1H), 2.15-2.09 (m, 1H), 2.07 (s, 3H), 1.98-1.91 (m, 1H), 1.83-1.72 (m, 1H), 1.66-1.54 (m, 1H), 1.47-1.43 (m, 3H), 1.42-1.33 (m, 2H), 1.19-1.15 (m, 4H), 1.15-1.12 (m, 3H), 1.00-0.92 (m, 6H), 0.48 (s, 3H).

### Example 36

### (1r,2R,3S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,29-tetrazahexacyclo[17.5.2.1^{2,5}.1^{9,13}.1^{10,12}.0^{22,26}]nonacosa-1(25),2,5(29),19,22(26),23-hexaen-7-yl]-2,3-dimethyl-cyclopropanecarboxamide

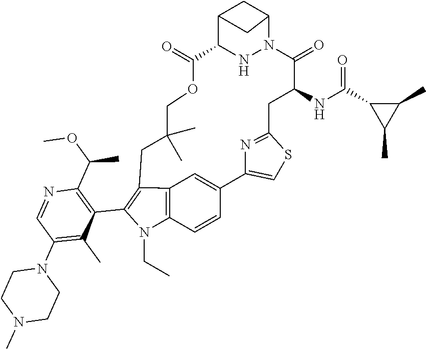

The title compound was prepared in analogy to the preparation of example 5 by using (7S,13S)-7-amino-21-ethyl-(20M)-20-[2-[(1S)-1-methoxyethyl]-4-methyl-5-(4-methylpiperazin-1-yl)-3-pyridyl]-17,17-dimethyl-15-oxa-4-thia-9,21,27,29-tetrazahexacyclo[17.5.2.1^{2,5}.1^{9,13}.1^{10,12}.0^{22,26}]nonacosa-1(25),2,5(29),19,22(26),23-hexaene-8,14-dione (intermediate N4) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 36 (9.9 mg) was obtained. MS calc'd 851.5 (MH⁺), measured 851.5 (MH⁺). ¹H NMR (400 MHz, Methanol-d₄) δ=8.46-8.40 (m, 2H), 7.72-7.67 (m, 1H), 7.62-7.44 (m, 2H), 5.60-5.52 (m, 1H), 4.70-4.60 (m, 2H), 4.26-4.06 (m, 3H), 3.81-3.75 (m, 1H), 3.71-3.65 (m, 1H), 3.49-3.37 (m, 1H), 3.26 (s, 3H), 3.22-3.15 (m, 2H), 3.12-3.02 (m, 3H), 2.74-2.63 (m, 4H), 2.61-2.56 (m, 1H), 2.49-2.42 (m, 1H), 2.39 (s, 3H), 2.23-2.17 (m, 2H), 2.09 (s, 3H), 1.60-1.54 (m, 1H), 1.47-1.44 (m, 3H), 1.43-1.36 (m, 2H), 1.35-1.28 (m, 3H), 1.18-1.16 (m, 3H), 1.15-1.12 (m, 3H), 1.02-0.96 (m, 3H), 0.91-0.88 (m, 3H), 0.50 (s, 3H).

### Example 37

### (1S,2S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide

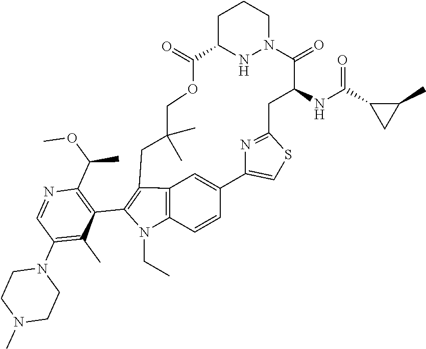

The title compound was prepared in analogy to the preparation of example 5 by using (7S,13S)-7-amino-21-ethyl-(20M)-20-[2-[(1S)-1-methoxyethyl]-4-methyl-5-(4-methylpiperazin-1-yl)-3-pyridyl]-17,17-dimethyl-15-oxa-4-thia-9,21,27,28-tetrazapentacyclo[17.5.2.1^{2,5}.1^{9,13}.0^{22,26}]octacosa-1(25),2,5(28),19,22(26),23-hexaene-8,14-dione (intermediate N3) and (1S,2S)-2-methylcyclopropanecarboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 37 (19.3 mg) was obtained. MS calc'd 825.4 (MH⁺), measured 825.4 (MH⁺). ¹H NMR (400 MHz, Methanol-d₄) δ=8.55 (s, 1H), 8.42 (s, 1H), 7.70 (dd, J=1.6, 8.8 Hz, 1H), 7.56 (s, 1H), 7.49 (d, J=8.8 Hz, 1H), 5.75-5.70 (m, 1H), 4.85-4.80 (m, 1H), 4.48-4.39 (m, 1H), 4.29-4.06 (m, 4H), 3.86-3.73 (m, 2H), 3.49-3.41 (m, 1H), 3.29-3.27 (m, 3H), 3.27-3.04 (m, 6H), 2.82-2.65 (m, 4H), 2.39 (s, 3H), 2.23-2.10 (m, 2H), 2.07 (s, 3H), 1.98-1.90 (m, 1H), 1.85-1.72 (m, 1H), 1.67-1.55 (m, 1H), 1.53-1.41 (m, 4H), 1.29-1.20 (m, 1H), 1.17-1.07 (m, 4H), 1.00-0.91 (m, 6H), 0.69-0.61 (m, 1H), 0.48 (s, 3H).

### Example 38

### cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide

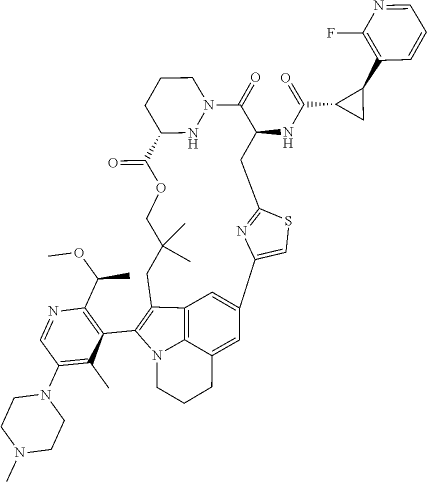

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 38 (25.3 mg) was obtained. MS calc'd 918.4 (MH⁺), measured 918.6 (MH⁺). ¹H NMR (400 MHz, Methanol-d₄) δ=8.48 (d, J=4.8 Hz, 1H), 8.40 (s, 1H), 8.04 (d, J=4.8 Hz, 1H), 7.68 (t, J=7.6 Hz, 1H), 7.52 (s, 1H), 7.43 (s, 1H), 7.27 (t, J=6.0 Hz, 1H), 5.86 (d, J=7.2 Hz, 1H), 4.43 (d, J=10.4 Hz, 2H), 4.23 (dd, J=3.2, 11.6 Hz, 1H), 4.08 (s, 1H), 3.85 (q, J=10.8 Hz, 2H), 3.68-3.61 (m, 2H), 3.54-3.45 (m, 4H), 3.43-3.36 (m, 3H), 3.29-3.20 (m, 5H), 3.07 (d, J=10.8 Hz, 1H), 3.01 (s, 4H), 2.84-2.76 (m, 1H), 2.51-2.44 (m, 1H), 2.33-2.23 (m, 2H), 2.20-2.14 (m, 3H), 2.08 (d, J=8.0 Hz, 3H), 1.93 (d, J=13.6 Hz, 1H), 1.75 (d, J=13.2 Hz, 1H), 1.67-1.48 (m, 6H), 1.44-1.38 (m, 1H), 0.93 (s, 3H), 0.62 (s, 3H).

### Example 39

### cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]cyclopropanecarboxamide

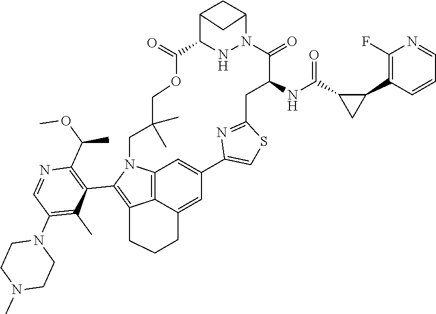

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate N2) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 39 (20.7 mg) was obtained. MS calc'd 930.4 (MH⁺), measured 930.6 (MH⁺). ¹H NMR (400 MHz, Methanol-d₄) δ=8.47 (s, 1H), 8.30 (s, 1H), 8.04 (d, J=4.8 Hz, 1H), 7.69 (t, J=8.8 Hz, 1H), 7.53 (s, 1H), 7.42 (s, 1H), 7.29-7.25 (m, 1H), 5.70 (s, 1H), 4.67 (d, J=5.6 Hz, 1H), 4.40 (d, J=6.0 Hz, 1H), 4.06 (d, J=13.2 Hz, 1H), 3.88 (d, J=10.4 Hz, 1H), 3.72 (d, J=10.8 Hz, 1H), 3.63 (d, J=12.0 Hz, 3H), 3.55-3.46 (m, 4H), 3.39 (d, J=1.2 Hz, 3H), 3.23 (s, 4H), 3.08 (s, 2H), 3.01 (s, 4H), 2.70-2.65 (m, 1H), 2.60-2.42 (m, 4H), 2.35-2.28 (m, 2H), 2.25-2.17 (m, 4H), 2.08 (s, 3H), 1.60 (dd, J=4.8, 9.6 Hz, 1H), 1.50 (d, J=6.0 Hz, 3H), 1.46-1.41 (m, 1H), 0.87 (s, 3H), 0.63 (s, 3H).

### Example 40

### cis-(1S,2S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-(2-fluoro-3-pyridyl)cyclopropanecarboxamide

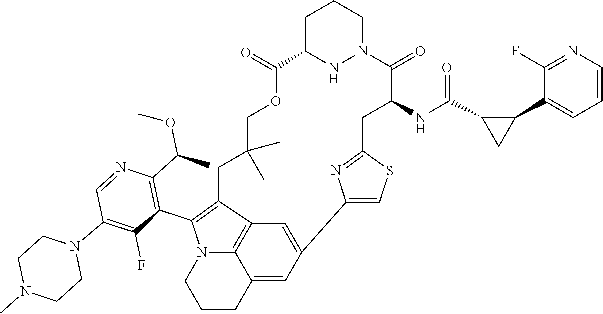

The title compound was prepared in analogy to the preparation of example 5 by using (7S,13S)-7-amino-(20P)-20-[4-fluoro-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate R1) and (1S,2S)-2-(2-fluoro-3-pyridyl)cyclopropanecarboxylic acid (intermediate O1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 40 (10.1 mg) was obtained. MS calc'd 922.4 (MH⁺), measured 922.4 (MH⁺). ¹H NMR (400 MHz, Methanol-d₄) δ=8.52 (d, J=10.4 Hz, 1H), 8.42 (s, 1H), 8.03 (d, J=4.0 Hz, 1H), 7.69-7.64 (m, 1H), 7.53-7.49 (m, 1H), 7.46-7.40 (m, 1H), 7.28-7.23 (m, 1H), 6.04-5.86 (m, 1H), 4.48-4.31 (m, 2H), 4.24-4.10 (m, 2H), 3.79-3.72 (m, 4H), 3.70-3.57 (m, 4H), 3.46-3.40 (m, 2H), 3.24 (s, 3H), 3.13-3.04 (m, 2H), 3.00 (s, 3H), 2.97-2.91 (m, 1H), 2.90-2.75 (m, 2H), 2.56-2.44 (m, 2H), 2.30-2.20 (m, 2H), 2.18-2.08 (m, 2H), 1.94-1.87 (m, 1H), 1.79-1.62 (m, 2H), 1.62-1.49 (m, 3H), 1.47 (d, J=6.4 Hz, 3H), 1.42-1.36 (m, 1H), 0.88 (s, 3H), 0.64 (s, 3H).

### Example 41

### (1r,2R,3S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

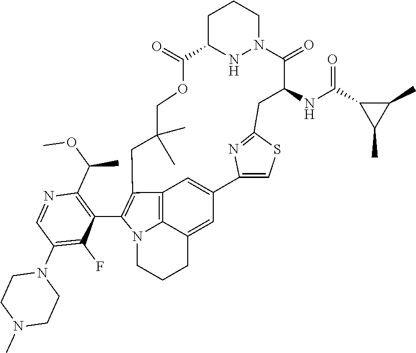

The title compound was prepared in analogy to the preparation of example 5 by using (7S,13S)-7-amino-(20P)-20-[4-fluoro-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate R1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 41 (11.9 mg) was obtained. MS calc'd 855.4 (MH⁺), measured 855.4 (MH⁺). ¹H NMR (400 MHz, Methanol-d₄) δ=8.50 (d, J=10.0 Hz, 1H), 8.40 (s, 1H), 7.50 (s, 1H), 7.43 (s, 1H), 5.94-5.81 (m, 1H), 4.46-4.30 (m, 2H), 4.24-4.08 (m, 2H), 3.82-3.72 (m, 4H), 3.68-3.59 (m, 3H), 3.50-3.34 (m, 4H), 3.28-3.26 (m, 1H), 3.24 (s, 3H), 3.15-3.04 (m, 2H), 3.00 (s, 3H), 2.96-2.89 (m, 1H), 2.82-2.70 (m, 1H), 2.57-2.42 (m, 1H), 2.31-2.19 (m, 2H), 2.14-2.03 (m, 1H), 1.95-1.85 (m, 1H), 1.71-1.54 (m, 2H), 1.47 (d, J=9.2 Hz, 3H), 1.41-1.31 (m, 2H), 1.18-1.06 (m, 8H), 0.87 (s, 3H), 0.63 (s, 3H).

### Example 42

### cis-(1S,2R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide

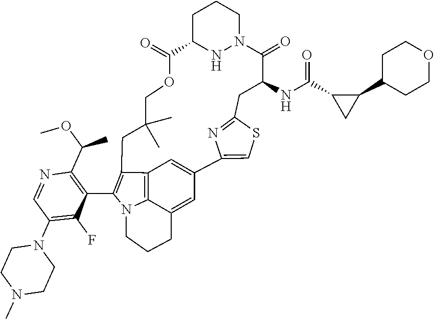

The title compound was prepared in analogy to the preparation of example 5 by using (7S,13S)-7-amino-(20P)-20-[4-fluoro-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate R1) and (1S,2R)-2-tetrahydropyran-4-ylcyclopropanecarboxylic acid (intermediate O5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 42 (21.1 mg) was obtained. MS calc'd 911.5 (MH⁺), measured 911.4 (MH⁺). ¹H NMR (400 MHz, Methanol-d₄) δ=8.51 (d, J=10.0 Hz, 1H), 8.40 (s, 1H), 7.49 (s, 1H), 7.42 (s, 1H), 5.96-5.85 (m, 1H), 4.51-4.32 (m, 2H), 4.23-4.09 (m, 2H), 3.97-3.90 (m, 2H), 3.85-3.71 (m, 4H), 3.71-3.47 (m, 4H), 3.43-3.35 (m, 4H), 3.29-3.25 (m, 1H), 3.24 (s, 3H), 3.15-3.03 (m, 2H), 3.00 (s, 3H), 2.97-2.90 (m, 1H), 2.90-2.64 (m, 2H), 2.58-2.44 (m, 1H), 2.31-2.16 (m, 2H), 2.14-2.05 (m, 1H), 1.95-1.85 (m, 1H), 1.79-1.64 (m, 3H), 1.63-1.54 (m, 2H), 1.54-1.22 (m, 6H), 1.16-1.09 (m, 1H), 1.08-0.98 (m, 2H), 0.88 (s, 3H), 0.79-0.72 (m, 1H), 0.63 (s, 3H).

### Example 43

### cis-(1r,2S,3R)—N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

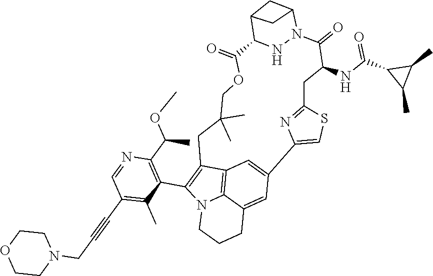

The title compound was prepared in analogy to the preparation of example 5 by using (7S,13S)-7-amino-(20M)-20-[2-[(1S)-1-methoxyethyl]-4-methyl-5-(3-morpholinoprop-1-ynyl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate S1) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 43 (2.6 mg) was obtained. MS calc'd 888.4 (MH⁺), measured 888.4 (MH⁺). ¹H NMR (400 MHz, Methanol-d₄) δ=8.68 (s, 1H), 8.29 (s, 1H), 7.50 (s, 1H), 7.40 (s, 1H), 5.72-5.61 (m, 1H), 4.67-4.62 (m, 1H), 4.60-4.56 (m, 1H), 4.42-4.37 (m, 1H), 4.12-3.99 (m, 1H), 3.89 (d, J=10.8 Hz, 1H), 3.76-3.72 (m, 4H), 3.68 (s, 1H), 3.66 (s, 2H), 3.59-3.51 (m, 1H), 3.47-3.37 (m, 1H), 3.29-3.23 (m, 1H), 3.20 (s, 3H), 3.11-2.99 (m, 2H), 2.98-2.91 (m, 1H), 2.72-2.64 (m, 5H), 2.54 (s, 1H), 2.42 (s, 1H), 2.34 (dd, J=2.0, 13.9 Hz, 1H), 2.24-2.14 (m, 6H), 2.06-1.99 (m, 1H), 1.50 (d, J=6.2 Hz, 4H), 1.15 (dd, J=5.4, 12.4 Hz, 8H), 0.87 (s, 3H), 0.64 (s, 3H).

### Example 44

### (1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide

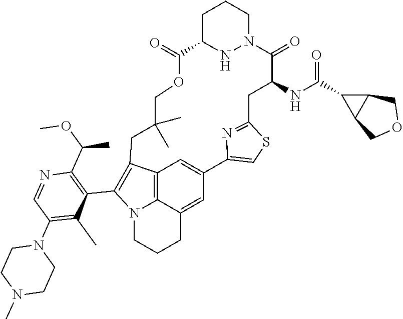

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and (1R,5S)-3-oxabicyclo[3.1.0]hexane-6-carboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 44 (28.5 mg) was obtained. MS calc'd 865.4 (MH⁺), measured 865.7 (MH⁺). ¹H NMR (400 MHz, Methanol-d₄) δ=8.48 (s, 1H), 8.38 (s, 1H), 7.50 (s, 1H), 7.42 (s, 1H), 5.79 (d, J=7.2 Hz, 1H), 4.42 (d, J=6.4 Hz, 2H), 4.22 (dd, J=3.2, 12.0 Hz, 1H), 4.13-4.04 (m, 1H), 3.96-3.68 (m, 7H), 3.68-3.61 (m, 2H), 3.49 (d, J=9.6 Hz, 3H), 3.44-3.36 (m, 3H), 3.29-3.25 (m, 4H), 3.13-3.04 (m, 2H), 3.01 (s, 4H), 2.82-2.73 (m, 1H), 2.32-2.05 (m, 10H), 1.93 (d, J=13.2 Hz, 1H), 1.78-1.56 (m, 3H), 1.50 (d, J=6.0 Hz, 3H), 0.92 (s, 3H), 0.61 (s, 3H).

### Example 45

### cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide

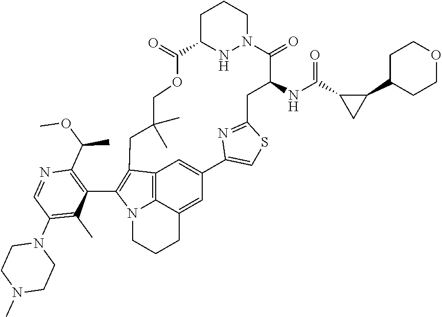

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and (1S,2R)-2-tetrahydropyran-4-ylcyclopropanecarboxylic acid (intermediate O5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 45 (24.6 mg) was obtained. MS calc'd 907.5 (MH⁺), measured 907.7 (MH⁺). ¹H NMR (400 MHz, Methanol-d₄) δ=8.48 (s, 1H), 8.38 (s, 1H), 7.50 (s, 1H), 7.42 (s, 1H), 5.81 (d, J=7.2 Hz, 1H), 4.42 (d, J=6.4 Hz, 2H), 4.22 (dd, J=2.8, 11.6 Hz, 1H), 4.13-4.05 (m, 1H), 3.95-3.81 (m, 4H), 3.67-3.61 (m, 2H), 3.52-3.46 (m, 3H), 3.42-3.35 (m, 5H), 3.29-3.26 (m, 4H), 3.07 (d, J=8.8 Hz, 2H), 3.01 (s, 4H), 2.82-2.74 (m, 1H), 2.28 (dd, J=5.2, 9.2 Hz, 1H), 2.24-2.13 (m, 3H), 2.10-2.06 (m, 3H), 1.93 (d, J=14.2 Hz, 1H), 1.78-1.57 (m, 6H), 1.52-1.39 (m, 6H), 1.17-1.11 (m, 1H), 1.06 (dd, J=4.2, 8.4 Hz, 2H), 0.92 (s, 3H), 0.78 (t, J=9.2 Hz, 1H), 0.61 (s, 3H).

### Example 46

### trans-(1S,2S)-2-(difluoromethyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide

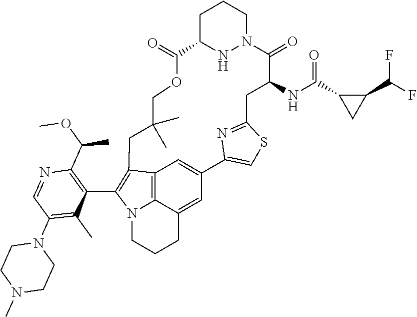

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and trans-(1S,2S)-2-(difluoromethyl)cyclopropanecarboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 46 (14.2 mg) was obtained. MS calc'd 873.4 (MH⁺), measured 873.5 (MH⁺). ¹H NMR (400 MHz, Methanol-d₄) δ=8.47 (s, 1H), 8.38 (s, 1H), 7.50 (s, 1H), 7.41 (s, 1H), 5.86-5.77 (m, 1H), 5.98-5.65 (m, 1H), 4.46-4.37 (m, 2H), 4.22 (dd, J=3.2, 12.0 Hz, 1H), 4.14-4.03 (m, 1H), 3.90-3.80 (m, 2H), 3.68-3.61 (m, 2H), 3.54-3.35 (m, 7H), 3.26 (s, 3H), 3.21 (s, 1H), 3.13-3.03 (m, 2H), 3.01 (s, 4H), 2.82-2.74 (m, 1H), 2.33-2.09 (m, 5H), 2.08-2.03 (m, 4H), 1.93 (d, J=13.2 Hz, 1H), 1.82-1.69 (m, 2H), 1.66-1.56 (m, 1H), 1.49 (d, J=6.4 Hz, 3H), 1.21-1.08 (m, 2H), 0.92 (s, 3H), 0.61 (s, 3H).

### Example 47

### (1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

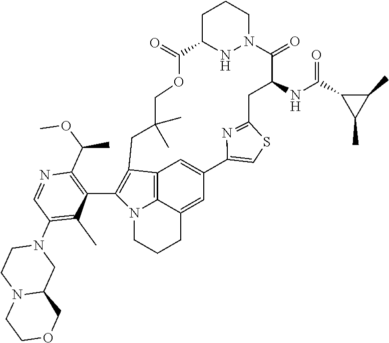

The title compound was prepared in analogy to the preparation of example 1 by using 3-[(2A4)-2-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol (intermediate D8) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1). Example 47 (25.1 mg) was obtained. MS calc'd 893.5 (MH⁺), measured 893.5 (MH⁺). ¹H NMR (400 MHz, Methanol-d₄) δ=8.51-8.45 (m, 1H), 8.41-8.37 (m, 1H), 7.50 (s, 1H), 7.43 (s, 1H), 5.83-5.76 (m, 1H), 4.47-4.38 (m, 2H), 4.24-4.19 (m, 1H), 4.18-4.11 (m, 2H), 4.08-4.03 (m, 1H), 3.88-3.79 (m, 3H), 3.77-3.72 (m, 1H), 3.66-3.60 (m, 2H), 3.56-3.50 (m, 3H), 3.49-3.44 (m, 3H), 3.44-3.41 (m, 1H), 3.40-3.35 (m, 2H), 3.29 (s, 3H), 3.27-3.24 (m, 1H), 3.09-3.06 (m, 1H), 3.05-3.01 (m, 2H), 2.81-2.73 (m, 1H), 2.27-2.17 (m, 3H), 2.12 (s, 3H), 1.95-1.89 (m, 1H), 1.78-1.69 (m, 1H), 1.64-1.57 (m, 1H), 1.53-1.49 (m, 3H), 1.41-1.29 (m, 3H), 1.17-1.11 (m, 7H), 0.95-0.90 (m, 3H), 0.62 (s, 3H).

### Example 48

### cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-tetrahydropyran-4-yl-cyclopropanecarboxamide

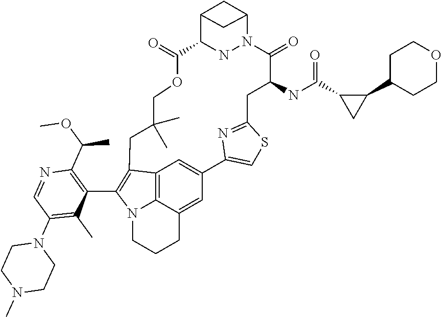

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and (1S,2R)-2-tetrahydropyran-4-ylcyclopropanecarboxylic acid (intermediate O5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 48 (27.8 mg) was obtained. MS calc'd 919.5 (MH⁺), measured 919.4 (MH⁺). ¹H NMR (400 MHz, Methanol-d₄) δ=8.52 (s, 1H), 8.30 (s, 1H), 7.55-7.50 (m, 1H), 7.44 (s, 1H), 5.74-5.59 (m, 1H), 4.68-4.63 (m, 1H), 4.59 (s, 1H), 4.49-4.40 (m, 1H), 4.07-3.99 (m, 1H), 3.96-3.91 (m, 2H), 3.90-3.85 (m, 1H), 3.74-3.48 (m, 7H), 3.45-3.35 (m, 6H), 3.29-3.24 (m, 4H), 3.15-2.96 (m, 7H), 2.71-2.64 (m, 1H), 2.60-2.52 (m, 1H), 2.49-2.42 (m, 1H), 2.38-2.32 (m, 1H), 2.28-2.21 (m, 2H), 2.19-2.16 (m, 3H), 1.78-1.62 (m, 3H), 1.53 (d, J=6.0 Hz, 3H), 1.50-1.37 (m, 3H), 1.21-1.13 (m, 1H), 1.12-1.03 (m, 2H), 0.89 (s, 3H), 0.83-0.76 (m, 1H), 0.65 (s, 3H).

### Example 49

### (1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[4.1.0]heptane-7-carboxamide

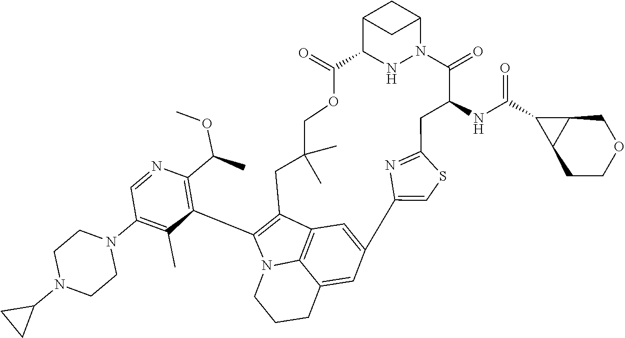

The title compound was prepared in analogy to the preparation of example 3 by using (7S,13S)-7-amino-(20M)-20-[5-(4-cyclopropylpiperazin-1-yl)-2-[(1S)-1-methoxyethyl]-4-methyl-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate N5) and trans-(1R,6S,7R)-3-oxabicyclo[4.1.0]heptane-7-carboxylic acid (Enamine, CAS #2763584-94-5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 49 (36.4 mg, faster eluted) was obtained. MS calc'd 917.5 (MH⁺), measured 917.5 (MH⁺). ¹H NMR (400 MHz, Methanol-d₄) δ=8.50 (s, 1H), 8.31 (s, 1H), 7.53 (s, 1H), 7.44 (s, 1H), 5.69-5.58 (m, 1H), 4.69-4.62 (m, 1H), 4.59 (s, 1H), 4.49-4.39 (m, 1H), 4.08-3.98 (m, 1H), 3.98-3.91 (m, 1H), 3.90-3.82 (m, 2H), 3.77-3.51 (m, 8H), 3.50-3.38 (m, 4H), 3.29-3.24 (m, 4H), 3.16-2.87 (m, 5H), 2.72-2.65 (m, 1H), 2.60-2.51 (m, 1H), 2.48-2.34 (m, 2H), 2.29-2.16 (m, 6H), 2.04-1.94 (m, 1H), 1.92-1.84 (m, 1H), 1.81 (t, J=4.4 Hz, 1H), 1.77-1.70 (m, 1H), 1.56-1.47 (m, 5H), 1.12-1.01 (m, 4H), 0.89 (s, 3H), 0.65 (s, 3H)

### Example 50

### (1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

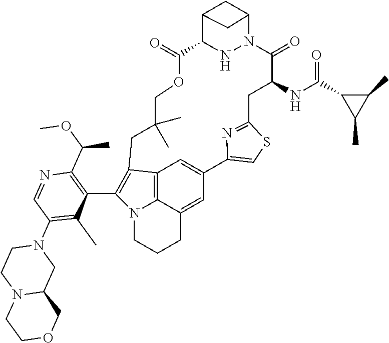

The title compound was prepared in analogy to the preparation of example 5 by using (7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-7-amino-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate N6) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 50 (15.5 mg) was obtained. MS calc'd 905.5 (MH⁺), measured 905.6 (MH⁺). ¹H NMR (400 MHz, Methanol-d₄) δ=8.42-8.36 (m, 1H), 8.30-8.24 (m, 1H), 7.54-7.47 (m, 1H), 7.42-7.38 (m, 1H), 5.72-5.58 (m, 1H), 4.67-4.63 (m, 1H), 4.62-4.59 (m, 2H), 4.40-4.34 (m, 1H), 4.09-4.03 (m, 1H), 3.90-3.84 (m, 2H), 3.80-3.76 (m, 1H), 3.75-3.68 (m, 2H), 3.54-3.48 (m, 1H), 3.44-3.39 (m, 1H), 3.29-3.24 (m, 1H), 3.20 (s, 3H), 3.19-3.17 (m, 1H), 3.09-2.99 (m, 3H), 2.99-2.90 (m, 2H), 2.78-2.75 (m, 1H), 2.69-2.65 (m, 1H), 2.65-2.65-2.60 (m, 1H), 2.59-2.51 (m, 3H), 2.48-2.41 (m, 2H), 2.35-2.29 (m, 1H), 2.26-2.17 (m, 3H), 2.02 (s, 3H), 1.52-1.46 (m, 4H), 1.46-1.26 (m, 3H), 1.19-1.12 (m, 7H), 0.91-0.83 (m, 3H), 0.62 (s, 3H).

### Example 51

### (1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide

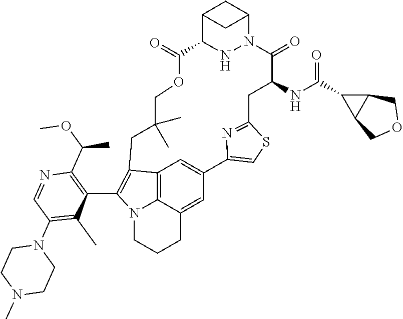

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate N2) and (1R,5S)-3-oxabicyclo[3.1.0]hexane-6-carboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 51 (29.9 mg) was obtained. MS calc'd 877.4 (MH⁺), measured 877.4 (MH⁺). ¹H NMR (400 MHz, Methanol-d₄) δ=8.52 (s, 1H), 8.32 (s, 1H), 7.54 (s, 1H), 7.45 (s, 1H), 5.73-5.55 (m, 1H), 4.72-4.59 (m, 2H), 4.51-4.41 (m, 1H), 4.11-4.01 (m, 1H), 4.00-3.88 (m, 3H), 3.80-3.65 (m, 5H), 3.61-3.50 (m, 3H), 3.48-3.38 (m, 4H), 3.31-3.22 (m, 5H), 3.16-3.05 (m, 2H), 3.03 (s, 4H), 2.74-2.67 (m, 1H), 2.62-2.54 (m, 1H), 2.51-2.43 (m, 1H), 2.40-2.18 (m, 5H), 2.17-2.15 (m, 3H), 2.13-2.09 (m, 1H), 1.72 (t, J=3.2 Hz, 1H), 1.56-1.49 (m, 4H), 0.91 (s, 3H), 0.65 (s, 3H).

### Example 52

### (1S,5R)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide

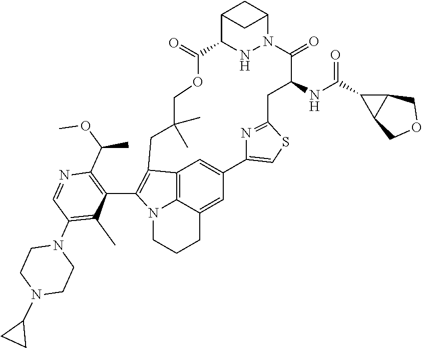

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate N2) and (1R,5S)-3-oxabicyclo[3.1.0]hexane-6-carboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 52 (22.8 mg) was obtained. MS calc'd 903.5 (MH⁺), measured 903.4 (MH⁺). ¹H NMR (400 MHz, Methanol-d₄) δ=8.47 (s, 1H), 8.29 (s, 1H), 7.52 (s, 1H), 7.42 (s, 1H), 5.68-5.56 (m, 1H), 4.70-4.58 (m, 2H), 4.41 (q, J=6.4 Hz, 1H), 4.11-4.01 (m, 1H), 3.98-3.85 (m, 3H), 3.81-3.50 (m, 9H), 3.46-3.37 (m, 3H), 3.28-3.23 (m, 4H), 3.20-2.92 (m, 5H), 2.73-2.65 (m, 1H), 2.60-2.52 (m, 1H), 2.49-2.41 (m, 1H), 2.34-2.15 (m, 5H), 2.14-2.06 (m, 4H), 1.70 (t, J=3.2 Hz, 1H), 1.55-1.47 (m, 4H), 1.10-1.01 (m, 4H), 0.88 (s, 3H), 0.62 (s, 3H).

### Example 53

### (1R,5S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide

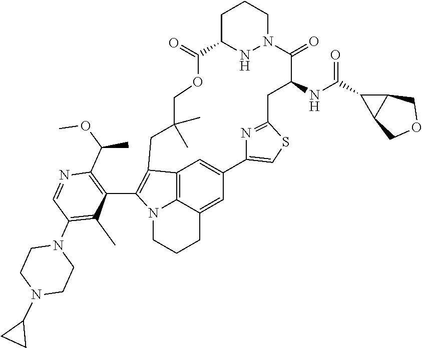

The title compound was prepared in analogy to the preparation of example 5 by using (7S,13S)-7-amino-(20M)-20-[5-(4-cyclopropylpiperazin-1-yl)-2-[(1S)-1-methoxyethyl]-4-methyl-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N7) and (1R,5S)-3-oxabicyclo[3.1.0]hexane-6-carboxylic acid instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 53 (11.8 mg) was obtained. MS calc'd 891.5 (MH⁺), measured 891.5 (MH⁺). ¹H NMR (400 MHz, Methanol-d₄) δ=8.38-8.35 (m, 2H), 7.49 (s, 1H), 7.39 (s, 1H), 5.78 (d, J=6.8 Hz, 1H), 4.87-4.82 (m, 1H), 4.45-4.33 (m, 2H), 4.28-4.19 (m, 1H), 4.16-4.05 (m, 1H), 3.95-3.80 (m, 4H), 3.75 (ddd, J=2.8, 5.6, 8.4 Hz, 2H), 3.52-3.39 (m, 2H), 3.27 (d, J=6.4 Hz, 1H), 3.24 (s, 3H), 3.14-3.06 (m, 3H), 3.05-2.98 (m, 4H), 2.86-2.77 (m, 4H), 2.27-2.14 (m, 4H), 2.13-2.05 (m, 2H), 2.00 (s, 3H), 1.93 (d, J=13.2 Hz, 1H), 1.79-1.68 (m, 3H), 1.65-1.55 (m, 1H), 1.47 (d, J=6.0 Hz, 3H), 0.92 (s, 3H), 0.60 (s, 3H), 0.55-0.47 (m, 4H).

### Example 54

### (1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[4.1.0]heptane-7-carboxamide

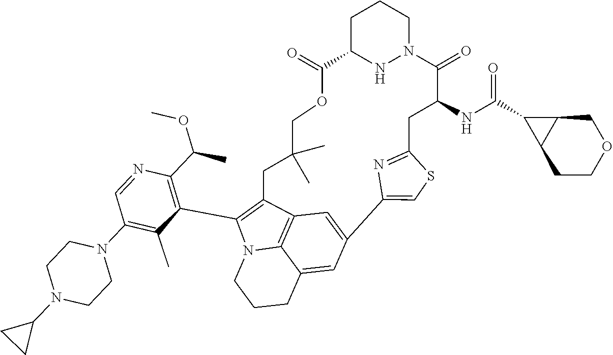

The title compound was prepared in analogy to the preparation of example 3 by using (7S,13S)-7-amino-(20M)-20-[5-(4-cyclopropylpiperazin-1-yl)-2-[(1S)-1-methoxyethyl]-4-methyl-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N7) and trans-(1R,6S,7R)-3-oxabicyclo[4.1.0]heptane-7-carboxylic acid (Enamine, CAS #2763584-94-5) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaene-8,14-dione (intermediate E) and norcarane-7-carboxylic acid. Example 54 (13.4 mg, faster eluted) was obtained. MS calc'd 905.5 (MH⁺), measured 905.5 (MH⁺). ¹H NMR (400 MHz, Methanol-d₄) δ=8.37 (s, 2H), 7.49 (s, 1H), 7.39 (s, 1H), 5.78 (d, J=7.2 Hz, 1H), 4.86-4.81 (m, 1H), 4.45-4.34 (m, 2H), 4.29-4.19 (m, 1H), 4.16-4.07 (m, 1H), 3.96-3.79 (m, 4H), 3.64-3.57 (m, 1H), 3.53-3.47 (m, 1H), 3.45-3.39 (m, 1H), 3.29-3.26 (m, 1H), 3.24 (s, 3H), 3.14-3.00 (m, 7H), 2.88-2.74 (m, 5H), 2.27-2.14 (m, 4H), 2.00 (s, 3H), 1.98-1.84 (m, 3H), 1.82-1.69 (m, 4H), 1.65-1.56 (m, 1H), 1.50-1.44 (m, 4H), 0.92 (s, 3H), 0.60 (s, 3H), 0.55-0.45 (m, 4H).

### Example 55

### (1r,2S,3R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

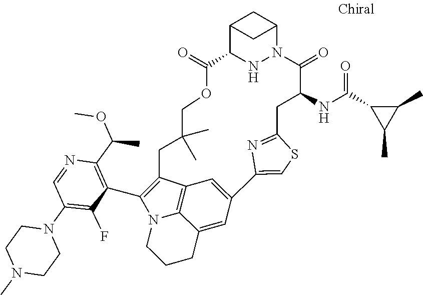

The title compound was prepared in analogy to the preparation of example 5 by using (7S,13S)-7-amino-(20P)-20-[4-fluoro-2-[(1S)-1-methoxyethyl]-5-(4-methylpiperazin-1-yl)-3-pyridyl]-17,17-dimethyl-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaene-8,14-dione (intermediate R2) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 55 (8.9 mg) was obtained. MS calc'd 867.4 (MH⁺), measured 867.7 (MH⁺). ¹H NMR (400 MHz, Methanol-d₄) δ=8.51 (d, J=10.2 Hz, 1H), 8.30 (s, 1H), 7.51 (s, 1H), 7.42 (s, 1H), 5.72 (s, 1H), 4.69-4.60 (m, 1H), 4.56-4.46 (m, 1H), 4.37 (q, J=6.0 Hz, 1H), 4.18-4.07 (m, 1H), 3.85-3.71 (m, 3H), 3.70-3.56 (m, 4H), 3.50-3.32 (m, 5H), 3.29-3.18 (m, 4H), 3.13-2.99 (m, 5H), 2.97-2.86 (m, 1H), 2.68-2.48 (m, 3H), 2.46-2.39 (m, 1H), 2.32-2.15 (m, 3H), 1.47 (d, J=6.0 Hz, 3H), 1.45-1.30 (m, 3H), 1.18-1.06 (m, 7H), 0.91-0.77 (m, 3H), 0.70-0.56 (m, 3H).

### Example 56

### (1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

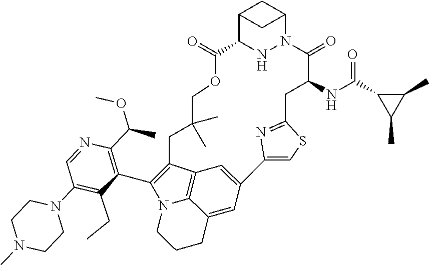

The title compound was prepared in analogy to the preparation of example 1 by using benzyl 4-[4-ethyl-(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate (intermediate D9) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1). Example 56 (38.6 mg) was obtained. MS calc'd 865.5 (MH⁺), measured 865.5 (MH⁺). ¹H NMR (400 MHz, Methanol-d₄) δ=8.62 (s, 1H), 8.40 (s, 1H), 7.55-7.50 (m, 1H), 7.45 (s, 1H), 5.77-5.69 (m, 1H), 4.51-4.40 (m, 2H), 4.33-4.26 (m, 1H), 4.24-4.15 (m, 1H), 3.89-3.80 (m, 2H), 3.71-3.61 (m, 2H), 3.59-3.52 (m, 1H), 3.50-3.44 (m, 2H), 3.37 (s, 6H), 3.30-3.25 (m, 1H), 3.21-3.07 (m, 3H), 3.04-2.99 (m, 3H), 2.96-2.88 (m, 1H), 2.84-2.73 (m, 1H), 2.46-2.36 (m, 1H), 2.33-2.21 (m, 2H), 2.13 (d, J=14.4 Hz, 2H), 1.97 (d, J=13.2 Hz, 1H), 1.88-1.75 (m, 1H), 1.69-1.57 (m, 1H), 1.51-1.48 (m, 3H), 1.45 (s, 3H), 1.22-1.10 (m, 8H), 1.04-0.98 (m, 3H), 0.80 (t, J=7.4 Hz, 3H), 0.55 (s, 3H).

### Example 57

### N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-methyl-norcarane-7-carboxamide

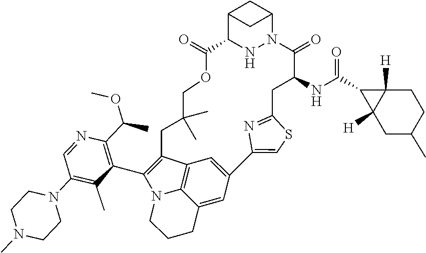

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and (1R,6S,7R)-3-methylnorcarane-7-carboxylic acid (intermediate O6) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid. Example 57 (20.9 mg) was obtained. MS calc'd 891.5 (MH⁺), measured 892.0 (MH⁺). ¹H NMR (400 MHz, Methanol-d₄) δ=8.46 (s, 1H), 8.38 (s, 1H), 7.49 (s, 1H), 7.41 (s, 1H), 5.79 (br d, J=8.4 Hz, 1H), 4.41 (q, J=5.8 Hz, 2H), 4.21 (dd, J=3.0, 11.9 Hz, 1H), 4.12-4.03 (m, 1H), 3.89-3.78 (m, 2H), 3.69-3.59 (m, 3H), 3.54-3.45 (m, 3H), 3.44-3.36 (m, 3H), 3.28-3.20 (m, 6H), 3.15-2.98 (m, 7H), 2.82-2.72 (m, 1H), 2.33-2.11 (m, 6H), 2.07 (s, 3H), 2.02-1.82 (m, 3H), 1.80-1.69 (m, 1H), 1.66-1.55 (m, 2H), 1.49 (br d, J=6.3 Hz, 5H), 0.95-0.87 (m, 5H), 0.84 (br dd, J=2.9, 6.5 Hz, 2H), 0.61 (s, 3H).

### Example 58 and example 59

### (1R,6R,7S)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide and (1R,6R,7R)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide

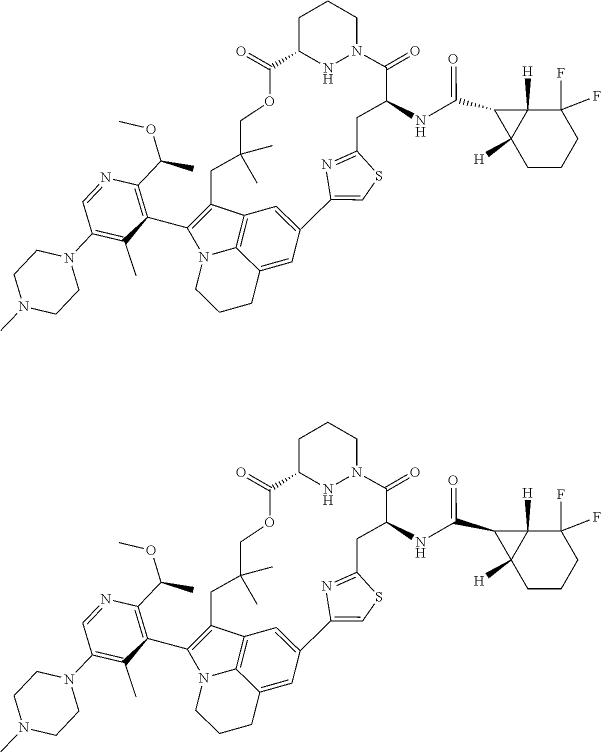

The title compound was prepared in analogy to the preparation of example 5 by using (7S,13S)-7-amino-(20M)-20-[2-[(1S)-1-methoxyethyl]-4-methyl-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N1) and 2,2-Difluorobicyclo[4.1.0]heptane-7-carboxylic acid (CAS #2361636-46-4, EN300-7433804) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G) and (1r,2R,3S)-2,3-dimethylcyclopropanecarboxylic acid and then purified by prep-HPLC and SFC.

SFC conditions: Instrument: SFC 150 MgM; Column: OJ, 250×30 mm I.D., 5 μm. Mobile phase: A for CO₂ and B for EtOH (0.1% NH₁₃H₂O); Gradient: B 45%; Flow rate: 65 mL/min; Back pressure: 100 bar; Column temperature: 35° C.

Example 58 (4.9 mg, SFC, slower eluted) was obtained. MS calc'd 913.5 (MH⁺), measured 913.5 (MH⁺). ¹H NMR (400 MHz, Methanol-d₄) δ=8.39 (s, 1H), 8.37 (s, 1H), 7.50 (s, 1H), 7.39 (s, 1H), 5.79 (dd, J=1.9, 8.6 Hz, 1H), 4.45-4.36 (m, 2H), 4.24 (br dd, J=3.1, 11.8 Hz, 1H), 4.15-4.06 (m, 2H), 3.90-3.85 (m, 1H), 3.83-3.78 (m, 1H), 3.64-3.61 (m, 2H), 3.47-3.40 (m, 2H), 3.25 (s, 3H), 3.19-3.14 (m, 2H), 3.09-3.03 (m, 4H), 2.82-2.74 (m, 2H), 2.68 (br d, J=1.3 Hz, 3H), 2.38 (s, 3H), 2.22-2.15 (m, 3H), 1.98 (s, 3H), 1.80-1.72 (m, 4H), 1.65-1.56 (m, 3H), 1.47 (d, J=6.1 Hz, 3H), 1.21 (s, 2H), 1.16 (d, J=6.3 Hz, 2H), 1.14-1.10 (m, 1H), 0.92 (s, 3H), 0.59 (s, 3H).

Example 59 (9.0 mg, SFC, slower eluted) was obtained. MS calc'd 913.5 (MH⁺), measured 913.5 (MH⁺). ¹H NMR (400 MHz, Methanol-d₄) δ=8.44 (s, 1H), 8.37 (s, 1H), 7.49 (s, 1H), 7.40 (s, 1H), 5.85 (br dd, J=2.1, 8.3 Hz, 1H), 4.45-4.37 (m, 2H), 4.21 (br dd, J=2.9, 11.6 Hz, 1H), 4.12-4.05 (m, 2H), 3.89-3.81 (m, 2H), 3.63 (s, 1H), 3.51-3.46 (m, 2H), 3.46-3.43 (m, 1H), 3.42-3.40 (m, 1H), 3.23 (s, 3H), 3.08-3.05 (m, 1H), 3.03-2.98 (m, 2H), 2.94 (s, 3H), 2.82-2.74 (m, 1H), 2.25 (br d, J=4.1 Hz, 1H), 2.22-2.19 (m, 1H), 2.16-2.10 (m, 1H), 2.04-2.03 (m, 2H), 1.95-1.91 (m, 2H), 1.80-1.72 (m, 4H), 1.56-1.54 (m, 1H), 1.48 (d, J=6.3 Hz, 4H), 1.26 (s, 3H), 1.22 (s, 3H), 1.16 (d, J=6.1 Hz, 4H), 0.90 (s, 4H), 0.61 (s, 3H).

### Example 60

### (1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

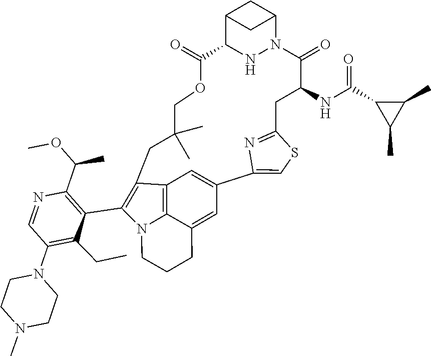

The title compound was prepared in analogy to the preparation of example 1 by using benzyl 4-[4-ethyl-(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate (intermediate D9) and methyl (4S)-2-[(2S)-3-(4-bromothiazol-2-yl)-2-[[(1r,2S,3R)-2,3-dimethylcyclopropanecarbonyl]amino]propanoyl]-2,3-diazabicyclo[3.1.1]heptane-4-carboxylate (intermediate C6) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3). Example 60 (14.5 mg) was obtained. MS calc'd 877.5 (MH⁺), measured 877.5 (MH⁺). ¹H NMR (400 MHz, Methanol-d₄) δ=8.60 (s, 1H), 8.27 (s, 1H), 7.54 (s, 1H), 7.44 (s, 1H), 5.55-5.47 (m, 1H), 4.74 (s, 1H), 4.71-4.66 (m, 1H), 4.48-4.41 (m, 1H), 4.24-4.16 (m, 1H), 3.85-3.73 (m, 2H), 3.67 (t, J=12.4 Hz, 2H), 3.60-3.53 (m, 1H), 3.49-3.35 (m, 9H), 3.31-3.25 (m, 2H), 3.19-3.09 (m, 3H), 3.03 (s, 3H), 2.96-2.88 (m, 1H), 2.78-2.72 (m, 1H), 2.66-2.60 (m, 1H), 2.52-2.45 (m, 1H), 2.43-2.37 (m, 1H), 2.34-2.28 (m, 1H), 2.24-2.13 (m, 3H), 1.65 (t, J=9.6 Hz, 1H), 1.50 (d, J=6.4 Hz, 3H), 1.46-1.31 (m, 2H), 1.21-1.13 (m, 7H), 0.98 (s, 3H), 0.83 (t, J=7.6 Hz, 3H), 0.55 (s, 3H).

### Example 61

### (1r,2S,3R)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

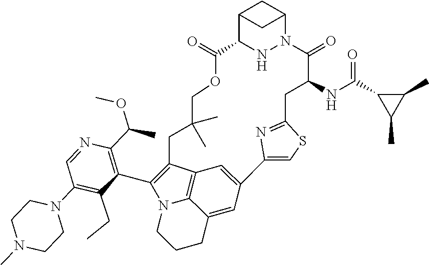

The title compound was prepared in analogy to the preparation of example 1 by using benzyl 4-[4-cyclopropyl-(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate (intermediate D10) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1). Example 61 (17.6 mg) was obtained. MS calc'd 877.5 (MH⁺), measured 877.5 (MH⁺). ¹H NMR (400 MHz, Methanol-d₄) δ=8.42 (s, 1H), 8.36 (s, 1H), 7.51 (s, 1H), 7.42 (s, 1H), 5.72-5.68 (m, 1H), 4.50-4.40 (m, 2H), 4.30-4.18 (m, 2H), 3.93-3.84 (m, 1H), 3.82 (s, 2H), 3.75-3.67 (m, 1H), 3.65-3.57 (m, 2H), 3.54-3.41 (m, 4H), 3.38 (s, 3H), 3.29-3.19 (m, 2H), 3.16-3.07 (m, 2H), 3.05-2.98 (m, 4H), 2.82-2.72 (m, 1H), 2.35-2.27 (m, 1H), 2.25-2.12 (m, 3H), 2.00-1.90 (m, 1H), 1.83-1.70 (m, 2H), 1.67-1.57 (m, 1H), 1.52-1.29 (m, 6H), 1.18-1.12 (m, 7H), 1.00 (s, 3H), 0.98-0.91 (m, 1H), 0.85-0.78 (m, 1H), 0.51 (s, 3H), 0.31-0.23 (m, 1H), −0.04-−0.11 (m, 1H).

### Example 62

### (1r,2R,3S)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

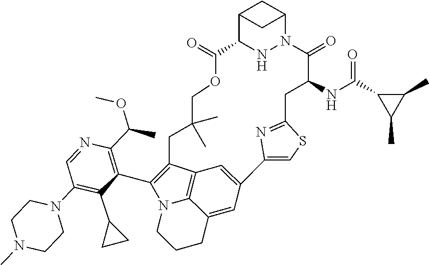

The title compound was prepared in analogy to the preparation of example 1 by using benzyl 4-[4-cyclopropyl-(5M)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate (intermediate D10) and methyl (4S)-2-[(2S)-3-(4-bromothiazol-2-yl)-2-[[(1r,2S,3R)-2,3-dimethylcyclopropanecarbonyl]amino]propanoyl]-2,3-diazabicyclo[3.1.1]heptane-4-carboxylate (intermediate C6) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3). Example 62 (11.5 mg) was obtained. MS calc'd 889.5 (MH⁺), measured 889.5 (MH⁺). ¹H NMR (400 MHz, Methanol-d₄) δ=8.42 (s, 1H), 8.24 (s, 1H), 7.52 (s, 1H), 7.42 (s, 1H), 5.52-5.45 (m, 1H), 4.73-4.65 (m, 2H), 4.49-4.41 (m, 1H), 4.26-4.16 (m, 1H), 3.86 (d, J=11.8 Hz, 1H), 3.80-3.68 (m, 3H), 3.65-3.57 (m, 2H), 3.54-3.47 (m, 2H), 3.43-3.39 (m, 1H), 3.38-3.35 (m, 4H), 3.30-3.08 (m, 5H), 3.06-2.99 (m, 4H), 2.78-2.68 (m, 1H), 2.65-2.55 (m, 1H), 2.51-2.41 (m, 1H), 2.35-2.27 (m, 1H), 2.26-2.09 (m, 3H), 1.78-1.69 (m, 1H), 1.65-1.59 (m, 1H), 1.48-1.28 (m, 6H), 1.20-1.13 (m, 7H), 0.97 (s, 3H), 0.87-0.78 (m, 1H), 0.50 (s, 3H), 0.33-0.25 (m, 1H), 0.00-−0.04 (m, 1H).

### Example 63

### (1r,2R,3S)—N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

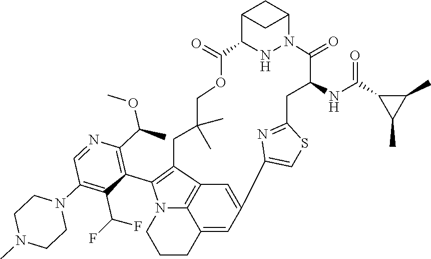

The title compound was prepared in analogy to the preparation of example 5 by using (7S,13S)-7-amino-(20M)-20-[4-(difluoromethyl)-2-[(1S)-1-methoxyethyl]-5-(4-methylpiperazin-1-yl)-3-pyridyl]-17,17-dimethyl-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaene-8,14-dione (intermediate N8) instead of (7S,13S)-7-amino-(20P)-20-[2-[(1S)-1-methoxyethyl]-4-methyl-5-(4-methylpiperazin-1-yl)-3-pyridyl]-17,17-dimethyl-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaene-8,14-dione (intermediate G). Example 63 (6.8 mg) was obtained. MS calc'd 887.4 (MH⁺), measured 887.4 (MH⁺). ¹H NMR (400 MHz, Methanol-d₄) δ=8.74 (s, 1H), 8.34 (s, 1H), 7.48 (s, 1H), 7.39 (s, 1H), 6.79 (t, J=53.8 Hz, 1H), 5.74-5.69 (m, 1H), 4.47-4.40 (m, 2H), 4.26 (dd, J=3.1, 11.9 Hz, 1H), 4.15-4.08 (m, 1H), 3.88-3.79 (m, 2H), 3.53-3.45 (m, 2H), 3.42 (br d, J=14.5 Hz, 1H), 3.29-3.29 (m, 3H), 3.27 (br s, 1H), 3.24-3.17 (m, 2H), 3.14-3.04 (m, 2H), 3.02-2.93 (m, 1H), 2.90-2.72 (m, 5H), 2.50 (s, 3H), 2.33-2.25 (m, 2H), 2.24-2.18 (m, 1H), 2.17-2.10 (m, 1H), 1.97-1.90 (m, 1H), 1.85-1.74 (m, 1H), 1.67-1.57 (m, 1H), 1.48 (d, J=6.1 Hz, 3H), 1.43-1.35 (m, 2H), 1.34-1.28 (m, 1H), 1.18-1.15 (m, 4H), 1.13 (d, J=6.0 Hz, 3H), 0.95 (s, 3H), 0.52 (s, 3H).

### Example 64

### (1r,2R,3S)—N-[(7S,13S)-(20P)-20-[4-chloro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide

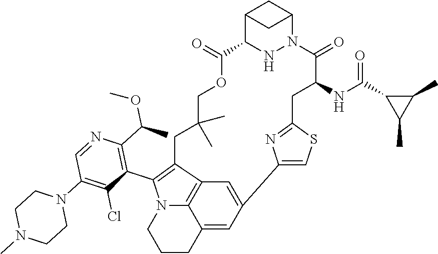

The title compound was prepared in analogy to the preparation of example 1 by using benzyl 4-[4-chloro-(5P)-5-[3-(3-hydroxy-2,2-dimethyl-propyl)-6-(4,4,5,5-tetramethyl-1,3,2-dioxaborolan-2-yl)-1-azatricyclo[6.3.1.0^{4,12}]dodeca-2,4(12),5,7-tetraen-2-yl]-6-[(1S)-1-methoxyethyl]-3-pyridyl]piperazine-1-carboxylate (intermediate D12) and methyl (4S)-2-[(2S)-3-(4-bromothiazol-2-yl)-2-[[(1r,2S,3R)-2,3-dimethylcyclopropanecarbonyl]amino]propanoyl]-2,3-diazabicyclo[3.1.1]heptane-4-carboxylate (intermediate C6) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3). Example 64 (3.6 mg, faster eluted) was obtained and purified using SFC. SFC conditions: column: DAICEL CHIRALCEL OD (250 mm×30 mm×10 um); mobile phase: [A: CO₂; B: MeOH (0.1% NH₃H₂O)]; B %: 40.00%-40.00%, 8.80 min; flow rate: 70.00 ml/min). MS calc'd 883.4 (MH⁺), measured 883.5 (MH⁺). ¹H NMR (400 MHz, Methanol-d₄) δ=8.49 (s, 1H), 8.32 (s, 1H), 7.49 (s, 1H), 7.40 (s, 1H), 5.95-5.80 (m, 1H), 5.38-5.32 (m, 1H), 4.45-4.37 (m, 1H), 4.36-4.28 (m, 1H), 4.01-3.88 (m, 2H), 3.72-3.62 (m, 2H), 3.50-3.42 (m, 1H), 3.09 (s, 3H), 2.82-2.77 (m, 1H), 2.72-2.62 (m, 5H), 2.57-2.52 (m, 1H), 2.49-2.44 (m, 1H), 2.38 (s, 4H), 2.27-2.15 (m, 5H), 2.06-2.00 (m, 2H), 1.66-1.54 (m, 2H), 1.50 (d, J=9.0 Hz, 3H), 1.15 (d, J=9.0 Hz, 3H), 1.12 (d, J=6.4 Hz, 4H), 0.93-0.88 (m, 3H), 0.78 (s, 2H), 0.73 (s, 2H), 0.10 (s, 3H).

### Example 65

### (1r, 2S,3R)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide

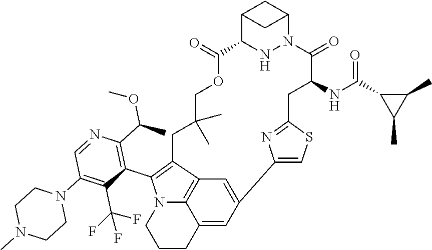

The title compound was prepare in analogy to the preparation o example by using 3-[(2M)-2-[2-[(1S)-1-methoxyethyl]-5-(4-methylpiperazin-1-yl)-4-(trifluoromethyl)-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol (intermediate D13) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1) and methyl (3S)-1-[(2S)-3-(4-bromothiazol-2-yl)-2-[[(1r,2S,3R)-2,3-dimethylcyclopropanecarbonyl]amino]propanoyl]hexahydropyridazine-3-carboxylate (intermediate C3). Example 65 (120.5 mg, faster eluted) was obtained and purified using SFC. SFC conditions: column: DAICEL CHIRALCEL OD (250 mm×30 mm×10 um); mobile phase: [A: CO₂; B: MeOH (0.1% NH₃H₂O)]; B %: 40.00%-40.00%, 8.80 min; flow rate: 70.00 ml/min). MS calc'd 905.4 (MH⁺), measured 905.5 (MH⁺). ¹H NMR (400 MHz, Methanol-d₄) δ=8.93 (s, 1H), 8.35 (s, 1H), 7.50 (s, 1H), 7.43-7.40 (m, 1H), 5.86-5.72 (m, 1H), 4.41-4.55 (m, 2H), 4.24-4.17 (m, 1H), 4.17-4.08 (m, 1H), 3.90-3.84 (m, 1H), 3.81 (s, 1H), 3.64 (s, 3H), 3.57 (s, 1H), 3.50 (d, J=8.0 Hz, 2H), 3.45-3.41 (m, 2H), 3.39 (s, 2H), 3.22 (s, 3H), 3.01 (s, 4H), 2.97-2.89 (m, 1H), 2.76 (s, 1H), 2.31 (s, 2H), 2.20-2.08 (m, 2H), 1.97-1.85 (m, 1H), 1.79-1.68 (m, 1H), 1.65-1.54 (m, 1H), 1.50 (d, J=6.0 Hz, 3H), 1.37 (s, 3H), 1.21-1.05 (m, 8H), 0.90 (s, 3H), 0.54 (s, 3H).

### Example 66

### (1r, 2R,3S)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,32-tetrazaheptacyclo[23.3.1.1^{2,5}.1^{19,13}.1^{10,12}.0^{19,27}.0^{21,26}]dotriaconta-1(28),2,5(32),19,25(29),26-hexaen-7-yl]-2,3-dimethyl-cyclopropanecarboxamide

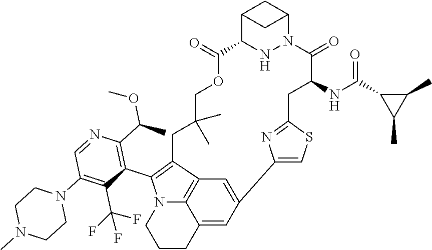

The title compound was prepare in analogy to the preparation of example 1 by using 3-[(2M)-2-[2-[(1S)-1-methoxyethyl]-5-(4-methylpiperazin-1-yl)-4-(trifluoromethyl)-3-pyridyl]-6-(4,4,5,5-tetramethyl-1,3,2-dioxaborolan-2-yl)-1-azatricyclo[6.3.1.0^{4,12}]dodeca-2,4(12),5,7-tetraen-3-yl]-2,2-dimethyl-propan-1-ol (intermediate D13) and methyl (4S)-2-[(2S)-3-(4-bromothiazol-2-yl)-2-[[(1r,2S,3R)-2,3-dimethylcyclopropanecarbonyl]amino]propanoyl]-2,3-diazabicyclo[3.1.1]heptane-4-carboxylate (intermediate C6) instead of benzyl 4-[(5P)-5-[1-(3-hydroxy-2,2-dimethyl-propyl)-7-(4,4,5,5-tetramethyl-1,3,2-dioxaborolan-2-yl)-4,5-dihydro-3H-benzo[cd]indol-2-yl]-6-[(1S)-1-methoxyethyl]-4-methyl-3-pyridyl]piperazine-1-carboxylate (intermediate D1). and purified by SFC. Example 66 (31.1 mg, faster eluted) was obtained. SFC conditions: column: DAICEL CHIRALCEL OD (250 mm×30 mm×10 um); mobile phase: [A: CO₂; B: MeOH (0.1% NH₃H₂O)]; B %: 40.00%-40.00%, 8.80 min; flow rate: 70.00 ml/min). MS calc'd 917.4 (MH⁺), measured 917.5 (MH⁺). ¹H NMR (400 MHz, Methanol-d₄) δ=8.94 (s, 1H), 8.25 (s, 1H), 7.50 (s, 1H), 7.41 (s, 1H), 5.66 (s, 1H), 5.68-5.62 (m, 1H), 4.67-4.62 (m, 1H), 4.56 (s, 1H), 4.42-4.35 (m, 1H), 4.15-4.05 (m, 1H), 3.88 (d, J=10.4 Hz, 1H), 3.73-3.55 (m, 5H), 3.54-3.44 (m, 3H), 3.44-3.35 (m, 4H), 3.18 (s, 3H), 3.07 (s, 1H), 3.01 (s, 5H), 2.92-2.85 (m, 1H), 2.64 (s, 1H), 2.54 (s, 1H), 2.46-2.36 (m, 2H), 2.34-2.25 (m, 1H), 2.24-2.16 (m, 2H), 1.50 (d, J=6.0 Hz, 3H), 1.1-1.10 (m, 8H), 0.85 (s, 3H), 0.56 (s, 3H).

### BIOLOGICAL EXAMPLE

Compound Example 24 (page 116) and compound Example 96 (page 175) from WO2024169914 were cited as reference compound 1 and reference compound 2 for this invention.

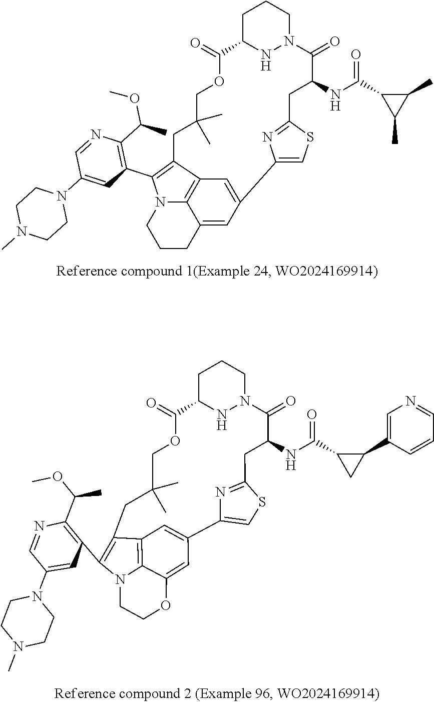

### Example 38

### Cell Viability Assay

The purpose of this cellular assay was to determine the effects of test compounds on the proliferation of human cancer cell lines SW620 (ATCC-CCL-227) over a 3-day treatment period by quantifying the amount of NADPH present at endpoint using Cell Counting Kit-8.

Cells were seeded at cells/well (SW620) in 96-well assay plates (Corning-3699) and incubated overnight. On the day of the assay, diluted compounds were then added with a final concentration of 0.500 DMSO. After 72 hrs incubation, a tenth of the volume of cell counting kit 8(Dnjindo-CK04) was added into each well. Read the signal (OD450 minus OD650) using EnVision after 2 hrs incubation. IC₅₀ was determined by fitting a 4-parameter sigmoidal concentration response model.

TABLE 1
Activity of Examples and Compounds of present
invention in KRAS Cell viability assay

| Examples | G12V IC50 (nM) |
|---|---|
| Example 1 | 6.7 |
| Example 2 | 4.5 |
| Example 3 | 6.2 |
| Example 4 | 2.5 |
| Example 5 | 5.8 |
| Example 6 | 2.4 |
| Example 7 | 1.5 |
| Example 8 | 5.9 |
| Example 9 | 5.4 |
| Example 10 | 3.7 |
| Example 11 | 1.7 |
| Example 12 | 2.4 |
| Example 13 | 8.6 |
| Example 14 | 4.5 |
| Example 15 | 3.7 |
| Example 16 | 8.1 |
| Example 17 | 5.3 |
| Example 18 | 8.2 |
| Example 19 | 8.3 |
| Example 20 | 3.7 |
| Example 21 | 8.4 |
| Example 22 | 3.8 |
| Example 23 | 4.9 |
| Example 24 | 4.0 |
| Example 25 | 6.1 |
| Example 26 | 3.0 |
| Example 27 | 3.7 |
| Example 28 | 2.2 |
| Example 29 | 0.7 |
| Example 30 | 1.2 |
| Example 31 | 5.8 |
| Example 32 | 1.6 |
| Example 33 | 5.2 |
| Example 34 | 1.4 |
| Example 35 | 0.6 |
| Example 36 | 0.7 |
| Example 37 | 1.9 |
| Example 38 | 0.5 |
| Example 39 | 0.3 |
| Example 40 | 0.4 |
| Example 41 | 1.2 |
| Example 42 | 0.5 |
| Example 43 | 1.4 |
| Example 44 | 4.2 |
| Example 45 | 1.1 |
| Example 46 | 3.5 |
| Example 47 | 4.6 |
| Example 48 | 1.2 |
| Example 49 | 3.8 |
| Example 51 | 3.1 |
| Example 54 | 1.5 |
| Example 55 | 0.9 |
| Example 57 | 2.5 |
| Example 58 | 1.2 |
| Example 59 | 0.5 |
| Example 60 | 6.5 |
| Example 61 | 5.8 |
| Example 63 | 1.6 |
| Example 64 | 4.6 |

### Example 39

Selectivity Profiles in Target Engagement Assays: Cellular RAS-BRAF NanoBiT G12V Vs. WT

The RAS-RAF Nanoflit assay is utilized to evaluate how a molecule disrupts or inhibits the RAS-RAF RBD interaction. It determines the potency of the molecule's engagement with wild-type or mutant RAS and its effectiveness in preventing RAS from binding to RAF effectors. A higher IC50 value signifies weaker binding and a diminished effect on downstream signal transduction.

The stable H293T cell line (ATCC) expressing BRAF_Lgflit and Smflit_K-RAS G12V was seeded into a 96-well assay plate (Revvity-6005181) at a density of 30,000 cells per well. Compounds were added to the wells in an 8-point, 4-fold dilution series, starting at a concentration of 1 μM. After a 4-hour incubation at 37° C., Nano-Glo® Live Cell Reagent (Promega-N1150) was added to the assay wells, followed by an additional 0.5-hour incubation. Luminescence signals were measured using a microplate reader (BMG PHERAstar FSX). Compounds that disrupted the KRAS:RAF complex were identified by a decrease in luminescence signal compared to DMSO control wells.

TABLE 2
Data of RAS-BRAF NanoBiT assay

| | RAS-RAF IC50 (nM) | | Selectivity index |
|---|---|---|---|
| | G12V | WT | (WT/G12V) |
| Reference Compound 1 | 4.1 | 73.2 | 18 |
| Reference Compound 2 | 10.7 | 66.7 | 6 |
| Example 29 | 9.5 | >5000 | >526 |
| Example 30 | 18.5 | >5000 | >270 |
| Example 12 | 57.1 | >5000 | >88 |

From Table 2, Example 29, Example 30 and Example 12 demonstrated significantly improved target engagement of G12V against WT KRAS than reference compound 1 and reference compound 2 in the cellular RAS-RAF NanoBit assay. The selectivity index of Example 29 is more than 29 folds (526/18=29) higher than that of reference compound 1. Similarly, the selectivity index of Example 12 is about 15 folds (88/6=15) higher than that of reference compound 2.

Example 29, Example 30 and Example 12 showed enhanced selectivity towards KRAS G12V vs WT in the RAS-RAF NanoBit assays measuring direct target engagement and signaling complex disruption.

The improved selectivity of G12V over WT KRAS, compared to reference compound 1 and reference compound 2, demonstrates that the compounds of this invention offer a higher therapeutic window and greater safety margin for treating cancers with the G12V RAS mutation in clinical settings.

### Example 40

### Single Dose Pharmacokinetics (PK) Study in Male Beagle Dogs

The purpose of this study was to determine the pharmacokinetics of selected compounds following single oral gavage administration in male beagle dogs. Briefly, one group of male beagle dogs (N=3/group) were treated with a single dose of compound orally at 3 mg/kg (PO). Blood samples were collected at pre-dose, and at 0.083 (Reference compound 1 only), 0.25, 0.5, 1, 2, 4, 8, 12 (Reference compound 1 only), 24 and 48 hours post-dose. Following collection, the blood samples were centrifuged to obtain plasma. The concentration of compound in plasma samples was determined using LC-MS/MS method. The pharmacokinetic parameters were calculated by non-compartmental analysis.

TABLE 3
Results of SDPK

3 mg/kg, PO

| | Cₘₐₓ | AUC₀₋ₗₐₛₜ |
|---|---|---|
| compound | (ng/mL) | (h × ng/mL) |
| Reference Compound 1 | 461 | 1930 |
| Example 29 | 4287 | 27012 |

From Table 3, it can be seen that Example 29 has excellent pharmacokinetic properties in dog model. Especially Example 29 has 9.3-fold increase in Cₘₐₓ and 14-fold increase in AUC₀₋ₗₐₛₜ compared with reference compound 1.

Above improvement over reference compound 1 demonstrated that the compounds of this invention are more suitable for treating cancers with RAS mutation as an orally therapeutic active ingredient in clinic.

### Example 41

### Cytotoxicity Profiles in RAS Dependent Normal Cells

Skin and gastrointestinal (GI) toxicity is among the most common target related adverse effects (TRAEs) associated with pan-RAS inhibitors, likely due to their strong engagement with not only mutant RAS but also wild-type (WT) RAS (ASCO-GI 2025; Elena Koltun et al., AACR 2024; ENA 2024). A molecule that increases selectivity over WT RAS, as indicated by target engagement assays, is expected to show significantly reduced skin and GI toxicity (ENA-LBA514-Hong-etal-2024). This reduction can be further validated in preclinical skin and GI models, as discussed in recent studies and methodologies (Matsui T et al. Front Genet. 2021. Danilenko D M et al. Toxicologic Pathology. 2016. Poumay Y et al. Arch Dermatol Res. 2004).

The viability of skin and GI organoids and cell systems is known to correlate with skin and GI toxicity. In example 39, we evaluated our molecule using a RAS (WT or G12V)-RAF protein-protein interaction (PPI) disruption assay to compare its activity between G12V and WT RAS. Here, we further assessed cell viability in human intestinal organoids, human colon epithelial mucin cells, human primary epidermal organoids, and HaCaT keratinocytes to measure cytotoxicity.

### Colon Cell & Organoid Viability

Drug-induced gastrointestinal (GI) toxicity is a common adverse event in clinical trials, characterized by symptoms such as diarrhea, ulceration, and inflammation due to impaired barrier function. The intestine is crucial in mediating drug absorption and metabolism. The cells of the NCM460 cell line (ATCC) are a normal human colon mucosal epithelial cell line. They are very close to the normal colonocytes and can be used for in vitro studies of the intestinal barrier for non-transformed colonocytes, but also to study absorption. In addition, intestinal organoids offer an advanced model that more accurately reflects the self-renewal, cellular composition, and function of the intestinal epithelium. Both normal colon mucosal epithelial cell and intestinal organoids serve as valuable tools for high-throughput screening of GI toxicity and assessing intestinal function in preclinical drug discovery.

### Intestinal Organoids

To evaluate toxicity, intestinal organoids were exposed to test compounds or the GI-toxic drug trametinib as a positive control, with post-treatment cell viability assessed for comparison. Intestinal organoids were derived from human ileal regions and cultured according to the manufacturer's protocols provided with the Human Intestinal Organoid Kit (K2002-HI) in 70% Corning® Matrigel® domes. They were incubated at 37° C. with 5% CO₂ for 6-8 days before passaging, with medium changes occurring 2-3 times per week. Organoids were passaged using Organoid Dissociation Solution followed by mechanical disruption to break up the domes. The suspensions were washed, resuspended in a 3:7 mixture of DMEM/F12 and Matrigel®, and seeded as droplets.

Organoids were harvested as described and seeded in a 40 clumps/8 μL Corning® Matrigel® dome format in a 96-well plate with Human Intestinal Organoid Expansion Medium. Plates were incubated at 37° C. and 5% CO₂ for 24 hours. On day 1, the expansion medium was replaced with differentiation medium. On day 4, compounds were added to plate wells in a 7-point, 3-fold dilution series starting at a concentration of ⅕ μM. Drugs were solubilized in DMSO and added to cultures with a final DMSO concentration of <0.1%. Cultures were treated with drugs for 5 days. After treatment, viability was measured using the CellTiter-Glo® 3D cell viability assay according to the manufacturer's protocols. Viability for each treatment condition was expressed as a percentage of the solvent control.

The IC50 concentration for intestinal organoid growth inhibition by each drug was calculated by plotting the log of drug concentration against the percent of maximum viability relative to solvent control cultures using GraphPad Prism® 8.0. The following equation was employed for curve fitting: log(inhibitor) vs. response−variable slope (4 parameters): Y=bottom+(top−bottom)/[1+10^{((Log IC50-X)×HillSlope)}].

TABLE 4
Data of intestinal organoids assay

| | IC₅₀ (nM) |
|---|---|
| Reference Compound 1 | 7.5 |
| Reference Compound 2 | 5.5 |
| Example 29 | 146.8 |
| Example 30 | 102.9 |
| Example 12 | 245.4 |

In Table 4, Example 29 demonstrated an IC50 value of 146.8 nM in intestinal organoids, which is 19 times higher than that of reference compound I. Similarly, Example 12 demonstrated an IC50 value of 245.4 nM in intestinal organoids, which is 44 times higher than that of reference compound 2.

The significantly reduced cytotoxicity compared to reference compound 1 and reference compound 2 demonstrated that the compounds of this invention offer a higher therapeutic window and a greater safety margin regarding GI toxicity for treating cancers with GV RAS mutation in clinic settings.

### NCM460

The NCM460 cell line, derived from normal human colon mucosal epithelial cells, serves as an essential in vitro model for studying human intestinal physiology and pathology. Established from histologically normal tissue from a transverse colon, NCM460 cells exhibit typical gastrointestinal epithelial characteristics, such as expressing villin and human secretory component, and maintaining a non-tumorigenic phenotype-they do not grow in soft agar and do not form tumors in nude mice. NCM460 cells reflect various stages of epithelial differentiation and demonstrate multilineage capabilities, including mucin-positive and neuroendocrine marker expression. These attributes make NCM460 an ideal model for evaluating GI toxicity, cell differentiation, drug transport, and epithelial barrier functions.

The NCM-460 cell line (IM-H445) was seeded into 384-well assay plate (Corning 3764) at 250 cells/well. Compound was present in plate wells as a 10-point 3-fold dilution series starting at a concentration of 1 μM. After incubation at 37° C., 5% CO₂ for 5 days, a mixture of CellTiter-Glo® 2.0 Cell Viability Assay (Promega-G9243) was added to assay sample wells and then incubated for an additional 30 minutes. Luminescence signal was read on a microplate reader (PE EnVision® 2105). Compounds that facilitate cytotoxicity to NCM-460 were identified as those eliciting a decrease in the Luminescence signal relative to DMSO control wells.

TABLE 5
Data of NCM460 colon cell line assay

| | IC₅₀ (nM) |
|---|---|
| Reference Compound 1 | 10.1 |
| Reference Compound 2 | 3.0 |
| Example 29 | >1000 |
| Example 30 | >1000 |
| Example 12 | >1000 |

In Table 5, Example 29, Example 30 and Example 12 showed minimal toxicity effects on the normal human colon mucosal epithelial cell line NCM460. Example 29 demonstrated an IC50 value 100 times higher than that of reference compound 1; similarly, Example 12 demonstrated an IC50 value 300 times higher than that of reference compound 2; indicating much lower cytotoxicity in those WT RAS-dependent normal colon cell lines.

The significantly reduced cytotoxicity compared to reference compound 1 and reference compound 2 demonstrated that the compounds of this invention offer a higher therapeutic window and a greater safety margin regarding GI toxicity for treating cancers with G12V RAS mutation in clinic settings.

### Skin Cell Line & Organoids

In vitro skin model systems are increasingly utilized for both early evaluation of therapeutic drug candidates and confirmatory mechanistic studies. Commonly used models, such as human primary epidermal organoids (HPEO) and the keratinocyte cell line HaCaT, are employed for high-throughput compound screening. These systems have proven to be quite predictive of in vivo pharmacological and toxicological effects on the skin, including clinical skin toxicity.

### HPEO

HEPOs, generated using primary keratinocytes from foreskin and adult skin, accurately mimic the human epidermis. They stratify from the basal layer to the stratum corneum and express markers such as Ki67, collagen 17, and several keratins. These cultures can be produced on a large scale, serving as a valuable model for screening therapeutic compounds and studying epidermal pathologies.

The purpose of this HPEO assay is to measure the ability of test compounds to cause cell damage or cell death.

Data is reported as IC50 values. Human primary epidermal organoids (HPEO) were seeded into a 96-well assay plate (Corning #3595) at 2000 cells per 6 μL per well. Compounds were present in plate wells as a 7-point, 5-fold dilution series starting at a concentration of 5 μM. After incubation at 37° C., 5% CO₂ for 96 hours, a mixture of CellTiter-Glo® Reagent (Promega-G9682) was added to assay sample wells, followed by an additional 30-minute incubation. Luminescence signals were read on a microplate reader (PerkinElmer, Ensight). Compounds that facilitate cytotoxicity to HPEO were identified as those eliciting a decrease in the luminescence signal relative to DMSO control wells. IC50 values were determined by fitting the data with Xlfit software.

TABLE 6
Data of HPEO skin organoid assay

| | IC₅₀ (nM) |
|---|---|
| Reference Compound 1 | 4.95 |
| Reference Compound 2 | <3.2 |
| Example 29 | >3000 |
| Example 30 | >3000 |
| Example 12 | >3000 |

In Table 6, Example 29 and Example 30 and Example 12 demonstrated IC50 values in human primary epidermal organoids, all exceeding 3000 nM. These values are more than 600 times higher than those for reference compound 1 and reference compound 2.

The significantly reduced cytotoxicity compared to reference compound 1 and reference compound 2 demonstrated that the compounds of this invention offer higher therapeutic window and a greater safety margin regarding skin toxicity for treating cancers with G12V RAS mutation in clinic.

### HaCaT

HaCaT cells are used to study skin biology and toxicity, forming stratified epithelial layers in 3D cultures and expressing markers like keratin 10/16 and involucrin. The HaCaSens Assay, which uses HaCaT cells, predicts skin sensitizers with 75% sensitivity and 83% specificity.

The objective of this HaCaT viability assay is to evaluate the ability of test compounds to induce changes in cell viability or cause cell death in HaCaT cells, with data reported as IC50 values.

HaCaT cells (from AddexBio) are seeded in 384-well plates at a density of 600 cells per well and incubated overnight. Test compounds are prepared as DMSO stock solutions and subjected to serial dilutions. Using liquid handlers, the compounds are transferred to the cell plates, with final concentrations of 3 μM for test compounds and 10 μM and 1 μM for reference compounds. Controls consist of 0.1% DMSO and medium alone. Following treatment, CellTiter-Glo reagent is added, and luminescence is measured to assess cell viability. Inhibition activity is calculated, and IC50 values are determined using Xlfit software.

TABLE 7
Data of HaCaT skin cell line assay

| | IC₅₀ (nM) |
|---|---|
| Reference Compound 1 | 8 |
| Reference Compound 2 | 1.5 |
| Example 29 | >3000 |
| Example 30 | >3000 |
| Example 12 | >3000 |

In Table 7, Example 29 and Example 30 and Example 12 showed minimal toxicity effects on the normal keratinocyte HaCaT, all exceeding 3000 nM. These values are more than 375 and 2000 times higher than those for reference compound 1 and reference compound 2, indicating much lower cytotoxicity in those WT RAS-dependent keratinocyte cell lines.

The significantly reduced cytotoxicity over reference compound 1 and reference compound 2 demonstrated that the compounds of this invention offer higher therapeutic window and a greater safety margin regarding skin toxicity for treating cancers with G12V RAS mutation in clinic.

Also provided herein are the following exemplary embodiments.
Embodiment 1. A compound of formula (I),

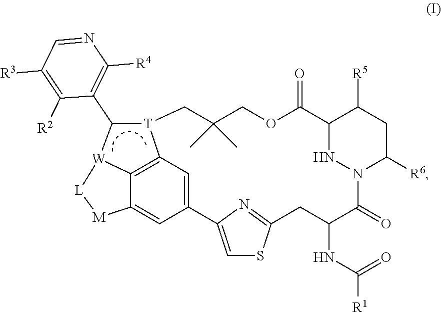

wherein
R¹ is 3-oxabicyclo[4.1.0]heptanyl,
   3-oxabicyclo[3.1.0]hexanyl,
   norcaranyl unsubstituted or substituted by one or two substituents independently selected from a group consisting of halogen and C₁₋₆alkyl, or
   C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is H, halogen, C₁₋₆alkyl, haloC₁₋₆alkyl or C₃₋₇cycloalkyl;
R³ is C₂₋₆alkynyl or 4-12 membered monocyclic or bicyclic heterocyclyl, wherein said C₂₋₆alkynyl and said heterocyclyl are unsubstituted or substituted by one or two substituents independently selected from a group consisting of halogen, hydroxy, C₁₋₆alkyl, haloC₁₋₆alkyl, C₁₋₆alkoxy, C₃₋₇cycloalkyl, morpholino and piperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.
Embodiment 2. A compound or a pharmaceutically acceptable salt thereof according to Embodiment 1, wherein R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl.
Embodiment 3. A compound of formula (Ia),

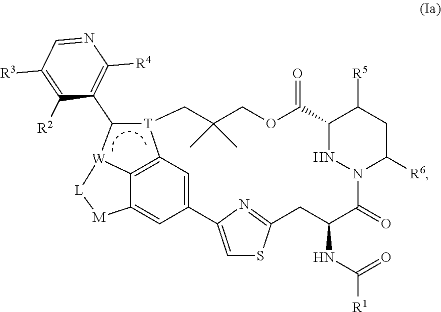

wherein
R¹ is 3-oxabicyclo[4.1.0]heptanyl,
   3-oxabicyclo[3.1.0]hexanyl,
   norcaranyl unsubstituted or substituted by one or two substituents independently selected from a group consisting of halogen and C₁₋₆alkyl, or
   C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is H, halogen, C₁₋₆alkyl, haloC₁₋₆alkyl or C₃₋₇cycloalkyl;
R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene or O;
L is C₁₋₆alkylene;
W is N and T is C; or W is C and T is N;
or a pharmaceutically acceptable salt thereof.
Embodiment 4. A compound or a pharmaceutically acceptable salt thereof according to any one of Embodiments 1-3, wherein R² is halogen or C₁₋₆alkyl.
Embodiment 5. A compound or a pharmaceutically acceptable salt thereof according to any one of Embodiments 1-4, wherein R² is fluoro or methyl.
Embodiment 6. A compound or a pharmaceutically acceptable salt thereof according to any one of Embodiments 1-5, wherein R¹ is C₃₋₇cycloalkyl substituted by one or two substituents independently selected from C₁₋₆alkyl and halopyridyl.
Embodiment 7. A compound or a pharmaceutically acceptable salt thereof according to any one of Embodiments 1-6, wherein R¹ is 2-(2-fluoro-3-pyridyl)cyclopropyl or 2,3-dimethylcyclopropyl.
Embodiment 8. A compound or a pharmaceutically acceptable salt thereof according to any one of Embodiments 1-7, wherein R³ is morpholinoC₂₋₆alkynyl or C₁₋₆alkylpiperazinyl.
Embodiment 9. A compound or a pharmaceutically acceptable salt thereof according to any one of Embodiments 1-8, wherein R³ is 3-morpholinoprop-1-ynyl or 4-methylpiperazin-1-yl.
Embodiment 10. A compound or a pharmaceutically acceptable salt thereof according to any one of Embodiments 1-9, wherein R⁴ is 1-methoxyethyl.
Embodiment 11. A compound or a pharmaceutically acceptable salt thereof according to any one of Embodiments 1-10, wherein R⁵ is H and R⁶ is H; or R⁵ and R⁶ together with the atoms to which they attached to form a cyclobutyl ring.
Embodiment 12. A compound or a pharmaceutically acceptable salt thereof according to any one of Embodiments 1-11, wherein M is C₁₋₆alkylene.
Embodiment 13. A compound or a pharmaceutically acceptable salt thereof according to any one of Embodiments 1-12, wherein M is methylene.
Embodiment 14. A compound or a pharmaceutically acceptable salt thereof according to any one of Embodiments 1-13, wherein L is ethylene.
Embodiment 15. A compound according to any one of Embodiments 1 to 3, wherein
R¹ is C₃₋₇cycloalkyl substituted by one or two substituents independently selected from C₁₋₆alkyl and halopyridyl;
   R² is halogen or C₁₋₆alkyl;
   R³ is morpholinoC₂₋₆alkynyl or C₁₋₆alkylpiperazinyl;
   R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
   M is C₁₋₆alkylene;
   L is C₁₋₆alkylene;
   W is N and T is C; or W is C and T is N;
   or a pharmaceutically acceptable salt thereof.
Embodiment 16. A compound according to Embodiment 15, wherein
R¹ is 2-(2-fluoro-3-pyridyl)cyclopropyl or 2,3-dimethylcyclopropyl;
   R² is fluoro or methyl;
   R³ is 3-morpholinoprop-1-ynyl or 4-methylpiperazin-1-yl;
   R⁴ is 1-methoxyethyl;
R⁵ is H and R⁶ is H; or R⁵ and R⁶ together with the atoms to which they attached to form a cyclobutyl ring;
   M is methylene;
   L is ethylene;
   W is N and T is C; or W is C and T is N;
   or a pharmaceutically acceptable salt thereof.
Embodiment 17. A compound of formula (Ia-1),

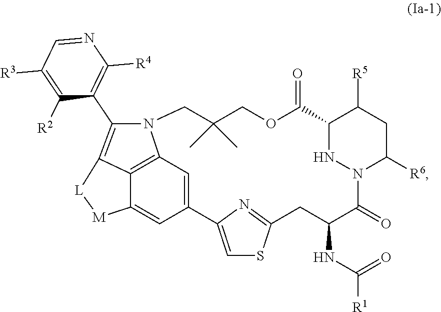

wherein
R¹ is 3-oxabicyclo[4.1.0]heptanyl, norcaranyl, or
   C₃₋₇cycloalkyl substituted by one or two substituents independently selected from C₁₋₆alkyl, C₁₋₆alkylpyridyl, halopyridyl, pyridyl and tetrahydropyranyl;
R² is H, halogen or C₁₋₆alkyl;
R³ is morpholinoC₂₋₆alkynyl, C₃₋₇cycloalkylpiperazinyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
   M is C₁₋₆alkylene or O;
   L is C₁₋₆alkylene;
   or a pharmaceutically acceptable salt thereof.
Embodiment 18. A compound or a pharmaceutically acceptable salt thereof according to Embodiment 17, wherein R¹ is C₃₋₇cycloalkyl substituted by halopyridyl or di-substituted by C₁₋₆alkyl.
Embodiment 19. A compound or a pharmaceutically acceptable salt thereof according to Embodiment 17 or 18, wherein R² is halogen or C₁₋₆alkyl.
Embodiment 20. A compound or a pharmaceutically acceptable salt thereof according to any one of Embodiments 17 to 19, wherein R³ is morpholinoC₂₋₆alkynyl or C₁₋₆alkylpiperazinyl.
Embodiment 21. A compound or a pharmaceutically acceptable salt thereof according to any one of Embodiments 17 to 19, wherein M is C₁₋₆alkylene.
Embodiment 22. A compound according to any one of Embodiments 17-21, wherein
R¹ is C₃₋₇cycloalkyl substituted by halopyridyl or di-substituted by C₁₋₆alkyl;
   R² is halogen or C₁₋₆alkyl;
   R³ is morpholinoC₂₋₆alkynyl or C₁₋₆alkylpiperazinyl;
   R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
   M is C₁₋₆alkylene;
   L is C₁₋₆alkylene;
   or a pharmaceutically acceptable salt thereof.
Embodiment 23. A compound according to any one of Embodiments 17-22, wherein
R¹ is 2-(2-fluoro-3-pyridyl)cyclopropyl or 2,3-dimethylcyclopropyl;
   R² is fluoro or methyl;
   R³ is 3-morpholinoprop-1-ynyl or 4-methylpiperazin-1-yl;
   R⁴ is 1-methoxyethyl;
R⁵ is H and R⁶ is H; or R⁵ and R⁶ together with the atoms to which they attached to form a cyclobutyl ring;
   M is methylene;
   L is ethylene;
   or a pharmaceutically acceptable salt thereof.
Embodiment 24. A compound of formula (Ia-2),

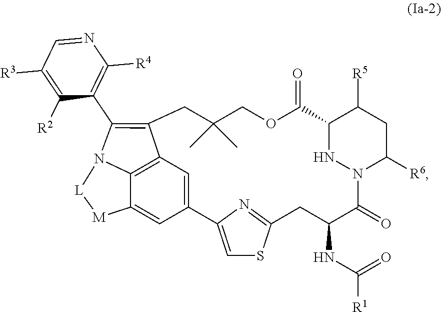

wherein
R¹ is 3-oxabicyclo[3.1.0]heptanyl,
   norcaranyl substituted by one or two substituents independently selected from a group consisting of halogen and C₁₋₆alkyl, or
C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is halogen, C₁₋₆alkyl, haloC₁₋₆alkyl or C₃₋₇cycloalkyl;
R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
   M is C₁₋₆alkylene;
   L is C₁₋₆alkylene;
   or a pharmaceutically acceptable salt thereof.
Embodiment 25. A compound according to Embodiment 24, wherein
R¹ is C₃₋₇cycloalkyl di-substituted by C₁₋₆alkyl;
   R² is C₁₋₆alkyl;
   R³ is C₁₋₆alkylpiperazinyl;
   R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
   M is C₁₋₆alkylene;
   L is C₁₋₆alkylene;
   or a pharmaceutically acceptable salt thereof.
Embodiment 26. A compound according to Embodiment 24 or 25, wherein
R¹ is 2,3-dimethylcyclopropyl;
   R² is methyl;
   R³ is 4-methylpiperazin-1-yl;
   R⁴ is 1-methoxyethyl;
R⁵ is H and R⁶ is H; or R⁵ and R⁶ together with the atoms to which they attached to form a cyclobutyl ring;
   M is methylene;
   L is ethylene;
   or a pharmaceutically acceptable salt thereof.
Embodiment 27. A compound selected from:
(1r,2R,3S)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1r,2R,3S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide;
(1S,6R,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]norcarane-7-carboxamide;
(1R,6S,7R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide;
(1r,2S,3R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide;
(1S,2S)-2-(2-fluoro-3-pyridyl)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]cyclopropanecarboxamide;
(1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(3-pyridyl)cyclopropanecarboxamide;
(1r,2S,3R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2,3-dimethyl-cyclopropanecarboxamide;
(1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-methyl-3-pyridyl)cyclopropanecarboxamide;
(1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-(2-fluoro-3-pyridyl)cyclopropanecarboxamide;
(1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-fluoro-3-pyridyl)cyclopropanecarboxamide;
(1r,2R,3S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide;
(1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1S,2S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2-(2-fluoro-3-pyridyl)cyclopropanecarboxamide;
(1R,6S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide;
(1R,6S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide;
(1R,6S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide;
(1R,6S,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]norcarane-7-carboxamide;
(1S,6R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-3-oxabicyclo[4.1.0]heptane-7-carboxamide;
(1R,6S,7r)-N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide;
(1R,6S,7R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-3-oxabicyclo[4.1.0]heptane-7-carboxamide;
(1R,6S,7R)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-3-oxabicyclo[4.1.0]heptane-7-carboxamide;
(1R,6S,7r)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide;
(1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2-(2-fluoro-3-pyridyl)cyclopropanecarboxamide;
(1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide;
(1S,2R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-tetrahydropyran-4-yl-cyclopropanecarboxamide;
(1r,2R,3S)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2S,3R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide;
(1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide;
cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide;
cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]cyclopropanecarboxamide;
cis-(1S,2S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-(2-fluoro-3-pyridyl)cyclopropanecarboxamide;
(1r,2R,3S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
cis-(1S,2R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide;
cis-(1r,2S,3R)—N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide;
cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide;
trans-(1S,2S)-2-(difluoromethyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide;
(1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-tetrahydropyran-4-yl-cyclopropanecarboxamide;
(1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[4.1.0]heptane-7-carboxamide;
(1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide;
(1S,5R)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide;
(1R,5S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide;
(1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[4.1.0]heptane-7-carboxamide;
(1r,2S,3R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-methyl-norcarane-7-carboxamide;
(1R,6R,7S)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide;
(1R,6R,7R)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide;
(1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2R,3S)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2R,3S)—N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2R,3S)—N-[(7S,13S)-(20P)-20-[4-chloro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide; and
(1r,2R,3S)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
   or a pharmaceutically acceptable salt thereof.
   Embodiment 28. A compound selected from:

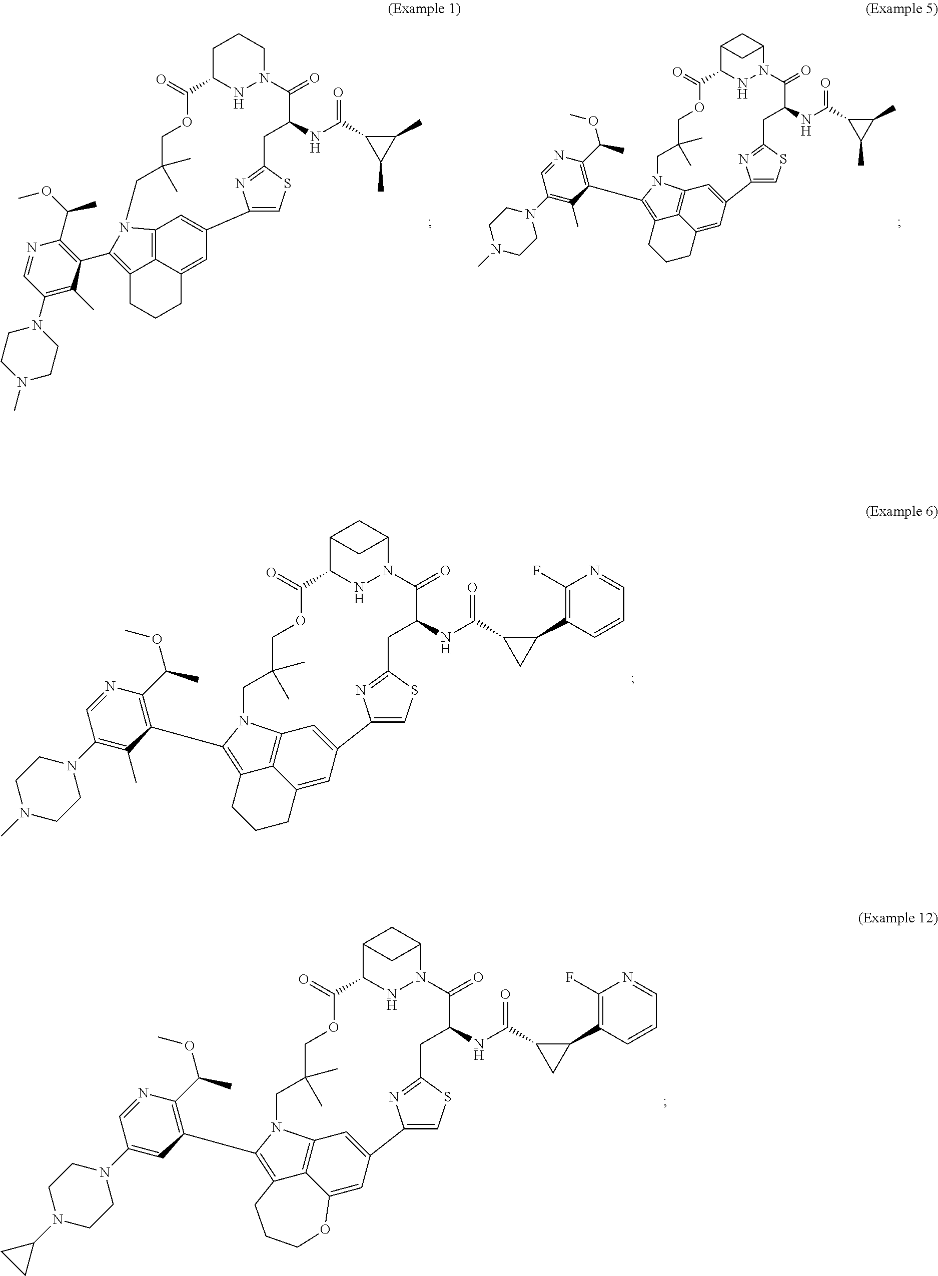

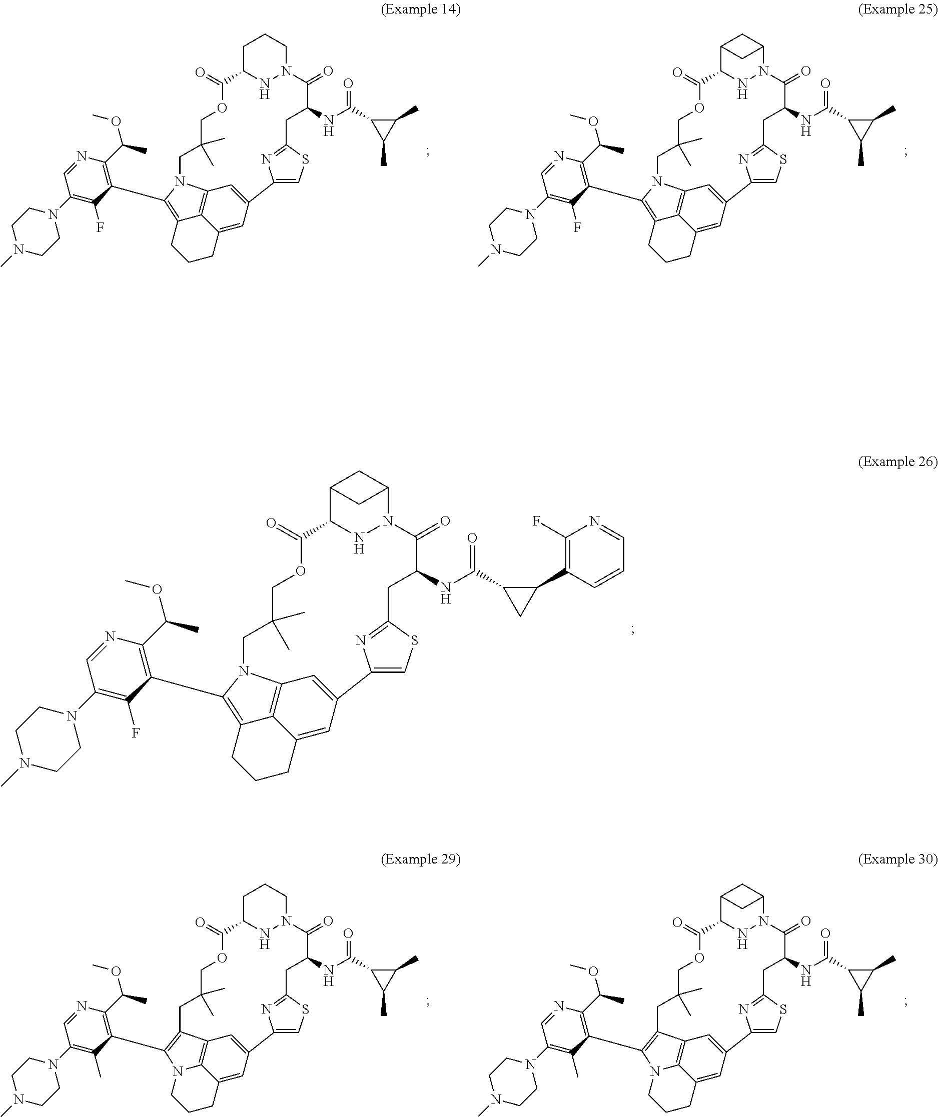

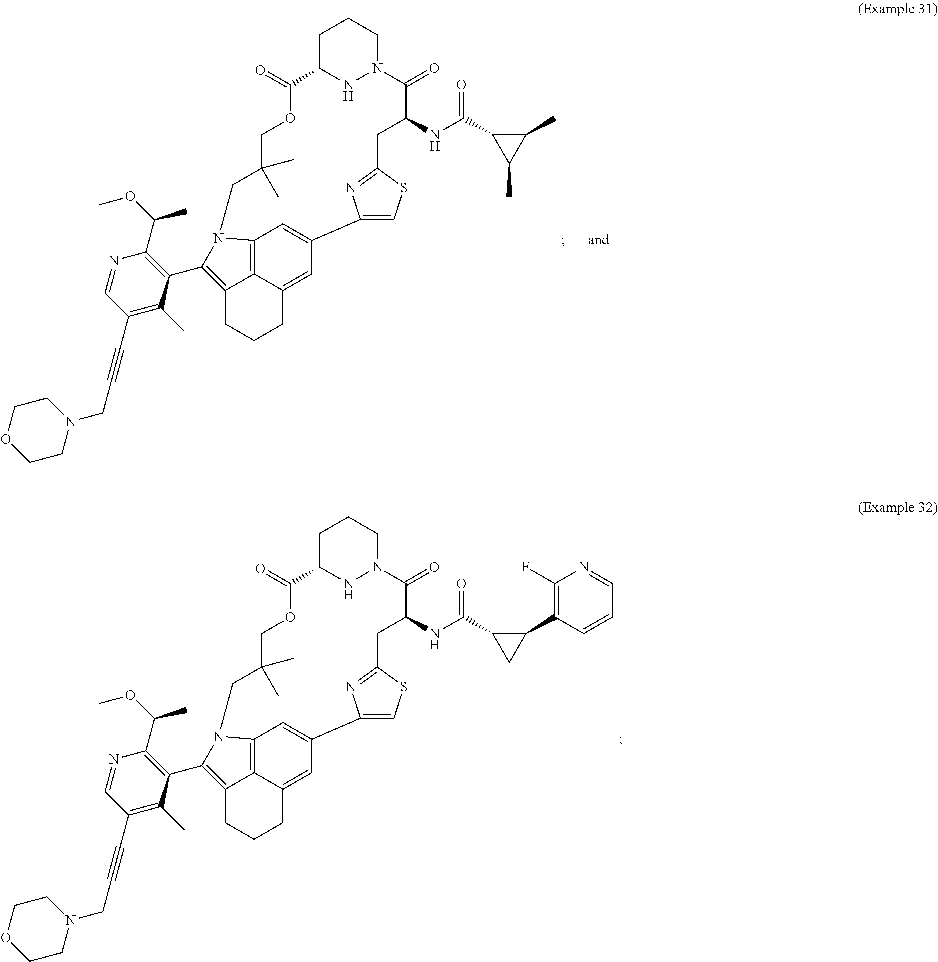

or a pharmaceutically acceptable salt thereof.
Embodiment 29. A process for the preparation of a compound according to any one of
Embodiments 1 to 28 comprising the following step:
   a) coupling reaction between compound of formula (II),

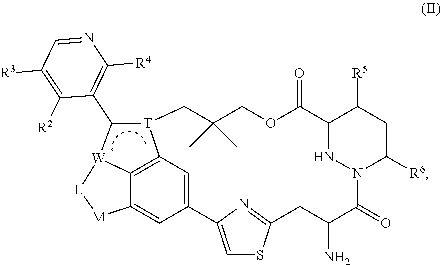

and acid (III),

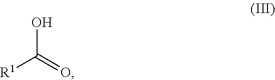

in the presence of a coupling reagent and a base to form the compound of formula (I); wherein R¹ to R⁶, M, L, T and W are defined as in any one of Embodiments 1 to 27; the coupling reagent is T3P, HATU, PyBOP or EDCI/HOBt; the base is TEA, DIEPA or DMAP.
Embodiment 30. A compound or pharmaceutically acceptable salt according to any one of Embodiments 1 to 28 for use as therapeutically active substance.
Embodiment 31. A pharmaceutical composition comprising a compound in accordance with any one of Embodiments 1 to 28 and a pharmaceutically acceptable excipient.
Embodiment 32. The use of a compound according to any one of Embodiments 1 to 28 for treating a KRAS G12V protein-related disease.
Embodiment 33. The use of a compound according to any one of Embodiments 1 to 28 for inhibiting RAS interaction with downstream effectors, wherein the downstream effectors are RAF and PI3K.
Embodiment 34. The use of a compound according to any one of Embodiments 1 to 28 for inhibiting the propagating oncogenic MAPK and PI3K signaling.
Embodiment 35. The use of a compound according to any one of Embodiments 1 to 28 for the treatment or prophylaxis of KRAS mutation driven cancers, wherein the cancer is selected from pancreatic cancer, colorectal cancer, lung cancer, esophageal cancer, gallbladder cancer, melanoma ovarian cancer and endometrial cancer.
Embodiment 36. The use of a compound according to any one of Embodiments 1 to 28 for the treatment or prophylaxis of KRAS mutation driven cancers, wherein the cancer is selected from pancreatic adenocarcinoma, colorectal cancer and non-small cell lung cancer.
Embodiment 37. The use of a compound according to any one of Embodiments 1 to 28 for the preparation of a medicament for the treatment or prophylaxis of KRAS mutation driven cancers, wherein the cancer is selected from pancreatic adenocarcinoma, colorectal cancer and non-small cell lung cancer.
Embodiment 38. A method for the treatment or prophylaxis of KRAS mutation driven cancers, wherein the cancer is selected from pancreatic adenocarcinoma, colorectal cancer and non-small cell lung cancer, which method comprises administering a therapeutically effective amount of a compound as defined in any one of Embodiments 1 to 28.
Embodiment 39. A compound or pharmaceutically acceptable salt according to any one of Embodiments 1 to 28, when manufactured according to a process of Embodiment 29.
Embodiment 40. A compound selected from:
(1r,2R,3S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2,3-dimethyl-cyclopropanecarboxamide;
(1r,2R,3S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,29-tetrazahexacyclo[17.5.2.1²,1^{9,13}.1^{10,12}.0^{22,26}]nonacosa-1(25),2,5(29),19,22(26),23-hexaen-7-yl]-2,3-dimethyl-cyclopropanecarboxamide; and
(1S,2S)—N-[(7S,13S)-21-ethyl-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,27,28-tetrazapentacyclo[17.5.2.1^{2,5}.1^{9,13}.0^{22,26}]octacosa-1(25),2,5(28),19,22(26),23-hexaen-7-yl]-2-methyl-cyclopropanecarboxamide;
   or a pharmaceutically acceptable salt thereof.

1. A compound of formula (I), 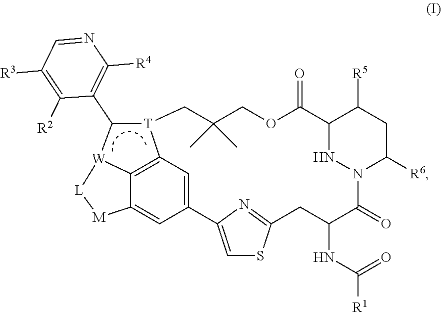
or a pharmaceutically acceptable salt thereof,
wherein:
R¹ is 3-oxabicyclo[4.1.0]heptanyl, 3-oxabicyclo[3.1.0]hexanyl,
norcaranyl unsubstituted or substituted by one or two substituents independently selected from a group consisting of halogen and C₁₋₆alkyl, or
C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is halogen, C₁₋₆alkyl, haloC₁₋₆alkyl or C₃₋₇cycloalkyl;
R³ is C₂₋₆alkynyl or 4-12 membered monocyclic or bicyclic heterocyclyl, wherein said C₂₋₆alkynyl and said heterocyclyl are unsubstituted or substituted by one or two substituents independently selected from a group consisting of halogen, hydroxy, C₁₋₆alkyl, haloC₁₋₆alkyl, C₁₋₆alkoxy, C₃₋₇cycloalkyl, morpholino and piperazinyl;
R⁴ is C₁₋₆alkoxyC₁₋₆alkyl;
R⁵ is H and R⁶ is H; or R⁵ and R⁶ together with the atoms to which they attached form a C₃₋₇cycloalkyl ring;
M is C₁₋₆alkylene;
L is C₁₋₆alkylene; and
W is N and T is C.

2. The compound according to claim 1, wherein the compound of formula (I) is a compound of formula (Ia), 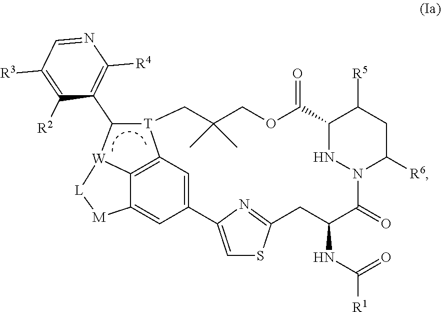
or a pharmaceutically acceptable salt thereof,
wherein:
R¹ is 3-oxabicyclo[4.1.0]heptanyl,
3-oxabicyclo[3.1.0]hexanyl,
norcaranyl unsubstituted or substituted by one or two substituents independently selected from a group consisting of halogen and C₁₋₆alkyl, or
C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is halogen, C₁₋₆alkyl, haloC₁₋₆alkyl or C₃₋₇cycloalkyl;
R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl;
R⁴ is C₁₋₆alkoxyC₁₋₆alkyl;
R⁵ is H and R⁶ is H; or R⁵ and R⁶ together with the atoms to which they attached form a C₃₋₇cycloalkyl ring;
M is C₁₋₆alkylene;
L is C₁₋₆alkylene; and
W is N and T is C.

3. The compound according claim 1, or a pharmaceutically acceptable salt thereof, wherein R² is halogen or C₁₋₆alkyl.

4. The compound according claim 1, or a pharmaceutically acceptable salt thereof, wherein R² is fluoro or methyl.

5. The compound according claim 1, or a pharmaceutically acceptable salt thereof, wherein R¹ is C₃₋₇cycloalkyl substituted by one or two substituents independently selected from C₁₋₆alkyl and halopyridyl.

6. The compound according claim 1, or a pharmaceutically acceptable salt thereof, wherein R¹ is 2-(2-fluoro-3-pyridyl)cyclopropyl or 2,3-dimethylcyclopropyl.

7. The compound according claim 1, or a pharmaceutically acceptable salt thereof, wherein R³ is morpholinoC₂₋₆alkynyl or C₁₋₆alkylpiperazinyl.

8. The compound according claim 1, or a pharmaceutically acceptable salt thereof, wherein R³ is 3-morpholinoprop-1-ynyl or 4-methylpiperazin-1-yl.

9. The compound according claim 1, or a pharmaceutically acceptable salt thereof, wherein R⁴ is 1-methoxyethyl.

10. The compound according claim 1, or a pharmaceutically acceptable salt thereof, wherein R⁵ is H and R⁶ is H; or R⁵ and R⁶ together with the atoms to which they attached form a cyclobutyl ring.

11. The compound according claim 1, or a pharmaceutically acceptable salt thereof, wherein M is methylene.

12. The compound according claim 1, or a pharmaceutically acceptable salt thereof, wherein L is ethylene.

13. The compound according claim 1, or a pharmaceutically acceptable salt thereof, wherein:
R¹ is C₃₋₇cycloalkyl substituted by one or two substituents independently selected from C₁₋₆alkyl and halopyridyl;
R² is halogen or C₁₋₆alkyl;
R³ is morpholinoC₂₋₆alkynyl or C₁₋₆alkylpiperazinyl;
R⁴ is C₁₋₆alkoxyC₁₋₆alkyl;
R⁵ is H and R⁶ is H; or R⁵ and R⁶ together with the atoms to which they attached form a C₃₋₇cycloalkyl ring;
M is C₁₋₆alkylene;
L is C₁₋₆alkylene; and
W is N and T is C.

14. The compound according claim 1, or a pharmaceutically acceptable salt thereof, wherein:
R¹ is 2-(2-fluoro-3-pyridyl)cyclopropyl or 2,3-dimethylcyclopropyl;
R² is fluoro or methyl;
R³ is 3-morpholinoprop-1-ynyl or 4-methylpiperazin-1-yl;
R⁴ is 1-methoxyethyl;
R⁵ is H and R⁶ is H; or R⁵ and R⁶ together with the atoms to which they attached form a cyclobutyl ring;
M is methylene;
L is ethylene; and
W is N and T is C.

15. The compound of Formula (I) wherein the compound is a compound of formula (Ia-2), 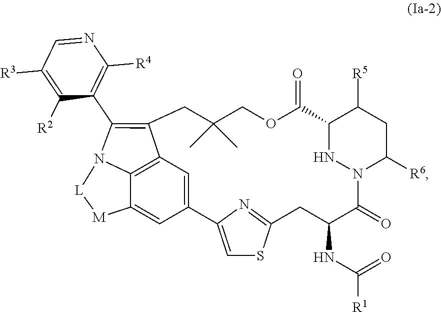
or a pharmaceutically acceptable salt thereof,
wherein:
R¹ is 3-oxabicyclo[3.1.0]heptanyl,
norcaranyl substituted by one or two substituents independently selected from a group consisting of halogen and C₁₋₆alkyl, or
C₃₋₇cycloalkyl substituted by one or two substituents independently selected from a group consisting of C₁₋₆alkyl, haloC₁₋₆alkyl, C₁₋₆alkylpyridyl, halopyridyl, pyridyl and tetrahydropyranyl;
R² is halogen, C₁₋₆alkyl, haloC₁₋₆alkyl or C₃₋₇cycloalkyl;
R³ is morpholinoC₂₋₆alkynyl, C₃₋₇cycloalkylpiperazinyl, C₁₋₆alkylpiperazinyl or 3,4,6,7,9,9a-hexahydro-1H-pyrazino[2,1-c][1,4]oxazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene; and
L is C₁₋₆alkylene.

16. The compound according claim 15, or a pharmaceutically acceptable salt thereof, wherein:
R¹ is C₃₋₇cycloalkyl substituted by two substituents each independently a C₁₋₆alkyl;
R² is C₁₋₆alkyl;
R³ is C₁₋₆alkylpiperazinyl;
R⁴ is C₁₋₆alkoxyC₁₋₆alkyl;
R⁵ is H and R⁶ is H; or R⁵ and R⁶ together with the atoms to which they attached to form a C₃₋₇cycloalkyl ring;
M is C₁₋₆alkylene; and
L is C₁₋₆alkylene.

17. The compound according claim 16, or a pharmaceutically acceptable salt thereof, wherein R² is methyl.

18. A compound selected from:
(1r,2R,3S)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1r,2R,3S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide;
(1S,6R,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]norcarane-7-carboxamide;
(1R,6S,7R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide;
(1r,2S,3R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide;
(1S,2S)-2-(2-fluoro-3-pyridyl)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]cyclopropanecarboxamide;
(1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(3-pyridyl)cyclopropanecarboxamide;
(1r,2S,3R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2,3-dimethyl-cyclopropanecarboxamide;
(1S,2S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-methyl-3-pyridyl)cyclopropanecarboxamide;
(1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-(2-fluoro-3-pyridyl)cyclopropanecarboxamide;
(1S,2S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-2-(2-fluoro-3-pyridyl)cyclopropanecarboxamide;
(1r,2R,3S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2,3-dimethyl-cyclopropanecarboxamide;
(1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1S,2S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2-(2-fluoro-3-pyridyl)cyclopropanecarboxamide;
(1R,6S)—N-[(7S,13S)-(20P)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-3-oxabicyclo[4.1.0]heptane-7-carboxamide;
(1R,6S)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide;
(1R,6S)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]norcarane-7-carboxamide;
(1R,6S,7r)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]norcarane-7-carboxamide;
(1S,6R)—N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-3-oxabicyclo[4.1.0]heptane-7-carboxamide;
(1R,6S,7r)-N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide;
(1R,6S,7R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,33-tetrazaheptacyclo[24.3.1.1^{2,5}.1^{9,13}.1^{10,12}.0^{19,28}.0^{21,27}]tritriaconta-1(29),2,5(33),20,26(30),27-hexaen-7-yl]-3-oxabicyclo[4.1.0]heptane-7-carboxamide;
(1R,6S,7R)—N-[(7S,13S)-(20P)-20-[5-(4-cyclopropylpiperazin-1-yl)-2-[(1S)-1-methoxyethyl]-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-3-oxabicyclo[4.1.0]heptane-7-carboxamide;
(1R,6S,7r)-N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]norcarane-7-carboxamide;
(1r,2S,3R)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)—N-[(7S,13S)-(20M)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2-(2-fluoro-3-pyridyl)cyclopropanecarboxamide;
(1S,2S)-2-(2-fluoro-3-pyridyl)-N-[(7S,13S)-(20P)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide;
(1S,2R)—N-[(7S,13S)-(20P)-20-[2-[(1S)-1-methoxyethyl]-5-(4-methylpiperazin-1-yl)-3-pyridyl]-17,17-dimethyl-8,14-dioxo-15,25-dioxa-4-thia-9,19,31,32-tetrazahexacyclo[24.3.1.1^{2,5}.1^{9,13}.0^{19,28}.0^{21,27}]dotriaconta-1(29),2,5(32),20,26(30),27-hexaen-7-yl]-2-tetrahydropyran-4-yl-cyclopropanecarboxamide;
(1r,2R,3S)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2S,3R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,31-tetrazahexacyclo[23.3.1.1^{2,5}.1^{9,13}.0^{19,27}.0^{21,26}]hentriaconta-1(28),2,5(31),20,25(29),26-hexaen-7-yl]cyclopropanecarboxamide;
(1r,2R,3S)—N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]-2,3-dimethyl-cyclopropanecarboxamide;
(1S,2S)-2-(2-fluoro-3-pyridyl)-N-[(7S,13S)-(20P)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,19,30,32-tetrazaheptacyclo[23.3.1.1^{2,5}.1^{9,13}.1^{10,12}.0^{19,27}.0^{21,26}]dotriaconta-1(28),2,5(32),20,25(29),26-hexaen-7-yl]cyclopropanecarboxamide;
cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide;
cis-(1S,2S)-2-(2-fluoro-3-pyridyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]cyclopropanecarboxamide;
cis-(1S,2S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-(2-fluoro-3-pyridyl)cyclopropanecarboxamide;
(1r,2R,3S)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
cis-(1S,2R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide;
cis-(1r,2S,3R)—N-[(7S,13S)-(20M)-20-[2-[(1S)-1-methoxyethyl]-4-methyl-5-(3-morpholinoprop-1-ynyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide;
cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2-tetrahydropyran-4-yl-cyclopropanecarboxamide;
trans-(1S,2S)-2-(difluoromethyl)-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]cyclopropanecarboxamide;
(1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
cis-(1S,2R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2-tetrahydropyran-4-yl-cyclopropanecarboxamide;
(1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[4.1.0]heptane-7-carboxamide;
(1r,2S,3R)—N-[(7S,13S)-(20M)-20-[5-[(9aS)-3,4,6,7,9,9a-hexahydro-1H-pyrazino[2,1-c][1,4]oxazin-8-yl]-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1S,5R)—N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide;
(1S,5R)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-3-oxabicyclo[3.1.0]hexane-6-carboxamide;
(1R,5S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[3.1.0]hexane-6-carboxamide;
(1S,6R,7S)—N-[(7S,13S)-(20M)-20-[5-(4-cyclopropylpiperazin-1-yl)-2-[(1S)-1-methoxyethyl]-4-methyl-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-oxabicyclo[4.1.0]heptane-7-carboxamide;
(1r,2S,3R)—N-[(7S,13S)-(20P)-20-[4-fluoro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-3-methyl-norcarane-7-carboxamide;
(1R,6R,7S)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide;
(1R,6R,7R)-2,2-difluoro-N-[(7S,13S)-(20M)-20-[2-[(1S)-1-methoxyethyl]-4-methyl-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]norcarane-7-carboxamide;
(1r,2S,3R)—N-[(7S,13S)-(20M)-20-[4-ethyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2R,3S)—N-[(7S,13S)-(20M)-20-[4-cyclopropyl-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2R,3S)—N-[(7S,13S)-(20M)-20-[4-(difluoromethyl)-2-[(1S)-1-methoxyethyl]-5-(4-methylpiperazin-1-yl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide;
(1r,2R,3S)—N-[(7S,13S)-(20P)-20-[4-chloro-2-[(1S)-1-methoxyethyl]-5-(4-methylpiperazin-1-yl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
(1r,2S,3R)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,31-tetrazahexacyclo[23.3.1.1^{2,5}.1^{9,13}.0^{19,27}.0^{21,26}]hentriaconta-1(28),2,5(31),19,25(29),26-hexaen-7-yl]-2,3-dimethyl-cyclopropanecarboxamide; and
(1r,2R,3S)—N-[(7S,13S)-(20M)-20-[2-[(1S)-1-methoxyethyl]-5-(4-methylpiperazin-1-yl)-4-(trifluoromethyl)-3-pyridyl]-17,17-dimethyl-8,14-dioxo-15-oxa-4-thia-9,21,30,32-tetrazaheptacyclo[23.3.1.1^{2,5}.1^{9,13}.1^{10,12}.0^{19,27}.0^{21,26}]dotriaconta-1(28),2,5(32),19,25(29),26-hexaen-7-yl]-2,3-dimethyl-cyclopropanecarboxamide;
or a pharmaceutically acceptable salt thereof.

19. A compound selected from: 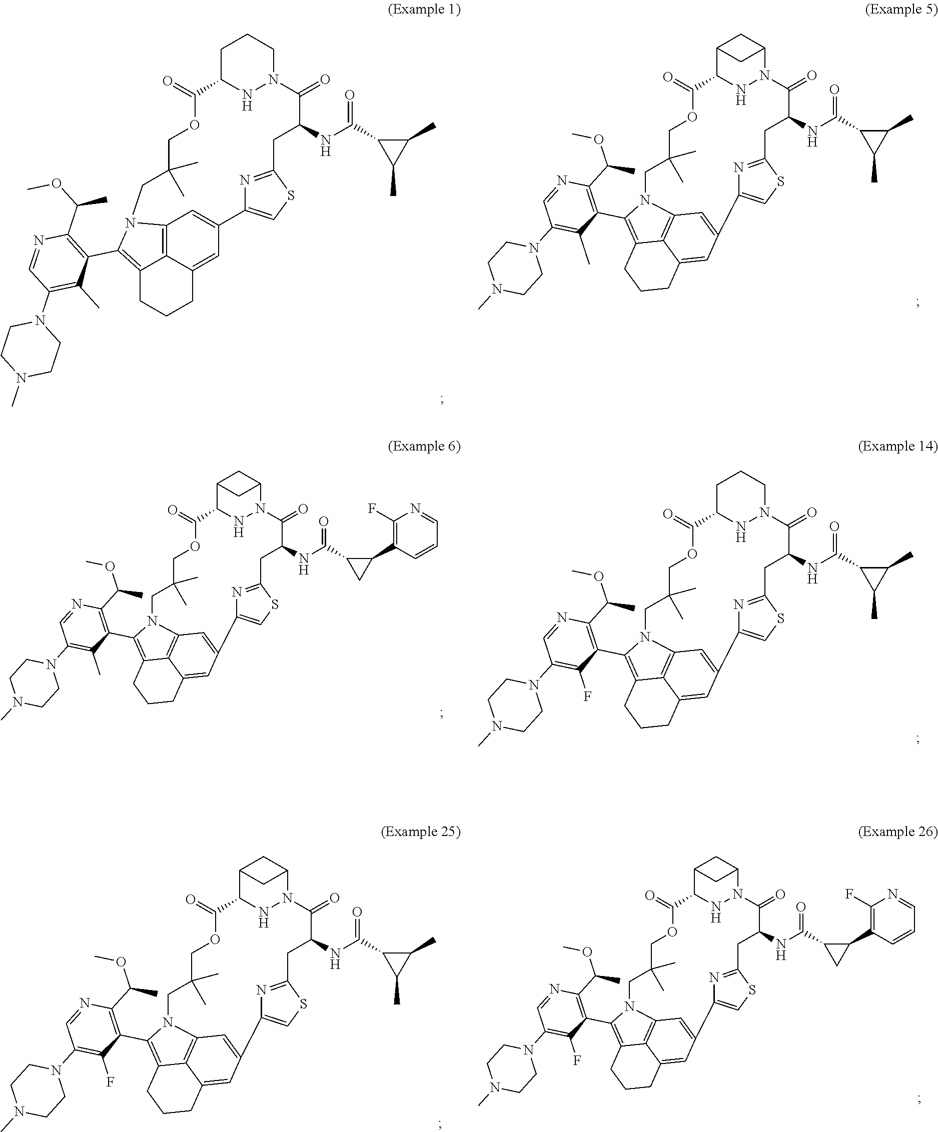 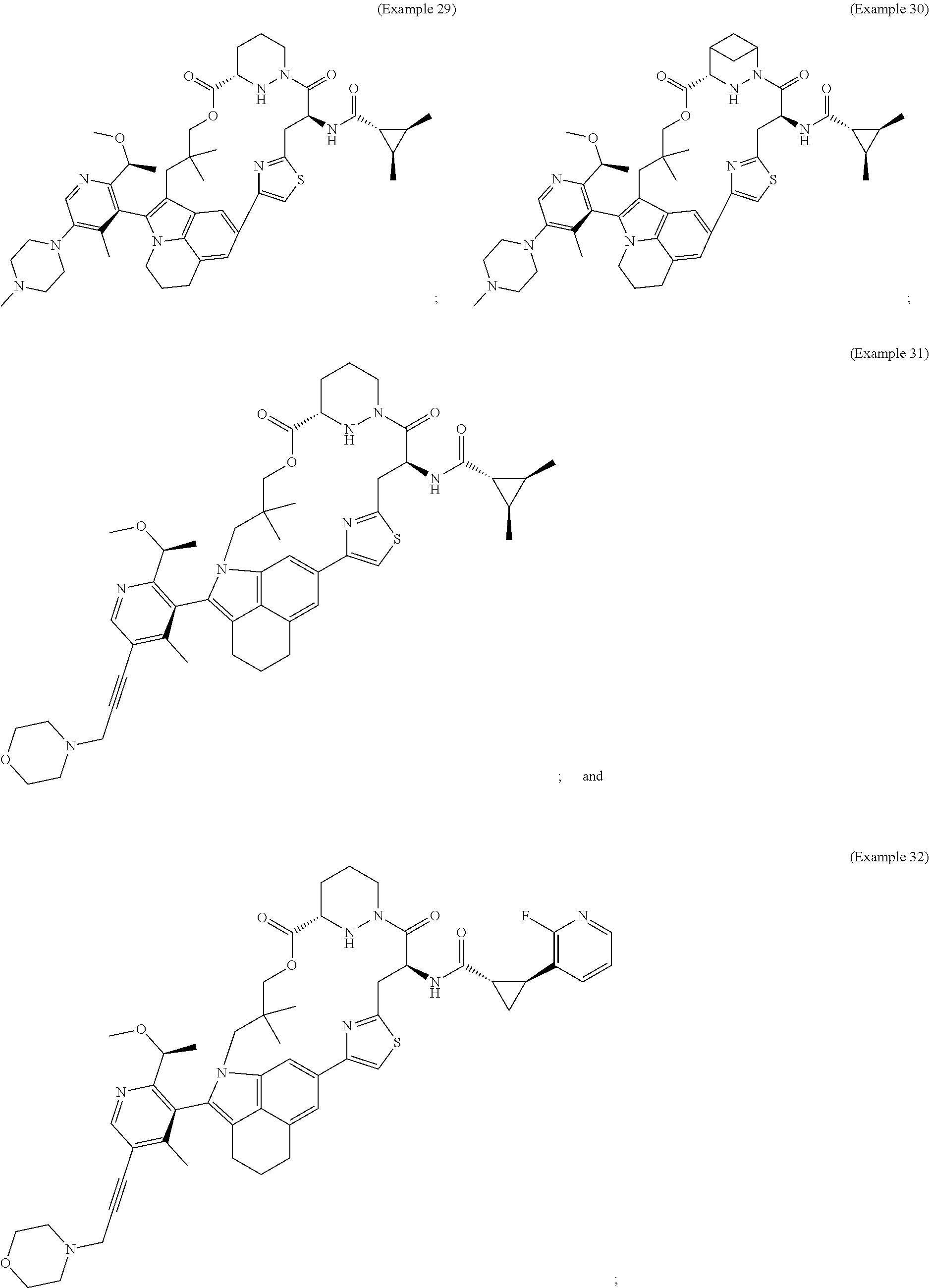
or a pharmaceutically acceptable salt thereof.

20. The compound according claim 19, wherein the compound is: 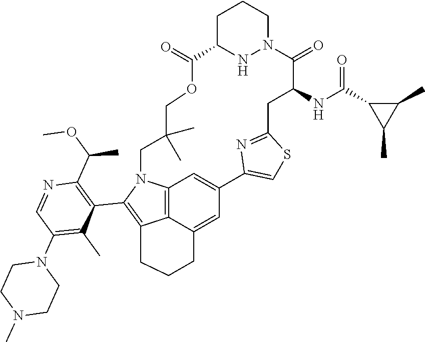
or a pharmaceutically acceptable salt thereof.

21. The compound according claim 19, wherein the compound is: 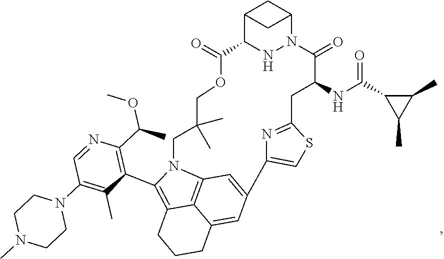
or a pharmaceutically acceptable salt thereof.

22. The compound according claim 19, wherein the compound is: 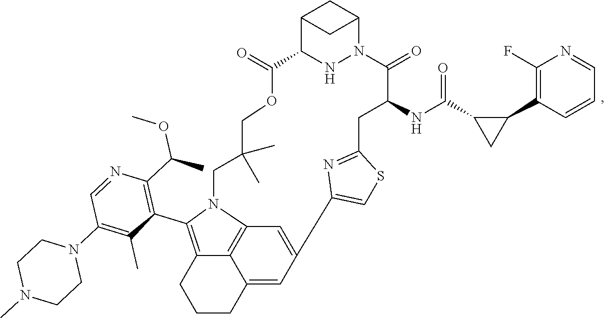
or a pharmaceutically acceptable salt thereof.

23. The compound according claim 19, wherein the compound is: 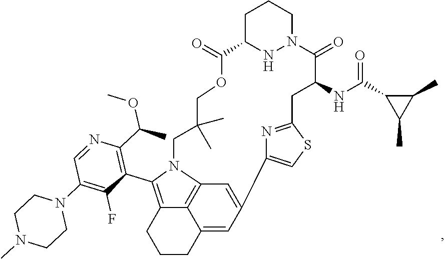
or a pharmaceutically acceptable salt thereof.

24. The compound according claim 19, wherein the compound is: 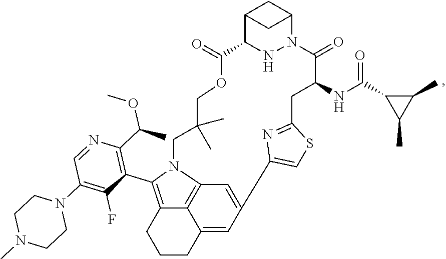
or a pharmaceutically acceptable salt thereof.

25. The compound according claim 19, wherein the compound is: 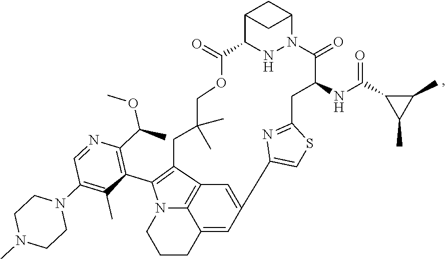
or a pharmaceutically acceptable salt thereof.

26. The compound according claim 19, wherein the compound is: 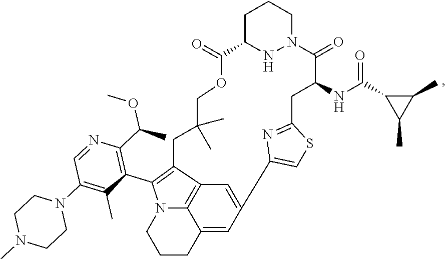
or a pharmaceutically acceptable salt thereof.

27. The compound according claim 19, wherein the compound is: 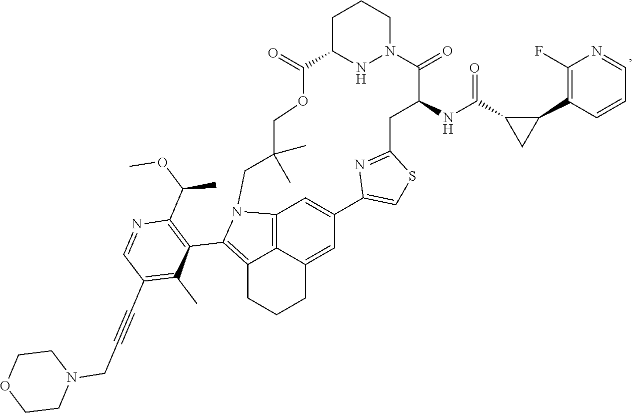
or a pharmaceutically acceptable salt thereof.

28. A pharmaceutical composition comprising a compound of claim 1, or a pharmaceutically acceptable salt thereof, and a pharmaceutically acceptable excipient.

29. A pharmaceutical composition comprising a compound of claim 19, or a pharmaceutically acceptable salt thereof, and a pharmaceutically acceptable excipient.

30. A method for the treatment of KRAS mutation driven cancers in a human subject in need thereof, wherein the cancer is selected from pancreatic adenocarcinoma, colorectal cancer and non-small cell lung cancer, which method comprises administering a therapeutically effective amount of a compound of claim 1, or a pharmaceutically acceptable salt thereof.
